(19) United States
(12) Patent Application Publication
(10) Pub. No.: US 2025/0084079 A1
(43) Pub. Date: Mar. 13, 2025

(54) COMPOSITIONS AND METHODS FOR INHIBITION OF RAS

(71) Applicants: TheRas, Inc., South San Francisco, CA (US); Leidos Biomedical Research, Inc., Frederick, MD (US); Lawrence Livermore National Security, LLC, Livermore, CA (US)

(72) Inventors: Bin Wang, Dallas, TX (US); Rui Xu, Dallas, TX (US); Eli Wallace, Loveland, CO (US); Zuhui Zhang, Millbrae, CA (US); Felice Lightstone, Fremont, CA (US); Yue Yang, Foster City, CA (US); David Michael Turner, Frederick, MD (US); Anna Elzbieta Maciag, Frederick, MD (US); Dhirendra Kumar Simanshu, Frederick, MD (US); Albert Hay Wah Chan, Frederick, MD (US)

(21) Appl. No.: 18/726,861

(22) PCT Filed: Jan. 5, 2023
(86) PCT No.: PCT/US23/10176

§ 371 Date: Jul. 5, 2024

Related U.S. Application Data
(60) Provisional application No. 63/297,124, filed on Jan. 6, 2022.

(51) Int. Cl. C07D 471/04; A61K 31/517; A61K 31/519; C07D 487/04

(52) U.S. Cl. CPC C07D 471/04; A61K 31/517; A61K 31/519; C07D 487/04

(57) ABSTRACT

Provided herein are compounds, or salts, esters, tautomers, prodrugs, zwitterionic forms, or stereoisomers thereof, as well as pharmaceutical compositions comprising the same. Also provided herein are methods of using the same in modulating (e.g., inhibiting) KRAS (e.g., KRAS having a G12D or G12V mutation or wild-type KRAS) and treating diseases or disorders such as cancers in subjects in need thereof.

### RELATED APPLICATIONS

This application claims priority to and benefit of U.S. Application No. 63/297,124, filed Jan. 6, 2022, the entire contents of which are hereby incorporated by reference.

### STATEMENT AS TO RIGHTS TO INVENTIONS MADE UNDER FEDERALLY SPONSORED RESEARCH AND DEVELOPMENT

This invention was made with government support under (1) Contract No.: 75N91019D00024 awarded by the National Institutes of Health and (2) Contract No. DE-AC52-07NA27344 awarded by the United States Department of Energy. The government has certain rights in the invention.

### BACKGROUND

RAS mutations occur in approximately 20-30% of human cancers, including the majority of pancreatic ductal adenocarcinoma (PDAC), half of colorectal cancers, and a third of all lung cancers. With the highest RAS mutation frequencies seen with the top three causes of cancer deaths in the United States (lung, colorectal, and pancreatic cancer), the development of anti-RAS therapies is a major priority and a major challenge for cancer research. RAS proteins did not appear to present suitable pockets to which drugs could bind, except for the GDP/GTP binding site. Unfortunately, RAS proteins bind to these nucleotides with very high (picomolar) affinities, making the development of effective nucleotide analogs virtually impossible. Attempts to block pathways downstream of RAS with a hope to provide clinical benefit for patients suffering from RAS-driven cancers have been generally disappointing. Very recently, allele-specific covalent KRAS G12C inhibitors entered clinical trials, and early clinical data show some effectiveness, at least in lung cancer.

The three RAS genes (HRAS, NRAS, and KRAS) encode four 188-189 amino acid proteins that share 82%-90% amino acid sequence identity and near-identical structural and biochemical properties. However, they are differentially expressed, and mutated with different frequencies in cancer. KRAS is the most frequently mutated oncogene in cancer and KRAS mutation is commonly associated with poor prognosis and resistance to therapy. Significant cancer type preferences exist among the RAS genes. KRAS mutations predominate in lung, colorectal, and pancreatic cancer, whereas NRAS mutations predominate in cutaneous melanomas and acute myelogenous leukemia, and HRAS mutations are found in bladder and head and neck squamous cell carcinomas.

KRAS is mutationally activated in 94% of pancreatic cancers. Pancreatic cancer (e.g., pancreatic ductal adenocarcinoma (PDAC)) became the third leading cause of cancer deaths in the United States in 2016. With a continued increase in incidence, pancreatic cancer is projected to become the second leading cause of cancer death by 2020. With no biomarkers for early detection, late onset of symptoms when the cancer has already reached the metastatic state, and the 5-year survival rate 8%, pancreatic cancer is the deadliest cancer in the US.

KRAS mutations are the initiating genetic step in pancreatic cancer; however, continued mutant KRAS function is required to maintain the growth of PDAC. RNA interference-mediated KRAS inactivation in KRAS G12D-driven PDAC showed rapid regression of tumor growth. These data support the significance of mutant KRAS as a therapeutic target in PDAC. Since 40% of PDAC are driven by KRAS G12D mutant, inhibitors targeting this mutation are highly desirable. KRAS G12D mutation also occurs in high frequency in lung and colorectal cancers, making KRAS G12D desirable therapeutic target for direct G12D allele-specific inhibitors.

Mutated KRAS is also observed at high frequency in other common tumors, including colorectal cancer (˜44%) and non-small cell lung cancer (NSCLC; ˜20-30%). The KRAS missense mutation G12D is the most predominant variant in human malignancies (35%), followed by G12V (29%).

The development of small molecule KRAS inhibitors has proven to be a challenge. Recent clinical development of covalent KRAS G12C inhibitors indicates potential of targeting the KRAS oncogenic protein directly. Results from clinical trials with the two covalent inhibitors, AMG510 (sotorasib) and MRTX849 (adagrasib), have been promising. These inhibitors demonstrated clinical activity primarily in NSCLC, where the KRAS G12C mutation frequency is highest. Unfortunately, they appeared less effective in KRAS G12C colorectal cancers. Both compounds only target the inactive (GDP-bound) form of KRAS G12C, and a lack of activity against active (GTP-bound) KRAS G12C may contribute to development of drug resistance. Moreover, these covalent inhibitors are limited to the specific G12C mutant that accounts for approximately 13% of all KRAS-driven cancers, leaving a large population of non-G12C KRAS cancers still undruggable. Therefore, KRAS therapeutics that target additional KRAS alterations or combinations thereof are an unmet clinical need. KRAS inhibitors that target other mutations and/or wild-type KRAS hold promise for impact across the majority of KRAS mutant alleles, including the most prevalent G12D and G12V, or KRAS wild-type-amplified cancers.

### SUMMARY

In an aspect, the present disclosure provides compositions comprising compounds according to Formula I or Formula II.

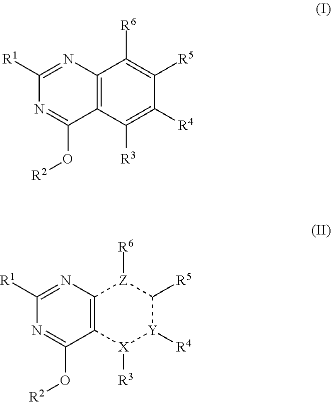

or a salt (e.g., pharmaceutically acceptable salt), ester, tautomer, prodrug, zwitterionic form, or stereoisomer thereof, wherein R¹, R², R³, R⁴, R⁵, R⁶, X, Y, and Z are as provided herein. In some embodiments, a compound provided herein, or a salt (e.g., pharmaceutically acceptable salt), ester, tautomer, zwitterionic form, or stereoisomer thereof, can modulate the activity of a KRAS protein, such as a KRAS protein having a G12D or G12V mutation or wild-type KRAS. In some embodiments, a compound provided herein, or a salt (e.g., pharmaceutically acceptable salt), ester, tautomer, zwitterionic form, or stereoisomer thereof, is capable of interacting with an aspartic acid residue at the 12 position of the KRAS protein (e.g., a G12D mutation). In some embodiments, a compound provided herein, or a salt (e.g., pharmaceutically acceptable salt), ester, tautomer, zwitterionic form, or stereoisomer thereof, is capable of interacting with a valine residue at the 12 position of the KRAS protein (e.g., a G12V mutation). In some embodiments, a compound provided herein, or a salt, ester, tautomer, zwitterionic form, or stereoisomer(s) thereof, has inhibitory activity against wild-type KRAS. In some embodiments, a compound provided herein, or a salt (e.g., pharmaceutically acceptable salt), ester, tautomer, zwitterionic form, or stereoisomer thereof, is capable of binding a KRAS protein in an active (GTP-bound) conformation. In some embodiments, a compound provided herein, or a salt (e.g., pharmaceutically acceptable salt), ester, tautomer, zwitterionic form, or stereoisomer thereof, is capable of binding a KRAS protein in an inactive (GDP-bound) conformation.

In another aspect, the present disclosure provides a pharmaceutical composition comprising a compound provided herein (e.g., a compound according to any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt (e.g., pharmaceutically acceptable salt), ester, tautomer, prodrug, zwitterionic form, or stereoisomer thereof, together with a pharmaceutically acceptable carrier.

In a further aspect, the present disclosure provides a method of inhibition of KRAS activity in a human or animal subject for the treatment of a disease such as cancer, including pancreatic cancer (e.g., pancreatic ductal adenocarcinoma (PDAC)), colorectal cancer, endometrial endometrioid adenocarcinoma, rectal adenocarcinoma, gastric cancer, and lung cancer, using, e.g., a compound provided herein (e.g., a compound according to any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt (e.g., pharmaceutically acceptable salt), ester, tautomer, prodrug, zwitterionic form, or stereoisomer thereof, or a pharmaceutical composition comprising the same.

In another aspect, the present disclosure provides a use of a compound provided herein (e.g., a compound according to any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt (e.g., pharmaceutically acceptable salt), ester, tautomer, zwitterionic form, or stereoisomer thereof, in the manufacture of a medicament for the treatment of a disease, disorder, or condition (e.g., a cancer) ameliorated, treated, inhibited, or reduced by inhibition of KRAS, including KRAS having a G12D or G12V mutation or wild-type KRAS. In some embodiments, the disease, disorder, or condition is pancreatic cancer (e.g., pancreatic ductal adenocarcinoma (PDAC)), colorectal cancer, or lung cancer.

In a further aspect, the present disclosure provides a compound as provided herein (e.g., a compound according to any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt (e.g., pharmaceutically acceptable salt), ester, tautomer, zwitterionic form, or stereoisomer thereof, for use as a medicament. In some embodiments, the medicament is used in the treatment of a disease, disorder, or condition (e.g., a cancer). In some embodiments, the disease, disorder, or condition is pancreatic cancer (e.g., pancreatic ductal adenocarcinoma (PDAC)), colorectal cancer, or lung cancer.

### DETAILED DESCRIPTION

The present disclosure provides compounds and methods of using such compounds and compositions comprising the same in inhibiting RAS (e.g., KRAS)-mediated cell signaling, such as in the treatment or prophylaxis of a disease, disorder, or condition in which KRAS plays an active role. The present disclosure provides compounds (e.g., compounds of any one of Formulas I, IA, IA1, IA2, IA3, IA4, IA5, IB, IB1, IB2, IB3, IB4, IB5, IB6, IB7, IB8, IB9, IC, II, IIA, IIB, or IIC) and forms thereof (e.g., a salt (e.g., pharmaceutically acceptable salt), ester, tautomer, zwitterionic form, or stereoisomer thereof) that may possess useful KRAS inhibitory activity and may be used in inhibiting KRAS activity in a cell and/or in the treatment or prophylaxis of a disease, disorder, or condition in which KRAS plays an active role. In particular, certain compounds and forms thereof provided herein may possess useful inhibitory activity of KRAS having a G12D or G12V mutation or wild-type KRAS, which KRAS protein is in an active (GTP-bound) or inactive (GDP-bound) conformation. The present disclosure also provides pharmaceutical compositions comprising one or more compounds or forms thereof provided herein together with a pharmaceutically acceptable carrier, as well as methods of making and using the compounds and compositions. The present disclosure also provides methods for inhibiting KRAS, including KRAS having a G12D or G12V mutation or wild-type KRAS, which KRAS is in an active or inactive conformation. In an aspect, the present disclosure provides a method for treating a disorder mediated by KRAS, including a KRAS having a G12D or G12V mutation or wild-type KRAS, in a subject in need of such treatment, which method comprises administering to the subject a therapeutically effective amount of a compound or composition provided herein. Also provided herein is the use of certain compounds provided herein in the manufacture of a medicament for the treatment of a disease, disorder, or condition ameliorated, treated, inhibited, or reduced by inhibition of KRAS, including KRAS having a G12D or G12V mutation or wild-type KRAS. In some embodiments, the disease, disorder, or condition is a cancer (e.g., as described herein).

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

“Alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

“Alkynyl” refers to either a straight chain or branched hydrocarbon having at least 2 carbon atoms and at least one triple bond and having the number of carbon atoms indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

“Alkoxy,” as used herein, alone or in combination, refers to an alkyl ether radical, wherein the term alkyl is as described herein. Examples of suitable alkyl ether radicals include methoxy, ethoxy, n-propoxy, isopropoxy, n-butoxy, iso-butoxy, sec-butoxy, tert-butoxy, and the like.

“Alkyl,” as used herein, alone or in combination, refers to a straight-chain or branched-chain alkyl radical containing from 1 to 20 carbon atoms (e.g., C₁₋₂₀ alkyl). In certain embodiments, said alkyl will comprise from 1 to 10 carbon atoms (e.g., C₁₋₁₀ alkyl). In further embodiments, said alkyl will comprise from 1 to 8 carbon atoms (e.g., C₁₋₈ alkyl). In further embodiments, said alkyl will comprise from 1 to 6 carbon atoms (e.g., C₁₋₆ alkyl). In further embodiments, said alkyl will comprise from 1 to 3 carbon atoms (e.g., C₁₋₃ alkyl). Alkyl groups are unsubstituted or substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

“Alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino, and the like.

“Alkylthio,” as used herein, alone or in combination, refers to an alkyl thioether (R—S—) radical wherein the term alkyl is as described herein and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

“Amido” and “carbamoyl,” as used herein, alone or in combination, refer to an amino group as described herein attached to the parent molecular moiety through a carbonyl group, or vice versa. The “amido” group as used herein incudes a “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes —C(O)NH₂, C₁₋₄alkylamido, and di(C₁₋₄alkyl)amido. The term “C₁₋₄alkylamido”, as used herein, refers to —C(O)NH(C₁₋₄alkyl), wherein C₁₋₄alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

“Amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be unsubstituted or substituted. Additionally, R and R′ may combine to form a heterocycloalkyl, which is unsubstituted or substituted. An “amino” group may be a primary amine (e.g., —NH₂), secondary or di-substituted amine (e.g., —NHR where R is not hydrogen), or tertiary or tri-substituted amine (e.g., —NRR′ where neither R nor R′ is hydrogen).

“Aryl,” as used herein, alone or in combination, means a carbocyclic aromatic system containing one, two, or three rings wherein such polycyclic ring systems are fused together. The term “aryl” embraces aromatic groups such as phenyl, naphthyl, anthracenyl, and phenanthryl. An aryl moiety may include, for example, between 5 to 20 carbon atoms, such as between 5 to 12 carbon atoms, such as 5 or 6 carbon atoms.

“Arylalkenyl” or “aralkenyl,” as used herein, alone or in combination, refers to an aryl group attached to the parent molecular moiety through an alkenyl group.

“Arylalkoxy” or “aralkoxy,” as used herein, alone or in combination, refers to an aryl group attached to the parent molecular moiety through an alkoxy group.

“Arylalkyl” or “aralkyl,” as used herein, alone or in combination, refers to an aryl group attached to the parent molecular moiety through an alkyl group.

“Aryloxy,” as used herein, alone or in combination, refers to an aryl group attached to the parent molecular moiety through an oxy.

“Carbamate,” as used herein, alone or in combination, refers to an ester of carbamic acid (—NHCOO—) which may be attached to the parent molecular moiety from either the nitrogen or acid end, and which is unsubstituted or substituted as defined herein.

“O-carbamyl” as used herein, alone or in combination, refers to a —OC(O)NRR′ group, with R and R′ as defined herein.

“N-carbamyl” as used herein, alone or in combination, refers to a ROC(O)NR′— group, with R and R′ as defined herein.

“Carbonyl,” as used herein, when alone includes formyl [—C(O)H] and in combination is a —C(O)— group.

“Carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

“Cyano,” as used herein, alone or in combination, refers to —CN.

“Cycloalkyl,” or, alternatively, “carbocycle,” as used herein, alone or in combination, refers to a saturated or partially saturated monocyclic, bicyclic, or tricyclic alkyl group wherein each cyclic moiety contains from 3 to 12 carbon atom ring members and which may optionally be a benzo fused ring system which is unsubstituted or substituted as defined herein. A carbocycle may comprise a bridged ring system and/or a spiro ring system (e.g., a system including two rings sharing a single carbon atom). The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene and octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

“Ester,” as used herein, alone or in combination, refers to a carboxy group bridging two moieties linked at carbon atoms.

“Ether,” as used herein, alone or in combination, refers to an oxy group bridging two moieties linked at carbon atoms.

“Halo,” or “halogen,” as used herein, alone or in combination, refers to fluorine, chlorine, bromine, or iodine.

“Haloalkoxy,” as used herein, alone or in combination, refers to a haloalkyl group attached to the parent molecular moiety through an oxygen atom.

“Haloalkyl,” as used herein, alone or in combination, refers to an alkyl radical having the meaning as described herein wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

“Heteroalkyl,” as used herein, alone or in combination, refers to a stable straight or branched hydrocarbon chain, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

“Heteroaryl,” as used herein, alone or in combination, refers to a 3 to 15 membered aromatic monocyclic ring, or a fused monocyclic, bicyclic, or tricyclic ring system in which at least one of the fused rings is aromatic, which ring or ring system contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, xanthenyl and the like.

“Heterocycloalkyl” and, interchangeably, “heterocycle,” as used herein, alone or in combination, each refers to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. A heterocycle may comprise a bridged ring system and/or a spiro ring system (e.g., a system including two rings sharing a single atom, such as a single carbon atom). “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups are unsubstituted or substituted unless specifically prohibited.

“Hydrazinyl” as used herein, alone or in combination, refers to two amino groups joined by a single bond, i.e., —N—N—.

“Hydroxy,” as used herein, alone or in combination, refers to —OH.

“Hydroxyalkyl,” as used herein, alone or in combination, refers to a hydroxy group attached to the parent molecular moiety through an alkyl group.

“Iminohydroxy,” as used herein, alone or in combination, refers to ═N(OH) and ═N—O—.

“Lower amino,” as used herein, alone or in combination, refers to —NRR′, wherein R and R′ are independently selected from hydrogen and lower alkyl, either of which is unsubstituted or substituted.

“Mercaptyl” as used herein, alone or in combination, refers to an RS— group, where R is as defined herein.

“Nitro,” as used herein, alone or in combination, refers to —NO₂.

“Oxy” or “oxa,” as used herein, alone or in combination, refer to —O—.

“Oxo,” as used herein, alone or in combination, refers to ═O.

“Perhaloalkoxy” refers to an alkoxy group where all of the hydrogen atoms are replaced by halogen atoms.

“Perhaloalkyl” as used herein, alone or in combination, refers to an alkyl group where all of the hydrogen atoms are replaced by halogen atoms.

“Ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), S(O)ₘ, wherein m is 0, 1, or 2. Unless specifically prohibited, a ring is unsubstituted or substituted. Two or more rings may be fused together (e.g., they may share a bond and two common atoms). Two or more rings may be linked together in a spiro arrangement such that only a single atom is shared between two rings. Two or more rings may also or alternatively be configured in a bridged arrangement such that three or more atoms are shared between two or more rings.

“Sulfonate,” “sulfonic acid,” and “sulfonic,” as used herein, alone or in combination, refer to the —SO₃H group and its anion as the sulfonic acid is used in salt formation.

“Sulfanyl,” as used herein, alone or in combination, refers to —S—.

“Sulfinyl,” as used herein, alone or in combination, refers to —S(O)—.

“Sulfonyl,” as used herein, alone or in combination, refers to —S(O)₂—.

“N-sulfonamido” refers to a RS(═O)₂NR′— group with R and R′ as defined herein.

“S-sulfonamido” refers to a —S(═O)₂NRR′, group, with R and R′ as defined herein.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers that are well known in the art include keto/enol, enamine/imine, and lactam/lactim tautomers. Examples of tautomers that are well known in the art also include 2-hydroxypyridine/2(1H)-pyridone and 2-aminopyridine/2(1H)-iminopyridone tautomers.

“Thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

“Thiol,” as used herein, alone or in combination, refers to an —SH group.

“Thiocarbonyl,” as used herein, when alone includes thioformyl —C(S)H and in combination is a —C(S)— group.

“N-thiocarbamyl” refers to an ROC(S)NR′— group, with R and R′ as defined herein.

“O-thiocarbamyl” refers to a —OC(S)NRR′ group with R and R′ as defined herein.

“Thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As described herein, groups may be substituted or unsubstituted (e.g., “optionally substituted”). Unless otherwise specified, any group may be substituted with one or more substituents, such as one or more substituents provided herein. Examples of substituents that may substitute a group include, but are not limited to, one or more substituents independently selected from the following groups or a particular designated set of groups, alone or in combination: alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), alkynyl (e.g., C₂₋₂₀ alkynyl, such as C₂₋₁₀ alkynyl, such as C₂₋₆ alkynyl), alkanoyl (e.g., C₁₋₂₀ alkanoyl, such as C₁₋₁₀ alkanoyl, such as C₁₋₆ alkanoyl), heteroalkyl (e.g., a heteroalkyl moiety including 1-20 carbon atoms and 1-6 heteroatoms, such as a heteroalkyl moiety including 1-6 carbon atoms and 1-3 heteroatoms), haloalkyl (e.g., a halo-substituted C₁₋₂₀ alkyl, such as a halo-substituted C₁₋₁₀ alkyl, a halo-substituted C₁₋₆ alkyl), haloalkenyl (e.g., a halo-substituted C₂₋₂₀ alkenyl, such as a halo-substituted C₂₋₆ alkenyl), haloalkynyl (e.g., a halo-substituted C₂₋₂₀ alkynyl, such as a halo-substituted C₂₋₆ alkynyl), perhaloalkyl (e.g., C₁₋₂₀ perhaloalkyl, such as C₁₋₆ perhaloalkyl, such as C₁₋₃ perhaloalkyl), perhaloalkoxy (e.g., C₁₋₂₀ perhaloalkoxy, such as C₁₋₆ perhaloalkoxy), phenyl, aryl (e.g., C₅₋₂₀ aryl, such as C₅₋₁₀ aryl, such as C₅₋₆ aryl), aryloxy (e.g., C₅₋₂₀ aryloxy, such as C₅₋₁₀ aryloxy, such as C₅₋₆ aryloxy), alkoxy (e.g., C₁₋₂₀ alkoxy, such as C₁₋₁₀ alkoxy, such as C₁₋₆ alkoxy), haloalkoxy (e.g., C₁₋₂₀ haloalkoxy, such as C₁₋₁₀ haloalkoxy, such as C₁₋₆ haloalkoxy), oxo, acyloxy (e.g., an acyloxy group including 1-20 carbon atoms, such as 1-10 carbon atoms, such as 1-6 carbon atoms), carbonyl (e.g., C(O) or C═O), carboxyl (e.g., C(O)O), alkylcarbonyl (e.g., C₁₋₂₀ alkylcarbonyl, such as C₁₋₁₀ alkylcarbonyl, such as C₁₋₆ alkylcarbonyl, such as C₁₋₃ alkylcarbonyl), carboxyester (e.g., C(O)OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), carboxamido, cyano (e.g., CN), hydrogen, halogen (e.g., iodine, bromine, chlorine, or fluorine), hydroxy, amino (e.g., NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), alkylamino (e.g., NR′R″ where R′ is alkyl (e.g., C₁₋₂₀ alkyl, such as C₁₋₁₀ alkyl, such as C₁₋₆ alkyl, such as C₁₋₃ alkyl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), arylamino (e.g., NR′R″ where R′ is aryl (e.g., C₅₋₂₀ aryl, such as C₅₋₁₀ aryl, such as C₅₋₆ aryl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), amido (e.g., C(O)NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), nitro (e.g., NO₂), thiol (e.g., SH), alkylthio (e.g., C₁₋₂₀ alkyl substituted with a thiol group, such as C₁₋₁₀ alkyl substituted with a thiol group, such as C₁₋₆ alkyl substituted with a thiol group, such as C₁₋₃ alkyl substituted with a thiol group), haloalkylthio (e.g., C₁₋₂₀ haloalkylthio, such as C₁₋₁₀ haloalkylthio, such as C₁₋₆ haloalkylthio, such as C₁₋₃ haloalkylthio), perhaloalkylthio (e.g., C₁₋₂₀ perhaloalkylthio, such as C₁₋₁₀ perhaloalkylthio, such as C₁₋₆ perhaloalkylthio, such as C₁₋₃ perhaloalkylthio), arylthiol (e.g., C₅₋₂₀ arylthiol, such as C₅₋₁₀ arylthiol, such as C₅₋₆ arylthiol), sulfonate (e.g., S(O)₂OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), sulfonic acid (e.g., S(O)₂OH), trisubstituted silyl (e.g., SiR′R″R* where R′, R″, and R* are independently selected from, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein; in some cases, a trisubstituted silyl can be trimethylsilyl), N₃, SCH₃, C(O)CH₃, CO₂CH₃, CO₂H, pyridinyl, thiophene, furanyl, carbamate, and urea. Additional groups may also be contemplated. Where structurally feasible, two substituents may be joined together to form a fused five-, six-, or seven-membered carbocyclic or heterocyclic ring consisting of zero to three heteroatoms (e.g., N, O, S, etc.), for example forming methylenedioxy or ethylenedioxy. An unsubstituted or substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “unsubstituted or substituted with.”

The terms R, R′, R″, R*, etc., appearing by themselves and without a number designation, unless otherwise defined, refer to a moiety selected from hydrogen, alkyl, cycloalkyl, heteroalkyl, aryl, heteroaryl and heterocycloalkyl, any of which is unsubstituted or substituted (e.g., as described herein). Such Rand R′ groups should be understood to be unsubstituted or substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)—may be attached to the parent moiety at either the carbon or the nitrogen.

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Asymmetric centers may exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds provided herein may comprise conformational isomers, which compounds comprise groups that can orient in different conformations in relation to another moiety. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Combination therapy” means the administration of two or more therapeutic agents to treat a therapeutic condition or disorder described in the present disclosure. Such administration encompasses co-administration of these therapeutic agents in a substantially simultaneous manner, such as in a single dose unit (e.g., capsule) having a fixed ratio of active ingredients or in multiple, separate dose units (e.g., capsules) for each active ingredient. In addition, such administration also encompasses use of each type of therapeutic agent in a sequential manner. In either case, the treatment regimen will provide beneficial effects of the drug combination in treating the conditions or disorders described herein.

“KRAS inhibitor” is used herein to refer to a compound that exhibits inhibitory activity of RAS (e.g., KRAS)-mediated signal transduction. Such activity may be assessed and demonstrated by various methods, including a showing of a decrease in the levels of signaling transduction molecules downstream in the RAS pathway (e.g., pMEK, pERK, or pAKT), and/or a decrease in binding of RAS complex to downstream signaling molecules such as Raf. A KRAS inhibitor may refer to a compound that exhibits an IC₅₀ with respect to KRAS activity of no more than about 100 μM and more typically not more than about 50 μM, as measured in the assays described generally herein, such as a surface plasmon resonance KRAS-G12D or KRAS-G12V or wild-type KRAS protein binding assay and/or a KRAS G12D, KRAS G12V, or wild-type KRAS protein-effector protein interaction disruption assay. “IC₅₀” is that concentration of inhibitor which reduces the activity of an enzyme (e.g., KRAS) to half-maximal level. Certain compounds disclosed herein have been discovered to exhibit inhibition against KRAS. In certain embodiments, compounds will exhibit an IC₅₀ with respect to KRAS (e.g., KRAS having a G12D or G12V mutation or wild-type KRAS) of no more than about 50 μM; in further embodiments, compounds will exhibit an IC₅₀ with respect to KRAS (e.g., KRAS having a G12D or G12V mutation or wild-type KRAS) of no more than about 10 μM; in yet further embodiments, compounds will exhibit an IC₅₀ with respect to KRAS (e.g., KRAS having a G12D or G12V mutation or wild-type KRAS) of not more than about 1 μM; in yet further embodiments, compounds will exhibit an IC₅₀ with respect to KRAS (e.g., KRAS having a G12D or G12V mutation or wild-type KRAS) of not more than about 200 nM, as measured in the KRAS assay described herein. In some embodiments, compounds will exhibit an IC₅₀ with respect to KRAS (e.g., KRAS having a G12D or G12V mutation or wild-type KRAS) of less than about 5 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In certain embodiments, compounds will exhibit an IC₅₀ with respect to KRAS (e.g., KRAS having a G12D or G12V mutation or wild-type KRAS) of less than about 1 μM, such as less than about 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, a KRAS inhibitor has inhibitory activity against KRAS having a G12D mutation that exceeds its inhibitory activity against wild-type KRAS or KRAS having another mutation, such as a G12C mutation, a G12V mutation, a G12S mutation, and/or a G12R mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to wild-type KRAS or KRAS having a G12C mutation, a G12V mutation, a G12S mutation, and/or a G12R mutation. In some embodiments, a KRAS inhibitor has inhibitory activity against KRAS having a G12V mutation that exceeds its inhibitory activity against wild-type KRAS or KRAS having another mutation, such as a G12C mutation, a G12D mutation, a G12S mutation, and/or a G12R mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12V mutation relative to wild-type KRAS or KRAS having a G12C mutation, a G12D mutation, a G12S mutation, and/or a G12R mutation. In some embodiments, a KRAS inhibitor has inhibitory activity against wild-type KRAS that exceeds its inhibitory activity against KRAS having a mutation, such as a G12C mutation, a G12D mutation, a G12V mutation, a G12S mutation, and/or a G12R mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against wild-type KRAS relative to KRAS having a G12C mutation, a G12D mutation, a G12V mutation, a G12S mutation, and/or a G12R mutation. In certain embodiments, compounds will exhibit an IC₅₀ with respect to KRAS (e.g., KRAS having a G12D or G12V mutation or wild-type KRAS) of less than about 1 μM. In some embodiments, a KRAS inhibitor as inhibitory activity against KRAS having a G12D or G12V mutation or wild-type KRAS that exceeds its inhibitory activity against KRAS having another mutation, such as a G12C mutation. For example, in some embodiments, a KRAS inhibitor provided herein has at least two-fold, five-fold, ten-fold, twenty-fold, or higher inhibitory activity against KRAS having a G12D or G12V mutation or wild-type KRAS relative to KRAS having a G12C mutation. In some embodiments, a KRAS inhibitor provided herein has greater inhibitory activity against active KRAS having a G12D mutation than against an inactive KRAS having a G12D mutation. In some embodiments, a KRAS inhibitor provided herein has lower inhibitory activity against active KRAS having a G12D mutation than against an inactive KRAS having a G12D mutation. In some embodiments, a KRAS inhibitor provided herein has similar inhibitory activity against active and inactive KRAS having a G12D mutation. In some embodiments, a KRAS inhibitor provided herein has greater inhibitory activity against active KRAS having a G12V mutation than against an inactive KRAS having a G12V mutation. In some embodiments, a KRAS inhibitor provided herein has lower inhibitory activity against active KRAS having a G12V mutation than against an inactive KRAS having a G12V mutation. In some embodiments, a KRAS inhibitor provided herein has similar inhibitory activity against active and inactive KRAS having a G12V mutation. In some embodiments, a KRAS inhibitor provided herein has greater inhibitory activity against active wild-type KRAS than against an inactive wild-type KRAS. In some embodiments, a KRAS inhibitor provided herein has lower inhibitory activity against active wild-type KRAS than against an inactive wild-type KRAS. In some embodiments, a KRAS inhibitor provided herein has similar inhibitory activity against active and inactive wild-type KRAS. In some embodiments, a KRAS inhibitor provided herein has inhibitory activity against a K-RAS4a splice variant. In some embodiments, a KRAS inhibitor provided herein has inhibitory activity against a K-RAS4b splice variant. In some embodiments, a KRAS inhibitor provided herein has inhibitory activity against both K-RAS4a and K-RAS4b splice variants.

“Therapeutically effective amount” refers to an amount of a compound or of a pharmaceutical composition useful for treating or ameliorating an identified disease, disorder, or condition, or for exhibiting a detectable therapeutic or inhibitory effect. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). A therapeutically effective amount may decrease KRAS activity by at least 1% compared to a control, such as at least 5%, 10%, 15%, 20%, 25%, 30%, 35%, 40%, 45%, 50%, 55%, 60%, 65%, 70%, 75%, 80%, 85%, 90%, 95%, or more.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

“Treat,” “treating,” and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition. Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level.

“Patient” or “subject” refers to a living organism suffering from or prone to a disease, disorder, or condition that can be treated by administration of a compound or pharmaceutical composition as provided herein. Non-limiting examples include humans, rats, mice, rabbits, hamsters, guinea pigs, hamsters, cats, dogs, non-human primates (e.g., monkeys), goats, pigs, sheep, cows, deer, horses, and other non-mammalian animals. Examples of mammals that can be treated by administration of a compound or pharmaceutical composition provided herein include, for example, rodents (e.g., rats, mice, squirrels, guinea pigs, hamsters, etc.), lagomorphs (e.g., rabbits, hares, etc.), primates (e.g., monkeys, apes, etc.), bovines (e.g., cattle), odd-toed ungulates (e.g., horses), even-toed ungulates (e.g., bovines such as cattle, ovine such as sheep, caprine such as goats, porcine such as pigs, etc.), and marsupials (e.g., kangaroo, wallaby, wallaroo, sugar glider, etc.). In some embodiments, the patient or subject is human. In some embodiments, the patient or subject is a companion animal such as a cat or dog. In some embodiments, the patient or subject is a farm animal such as a goat, sheep, cow, pig, or horse. In some embodiments, the patient or subject is an exotic animal such as a primate (e.g., monkey), marsupial (e.g., kangaroo, wallaby, wallaroo, sugar glider, etc.), or a non-domesticated or hybrid cat or dog.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent, or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug.

The compounds disclosed herein can exist as therapeutically acceptable salts (also referred to herein as “pharmaceutically acceptable salts”). The present disclosure includes compounds provided herein in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt” or “pharmaceutically acceptable salt” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, NN-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

“KRAS positive cancer” refers to a cancer characterized by a KRAS mutation, such as a KRAS Q61H, G12C, G12D, G12R, G12A, G12S, G12V, or G13D mutation, and/or by amplified wild-type KRAS activity. In some embodiments, “KRAS positive cancer” refers to a cancer that may benefit from inhibition of KRAS, such as wild-type KRAS or KRAS having a Q61H, G12C, G12D, G12R, G12A, G12S, G12V, or G13D mutation. In some embodiments, “KRAS positive cancer” refers to a cancer that may benefit from inhibition of wild-type KRAS or KRAS having a G12D or G12V mutation.

“KRAS G12D-positive cancer” refers to a cancer characterized by a KRAS G12D mutation.

“KRAS G12V-positive cancer” refers to a cancer characterized by a KRAS G12V mutation.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a KRAS G12D inhibitor, as defined herein, and an additional agent, which additional agent may be an agent configured to treat a disease, disorder, or condition or a symptom thereof. The effect can be, for example, slowing the symptomatic progression of a proliferative disease, such as cancer, particularly lung cancer, or symptoms thereof. Analogously, a “synergistically effective amount” refers to the amount needed to obtain a synergistic effect

“A,” “an,” or “a(n)”, when used in reference to a group of substituents or “substituent group” herein, mean at least one. For example, where a compound is substituted with “an” alkyl or aryl, the compound is unsubstituted or substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### Compounds

In an aspect, the present disclosure provides a compound according to Formula I:

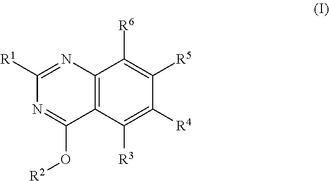

or a salt (e.g., pharmaceutically acceptable salt), ester, tautomer, prodrug, zwitterionic form, or stereoisomer thereof, wherein:

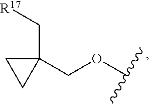

R¹ is selected from —OR⁷, and a 4-6 membered heterocycle comprising a nitrogen atom, wherein the heterocycle is unsubstituted or substituted with one or more R⁵;
R² is a 4-11 membered heterocycle containing one or more nitrogen atoms, wherein the heterocycle is unsubstituted or is substituted with one or more R⁸;
R³ is H;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is independently selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen; and
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula I, or a salt (e.g., a pharmaceutically acceptable salt) thereof.

In some embodiments, for a compound according to Formula I, R² is a 4-11 membered heterocycle containing one or more nitrogen atoms, which heterocycle is unsubstituted. In some embodiments, R² is a 4-11 membered heterocycle containing one or more nitrogen atoms, which heterocycle is substituted with one or more R⁸. In some embodiments, the one or more R⁸ are selected from C₁₋₆alkyl that is unsubstituted or substituted with one or more R¹¹.

In some embodiments, for a compound according to Formula I, R² is a 4-11 membered heterocycle containing one nitrogen atom, wherein the heterocycle is unsubstituted or is substituted with one or more R⁸. In some embodiments, R² is a 4-11 membered heterocycle containing one nitrogen atom, wherein the heterocycle is unsubstituted. In some embodiments, R² is a 4-11 membered heterocycle containing one nitrogen atom, wherein the heterocycle is substituted with one or more R⁸. In some embodiments, R² is a 4-11 membered heterocycle containing one nitrogen atom, wherein the heterocycle is substituted with one or more halogens. In some embodiments, R² is a 4-11 membered heterocycle containing one nitrogen atom, wherein the heterocycle is substituted with one or more C₁₋₆alkyl that is independently unsubstituted or substituted with one or more R¹¹.

In some embodiments, for a compound according to Formula I, R² is a 4-6 membered heterocycle containing one nitrogen atom, wherein the heterocycle is unsubstituted or is substituted with one or more R⁸. In some embodiments, R² is a heterocycle selected from azetidine and pyrrolidine, wherein the heterocycle is unsubstituted or is substituted with one or more R⁸. In some embodiments, R² is azetidine, wherein the azetidine is unsubstituted or is substituted with one or more R⁸. In some embodiments, R² is pyrrolidine, wherein the pyrrolidine is unsubstituted or is substituted with one or more R⁸. In some embodiments, R² is piperidine, wherein the piperidine is unsubstituted or is substituted with one or more R⁸. In some embodiments, R² is a 4-6 membered heterocycle containing one nitrogen atom, wherein the heterocycle is substituted with one or more R⁸. In some embodiments, R² is a 4-6 membered heterocycle containing one nitrogen atom, wherein the heterocycle is substituted with one or more R⁸, wherein the one or more R⁸ are independently selected from halogen and C₁₋₆alkyl, wherein any C₁₋₆alkyl is unsubstituted. In some embodiments, R² is a 4-6 membered heterocycle containing one nitrogen atom, wherein the heterocycle is substituted with one or more R⁸, wherein the one or more R⁸ are independently selected from halogen and C₁₋₆alkyl, wherein any C₁₋₆alkyl is substituted with one or more R¹¹. In some embodiments, R² is a 4-6 membered heterocycle containing one nitrogen atom, wherein the heterocycle is substituted with one or more halogens. In some embodiments, R² is a 4-6 membered heterocycle containing one nitrogen atom, wherein the heterocycle is substituted with one or more C₁₋₆alkyl that is independently unsubstituted or substituted with one or more R¹¹.

In some embodiments, for a compound according to Formula I, R² is a bridged heterocycle containing one nitrogen atom, wherein the heterocycle is unsubstituted or is substituted with one or more R⁸. In some embodiments, R² is a bridged heterocycle containing one nitrogen atom, wherein the heterocycle is unsubstituted. In some embodiments, R² is a bridged heterocycle containing one nitrogen atom, wherein the heterocycle is substituted with one or more R⁸.

In some embodiments, for a compound according to Formula I, R² is a spirocyclic heterocycle containing one or more nitrogen atoms, wherein the heterocycle is unsubstituted or substituted with one or more R⁸. In some embodiments, R² is a spirocycle including a first ring that is a carbocycle and a second ring that is a heterocycle containing a nitrogen atom, wherein the spirocycle is unsubstituted or is substituted with one or more R⁸. In some embodiments, R² is a spirocycle including a first ring that is a carbocycle and a second ring that is a heterocycle containing a nitrogen atom, wherein the spirocycle is unsubstituted. In some embodiments, R² is a spirocycle including a first ring that is a carbocycle and a second ring that is a heterocycle containing a nitrogen atom, wherein the spirocycle is substituted with one or more R⁸. In some embodiments, R² is a spirocycle including a first ring that is a 4-6 membered ring and a second ring that is a 4-6 membered ring. In some embodiments, R² is a spirocycle including a first ring that is a 4-membered ring and a second ring that is a 4-membered ring. In some embodiments, R² is a spirocycle including a first ring that is a 4-membered ring and a second ring that is a 5-membered ring. In some embodiments, R² is a spirocycle including a first ring that is a 4-membered ring and a second ring that is a 6-membered ring. In some embodiments, R² is a spirocycle including a first ring that is a 5-membered ring and a second ring that is a 6-membered ring.

In some embodiments, the compound is a compound according to Formula IA:

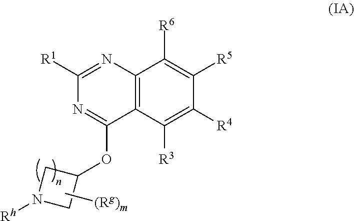

or a salt (e.g., pharmaceutically acceptable salt), ester, tautomer, prodrug, zwitterionic form, or stereoisomer thereof, wherein:
R¹ is selected from —OR⁷,

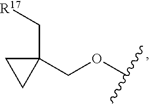

and a 4-6 membered heterocycle comprising a nitrogen atom, wherein the heterocycle is unsubstituted or substituted with one or more R⁵;
R³ is H;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen;
n is 1, 2, or 3;
m is 0, 1, 2, 3, or 4;
each R^{g} is independently selected from halogen, C₁₋₆alkyl, and —OR¹⁰, wherein any C₁₋₆alkyl is unsubstituted or substituted with one or more R¹¹, or two R^{g}, together with the atoms to which they are attached, join to form a 5-7 membered ring;
R^{h} is selected from H and C₁₋₆alkyl, wherein any C₁₋₆alkyl is unsubstituted or substituted with one or more R¹¹; and
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula IA, or a salt (e.g., a pharmaceutically acceptable salt) thereof.

In some embodiments, the compound is a compound according to Formula IA1:

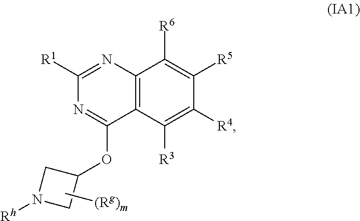

or a salt (e.g., pharmaceutically acceptable salt), ester, tautomer, prodrug, zwitterionic form, or stereoisomer thereof. In some embodiments, the compound is a compound according to Formula IA1, or a salt (e.g., a pharmaceutically acceptable salt) thereof.

In some embodiments, the compound is a compound according to Formula IA2:

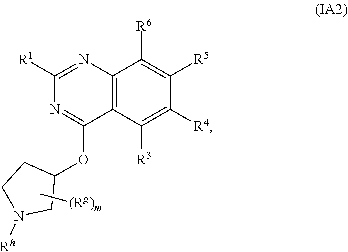

or a salt (e.g., pharmaceutically acceptable salt), ester, tautomer, prodrug, zwitterionic form, or stereoisomer thereof. In some embodiments, the compound is a compound according to Formula IA2, or a salt (e.g., a pharmaceutically acceptable salt) thereof.

In some embodiments, the compound is a compound according to Formula IA3:

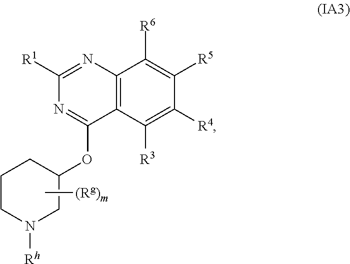

or a salt (e.g., pharmaceutically acceptable salt), ester, tautomer, prodrug, zwitterionic form, or stereoisomer thereof. In some embodiments, the compound is a compound according to Formula IA3, or a salt (e.g., a pharmaceutically acceptable salt) thereof.

In some embodiments, the compound is a compound according to Formula IA4:

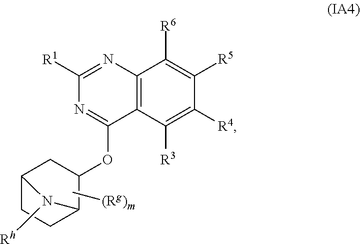

or a salt (e.g., pharmaceutically acceptable salt), ester, tautomer, prodrug, zwitterionic form, or stereoisomer thereof. In some embodiments, the compound is a compound according to Formula IA4, or a salt (e.g., a pharmaceutically acceptable salt) thereof.

In some embodiments, the compound is a compound according to Formula IA5:

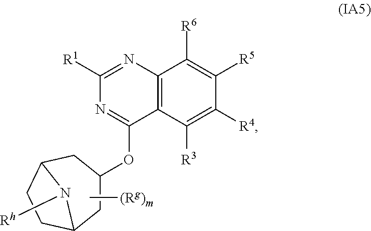

or a salt (e.g., pharmaceutically acceptable salt), ester, tautomer, prodrug, zwitterionic form, or stereoisomer thereof. In some embodiments, the compound is a compound according to Formula IA5, or a salt (e.g., a pharmaceutically acceptable salt) thereof.

In some embodiments, for a compound according to any one of Formulas IA, IA1, IA2, IA3, IA4, and IA5, R^{h} is H.

In some embodiments, for a compound according to any one of Formulas IA, IA1, IA2, IA3, IA4, and IA5, m is 0. In some embodiments, m is 1 or 2, and each R^{g} is independently selected from halogen and C₁₋₆alkyl, wherein any C₁₋₆alkyl is unsubstituted.

In some embodiments, for a compound according to any one of Formulas IA, IA1, IA2, IA3, IA4, and IA5, each R^{g} is independently selected from halogen and C₁₋₆alkyl, wherein any C₁₋₆alkyl is unsubstituted or substituted with one or more R¹¹.

In some embodiments, the compound is a compound according to Formula IB:

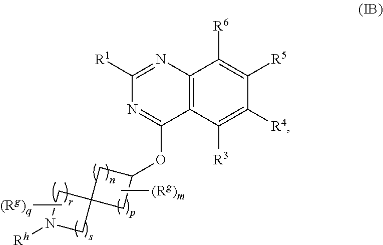

or a salt (e.g., pharmaceutically acceptable salt), ester, tautomer, prodrug, zwitterionic form, or stereoisomer thereof, wherein:
R¹ is selected from —OR⁷,

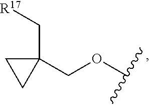

and a 4-6 membered heterocycle comprising a nitrogen atom, wherein the heterocycle is unsubstituted or substituted with one or more R⁵;
R³ is H;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁶;
each R¹⁶ is independently selected from C₁₋₆alkyl and halogen;
m and q are each independently selected from 0, 1, 2, 3, and 4;
n and p are each independently selected from 1 and 2;
r and s are each independently selected from 1 and 2;
each R⁹ is independently selected from halogen, C₁₋₆alkyl, and —OR¹⁰, wherein any C₁₋₆alkyl is unsubstituted or substituted with one or more R¹¹;
R^{h} is selected from H and C₁₋₆alkyl, wherein any C₁₋₆alkyl is unsubstituted or substituted with one or more R¹¹; and
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula IB, or a salt (e.g., a pharmaceutically acceptable salt) thereof.

In some embodiments, the compound is a compound according to Formula IB1, IB2, or IB3:

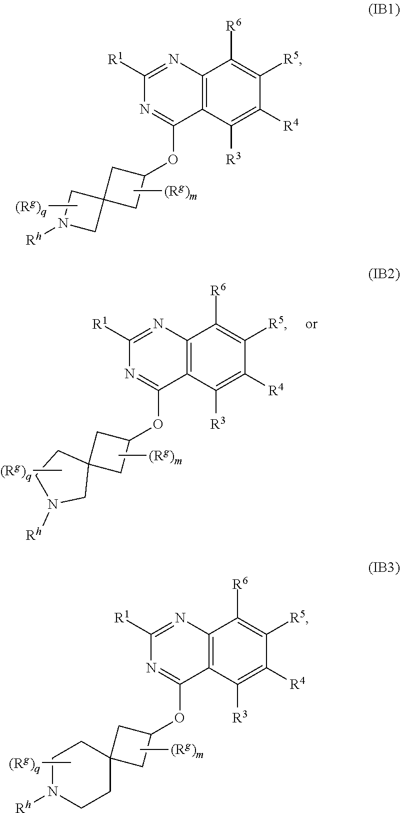

or a salt (e.g., pharmaceutically acceptable salt), ester, tautomer, prodrug, zwitterionic form, or stereoisomer thereof. In some embodiments, the compound is a compound according to Formula IB1, or a salt (e.g., a pharmaceutically acceptable salt) thereof. In some embodiments, the compound is a compound according to Formula IB2, or a salt (e.g., a pharmaceutically acceptable salt) thereof. In some embodiments, the compound is a compound according to Formula IB3, or a salt (e.g., a pharmaceutically acceptable salt) thereof.

In some embodiments, the compound is a compound according to Formula IB4, IB5, or IB6:

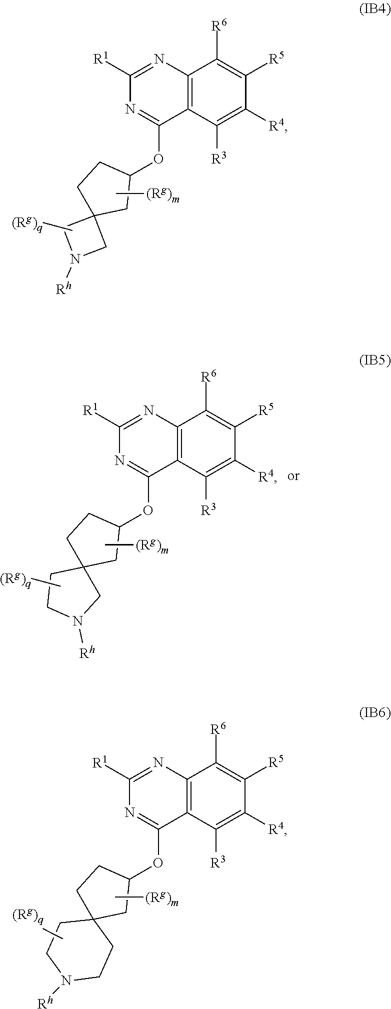

or a salt (e.g., pharmaceutically acceptable salt), ester, tautomer, prodrug, zwitterionic form, or stereoisomer thereof. In some embodiments, the compound is a compound according to Formula IB4, or a salt (e.g., a pharmaceutically acceptable salt) thereof. In some embodiments, the compound is a compound according to Formula IB5, or a salt (e.g., a pharmaceutically acceptable salt) thereof. In some embodiments, the compound is a compound according to Formula IB6, or a salt (e.g., a pharmaceutically acceptable salt) thereof.

In some embodiments, the compound is a compound according to Formula IB7, IB8, or IB9:

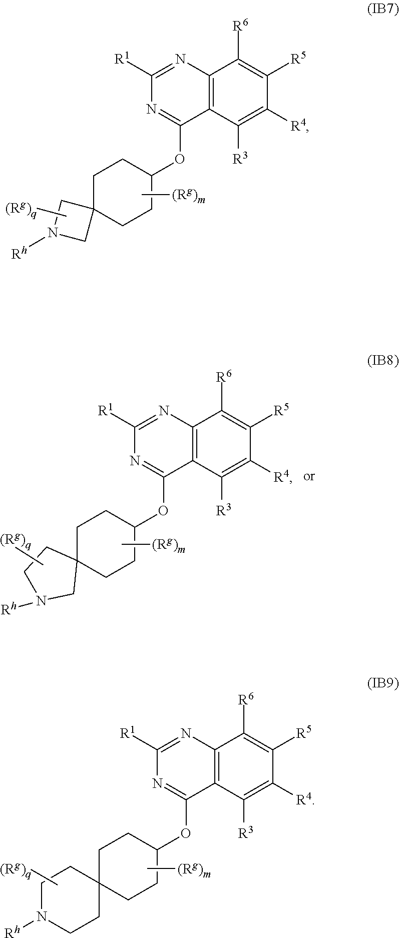

or a salt (e.g., pharmaceutically acceptable salt), ester, tautomer, prodrug, zwitterionic form, or stereoisomer thereof. In some embodiments, the compound is a compound according to Formula IB7, or a salt (e.g., a pharmaceutically acceptable salt) thereof. In some embodiments, the compound is a compound according to Formula IB8, or a salt (e.g., a pharmaceutically acceptable salt) thereof. In some embodiments, the compound is a compound according to Formula IB9, or a salt (e.g., a pharmaceutically acceptable salt) thereof.

In some embodiments, for a compound according to any one of Formulas IB, IB1, IB2, IB3, IB4, IB5, IB6, IB7, IB8, and IB9, R^{h} is H.

In some embodiments, for a compound according to any one of Formulas IB, IB1, IB2, IB3, IB4, IB5, IB6, IB7, IB8, and IB9, each R^{E} is independently selected from halogen and C₁₋₆alkyl, wherein any C₁₋₆alkyl is unsubstituted or substituted with one or more R¹¹.

In some embodiments, for a compound according to any one of Formulas IB, IB1, IB2, IB3, IB4, IB5, IB6, IB7, IB8, and IB9, m is 0. In some embodiments, q is 0. In some embodiments, m and q are 0. In some embodiments, m is 1 or 2. In some embodiments, q is 1 or 2. In some embodiments, m is 0 and q is 1 or 2. In some embodiments, m is 1 or 2 and q is 0.

In some embodiments, for a compound according to any one of Formulas I, IA, IA1, IA2, IA3, IA4, IA5, IB, IB1, IB2, IB3, IB4, IB5, IB6, IB7, IB8, and IB9, R¹ is selected from —OR⁷, wherein R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁷ is a heterocycle or an alkylheterocycle, wherein any heterocycle contains 4-8 members and is substituted with one or more R^{a} or R^{b}. In some embodiments, R⁷ is a heterocycle that is unsubstituted or substituted with one or more R^{a} or R^{b}. In some embodiments, R⁷ is an alkylheterocycle that is unsubstituted or substituted with one or more R^{a} or R^{b}. In some embodiments, R⁷ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR² (e.g., —OCH₃).

In some embodiments, for a compound according to any one of Formulas I, IA, IA1, IA2, IA3, IA4, IA5, IB, IB1, IB2, IB3, IB4, IB5, IB6, IB7, IB8, and IB9, R¹ is selected from:

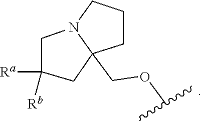

wherein R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³. In some embodiments, R^{a} and R^{b} are each independently selected from halogen, C₁₋₆alkyl, —OR², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

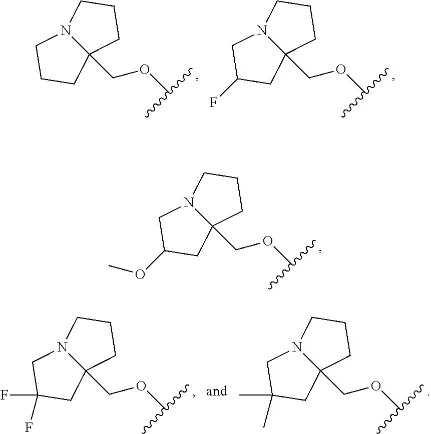

In some embodiments, R¹ is selected from:

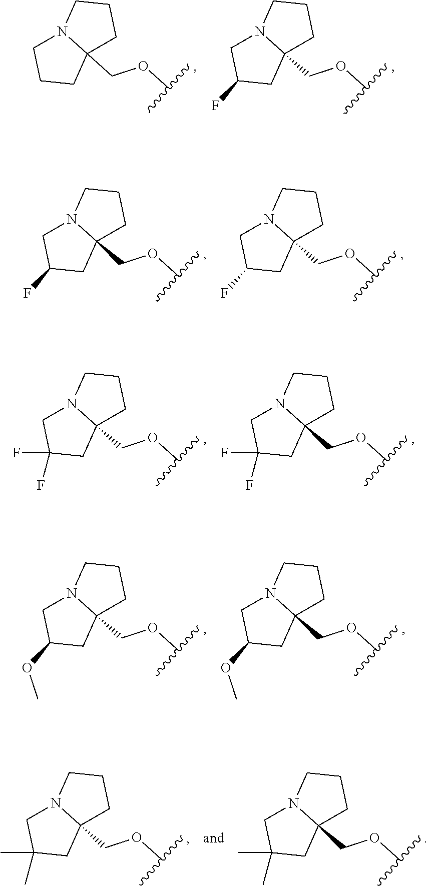

In some embodiments, for a compound according to any one of Formulas I, IA, IA1, IA2, IA3, IA4, IA5, IB, IB1, IB2, IB3, IB4, IB5, IB6, IB7, IB8, and IB9, R¹ is selected from:

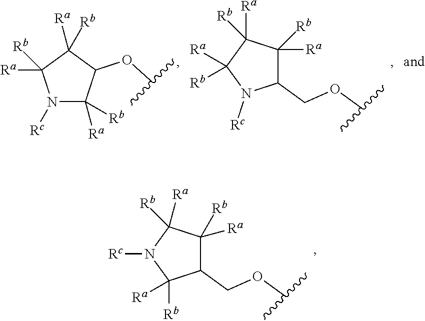

wherein each R^{a} and R^{b} is independently selected from halogen, C₁₋₆ alkyl, —OR¹, and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H, wherein an R^{a} and R^{c} join together to form a 3-6 membered heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆ alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR² (e.g., —OCH₃ or —CHF₂). In some embodiments, one R^{a} or R^{b} is C₁₋₆ alkyl (e.g., methyl). In some embodiments, two R^{a} groups, two R^{b} groups, or an R^{a} and an R^{b} are C₁₋₆ alkyl (e.g., methyl). In some embodiments, R^{c} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{c} join together to form a 3-6 membered heterocycle. In some embodiments, R¹ is selected from:

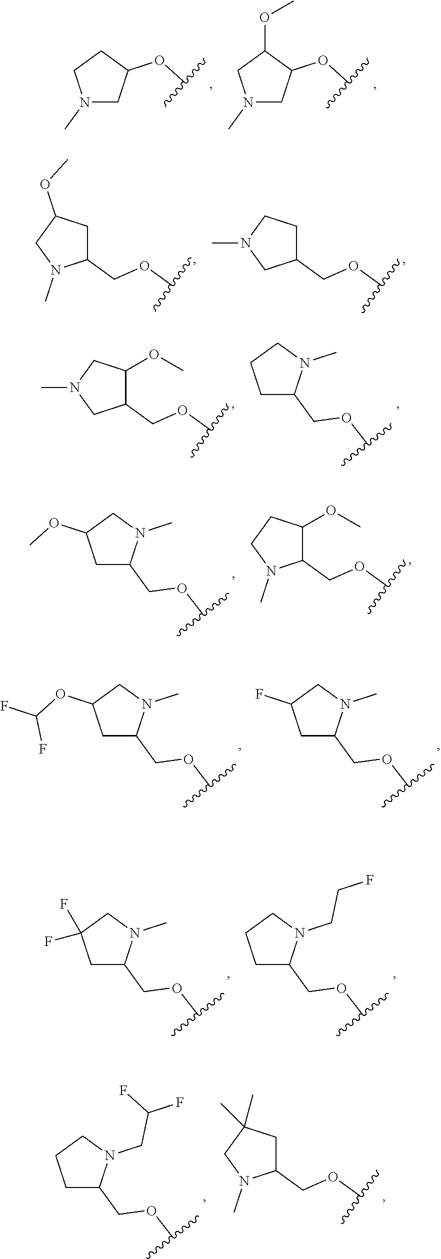

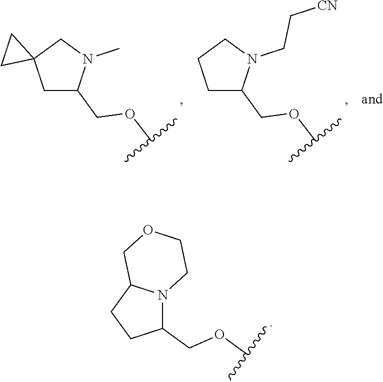 [SMILES: *OCC1CC2(CC2)CN1C ; *OCC1CCCN1CCC#N ; *OCC1CCC2COCCN12]

In some embodiments, R¹ is selected from:

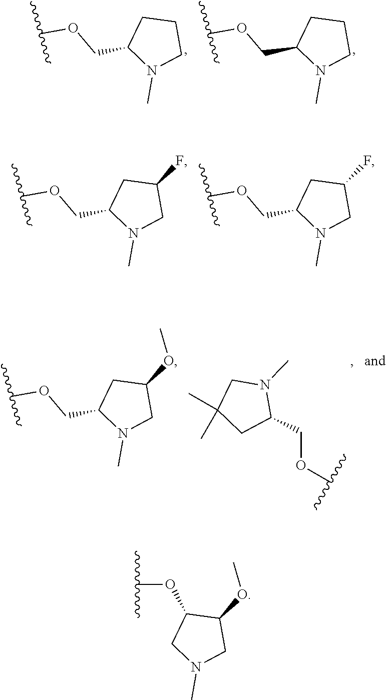

In some embodiments, for a compound according to any one of Formulas I, IA, IA1, IA2, IA3, IA4, IA5, IB, IB1, IB2, IB3, IB4, IB5, IB6, IB7, IB8, and IB9, R¹ is

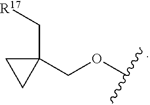

selected from:

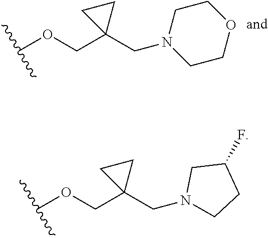 [SMILES: *OCC1(CN2CCOCC2)CC1 ; *OCC1(CN2CC[C@@H](F)C2)CC1]

In some embodiments, for a compound according to any one of Formulas I, IA, IA1, IA2, IA3, IA4, IA5, IB, IB1, IB2, IB3, IB4, IB5, IB6, IB7, IB8, and IB9, R¹ is a 4-6 membered heterocycle comprising a nitrogen atom, wherein the heterocycle is unsubstituted or substituted with one or more R¹⁵. In some embodiments, R¹ is a 4-membered heterocycle comprising a nitrogen atom, wherein the heterocycle is unsubstituted or substituted with one or more R¹⁵. In some embodiments, R¹ is selected from:

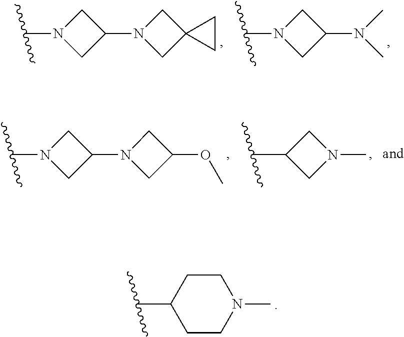

In some embodiments, for a compound according to any one of Formulas I, IA, IA1, IA2, IA3, IA4, IA5, IB, IB1, IB2, IB3, IB4, IB5, IB6, IB7, IB8, and IB9, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is —CN. In some embodiments, R⁴ is a halogen (e.g., F or Cl). In some embodiments, R⁴ is C₁₋₆alkyl that is unsubstituted, such as methyl or ethyl. In some embodiments, R⁴ is C₁₋₆alkyl that is substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴)₂. In some embodiments, R⁴ is —CH₂CN.

In some embodiments, for a compound according to any one of Formulas I, IA, IA1, IA2, IA3, IA4, IA5, IB, IB1, IB2, IB3, IB4, IB5, IB6, IB7, IB8, and IB9, R⁵ is:

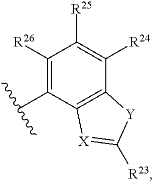

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂.

In some embodiments, for a compound according to any one of Formulas I, IA, IA1, IA2, IA3, IA4, IA5, IB, IB1, IB2, IB3, IB4, IB5, IB6, IB7, IB8, and IB9, R⁵ is selected from:

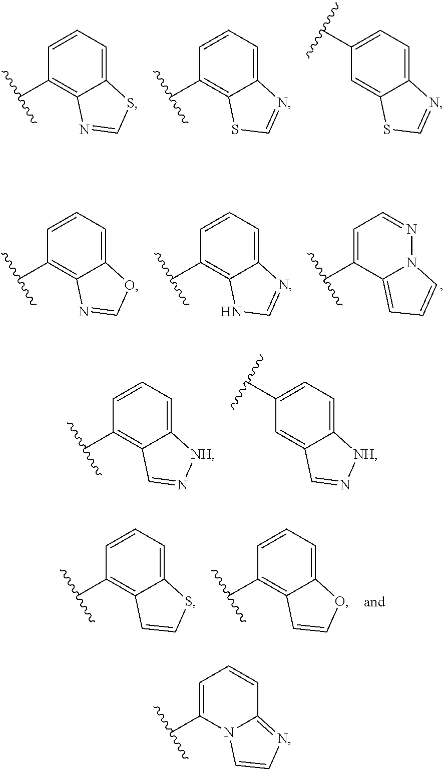

any of which is substituted with one or more R⁹.

In some embodiments, for a compound according to any one of Formulas I, IA, IA1, IA2, IA3, IA4, IA5, IB, IB1, IB2, IB3, IB4, IB5, IB6, IB7, IB8, and IB9, R⁵ is selected from:

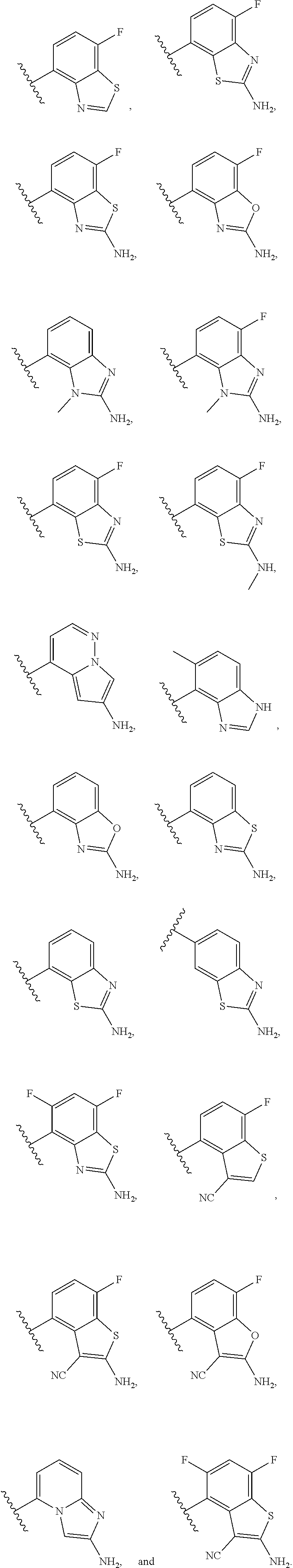

In some embodiments, for a compound according to any one of Formulas I, IA, IA1, IA2, IA3, IA4, IA5, IB, IB1, IB2, IB3, IB4, IB5, IB6, IB7, IB8, and IB9, R⁵ is selected from:

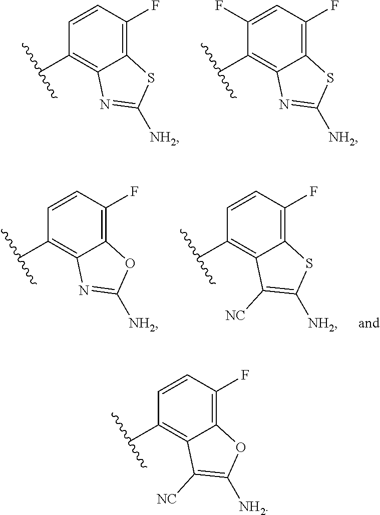 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12]

In some embodiments, for a compound according to any one of Formulas I, IA, IA1, IA2, IA3, IA4, IA5, IB, IB1, IB2, IB3, IB4, IB5, IB6, IB7, IB8, and IB9, R⁶ is selected from halogen, —CN, and H. In some embodiments, R⁶ is a halogen (e.g., Cl or F). In some embodiments, R⁶ is —CN. In some embodiments, R⁶ is H.

In some embodiments, the present disclosure provides a compound according to Formula IC:

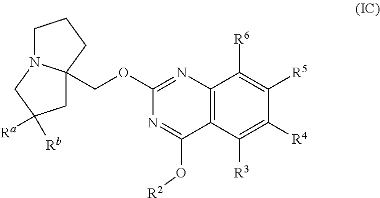

or a salt (e.g., pharmaceutically acceptable salt), ester, tautomer, prodrug, zwitterionic form, or stereoisomer thereof, wherein:
R² is a 4-11 membered heterocycle containing one or more nitrogen atoms, wherein the heterocycle is unsubstituted or is substituted with one or more R⁸;
R³ is H;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H; each R⁸ is independently selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H; and
R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula IC, or a salt (e.g., a pharmaceutically acceptable salt) thereof.

In some embodiments, for a compound according to Formula IC, R^{a} is a halogen or —OR¹². In some embodiments, R^{a} is a halogen, such as F. In some embodiments, R^{a} is —OR¹². In some embodiments, R^{a} is a C₁₋₆ alkyl that is unsubstituted or is substituted with one or more R¹³. In some embodiments, R^{b} is H. In some embodiments, R^{a} is a halogen, such as F, and R^{b} is H. In some embodiments, R^{a} is a —OR¹² and R^{b} is H.

In some embodiments, for a compound according to Formula IC, R² is a 4-11 membered heterocycle containing one or more nitrogen atoms, which heterocycle is unsubstituted. In some embodiments, R² is a 4-11 membered heterocycle containing one or more nitrogen atoms, which heterocycle is substituted with one or more R⁸. In some embodiments, the one or more R⁸ are selected from C₁₋₆alkyl that is unsubstituted or substituted with one or more R¹¹.

In some embodiments, for a compound according to Formula IC, R² is a 4-11 membered heterocycle containing one nitrogen atom, wherein the heterocycle is unsubstituted or is substituted with one or more R⁸. In some embodiments, R² is a 4-11 membered heterocycle containing one nitrogen atom, wherein the heterocycle is unsubstituted. In some embodiments, R² is a 4-11 membered heterocycle containing one nitrogen atom, wherein the heterocycle is substituted with one or more R⁸. In some embodiments, R² is a 4-11 membered heterocycle containing one nitrogen atom, wherein the heterocycle is substituted with one or more halogens. In some embodiments, R² is a 4-11 membered heterocycle containing one nitrogen atom, wherein the heterocycle is substituted with one or more C₁₋₆alkyl that is independently unsubstituted or substituted with one or more R¹¹.

In some embodiments, for a compound according to Formula IC, R² is a 4-6 membered heterocycle containing one nitrogen atom, wherein the heterocycle is unsubstituted or is substituted with one or more R⁸. In some embodiments, R² is a heterocycle selected from azetidine and pyrrolidine, wherein the heterocycle is unsubstituted or is substituted with one or more R⁸. In some embodiments, R² is azetidine, wherein the azetidine is unsubstituted or is substituted with one or more R⁸. In some embodiments, R² is pyrrolidine, wherein the pyrrolidine is unsubstituted or is substituted with one or more R⁸. In some embodiments, R² is piperidine, wherein the piperidine is unsubstituted or is substituted with one or more R⁸. In some embodiments, R² is a 4-6 membered heterocycle containing one nitrogen atom, wherein the heterocycle is substituted with one or more R⁸. In some embodiments, R² is a 4-6 membered heterocycle containing one nitrogen atom, wherein the heterocycle is substituted with one or more R⁸, wherein the one or more R⁸ are independently selected from halogen and C₁₋₆alkyl, wherein any C₁₋₆alkyl is unsubstituted. In some embodiments, R² is a 4-6 membered heterocycle containing one nitrogen atom, wherein the heterocycle is substituted with one or more R⁸, wherein the one or more R⁸ are independently selected from halogen and C₁₋₆alkyl, wherein any C₁₋₆alkyl is substituted with one or more R¹¹. In some embodiments, R² is a 4-6 membered heterocycle containing one nitrogen atom, wherein the heterocycle is substituted with one or more halogens. In some embodiments, R² is a 4-6 membered heterocycle containing one nitrogen atom, wherein the heterocycle is substituted with one or more C₁₋₆alkyl that is independently unsubstituted or substituted with one or more R¹¹.

In some embodiments, for a compound according to Formula IC, R² is a bridged heterocycle containing one nitrogen atom, wherein the heterocycle is unsubstituted or is substituted with one or more R⁸. In some embodiments, R² is a bridged heterocycle containing one nitrogen atom, wherein the heterocycle is unsubstituted. In some embodiments, R² is a bridged heterocycle containing one nitrogen atom, wherein the heterocycle is substituted with one or more R⁸.

In some embodiments, for a compound according to Formula IC, R² is a spirocyclic heterocycle containing one or more nitrogen atoms, wherein the heterocycle is unsubstituted or substituted with one or more R⁸. In some embodiments, R² is a spirocycle including a first ring that is a carbocycle and a second ring that is a heterocycle containing a nitrogen atom, wherein the spirocycle is unsubstituted or is substituted with one or more R⁸. In some embodiments, R² is a spirocycle including a first ring that is a carbocycle and a second ring that is a heterocycle containing a nitrogen atom, wherein the spirocycle is unsubstituted. In some embodiments, R² is a spirocycle including a first ring that is a carbocycle and a second ring that is a heterocycle containing a nitrogen atom, wherein the spirocycle is substituted with one or more R⁸. In some embodiments, R² is a spirocycle including a first ring that is a 4-6 membered ring and a second ring that is a 4-6 membered ring. In some embodiments, R² is a spirocycle including a first ring that is a 4-membered ring and a second ring that is a 4-membered ring. In some embodiments, R² is a spirocycle including a first ring that is a 4-membered ring and a second ring that is a 5-membered ring. In some embodiments, R² is a spirocycle including a first ring that is a 4-membered ring and a second ring that is a 6-membered ring. In some embodiments, R² is a spirocycle including a first ring that is a 5-membered ring and a second ring that is a 6-membered ring.

In some embodiments, for a compound according to Formula IC, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is —CN. In some embodiments, R⁴ is a halogen (e.g., F or Cl). In some embodiments, R⁴ is C₁₋₆alkyl that is unsubstituted, such as methyl or ethyl. In some embodiments, R⁴ is C₁₋₆alkyl that is substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴)₂. In some embodiments, R⁴ is —CH₂CN.

In some embodiments, for a compound according to Formula IC, R⁵ is.

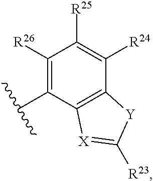

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂.

In some embodiments, for a compound according to Formula IC, R⁵ is selected from:

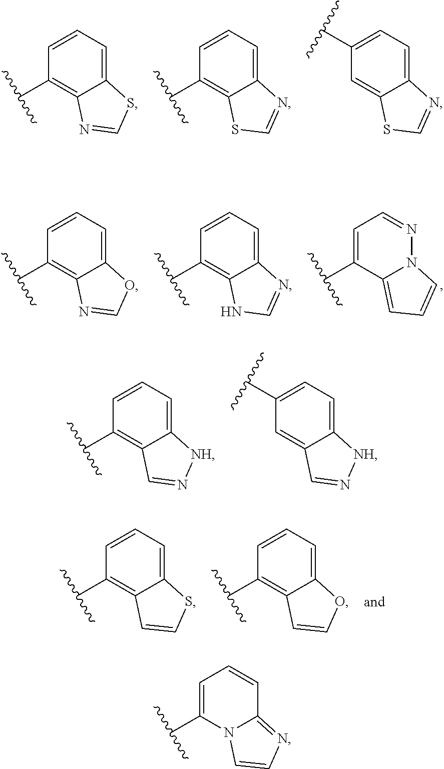

any of which is substituted with one or more R⁹.

In some embodiments, for a compound according to Formula IC, R⁵ is selected from:

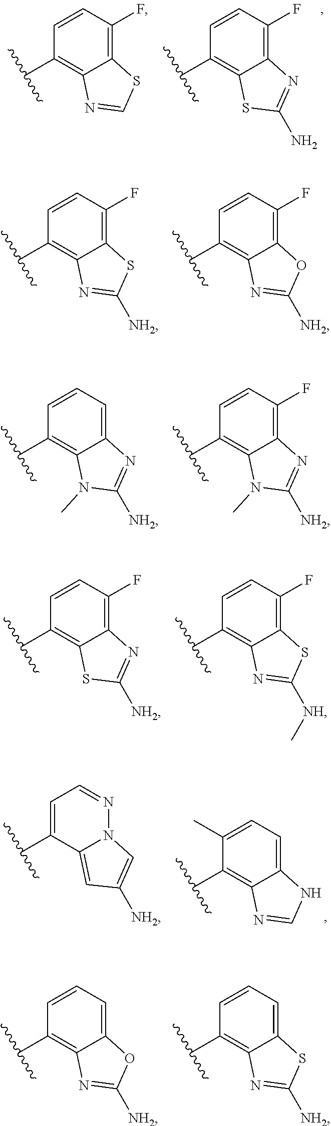

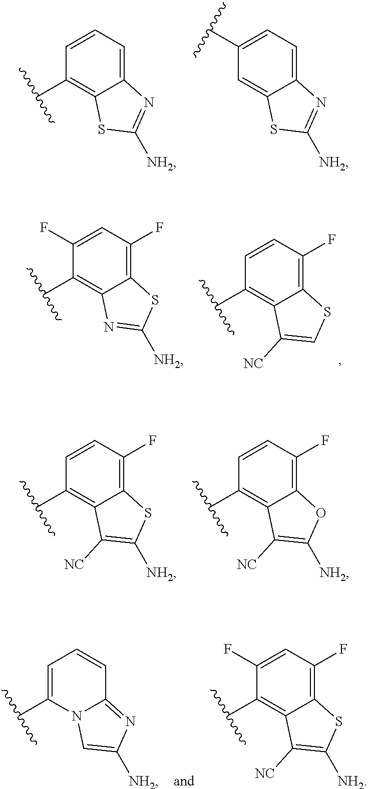

In some embodiments, for a compound according to Formula IC, R⁵ is selected from:

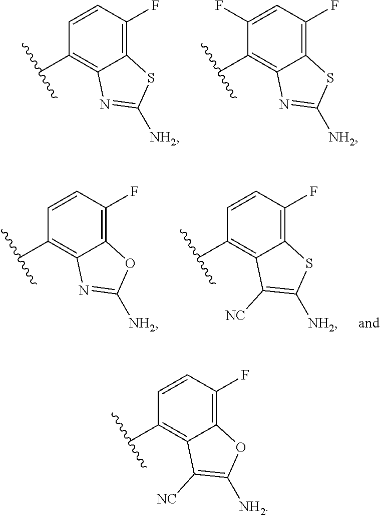 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12]

In some embodiments, for a compound according to Formula IC, R⁶ is selected from halogen, —CN, and H. In some embodiments, R⁶ is a halogen (e.g., Cl or F). In some embodiments, R⁶ is —CN. In some embodiments, R⁶ is H.

In another aspect, the present disclosure provides a compound according to Formula II:

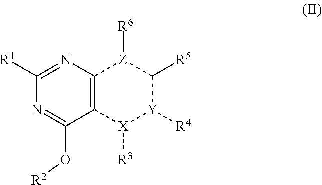

or a salt (e.g., pharmaceutically acceptable salt), ester, tautomer, prodrug, zwitterionic form, or stereoisomer thereof, wherein:
each dashed line represents a single or double bond;
X, Y, and Z are selected from N and C, wherein one and only one of X, Y, and Z is N;
R¹ is selected from —OR⁷,

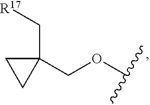

and a 4-6 membered heterocycle comprising a nitrogen atom, wherein the heterocycle is unsubstituted or substituted with one or more R¹⁵;
R² is a 4-6 membered heterocycle containing one or more nitrogen atoms, wherein the heterocycle is unsubstituted or is substituted with one or more R⁸;
when X is C, R³ is H, and when X is N, R³ is absent;
when Y is C, R⁴ is selected from H, halogen, —CN, —OR¹², and C₁₋₆alkyl, wherein any C₁₋₆alkyl is unsubstituted or substituted with one or more R¹¹, and when Y is N, R⁴ is absent;
R⁵ is a bicyclic heteroaryl substituted with one or more R⁹;
when Z is C, R⁶ is selected from halogen, —OR¹², —CN, and H, and when Z is N, R⁶ is absent;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is selected from halogen and C₁₋₆alkyl, wherein any C₁₋₆alkyl is unsubstituted or substituted with one or more R¹¹;
each R⁹ is independently selected from halogen, N(R¹²)₂, —CN, and C₁₋₆alkyl, wherein any C₁₋₆alkyl is unsubstituted or substituted with one or more R¹³;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen; and
each R^{a} and R^{b} is independently selected from halogen, C₁₋₆ alkyl, —OR¹², and H, wherein an W and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula II, or a salt (e.g., a pharmaceutically acceptable salt) thereof.

In some embodiments, for a compound according to Formula II, the ring comprising X, Y, and Z is aromatic.

In some embodiments, for a compound according to Formula II, X is N, and Y and Z are C. In some embodiments, the compound is a compound according to Formula IIA:

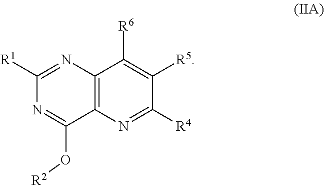

or a salt (e.g., pharmaceutically acceptable salt), ester, tautomer, prodrug, zwitterionic form, or stereoisomer thereof. In some embodiments, the compound is a compound according to Formula IIA, or a salt (e.g., a pharmaceutically acceptable salt) thereof. In some embodiments, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is halogen (e.g., Cl or F). In some embodiments, R⁴ is —CN. In some embodiments, R⁴ is —OR¹². In some embodiments, R⁴ is C₁₋₆alkyl that is unsubstituted or substituted with one or more R¹¹. In some embodiments, R⁴ is C₁₋₆alkyl that is substituted with one or more halogens or —CN.

In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴)₂. In some embodiments, R⁴ is —CH₂CN. In some embodiments, R⁶ is selected from halogen, —CN, and H. In some embodiments, R⁶ is halogen (e.g., Cl or F). In some embodiments, R⁶ is —CN. In some embodiments, R⁶ is H.

In some embodiments, for a compound according to Formula II, Y is N, and X and Z are C. In some embodiments, the compound is a compound according to Formula IIB:

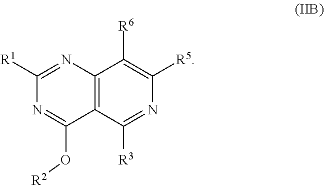

or a salt (e.g., pharmaceutically acceptable salt), ester, tautomer, prodrug, zwitterionic form, or stereoisomer thereof. In some embodiments, the compound is a compound according to Formula IIB, or a salt (e.g., a pharmaceutically acceptable salt) thereof. In some embodiments, R³ is H. In some embodiments, R⁶ is selected from halogen, —CN, and H. In some embodiments, R⁶ is a halogen (e.g., Cl or F). In some embodiments, R⁶ is —CN. In some embodiments, R⁶ is H.

In some embodiments, for a compound according to Formula II, Z is N, and X and Y are C. In some embodiments, the compound is a compound according to Formula IIC:

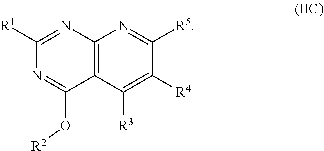

or a salt (e.g., pharmaceutically acceptable salt), ester, tautomer, prodrug, zwitterionic form, or stereoisomer thereof. In some embodiments, the compound is a compound according to Formula IIC, or a salt (e.g., a pharmaceutically acceptable salt) thereof. In some embodiments, R³ is H. In some embodiments, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is halogen (e.g., Cl or F). In some embodiments, R⁴ is —CN. In some embodiments, R⁴ is —OR¹². In some embodiments, R⁴ is C₁₋₆alkyl that is unsubstituted or substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴)₂. In some embodiments, R⁴ is —CH₂CN.

In some embodiments, for a compound according to any one of Formulas II, IIA, IIB, and IIC, R² is a 4-6 membered heterocycle containing one nitrogen atom, wherein the heterocycle is unsubstituted or is substituted with one or more R⁸. In some embodiments, R² is a heterocycle selected from azetidine and pyrrolidine, wherein the heterocycle is unsubstituted or is substituted with one or more R⁸. In some embodiments, R² is azetidine, wherein the azetidine is unsubstituted or is substituted with one or more R⁸. In some embodiments, R² is pyrrolidine, wherein the pyrrolidine is unsubstituted or is substituted with one or more R⁸. In some embodiments, R² is piperidine, wherein the piperidine is unsubstituted or is substituted with one or more R⁸. In some embodiments, R² is a 4-6 membered heterocycle containing one nitrogen atom, wherein the heterocycle is unsubstituted. In some embodiments, R² is a 4-6 membered heterocycle containing one nitrogen atom, wherein the heterocycle is substituted with one or more R⁸. In some embodiments, the one or more R⁸ are selected from C₁₋₆alkyl that is unsubstituted or substituted with one or more R¹¹. In some embodiments, at least one R⁸ is a halogen (e.g., F or Cl). In some embodiments, at least one R⁸ is C₁₋₆alkyl that is unsubstituted or substituted with one or more R¹¹. In some embodiments, at least one R⁸ is C₁₋₆alkyl that is unsubstituted. In some embodiments, at least one R⁸ is C₁₋₆alkyl that is substituted with an R¹¹ that is —OR¹², such as —OCH₃.

In some embodiments, for a compound according to any one of Formulas II, IIA, IIB, and IIC, R¹ is —OR⁷, wherein R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁷ is a heterocycle or an alkylheterocycle, wherein any heterocycle contains 4-8 members and is substituted with one or more R^{a} or R^{b}. In some embodiments, R⁷ is a heterocycle that is unsubstituted or substituted with one or more R^{a} or R^{b}. In some embodiments, R⁷ is an alkylheterocycle that is unsubstituted or substituted with one or more R^{a} or R^{b}. In some embodiments, R⁷ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR¹² (e.g., —OCH₃).

In some embodiments, for a compound according to any one of Formulas II, IIA, IIB, and IIC, R¹ is:

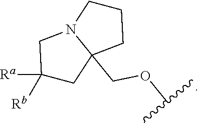

wherein R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³. In some embodiments, R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

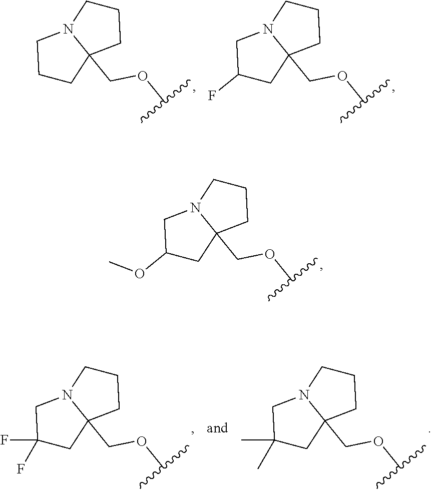 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

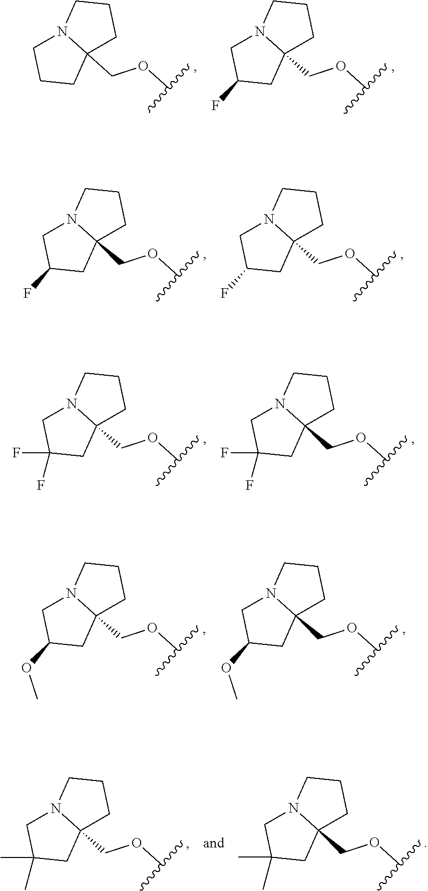

In some embodiments, for a compound according to any one of Formulas II, IIA, IIB, and IIC, R¹ is selected from:

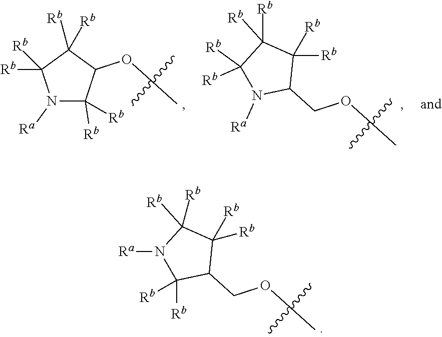

wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H, wherein an R^{a} and R^{c} join together to form a 3-6 membered heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆ alkyl, and —OR¹², and the other R^{a} and R^{b} groups are H. In some embodiments, one R^{a} or R^{b} is halogen (e.g., F). In some embodiments, two R^{a} groups, two R^{b} groups, or an W and an R^{b} are halogen (e.g., F). In some embodiments, one R^{a} or R^{b} is —OR¹² (e.g., —OCH₃ or —CHF₂). In some embodiments, one R^{a} or R^{b} is C₁₋₆ alkyl (e.g., methyl). In some embodiments, two R^{a} groups, two R^{b} groups, or an R^{a} and an R^{b} are C₁₋₆ alkyl (e.g., methyl). In some embodiments, R^{c} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{c} join together to form a 3-6 membered heterocycle. In some embodiments, R¹ is selected from:

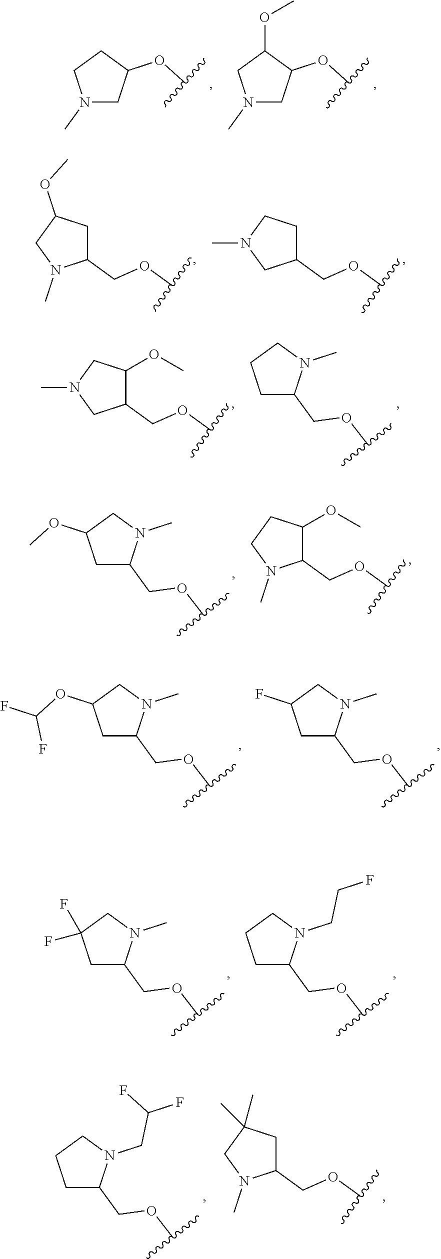

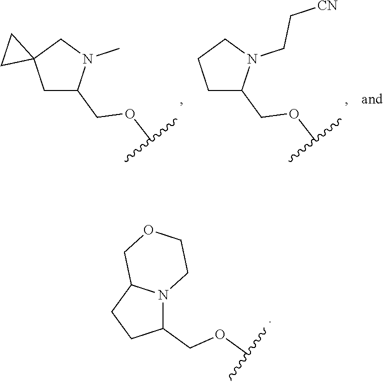 [SMILES: *OCC1CC2(CC2)CN1C ; *OCC1CCCN1CCC#N ; *OCC1CCC2COCCN12]

In some embodiments, R¹ is selected from:

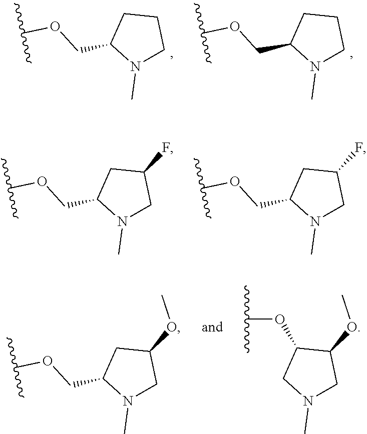

In some embodiments, for a compound according to any one of Formulas II, IIA, IIB, and IIC, R¹ is

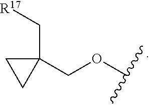

In some embodiments, R¹ is selected from:

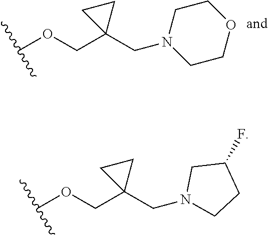 [SMILES: *OCC1(CN2CCOCC2)CC1 ; *OCC1(CN2CC[C@@H](F)C2)CC1]

In some embodiments, for a compound according to any one of Formulas II, IIA, IIB, and IIC, R¹ is a 4-6 membered heterocycle comprising a nitrogen atom, wherein the heterocycle is unsubstituted or substituted with one or more R¹⁵. In some embodiments, R¹ is a 4-membered heterocycle comprising a nitrogen atom, wherein the heterocycle is unsubstituted or substituted with one or more R¹⁵. In some embodiments, R¹ is selected from:

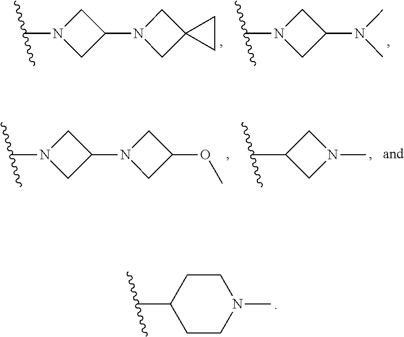

In some embodiments, for a compound according to any one of Formulas II, IIA, IIB, and IIC, R⁵ is selected from:

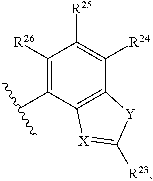

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂.

In some embodiments, for a compound according to any one of Formulas II, IIA, IIB, and IIC, R⁵ is selected from:

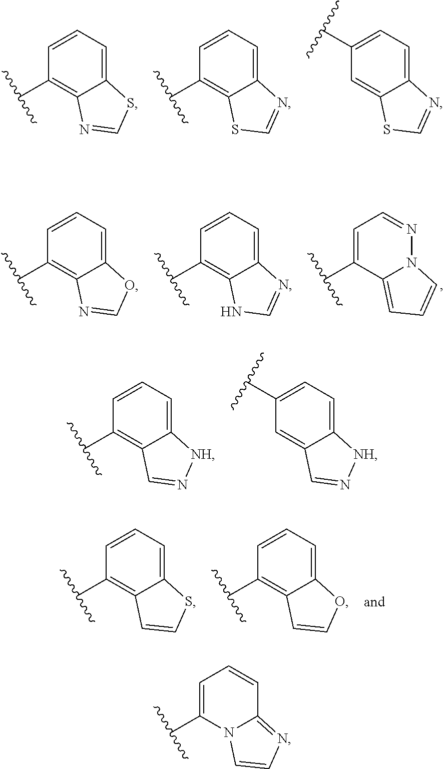

any of which is substituted with one or more R⁹.

In some embodiments, for a compound according to any one of Formulas II, IIA, IIB, and IIC, R⁵ is selected from:

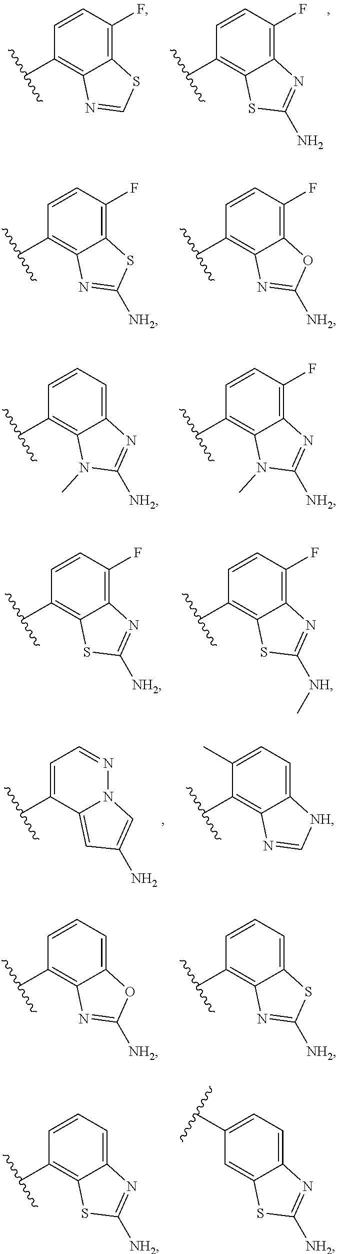

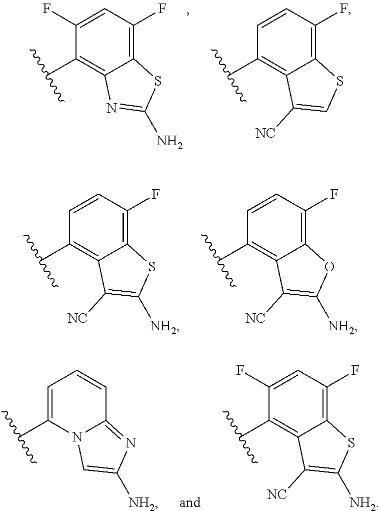 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12 ; *c1c(F)cc(F)c2sc(N)c(C#N)c12]

In some embodiments, for a compound according to any one of Formulas II, IIA, IIB, and IIC, R⁵ is selected from:

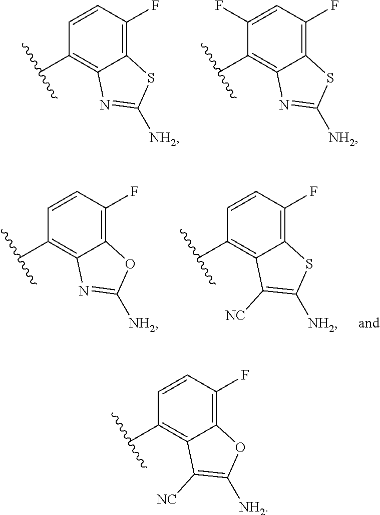 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12]

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combine to form a ring is mutually exclusive with an embodiment in which one group is ethyl and the other group is hydrogen. Similarly, an embodiment wherein one group is CH₂ is mutually exclusive with an embodiment wherein the same group is NH.

In some embodiments of any of the preceding aspects, the compound is a compound included in Table 2, or a salt (e.g., pharmaceutically acceptable salt), ester, tautomer, prodrug, zwitterionic form, or stereoisomer thereof. In some embodiments, the compound is a compound included in Table 2, or a salt (e.g., pharmaceutically acceptable salt) thereof. In some embodiments, the compound is a compound included in Table 2.

In some embodiments of any of the preceding aspects, the compound is a compound included in Table 3, or a salt (e.g., pharmaceutically acceptable salt), ester, tautomer, prodrug, zwitterionic form, or stereoisomer thereof. In some embodiments, the compound is a compound included in Table 3, or a salt (e.g., pharmaceutically acceptable salt) thereof. In some embodiments, the compound is a compound included in Table 3.

Also provided herein is a compound selected from Table 2 or Table 3 or any of the Examples provided herein, or a salt (e.g., pharmaceutically acceptable salt), ester, tautomer, prodrug, zwitterionic form, or stereoisomer thereof. In some embodiments, the present disclosure provides a compound selected from Table 2 or Table 3 or any of the Examples provided herein, or a salt (e.g., pharmaceutically acceptable salt) thereof.

In some embodiments of any of the preceding aspects, a compound provided herein, or a salt, ester, tautomer, zwitterionic form, or stereoisomer thereof, is capable of interacting with an aspartic acid (D) residue at the 12 position of the KRAS protein (e.g., a G12D mutation) (e.g., via an ion pair or other interaction). In some embodiments of any of the preceding aspects, a compound provided herein, or a salt, ester, tautomer, zwitterionic form, or stereoisomer thereof, binds selectively to KRAS having a G12D mutation relative to KRAS having other residues at the 12 position of the P loop, such as glycine (G), valine (V), arginine (R), serine (S), alanine (A), and cysteine (C). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12D mutation relative to KRAS having other residues at the 12 position of the P loop, such as glycine (G), valine (V), arginine (R), serine (S), alanine (A), and cysteine (C). In some embodiments of any of the preceding aspects, a compound provided herein, or a salt, ester, tautomer, zwitterionic form, or stereoisomer thereof, binds selectively to KRAS having a G12D mutation relative to wild-type KRAS. For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12D mutation relative to wild-type KRAS. In some embodiments of any of the preceding aspects, a compound provided herein, or a salt, ester, tautomer, zwitterionic form, or stereoisomer thereof, binds selectively to KRAS having a G12D mutation relative to other forms of RAS (e.g., HRAS and NRAS). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12D mutation relative to another form of RAS (e.g., HRAS or NRAS). In some embodiments of any of the preceding aspects, a compound provided herein, or a salt, ester, tautomer, zwitterionic form, or stereoisomer thereof, is capable of binding to a KRAS protein having a G12D mutation and one or more additional mutations, such as a mutation at the 13 position (to, e.g., D, A, R, S, V, or C).

In some embodiments of any of the preceding aspects, a compound provided herein, or a salt, ester, tautomer, zwitterionic form, or stereoisomer thereof, is capable of interacting with valine (V) residue at the 12 position of the KRAS protein (e.g., a G12V mutation) (e.g., via an ion pair or other interaction). In some embodiments of any of the preceding aspects, a compound provided herein, or a salt, ester, tautomer, zwitterionic form, or stereoisomer thereof, binds selectively to KRAS having a G12V mutation relative to KRAS having other residues at the 12 position of the P loop, such as glycine (G), aspartic acid (D), arginine (R), serine (S), alanine (A), and cysteine (C). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12V mutation relative to KRAS having other residues at the 12 position of the P loop, such as glycine (G), aspartic acid (D), arginine (R), serine (S), alanine (A), and cysteine (C). In some embodiments of any of the preceding aspects, a compound provided herein, or a salt, ester, tautomer, zwitterionic form, or stereoisomer thereof, binds selectively to KRAS having a G12V mutation relative to wild-type KRAS. For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12V mutation relative to wild-type KRAS. In some embodiments of any of the preceding aspects, a compound provided herein, or a salt, ester, tautomer, zwitterionic form, or stereoisomer thereof, binds selectively to KRAS having a G12V mutation relative to other forms of RAS (e.g., HRAS and NRAS). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12V mutation relative to another form of RAS (e.g., HRAS or NRAS). In some embodiments of any of the preceding aspects, a compound provided herein, or a salt, ester, tautomer, zwitterionic form, or stereoisomer thereof, is capable of binding to a KRAS protein having a G12V mutation and one or more additional mutations, such as a mutation at the 13 position (to, e.g., D, A, R, S, V, or C).

In some embodiments of any of the preceding aspects, a compound provided herein, or a salt, ester, tautomer, zwitterionic form, or stereoisomer thereof, is capable of interacting with glycine (G) residue at the 12 position of the KRAS protein (e.g., wild-type KRAS) (e.g., via an ion pair or other interaction). In some embodiments of any of the preceding aspects, a compound provided herein, or a salt, ester, tautomer, zwitterionic form, or stereoisomer thereof, binds selectively to wild-type KRAS relative to KRAS having other residues at the 12 position of the P loop, such as valine (V), aspartic acid (D), arginine (R), serine (S), alanine (A), and cysteine (C). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for wild-type KRAS relative to KRAS having other residues at the 12 position of the P loop, such as valine (V), aspartic acid (D), arginine (R), serine (S), alanine (A), and cysteine (C). In some embodiments of any of the preceding aspects, a compound provided herein, or a salt, ester, tautomer, zwitterionic form, or stereoisomer thereof, binds selectively to wild-type relative to a mutated form of KRAS. For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for wild-type KRAS relative to a mutated form of KRAS. In some embodiments of any of the preceding aspects, a compound provided herein, or a salt, ester, tautomer, zwitterionic form, or stereoisomer thereof, binds selectively to wild-type KRAS relative to other forms of RAS (e.g., HRAS and NRAS). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for wild-type KRAS relative to another form of RAS (e.g., HRAS or NRAS). In some embodiments of any of the preceding aspects, a compound provided herein, or a salt, ester, tautomer, zwitterionic form, or stereoisomer thereof, is capable of binding to wild-type KRAS protein and KRAS having one or more mutations, such as a mutation at the 12 position (to, e.g., D, A, R, S, V, or C) or a mutation at the 13 position (to, e.g., D, A, R, S, V, or C).

In some embodiments of any of the preceding aspects, a compound provided herein, or a salt (e.g., pharmaceutically acceptable salt), ester, tautomer, zwitterionic form, or stereoisomer thereof, is capable of selectively binding a KRAS protein in an active (GTP-bound) conformation. In some embodiments of any of the preceding aspects, a compound provided herein, or a salt, ester, tautomer, zwitterionic form, or stereoisomer thereof, is capable of selectively binding a KRAS protein in an inactive (GDP-bound) conformation. In some embodiments of any of the preceding aspects, a compound provided herein, or a salt, ester, tautomer, zwitterionic form, or stereoisomer thereof, is capable of selectively binding a KRAS protein in both active (GTP-bound) and inactive (GDP-bound) conformations. In some embodiments of any of the preceding aspects, a compound provided herein, or a salt, ester, tautomer, zwitterionic form, or stereoisomer(s) thereof, has higher selectivity for a KRAS protein in its active (GTP-bound) conformation than in its inactive (GDP-bound) conformation. In some embodiments of any of the preceding aspects, a compound provided herein, or a salt, ester, tautomer, zwitterionic form, or stereoisomer(s) thereof, has higher selectivity for a KRAS protein in its inactive (GDP-bound) conformation than in its active (GTP-bound) conformation.

### Compositions

The present disclosure also provides a composition (e.g., a pharmaceutical composition) comprising a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof. For example, the present disclosure provides a pharmaceutical composition comprising a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a pharmaceutically acceptable salt, ester, amide, prodrug or solvate thereof (“active ingredient”), with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, IA, IA1, IA2, IA3, IA4, IA5, IB, IB1, IB2, IB3, IB4, IB5, IB6, IB7, IB8, IB9, IC, II, IIA, IIB, or IIC in any available form (e.g., salt, ester, tautomer, prodrug, zwitterionic form, stereoisomer etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, sealed capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compounds may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof (e.g., salt, ester, tautomer, prodrug, zwitterionic form, stereoisomer, etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof (e.g., salt, ester, tautomer, prodrug, zwitterionic form, stereoisomer etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, IA, IA1, IA2, IA3, IA4, IA5, IB, IB1, IB2, IB3, IB4, IB5, IB6, IB7, IB8, IB9, IC, II, IIA, IIB, or IIC in any suitable form (e.g., salt, ester, tautomer, prodrug, zwitterionic form, stereoisomer, etc.)), may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC) or a form thereof (e.g., salt, ester, tautomer, prodrug, zwitterionic form, stereoisomer, etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer, etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC or any suitable forms thereof (e.g., salt, ester, tautomer, prodrug, zwitterionic form, stereoisomer, etc.)) may be formulated for non systematic administration, such as topical administration. This includes the application of a compound disclosed herein, or a form thereof, externally to the epidermis or the buccal cavity and the instillation of such a compound, or a form thereof, into the ear, eye and nose, such that the compound, or an alternative thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear, or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, IA, IA1, IA2, IA3, IA4, IA5, IB, IB1, IB2, IB3, IB4, IB5, IB6, IB7, IB8, IB9, IC, II, IIA, IIB, or IIC or any suitable forms thereof (e.g., salt, ester, tautomer, prodrug, zwitterionic form, stereoisomer, etc.)) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as described herein, or an appropriate fraction thereof, of the active ingredient (e.g., a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof).

It should be understood that in addition to the ingredients particularly described elsewhere herein, the formulations described herein may include other useful agents having regard to the type of formulation in question, for example those suitable for oral administration may include flavoring agents.

Compounds (e.g., compounds of any one of Formulas I, IA, IA1, IA2, IA3, IA4, IA5, IB, IB1, IB2, IB3, IB4, IB5, IB6, IB7, IB8, IB9, IC, II, IIA, IIB, or IIC) or forms thereof (e.g., salt, ester, tautomer, prodrug, zwitterionic form, stereoisomer, etc.) may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

### Methods

The present disclosure also provides a method of modulating (e.g., inhibiting) KRAS (e.g., KRAS having a G12D or G12V mutation or wild-type KRAS) comprising contacting KRAS with a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof. For example, the present disclosure may provide a method of altering a cell phenotype, cell proliferation, KRAS activity, biochemical output produced by active or inactive KRAS, expression of KRAS, and/or binding of KRAS with a natural binding partner. Any such feature may be monitored and may be altered upon contacting KRAS with a compound provided herein, or a form thereof (e.g., a salt, ester, tautomer, zwittenonic form, or stereoisomer thereof). A method of modulating (e.g., inhibiting) KRAS (e.g., KRAS having a G12D or G12V mutation or wild-type KRAS) may be a mode of treatment of a disease, disorder, or condition (e.g., a cancer), a biological assay, a cellular assay, a biochemical assay, etc. In some embodiments, a method of modulating (e.g., inhibiting) KRAS (e.g., KRAS having a G12D or G12V mutation or wild-type KRAS) comprises contacting a KRAS protein with a compound provided herein, or a salt, ester, tautomer, zwitterionic form, or stereoisomer thereof, where the KRAS protein is in the active (GTP-bound) conformation. In some embodiments, a method of modulating (e.g., inhibiting) KRAS (e.g., KRAS having a G12D or G12V mutation or wild-type KRAS) comprises contacting a KRAS protein with a compound provided herein, or a salt, ester, tautomer, zwitterionic form, or stereoisomer thereof, where the KRAS protein is in the inactive (GDP-bound) conformation. In some embodiments, contacting a KRAS protein with a compound provided herein, or a salt, ester, tautomer, zwitterionic form, or stereoisomer thereof, comprises incubating the KRAS protein with the compound or form thereof. In some embodiments, contacting a KRAS protein with a compound provided herein, or a salt, ester, tautomer, zwittenonic form, or stereoisomer thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a G12D or G12V mutation or by amplified wild-type KRAS.

The present disclosure also provides methods of treating a disease, disorder, or condition in a subject in need thereof using a compound provided herein, (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof. For example, the present disclosure provides a method comprising providing (e.g., administering) to a subject (e.g., patient) in need thereof an effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof. The present disclosure also provides methods of treating a disease, disorder, or condition in a subject in need thereof using a pharmaceutical composition comprising a compound provided herein, (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof. For example, the present disclosure provides a method comprising providing (e.g., administering) to a subject (e.g., patient) in need thereof a pharmaceutical composition comprising an effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof. In some embodiments, the subject is known to have (e.g., has previously been diagnosed with) a disease, disorder, or condition such as a cancer. The disease, disorder, or condition may be a KRAS-mediated disease, such as a cancer characterized by a G12D or G12V mutation in KRAS or by amplified wild-type KRAS. In some embodiments, the compound administered to the subject in need thereof according to the methods described herein is a compound described in an embodiment, example, figure, or table herein, or a stereoisomer or pharmaceutically acceptable salt thereof.

The present disclosure also provides a compound as provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, for use as a medicament, such as a medicament for the treatment of a disease, disorder, or condition (e.g., a cancer).

The present disclosure also provides a compound as provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for the treatment of a disease, disorder, or condition (e.g., a cancer) in a subject in need thereof.

The present disclosure also provides the use of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, for the treatment of a disease, disorder, or condition (e.g., a cancer) in a subject in need thereof.

The present disclosure also provides the use of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for treating a disease, disorder, or condition (e.g., a cancer) in a subject in need thereof.

The present disclosure also provides a method of inhibiting KRAS (e.g., KRAS having a G12D or G12V mutation or wild-type KRAS) (e.g., in a subject in need thereof) comprising contacting KRAS with a compound as provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwittenonic form, or stereoisomer thereof, or a pharmaceutical composition comprising any of the foregoing compounds and a pharmaceutically acceptable excipient. In some embodiments, a method of inhibiting KRAS (e.g., KRAS having a G12D or G12V mutation or wild-type KRAS) comprises contacting a KRAS protein with a compound provided herein, or a salt, ester, tautomer, zwittenonic form, or stereoisomer thereof, where the KRAS protein is in the active (GTP-bound) conformation. In some embodiments, a method of inhibiting KRAS (e.g., KRAS having a G12D or G12V mutation or wild-type KRAS) comprises contacting a KRAS protein with a compound provided herein, or a salt, ester, tautomer, zwitterionic form, or stereoisomer thereof, where the KRAS protein is in the inactive (GDP-bound) conformation. In some embodiments, contacting a KRAS protein with a compound provided herein, or a salt, ester, tautomer, zwitterionic form, or stereoisomer thereof, comprises incubating the KRAS protein with the compound or form thereof. In some embodiments, contacting a KRAS protein with a compound provided herein, or a salt, ester, tautomer, zwitterionic form, or stereoisomer thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a G12D or G12V mutation or by amplified wild-type KRAS.

The present disclosure also provides a compound as provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, for use in inhibiting KRAS (e.g., KRAS having a G12D or G12V mutation or wild-type KRAS) (e.g., in a subject in need thereof). The present disclosure also provides a compound as provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for inhibiting KRAS (e.g., KRAS having a G12D or G12V mutation or wild-type KRAS) in a subject in need thereof.

The present disclosure also provides the use of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, for inhibiting KRAS (e.g., KRAS having a G12D or G12V mutation or wild-type KRAS) in a subject in need thereof.

The present disclosure also provides the use of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for inhibiting KRAS (e.g., KRAS having a G12D or G12V mutation or wild-type KRAS) in a subject in need thereof.

The present disclosure also provides a method comprising administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof to a subject (e.g., patient), thereby ameliorating, reducing, eliminating, ceasing, or improving one or more symptoms of the subject, such as one or more symptoms of a disease, disorder, or condition (e.g., a cancer). In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, slows or prevents growth of a tumor. In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer thereof, results in shrinkage of a tumor (e.g., tumor regression). In some embodiments, the subject has a cancer characterized by a mutant KRAS (e.g., KRAS having a G12D or G12V mutation) or by amplified wild-type KRAS.

In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer(s) thereof, slows or prevents growth of a tumor. In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer(s) thereof, results in shrinkage of a tumor (e.g., tumor regression). In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer(s) thereof, results in at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% regression of a tumor, such as for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer(s) thereof, stabilizes a tumor. In some embodiments, administering a therapeutically effective amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a salt, ester, tautomer, prodrug, zwitterionic form, or stereoisomer(s) thereof, stabilizes a tumor for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, the subject has a cancer characterized by a mutant KRAS (e.g., KRAS protein having a G12D or G12V mutation) or by amplified wild-type KRAS.

In some embodiments of any of the methods, uses, and medicaments provided herein, the disease, disorder, or condition is a cancer. In some embodiments, the cancer is pancreatic cancer (e.g., pancreatic ductal adenocarcinoma), lung cancer (e.g., non-small cell lung cancer (NSCLC)), colorectal cancer (CRC), endometrial cancer, uterine carcinosarcoma, Ewing sarcoma, osteosarcoma, Rhabdomyosarcoma, adrenocortical carcinoma, neuroblastoma, Wilm tumor, retinoblastoma, skin cancer, breast cancer, prostate cancer, head and neck cancer, or ovarian cancer. In some embodiments, the cancer is endometrial endometrioid adenocarcinoma, rectal adenocarcinoma, or gastric cancer. In some embodiments, the cancer is pancreatic cancer (e.g., pancreatic ductal adenocarcinoma), lung cancer (e.g., non-small cell lung cancer adenocarcinoma), or colorectal cancer (CRC). In some embodiments, the cancer is pancreatic cancer (e.g., pancreatic ductal adenocarcinoma). In some embodiments, the cancer is lung cancer (e.g., non-small cell lung cancer adenocarcinoma). In some embodiments, the cancer is colorectal cancer (CRC). In some embodiments, the cancer is or comprises a solid tumor.

In some embodiments of any of the methods, uses, and medicaments provided herein, the disease, disorder, or condition is a RASopathy (e.g., a genetic syndrome caused by a germline mutation in a gene that encodes a component or regulator of the RAS/MAPK pathway). In some embodiments, the RASopathy is selected from the group consisting of neurofibromatosis type 1, Noonan syndrome, Noonan syndrome with multiple lentigines, capillary malformation-arteriovenous malformation syndrome, Costello syndrome, cardio-facio-cutaneous syndrome, and Legius syndrome.

In some embodiments of any of the methods, uses, and medicaments provided herein, the disease, disorder, or condition is related to KRAS, such as a disorder associated with a mutation of KRAS or dysregulation of KRAS. In some embodiments, the disease, disorder, or condition is related to the KRAS gene, such as a disease, disorder, or condition associated with a mutation of the KRAS gene or dysregulation of the KRAS gene. Mutation or dysregulation of KRAS or KRAS may include mutation or dysregulation of human K-Ras4a and/or human K-Ras4b. In some embodiments, the disease, disorder, or condition is related to the KRAS (e.g., human K-Ras4a or K-Ras4b) signaling pathway activity, such as a disease, disorder, or condition related to aberrant KRAS signaling pathway activity. In some embodiments, the disease, disorder, or condition is related to mutation or dysregulation of human K-Ras4b. In some embodiments, the disease, disorder, or condition is related to aberrant K-Ras4b signaling pathway activity. In some embodiments, the disease, disorder, or condition is related to mutation or dysregulation of human K-Ras4a. In some embodiments, the disease, disorder, or condition is related to aberrant K-Ras4a signaling pathway activity.

### Administration and Combination Therapy

The compounds provided herein (e.g., compounds of any one of Formulas I, IA, IA1, IA2, IA3, IA4, IA5, IB, IB1, IB2, IB3, IB4, IB5, IB6, IB7, IB8, IB9, IC, II, IIA, IIB, or IIC or any suitable form thereof (e.g., salt, ester, tautomer, prodrug, zwitterionic form, stereoisomer, etc.)), or compositions (e.g., pharmaceutical compositions) comprising the same, can be administered in various modes (e.g., orally, topically, or by injection). The amount of active ingredient (e.g., compound provided herein or a form thereof) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof (e.g., salt, ester, tautomer, prodrug, zwitterionic form, stereoisomer, etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof, may be enhanced by administration of the compound, or a form thereof, and an additional agent (which may comprise an additional therapeutic regimen) that also provides a therapeutic benefit. For example, a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof, may be administered in combination with an additional agent that may be effective in the treatment of a disease, disorder, or condition such as a cancer. Generally, the combination of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof, and one or more additional agents (e.g., therapeutic agents) may enhance the overall benefit experienced by the subject upon either component individually. In some embodiments, the effect may be additive. In some embodiments, the effect may be synergistic.

In some embodiments, a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof (e.g., salt, ester, tautomer, prodrug, zwitterionic form, stereoisomer, etc.) is administered in combination with an anti-cancer agent (e.g., chemotherapeutic agent). An anti-cancer agent may be, for example, an alkylating agent, an antimitotic, a checkpoint inhibitor, an anti-metabolite, a plant alkaloid, a terpenoid, a cytotoxic an antibiotic, a topoisomerase inhibitor, an aromatase inhibitor, an angiogenesis inhibitor, an anti-steroid, an anti-androgen, an mTOR inhibitor, monoclonal antibodies, or a tyrosine kinase inhibitor. An alkylating agent may be, for example, armustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, or cyclophosphamide (ENDOXAN). An anti-metabolite may be, for example, cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEY), methotrexate (RHEUMATREX), or raltitrexed. An antimitotic may be, for example, a taxane such as docetaxel (TAXITERE) or paclitaxel (ABRAXANE, TAXOL), or a vinca alkaloid such as vincristine (ONCOVIN), vinblastine, vindesine, or vinorelbine (NAVELBINE). A checkpoint inhibitor may be an anti-PD-1 or anti-PD-L1 antibody such as pembrolizumab (KEYTRUDA), nivolumab (OPDIVO), MEDI4736, or MPDL3280A; anti-CTLA-4 antibody ipilimumab (YERVOY); or an agent that targets LAG3 (lymphocyte activation gene 3 protein), KIR (killer cell immunoglobulin-like receptor), 4-IBB (tumor necrosis factor receptor superfamily member 9), TIM3 (T-cell immunoglobulin and mucin-domain containing-3), or 0X40 (tumor necrosis factor receptor superfamily member 4). A topoisomerase inhibitor may be, for example, camptothecin (CTP), irinotecan (CAMPTOSAR), topotecan (HYCAMTIN), teniposide (VUMON), or etoposide (EPOSIN). A cytotoxic antibiotic may be, for example, actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOYANTRONE), or plicamycin. An aromatase inhibitor may be, for example, aminoglutethimide, anastrozole (ARIMIDEX), letrozole (FEMARA), vorozole (RIYIZOR), or exemestane (AROMASIN). An angiogenesis inhibitor may be, for example, genistein, sunitinib (SUTENT), or bevacizumab (AYASTIN). An anti-steroid or anti-androgen may be, for example, aminoglutethimide (CYTADREN), bicalutamide (CASODEX), cyproterone, flutamide (EULEXIN), or nilutamide (NILANDRON). A tyrosine kinase inhibitor may be, for example, imatinib (GLEEVEC), erlotinib (TARCEVA), afatinib (GILOTRIF), lapatinib (TYKERB), sorafenib (NEXAVAR), or axitinib (INLYTA). An mTOR inhibitor may be, for example, everolimus, temsirolimus (TORISEL), or sirolimus. Monoclonal antibody may be, for example, trastuzumab (HERCEPTIN) or rituximab (RITUXAN). Additional examples of agents that may be useful in combination with a compound provided herein, or a form thereof (e.g., salt, ester, tautomer, prodrug, zwitterionic form, stereoisomer, etc.), include, but are not limited to, amsacrine; Bacillus Calmette-Guerin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,r′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

Two or more therapeutic agents, one of which is a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC) or a form thereof, may be administered in any order or may be administered simultaneously. If administered simultaneously, the multiple therapeutic agents may be provided in a single, unified form, or in multiple forms (such as, for example, as a single pill or as two separate pills). One of the therapeutic agents may be given in multiple doses, or both may be given as multiple doses. If not administered simultaneously, the timing between the multiple doses may be any duration of time ranging from a few minutes to four weeks.

Accordingly, in another aspect, the present disclosure provides a method for treating a disease, disorder, or condition (e.g., a cancer) in a subject (e.g., a human or animal subject) in need of such treatment comprising administering to the subject an amount of a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof (e.g., salt, ester, tautomer, prodrug, zwitterionic form, stereoisomer, etc.), in combination with at least one additional agent for the treatment of the disease, disorder, or condition. In a related aspect, the present disclosure provides a composition (e.g., pharmaceutical composition) comprising a compound provided herein (e.g., a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC), or a form thereof (e.g., salt, ester, tautomer, prodrug, zwitterionic form, stereoisomer, etc.), and at least one additional agent for use in the treatment of a disease, disorder, or condition (e.g., a cancer).

In some embodiments, a method provided herein is used to treat a disease, disorder, or condition (e.g., a cancer) comprising administering to a subject in need thereof a therapeutically effective amount of a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC or a pharmaceutically acceptable salt thereof, wherein the disease, disorder, or condition is a cancer that has developed a resistance to one or more chemotherapeutic drugs and/or ionizing radiation. In some embodiments, a method provided herein is used to treat a disease, disorder, or condition (e.g., a cancer) comprising administering to a subject in need thereof a therapeutically effective amount of a compound of any one of Formulas I, IA, IA1, IA2, IA3, IA4, IA5, IB, IB1, IB2, IB3, IB4, IB5, IB6, IB7, IB8, IB9, IC, II, IIA, IIB, or IIC or a pharmaceutically acceptable salt thereof, in combination with an additional agent, wherein the disease, disorder, or condition is a cancer that has developed a resistance to one or more chemotherapeutic drugs and/or ionizing radiation.

The compounds, compositions, and methods disclosed herein are useful for the treatment of a disease, disorder, or condition, such as a cancer. In certain embodiments, the disease is one of dysregulated cellular proliferation, including cancer. The cancer may be hormone-dependent or hormone-resistant, such as in the case of breast cancers. In certain embodiments, the cancer is or comprises a solid tumor. In other embodiments, the cancer is a lymphoma or leukemia. In certain embodiments, the cancer is a drug resistant phenotype of a cancer disclosed herein or otherwise known. Tumor invasion, tumor growth, tumor metastasis, and angiogenesis may also be treated using the compositions and methods disclosed herein. In some embodiments, the compounds, compositions, and methods provided herein are also useful in the treatment of precancerous neoplasias.

Cancers that may be treated by the methods disclosed herein include, but are not limited to, pancreatic cancer, colon cancer, rectal cancer, colorectal cancer, breast cancer, ovarian cancer, endometrial cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas. Additional types of cancers which may be treated using the compounds and methods provided herein include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. Additional diseases and disorders that may be treated by the methods disclosed herein include, but are not limited to, diseases or disorders related to KRAS, such as diseases or disorders associated with a mutation of KRAS or dysregulation of KRAS, and diseases or disorders related to the KRAS gene, such as diseases or disorders associated with a mutation of the KRAS gene or dysregulation of the KRAS gene.

In some embodiments, the compounds, compositions, and methods provided herein are useful in the prevention and/or reduction of tumor invasion, growth, and/or metastasis.

The compounds, compositions, and methods provided herein may be useful in the treatment of humans as well as in the veterinary treatment of non-human animals including companion animals, exotic animals, and farm animals (e.g., as described herein), including mammals, rodents, and the like. For example, the compounds, compositions, and methods provided herein may be useful in the treatment of horses, dogs, or cats.

### Exemplary Embodiments

1. A compound according to Formula I:

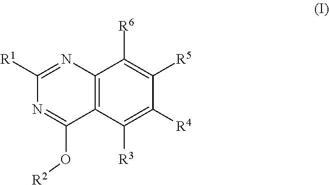

or a salt (e.g., pharmaceutically acceptable salt), ester, tautomer, prodrug, zwitterionic form, or stereoisomer thereof, wherein:
   R¹ is selected from —OR⁷;
   R² is a 4-11 membered heterocycle containing one or more nitrogen atoms, wherein the heterocycle is unsubstituted or is substituted with one or more R⁸;
   R³ is H;
   R⁴ is selected from H, halogen, —CN, —OR¹², and C₁₋₆alkyl, wherein any C₁₋₆alkyl is unsubstituted or substituted with one or more R¹¹;
   R⁵ is a bicyclic heteroaryl substituted with one or more R⁹;
   R⁶ is selected from halogen, —OR¹², —CN, and H;
   R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
   each R⁸ is independently selected from halogen and C₁₋₆alkyl, wherein any C₁₋₆alkyl is unsubstituted or substituted with one or more R¹¹;
   each R⁹ is independently selected from halogen, N(R¹²)₂, —CN, and C₁₋₆alkyl, wherein any C₁₋₆alkyl is unsubstituted or substituted with one or more R¹³;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H; and R^{a} and R^{b} are each independently selected from halogen, C₁₋₆ alkyl, —OR¹², and H, wherein any C₁₋₆alkyl is unsubstituted or is substituted with one or more R¹³
2. The compound of embodiment 1, wherein R² is a 4-6 membered heterocycle containing one nitrogen atom, wherein the heterocycle is unsubstituted or is substituted with one or more R⁸.
3. The compound of embodiment 2, wherein R² is a heterocycle selected from azetidine and pyrrolidine, wherein the heterocycle is unsubstituted or is substituted with one or more R⁸.
4. The compound of embodiment 1, wherein R² is a bridged heterocycle containing one nitrogen atom, wherein the heterocycle is unsubstituted or is substituted with one or more R⁸.
5. The compound of embodiment 1, wherein R² is a spirocycle including a first ring that is a carbocycle and a second ring that is a heterocycle containing a nitrogen atom, wherein the spirocycle is unsubstituted or is substituted with one or more R⁸.
6. The compound of any one of embodiments 1-5, wherein R² is unsubstituted.
7. The compound of any one of embodiments 1-5, wherein R² is substituted with one or more R⁸, wherein the one or more R⁸ are each independently selected from halogen and C₁₋₆alkyl, wherein any C₁₋₆alkyl is unsubstituted.
8. The compound of any one of embodiments 1-7, wherein R¹ is selected from:

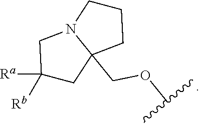

9. The compound of embodiment 8, wherein R¹ is selected from:

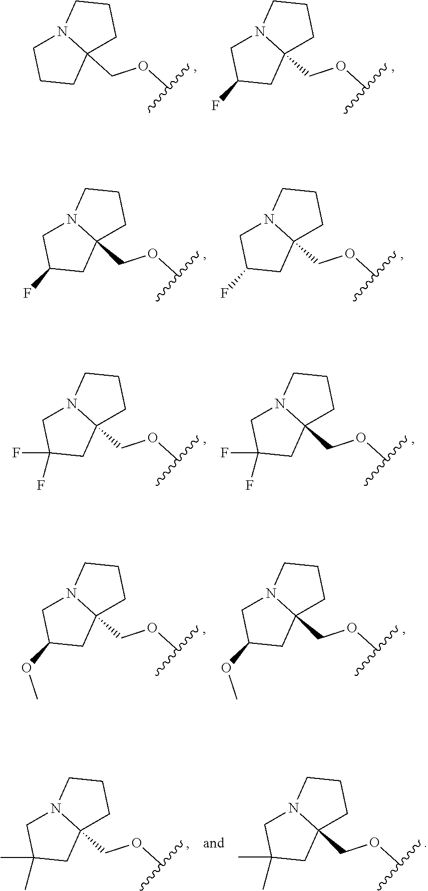

10. The compound of any one of embodiments 1-7, wherein R¹ is selected from:

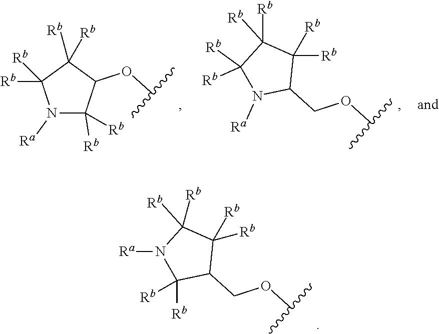

11. The compound of embodiment 10, wherein R¹ is selected from:

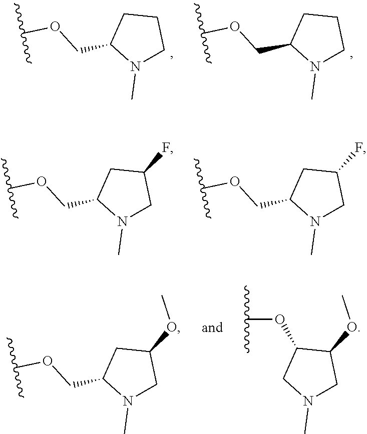

12. The compound of any one of embodiments 1-7, wherein R¹ is selected from:

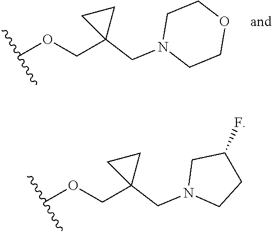 [SMILES: *OCC1(CN2CCOCC2)CC1 ; *OCC1(CN2CC[C@@H](F)C2)CC1]

13. The compound of any one of embodiments 1-12, wherein R⁴ is H.
14. The compound of any one of embodiments 1-12, wherein R⁴ is selected from halogen, —CN, —OR¹², and C₁₋₆alkyl, wherein any C₁₋₆alkyl is unsubstituted or substituted with one or more R¹¹.
15. The compound of embodiment 14, wherein R⁴ is —CN.
16. The compound of embodiment 14, wherein R⁴ is a halogen.
17. The compound of embodiment 14, wherein R⁴ is C₁₋₆alkyl that is substituted with one or more R¹¹.
18. The compound of any one of embodiments 1-17, wherein R⁵ is selected from:

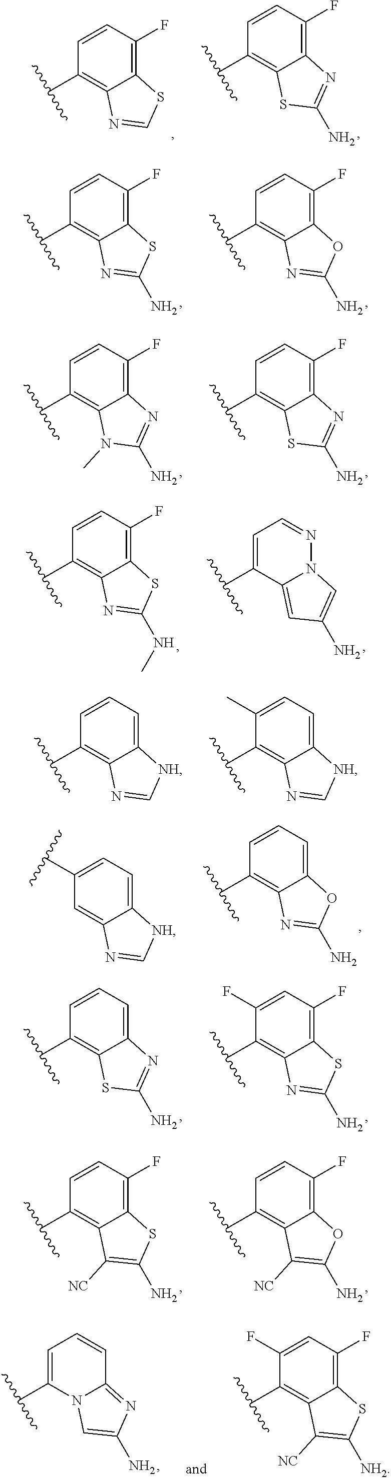

19. The compound of embodiment 18, wherein R⁵ is selected from:

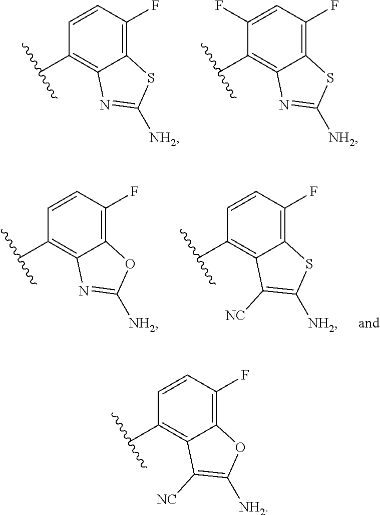 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12]

20. The compound of any one of embodiments 1-19, wherein R⁶ is selected from halogen, —CN, and H.
21. The compound of embodiment 20, wherein R⁶ is a halogen.
22. A compound according to Formula IA:

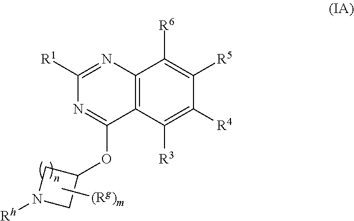

or a salt (e.g., pharmaceutically acceptable salt), ester, tautomer, prodrug, zwitterionic form, or stereoisomer thereof, wherein:
   R¹ is selected from —OR⁷;
   R³ is H;
   R⁴ is selected from H, halogen, —CN, —OR¹², and C₁₋₆alkyl, wherein any C₁₋₆alkyl is unsubstituted or substituted with one or more R¹¹;
   R⁵ is a bicyclic heteroaryl substituted with one or more R⁹;
   R⁶ is selected from halogen, —OR¹², —CN, and H;
   R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
   each R⁹ is independently selected from halogen, N(R¹²)₂, —CN, and C₁₋₆alkyl, wherein any C₁₋₆alkyl is unsubstituted or substituted with one or more R¹³;
   each R¹⁰ is independently selected from C₁₋₆ alkyl, C₂₋₆ alkenyl, and H;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H;
   n is 1, 2, or 3;
   m is 0, 1, 2, 3, or 4;
   each R⁹ is independently selected from halogen, C₁₋₆alkyl, and —OR¹⁰, wherein any C₁₋₆alkyl is unsubstituted or substituted with one or more R¹¹, or two R^{g}, together with the atoms to which they are attached, join to form a 5-7 membered ring;
   R^{h} is selected from H and C₁₋₆alkyl, wherein any C₁₋₆alkyl is unsubstituted or substituted with one or more R¹¹; and
   R^{a} and R^{b} are each independently selected from halogen, C₁₋₆ alkyl, —OR¹², and H, wherein any C₁₋₆alkyl is unsubstituted or is substituted with one or more R¹³.
23. The compound of embodiment 22, wherein the compound has the structure:

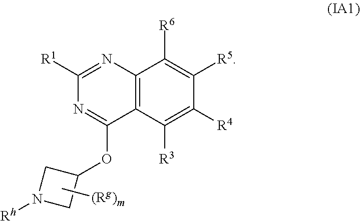

24. The compound of embodiment 22, wherein the compound has the structure:

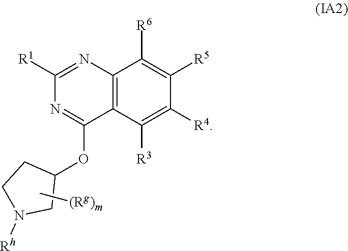

25. The compound of embodiment 22, wherein the compound has the structure:

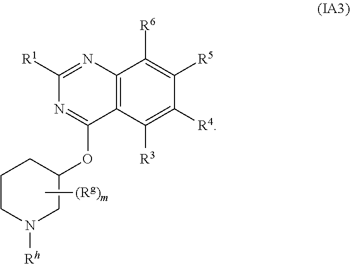

26. The compound of embodiment 22, wherein the compound has the structure:

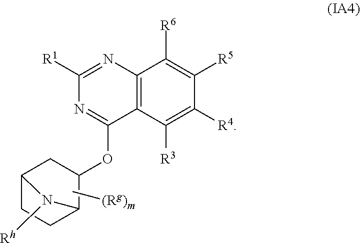

27. The compound of embodiment 22, wherein the compound has the structure:

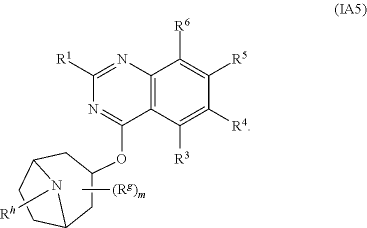

28. The compound of any one of embodiments 22-27, wherein R¹¹ is H.
29. The compound of any one of embodiments 22-28, wherein m is 1 or 2, and each R^{g} is independently selected from halogen and C₁₋₆alkyl, wherein any C₁₋₆alkyl is unsubstituted.
30. The compound of any one of embodiments 22-29, wherein R¹ is selected from:

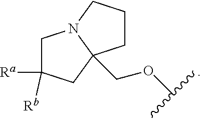

31. The compound of embodiment 30, wherein R¹ is selected from:

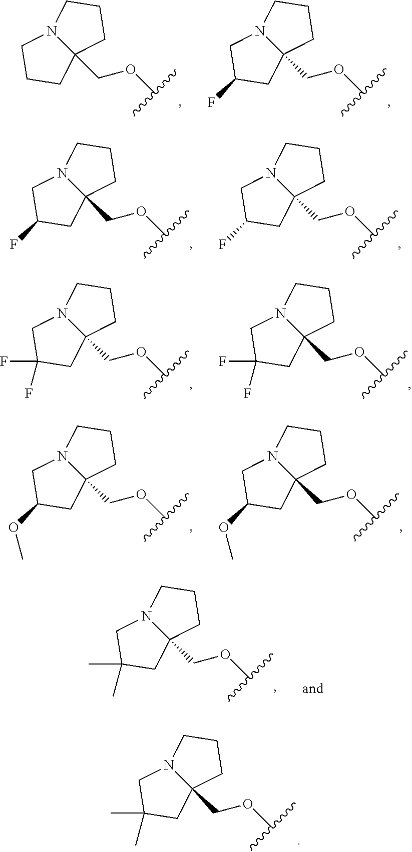

32. The compound of any one of embodiments 22-29, wherein R¹ is selected from:

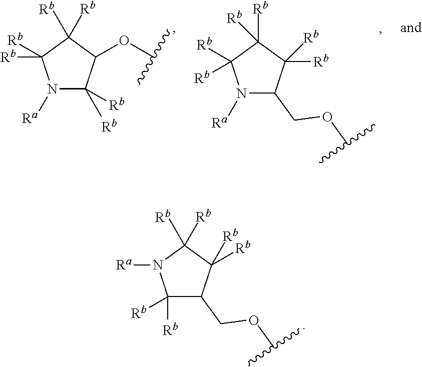

33. The compound of embodiment 32, wherein R¹ is selected from:

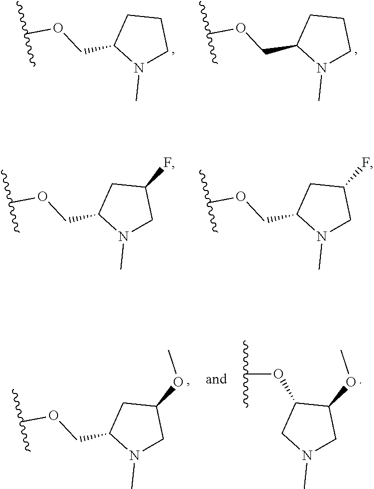

34. The compound of any one of embodiments 22-29, wherein R¹ is selected from:

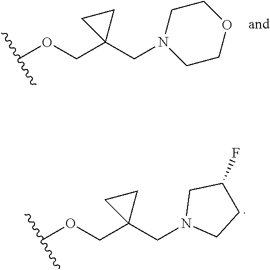 [SMILES: *OCC1(CN2CCOCC2)CC1 ; *OCC1(CN2CC[C@@H](F)C2)CC1]

35. The compound of any one of embodiments 22-34, wherein R⁴ is H.
36. The compound of any one of embodiments 22-34, wherein R⁴ is selected from halogen, —CN, —OR¹², and C₁₋₆alkyl, wherein any C₁₋₆alkyl is unsubstituted or substituted with one or more R¹¹.
37. The compound of embodiment 36, wherein R⁴ is —CN.
38. The compound of embodiment 36, wherein R⁴ is a halogen.
39. The compound of embodiment 36, wherein R⁴ is C₁₋₆alkyl that is substituted with one or more R¹¹.
40. The compound of any one of embodiments 22-39, wherein R⁵ is selected from:

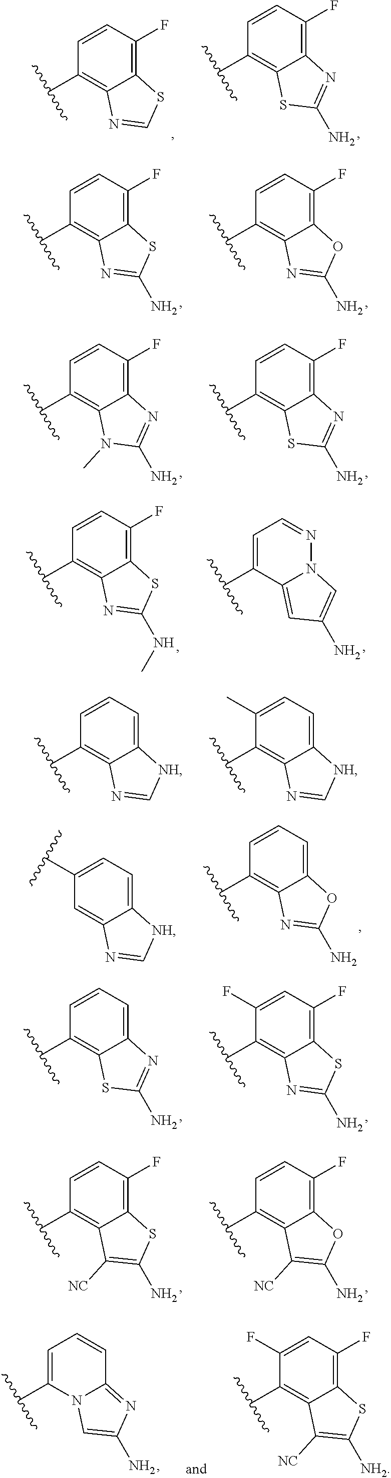

41. The compound of embodiment 40, wherein R⁵ is selected from:

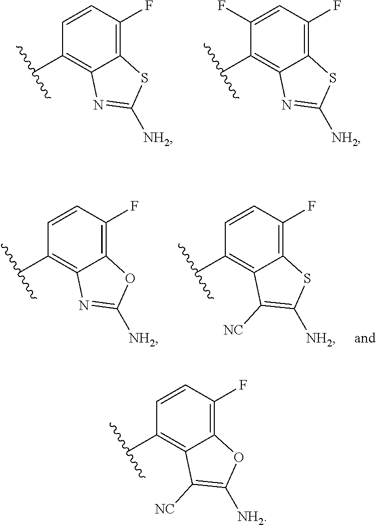 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; "and" ; *c1ccc(F)c2oc(N)c(C#N)c12]

42. The compound of any one of embodiments 22-41, wherein R⁶ is selected from halogen, —CN, and H.
43. The compound of embodiment 42, wherein R⁶ is a halogen.
44. A compound according to Formula IB:

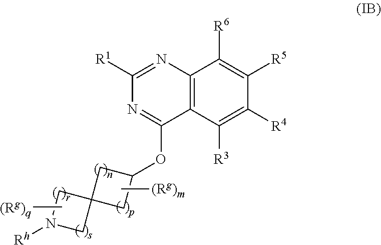

R¹ is selected from —OR⁷;
R³ is H;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁹ is independently selected from halogen, N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OH, and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
m and q are each independently selected from 0, 1, 2, 3, and 4;
n and p are each independently selected from 1 and 2;
r and s are each independently selected from 1 and 2;
each R⁹ is independently selected from halogen, C₁₋₆alkyl, and —OR¹⁰, wherein any C₁₋₆alkyl is unsubstituted or substituted with one or more R¹¹;
R^{h} is selected from H and C₁₋₆alkyl, wherein any C₁₋₆alkyl is unsubstituted or substituted with one or more R¹¹; and
R^{a} and R^{b} are each independently selected from halogen, C₁₋₆ alkyl, —OR¹², and H, wherein any C₁₋₆alkyl is unsubstituted or is substituted with one or more R¹³.
45. The compound of embodiment 44, wherein the compound has the structure:

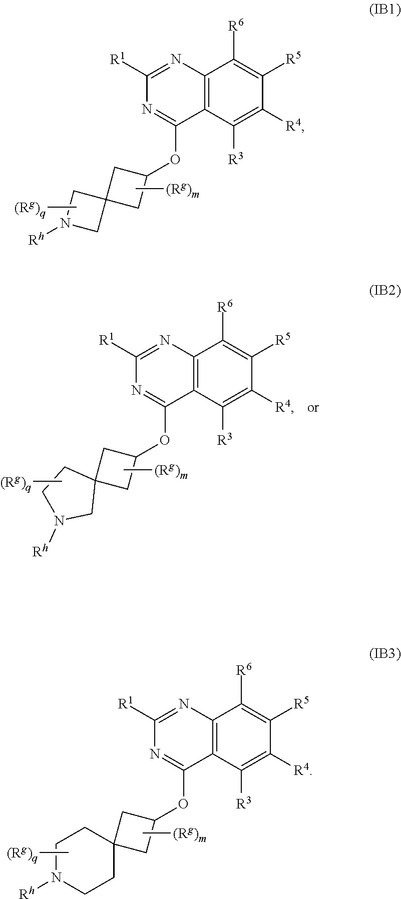

46. The compound of embodiment 44, wherein the compound has the structure:

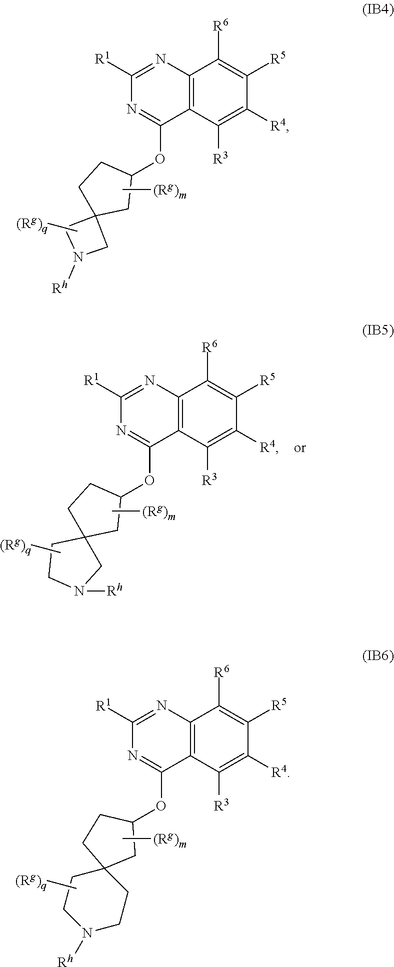

47. The compound of embodiment 44, wherein the compound has the structure:

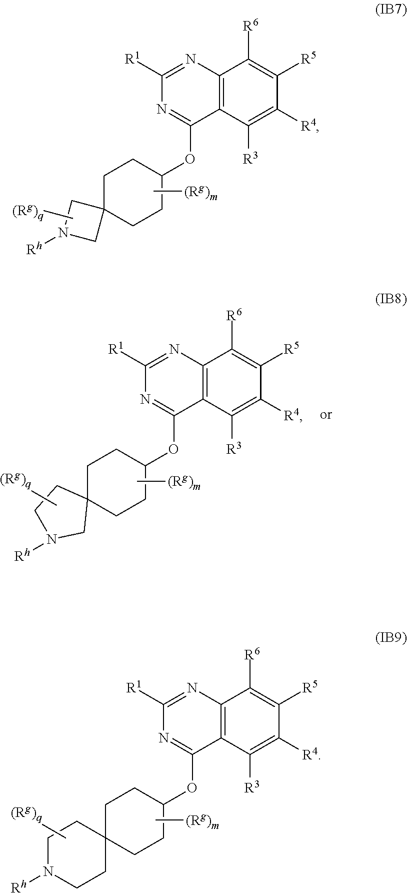

48. The compound of any one of embodiments 44-47, wherein R^{h} is H.
49. The compound of any one of embodiments 44-48, wherein m and q are 0.
50. The compound of any one of embodiments 44-49, wherein R¹ is selected from:

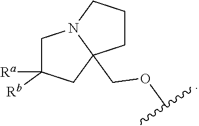

51. The compound of embodiment 50, wherein R¹ is selected from:

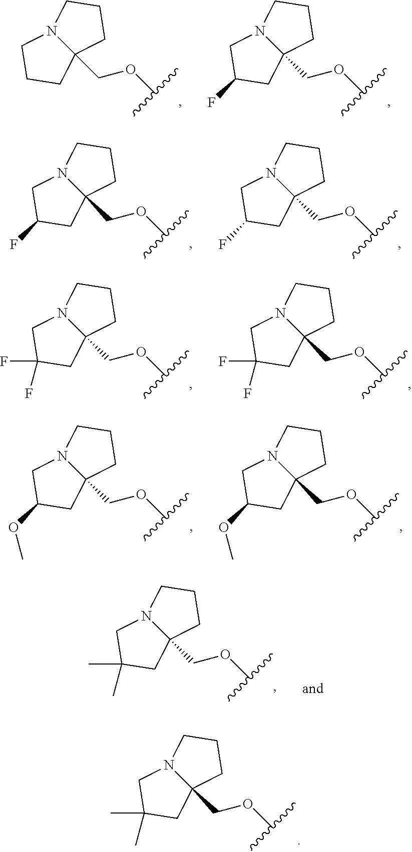

52. The compound of any one of embodiments 44-49, wherein R¹ is selected from:

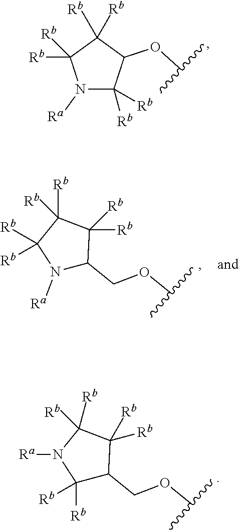

53. The compound of embodiment 52, wherein R¹ is selected from:

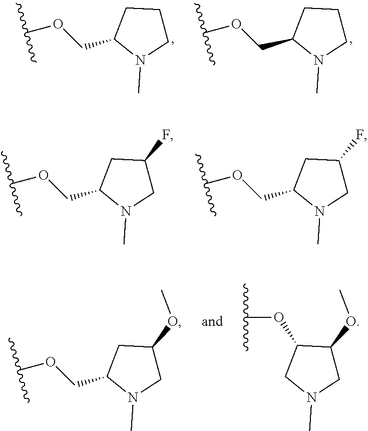

54. The compound of any one of embodiments 44-49, wherein R¹ is selected from:

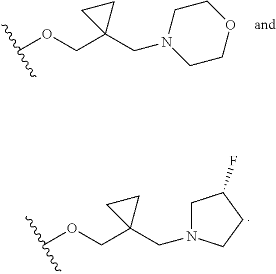 [SMILES: *OCC1(CN2CCOCC2)CC1 ; *OCC1(CN2CC[C@@H](F)C2)CC1]

55. The compound of any one of embodiments 44-54, wherein R⁴ is H.
56. The compound of any one of embodiments 44-54, wherein R⁴ is selected from halogen, —CN, —OR¹², and C₁₋₆alkyl, wherein any C₁₋₆alkyl is unsubstituted or substituted with one or more R¹¹.
57. The compound of embodiment 56, wherein R⁴ is —CN.
58. The compound of embodiment 56, wherein R⁴ is a halogen.
59. The compound of embodiment 56, wherein R⁴ is C₁₋₆alkyl that is substituted with one or more R¹¹.
60. The compound of any one of embodiments 44-59, wherein R⁵ is selected from:

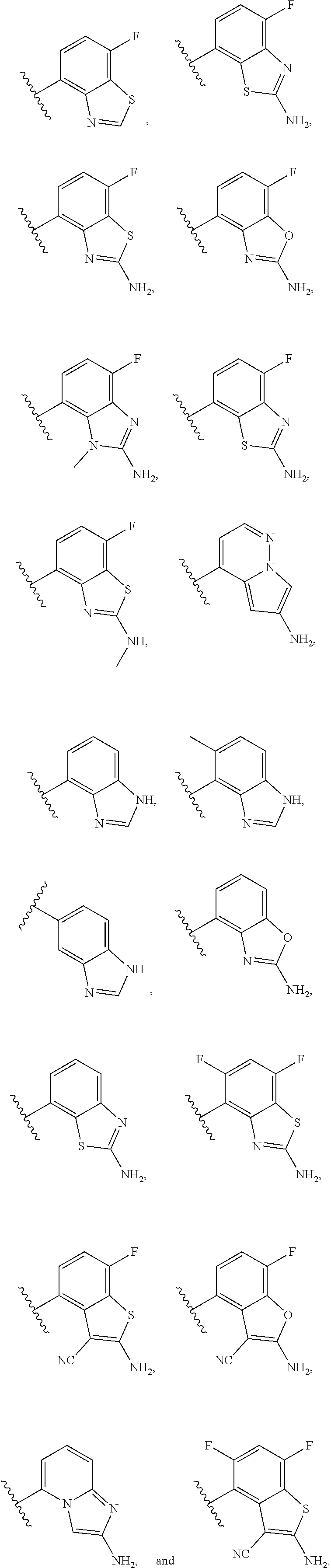

61. The compound of embodiment 60, wherein R⁵ is selected from:

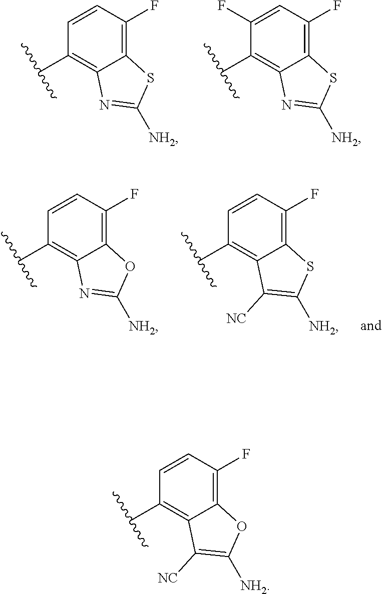 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; "and" ; *c1ccc(F)c2oc(N)c(C#N)c12]

62. The compound of any one of embodiments 44-61, wherein R⁶ is selected from halogen, —CN, and H.
63. The compound of embodiment 62, wherein R⁶ is a halogen.
64. A compound according to Formula IC:

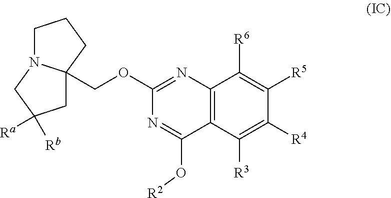

or a salt (e.g., pharmaceutically acceptable salt), ester, tautomer, prodrug, zwitterionic form, or stereoisomer thereof, wherein:
   R² is a 4-11 membered heterocycle containing one or more nitrogen atoms, wherein the heterocycle is unsubstituted or is substituted with one or more R⁸;
   R³ is H;
   R⁴ is selected from H, halogen, —CN, —OR¹², and C₁₋₆alkyl, wherein any C₁₋₆alkyl is unsubstituted or substituted with one or more R¹¹;
   R⁵ is a bicyclic heteroaryl substituted with one or more R⁹;
   R⁶ is selected from halogen, —OR¹², —CN, and H;
   each R⁸ is independently selected from halogen and C₁₋₆alkyl, wherein any C₁₋₆alkyl is unsubstituted or substituted with one or more R¹¹;
   each R⁹ is independently selected from halogen, N(R¹²)₂, —CN, and C₁₋₆alkyl, wherein any C₁₋₆alkyl is unsubstituted or substituted with one or more R¹³;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H; and
   R^{a} and R^{b} are each independently selected from halogen, C₁₋₆ alkyl, —OR¹², and H, wherein any C₁₋₆alkyl is unsubstituted or is substituted with one or more R¹³
65. The compound of embodiment 64, wherein R^{a} is a halogen or —OR¹².
66. The compound of embodiment 64 or 65, wherein R^{b} is H.
67. The compound of any one of embodiments 64-66, wherein R² is a 4-6 membered heterocycle containing one nitrogen atom, wherein the heterocycle is unsubstituted or is substituted with one or more R⁸.
68. The compound of embodiment 67, wherein R² is a heterocycle selected from azetidine, pyrrolidine, and piperidine, wherein the heterocycle is unsubstituted or is substituted with one or more R⁸.
69. The compound of any one of embodiments 64-68, wherein R² is unsubstituted.
70. The compound of any one of embodiments 64-68, wherein R² is substituted with one or more R⁸.
71. The compound of embodiment 70, wherein the one or more R⁸ are each independently selected from C₁₋₆alkyl, wherein any C₁₋₆alkyl is unsubstituted.
72. The compound of any one of embodiments 64-71, wherein R⁴ is H.
73. The compound of any one of embodiments 64-71, wherein R⁴ is selected from halogen, —CN, —OR¹², and C₁₋₆alkyl, wherein any C₁₋₆alkyl is unsubstituted or substituted with one or more R¹¹.
74. The compound of embodiment 73, wherein R⁴ is —CN.
75. The compound of embodiment 73, wherein R⁴ is a halogen.
76. The compound of embodiment 73, wherein R⁴ is C₁₋₆alkyl that is substituted with one or more R¹¹.
77. The compound of any one of embodiments 64-76, wherein R⁵ is selected from:

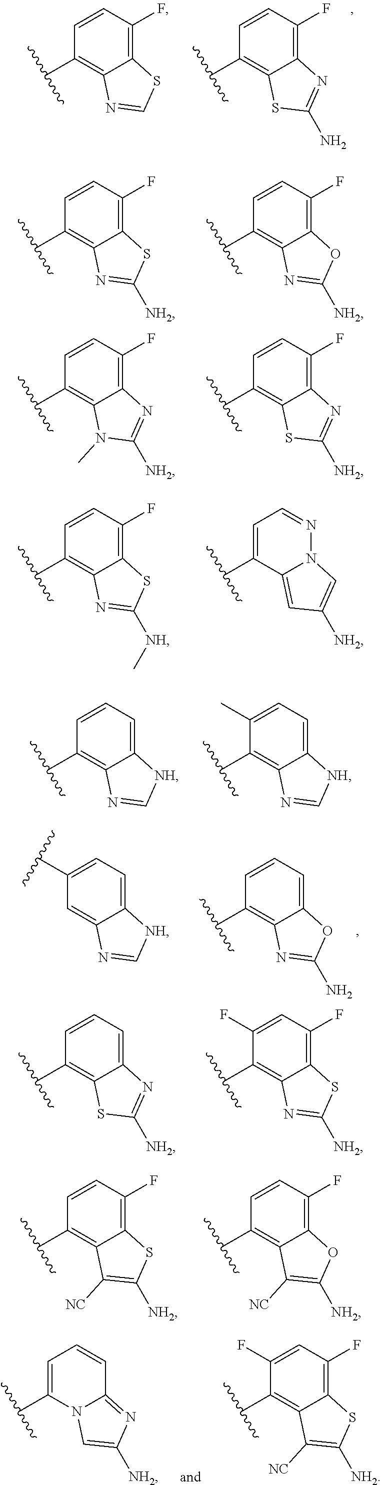

78. The compound of embodiment 77, wherein R⁵ is selected from:

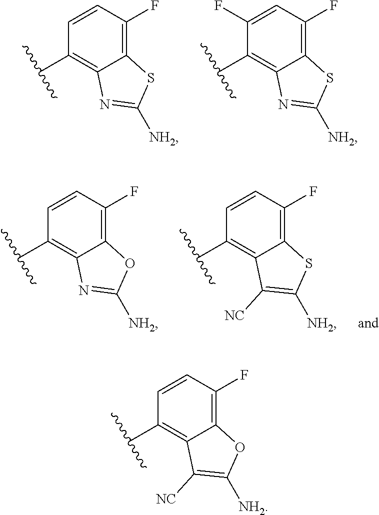 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12]

79. The compound of any one of embodiments 64-78, wherein R⁶ is selected from halogen, —CN, and H.
80. The compound of embodiment 79, wherein R⁶ is a halogen.
81. A compound according to Formula II:

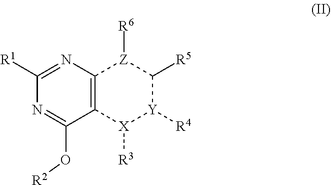

or a salt (e.g., pharmaceutically acceptable salt), ester, tautomer, prodrug, zwitterionic form, or stereoisomer thereof, wherein:
   each dashed line represents a single, double, or mixed bond;
   X, Y, and Z are selected from N and C, wherein one and only one of X, Y, and Z is N;
   R¹ is selected from —OR⁷;
   R² is a 4-6 membered heterocycle containing one or more nitrogen atoms, wherein the heterocycle is unsubstituted or is substituted with one or more R⁸;
   when X is C, R³ is H, and when X is N, R³ is absent;
   when Y is C, R⁴ is selected from H, halogen, —CN, —OR¹², and C₁₋₆alkyl, wherein any C₁₋₆alkyl is unsubstituted or substituted with one or more R¹¹, and when Y is N, R⁴ is absent;
   R⁵ is a bicyclic heteroaryl substituted with one or more R⁹;
   when Z is C, R⁶ is selected from halogen, —OR¹², —CN, and H, and when Z is N, R⁶ is absent;
   R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
   each R⁸ is selected from halogen and C₁₋₆alkyl, wherein any C₁₋₆alkyl is unsubstituted or substituted with one or more R¹¹;
   each R⁹ is independently selected from halogen, N(R¹²)₂, —CN, and C₁₋₆alkyl, wherein any C₁₋₆alkyl is unsubstituted or substituted with one or more R¹³;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H; and
   R^{a} and R^{b} are each independently selected from halogen, C₁₋₆ alkyl, —OR¹², and H, wherein any C₁₋₆alkyl is unsubstituted or is substituted with one or more R¹³.
82. The compound of embodiment 81, wherein X is N, and wherein Y and Z are C.
83. The compound of embodiment 82, wherein the compound is a compound according to Formula IIA:

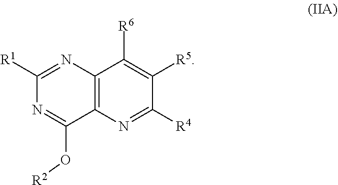

84. The compound of embodiment 82 or 83, wherein R⁴ is H.
85. The compound of embodiment 82 or 83, wherein R⁴ is selected from halogen, —CN, —OR¹², and C₁₋₆alkyl, wherein any C₁₋₆alkyl is unsubstituted or substituted with one or more R¹¹
86. The compound of any one of embodiments 82-85, wherein R⁶ is selected from halogen, —CN, and H.
87. The compound of embodiment 81, wherein Y is N, and X and Z are C.
88. The compound of embodiment 87, wherein the compound is a compound according to Formula IIB:

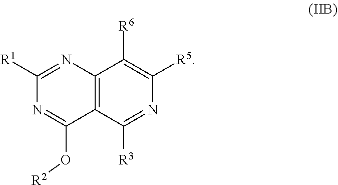

89. The compound of embodiment 87 or 88, wherein R⁶ is a halogen.
90. The compound of embodiment 81, wherein Z is N, and X and Y are C.
91. The compound of embodiment 90, wherein the compound is a compound according to Formula IIC:

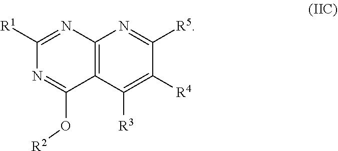

92. The compound of embodiment 90 or 91, wherein R⁴ is H.
93. The compound of embodiment 90 or 91, wherein R⁴ is selected from halogen, —CN, —OR¹², and C₁₋₆alkyl, wherein any C₁₋₆alkyl is unsubstituted or substituted with one or more R¹¹.
94. The compound of any one of embodiments 81-93, wherein R² is a 4-6 membered heterocycle containing one nitrogen atom, wherein the heterocycle is unsubstituted or is substituted with one or more R⁸.
95. The compound of embodiment 94, wherein R² is a heterocycle selected from azetidine, pyrrolidine, and piperidine, wherein the heterocycle is unsubstituted or is substituted with one or more R⁸.
96. The compound of any one of embodiments 81-95, wherein R² is unsubstituted.
97. The compound of any one of embodiments 81-95, wherein R² is substituted with one or more R⁸.
98. The compound of embodiment 97, wherein the one or more R⁸ are each independently selected from C₁₋₆alkyl, wherein any C₁₋₆alkyl is unsubstituted.
99. The compound of any one of embodiments 81-98, wherein R¹ is selected from:

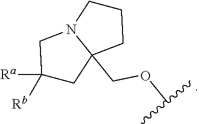

100. The compound of embodiment 99, wherein R1 is selected from:

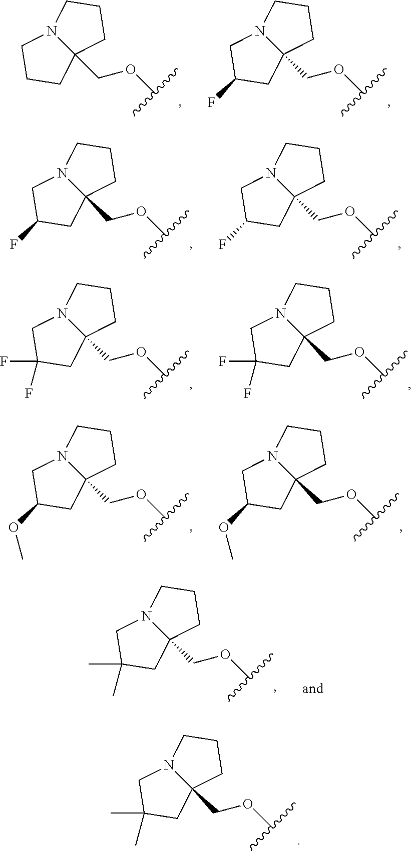

101. The compound of any one of embodiments 81-98, wherein R¹ is selected from:

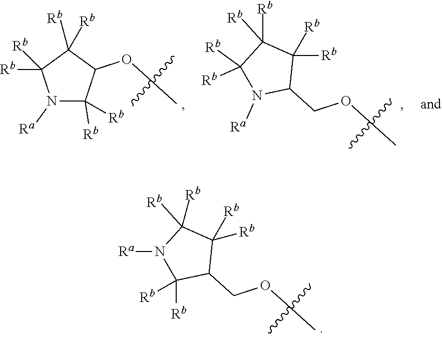

102. The compound of embodiment 101, wherein R¹ is selected from:

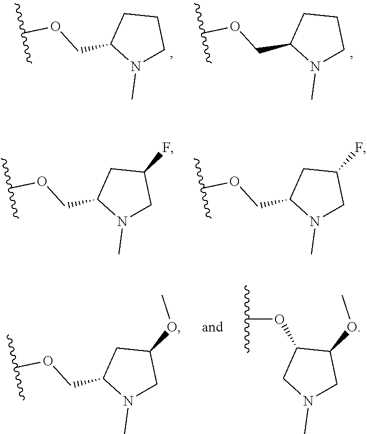

103. The compound of any one of embodiments 81-98, wherein R¹ is selected from:

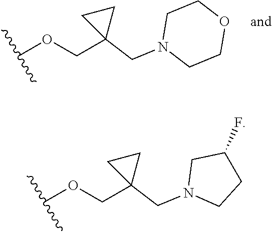 [SMILES: *OCC1(CN2CCOCC2)CC1 ; *OCC1(CN2CC[C@@H](F)C2)CC1]

104. The compound of any one of embodiments 81-103, wherein R⁵ is selected from:

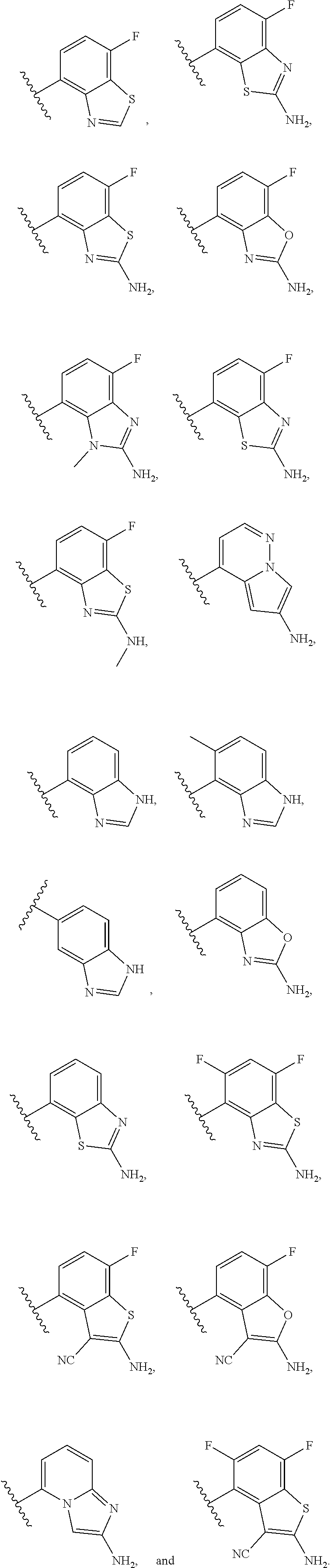

105. The compound of embodiment 104, wherein R⁵ is selected from:

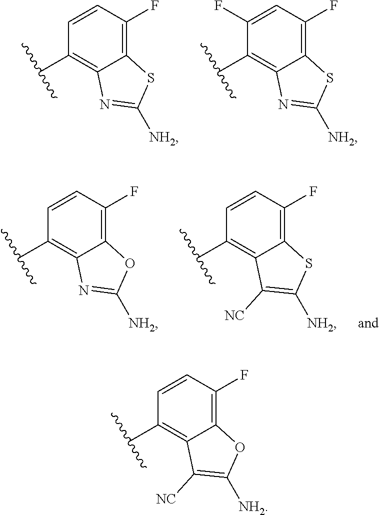 [SMILES: *c1ccc(F)c2sc(N)nc12 ; *c1c(F)cc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12]

106. A compound shown in Table 2, or a salt (e.g., pharmaceutically acceptable salt), ester, tautomer, prodrug, zwitterionic form, or stereoisomer thereof.
107. A pharmaceutical composition comprising a compound of any one of embodiments 1-106, or a salt (e.g., pharmaceutically acceptable salt), ester, tautomer, prodrug, zwitterionic form, or stereoisomer thereof, and a pharmaceutically acceptable excipient.
108. A compound of any one of embodiments 1-106, or a salt (e.g., pharmaceutically acceptable salt), ester, tautomer, prodrug, zwitterionic form, or stereoisomer thereof, for use as a medicament.
109. The compound of embodiment 108, wherein the medicament is useful in the prevention or treatment of a disease, disorder, or condition ameliorated by the inhibition of KRAS having a G12D mutation.
110. The compound of embodiment 108 or 109, wherein the medicament is useful in the prevention or treatment of a cancer.
111. The compound of embodiment 110, wherein the cancer is selected from the group consisting of pancreatic cancer, colorectal cancer, and lung cancer.
112. A compound of any one of embodiments 1-106, or a salt (e.g., pharmaceutically acceptable salt), ester, tautomer, prodrug, zwitterionic form, or stereoisomer thereof, for use in the treatment of a disease, disorder, or condition.
113. The compound of embodiment 112, wherein the disease, disorder, or condition is a cancer.
114. The compound of embodiment 113, wherein the cancer is selected from the group consisting of pancreatic cancer, colorectal cancer, and lung cancer.
115. The compound of any one of embodiments 112-114, wherein the compound is used in the treatment of a disease, disorder, or condition in a subject in need thereof.
116. A compound of any one of embodiments 1-106, or a salt (e.g., pharmaceutically acceptable salt), ester, tautomer, prodrug, zwitterionic form, or stereoisomer thereof, for use in the manufacture of a medicament.
117. The compound of embodiment 116, wherein the medicament is useful in the prevention or treatment of a disease, disorder, or condition ameliorated by the inhibition of KRAS having a G12D mutation.
118. The compound of embodiment 116 or 117, wherein the medicament is useful in the treatment of a cancer.
119. The compound of embodiment 118, wherein the cancer is selected from the group consisting of pancreatic cancer, colorectal cancer, and lung cancer.
120. A method, comprising administering a therapeutically effective amount of a compound of any one of embodiments 1-106, or a salt (e.g., pharmaceutically acceptable salt), ester, tautomer, prodrug, zwitterionic form, or stereoisomer thereof, to a subject in need thereof.
121. The method of embodiment 120, wherein the subject has a disease, disorder, or condition ameliorated by the inhibition of KRAS having a G12D mutation.
122 The method of embodiment 120 or 121, wherein the subject has a cancer.
123. The method of embodiment 122, wherein the subject was previously diagnosed with the cancer.
124 The method of embodiment 122, wherein the subject has previously undergone a treatment regimen for the cancer.
125 The method of embodiment 122, wherein the subject has previously entered remission from the cancer.
126. The method of any one of embodiments 122-125, wherein the cancer is selected from the group consisting of pancreatic cancer, colorectal cancer, and lung cancer.
127. The method of any one of embodiments 120-126, wherein the compound, or the salt, ester, tautomer, prodrug, zwitterionic form, or stereoisomer thereof, is administered in combination with an additional therapeutic agent.
128. The use of a compound of any one of embodiments 1-106, or a salt (e.g., pharmaceutically acceptable salt), ester, tautomer, prodrug, zwitterionic form, or stereoisomer thereof, for the manufacture of a medicament for the treatment of a cancer.
129. The use of embodiment 128, wherein the cancer is selected from the group consisting of pancreatic cancer, colorectal cancer, and lung cancer.
130. A method, comprising contacting a KRAS protein with a compound of any one of embodiments 1-106, or a salt (e.g., pharmaceutically acceptable salt), ester, tautomer, prodrug, zwitterionic form, or stereoisomer thereof.
131. The method of embodiment 130, wherein contacting the KRAS protein with the compound modulates KRAS.
132. The method of embodiment 130 or 131, wherein the KRAS protein has a G12D mutation.
133. The method of any one of embodiments 130-132, wherein the KRAS protein is in an active state.
134. The method of any one of embodiments 130-132, wherein the KRAS protein is in an inactive state.

### Examples

Selected abbreviations used in the preceding sections and the Examples are summarized in Table 1.

TABLE 1
Abbreviations.

| Abbreviation | Term |
|---|---|
| MeCN | acetonitrile |
| cm | centimeter |
| ° C. | degrees Celsius |
| ° K. | degrees Kelvin |
| BINAP | [1,1′-Binaphthalene]-2,2′- |
| | diyl)bis(diphenylphosphane) |
| DCM | dichloromethane |
| DIEA | N,N-diisopropylethylamine (Hunig's base) |
| DIPEA | N-N-diisopropylethylamine |
| DMF | dimethylformamide |
| DMSO | dimethyl sulfoxide |
| DPPA | Diphenylphosphoryl azide |
| ESI | electrospray ion-mass spectrometry |
| EtOH | ethanol |
| EtOAc, EA | ethyl acetate |
| g | gram |
| Hz | hertz |
| HATU | Hexafluorophosphate azabenzotriazole |
| | tetramethyl uronium |
| HPLC | high performance liquid chromatography |
| HTRF | homogenous time-resolved fluorescence |
| h | hour |
| kDa | kilodalton |
| LC | liquid chromatography |
| LCMS | liquid chromatography-mass spectrometry |
| L | liter |
| MS | mass spectra |
| MHz | megahertz |
| MeOH | methanol |
| MTBE | methyl tert-butyl ether |
| μg | microgram |
| μL | microliter |
| μM | micromolar |
| μm | micron |
| μs | microsecond |
| mg | milligram |
| mL | milliliter |
| mm | millimeter |
| mM | millimolar |
| mmol | millimole |
| min | minute |
| M | molar |
| nL | nanoliter |
| nm | nanometer |
| NCS | N-chlorosuccinimide |
| NIS | N-iodosuccinimide |
| NMP | N-methyl-2-pyrrolidone |
| PE:EA | petroleum ether:ethyl acetate |
| ppm | parts per million |
| PTLC | preparative thin layer chromatography |
| ¹HNMR | proton nuclear magnetic resonance |
| RBD | receptor binding domain |
| RP | reverse phase |
| rpm | revolutions per minute |
| SPR | surface plasmon resonance |
| THF | tetrahydrofuran |
| TMS | tetramethylsilane |
| TLC | thin layer chromatography |
| TsOH | tosylic acid |
| SOCl₂ | thionyl chloride |
| TEA | triethylamine |
| TFA | trifluoroacetic acid |
| TCEP | tris(2-carboxyethyl)phosphine |
| UV | ultraviolet |
| UV/Vis | ultraviolet/visible |

### Materials and Methods

Preparative thin layer chromatography (PTLC) separations described herein were typically performed on 20×20 cm plates (500-μm thick silica gel).

Chromatographic purifications were typically performed using Biotage Isolera. One automated system running Biotage Isolera One 2.0.6 software (Biotage LLC, Charlotte, NC). Flow rates were the default values specified for the column in use. Reverse phase chromatography was performed using elution gradients of water and acetonitrile on KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g., hexane, ethyl acetate, methylene chloride, methanol, acetone, chloroform, MTBE, etc.). The columns were SNAP Cartridges containing KP-SIL or SNAP Ultra (25 pm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹HNMR analyses of intermediates and exemplified compounds were typically performed on a Bruker Ascend™ 400 spectrometer (operating at 400 MHz), Bruker Ascend 700 MHz Advance Neo Spectrometer (Bruker-Biospin) or Bruker Advance ultrashield 300/54 (operating at 300 MHz) at 298° K following standard operating procedure suggested by manufacturer. Reference frequency was set using TMS as an internal standard. Chemical shift values (S) are reported in parts per million (ppm) with splitting patterns abbreviated to: s (singlet), br. s (broad singlet), d (doublet), dd (double doublet), t (triplet), and m (multiplet). The coupling constant (J) is given in Hz. Typical deuterated solvents were utilized as indicated in the individual examples.

LCMS analyses were typically performed using one of the following conditions:
(1) LCMS spectra were taken on an Agilent Technologies 6120B Quadrupole spectrometer. The mobile phase for the LC was acetonitrile (A) with 0.1% formic acid, and water (B) with 0.1% formic acid, and the eluent gradient was from 5-95% A in 6.0 min, 5%-40% A in 6.0 min, 80-100% A in 6.0 min. using a poroshell 120 EC-C18 50 mm×3.0 mm×2.7 μm capillary column; Flow Rate: 0.7 mL/min. Mass spectra (MS) were measured by electrospray ion-mass spectroscopy (ESI). All temperatures are in degrees Celsius (° C.) unless otherwise noted.
(2) LCMS spectra were taken on an Agilent Technologies 1290-6420 Triple Quadrupole spectrometer: The mobile phase for the LC was acetonitrile (A) with 0.05% formic acid, and water (B) with 0.05% formic acid, and the eluent gradient was from 5-95% A in 5.0 min, using a ZORBAX SB—C18 50 mm×2.1 mm×1.8 μm capillary column; Flow Rate: 0.3 mL/min. Mass spectra (MS) were measured by electrospray ion-mass spectroscopy (ESI). All temperatures are in degrees Celsius unless otherwise noted.
(3) LC-MS analysis was performed using an Agilent 6120b single quadrupole mass spectrometer with an Agilent 1260 infinity II chromatography separations module and Agilent 1260 infinity II photodiode array detector controlled by Agilent Chemstation software. The HPLC column used was an Agilent ZORBAX Eclipse XDB-C18 4.6 mm×150 mm×3.5 μm RapidResol column with a mobile phase of water (0.1% formic acid)/MeCN (0.1% formic acid) and a gradient of 5-95% MeCN over 10 minutes at a flow rate of 1 mL/min. Accurate mass data was obtained using a Thermo Fisher extractive plus EMR orbitrap LCMS system. Exact mass values were calculated by ChemCalc.
(4) LCMS spectra were taken on an alliance Waters 2695 coupled to a dual absorbance detector waters 2487 and a waters micro mass ZQ-2000 single quadrupole spectrometer. The mobile phase for the LC was acetonitrile (A) and water (B) with 0.01% formic acid, and the eluent gradient was from 5-100% A in 10.0 minute using a Kromasil 100-5-C18 150 mm×4.6 mm×5 μm column. Mass spectra (MS) were measured by electrospray ion-mass spectroscopy (ESI). All temperatures are in degrees Celsius unless otherwise noted.

Typically, analytical HPLC mass spectrometry conditions were as follows:
LC1: Agilent Technologies 1260 Infinity coupled, Column: poroshell 120 EC-C18 150 mm×4.6 mm×4 μm; Temperature: 40° C.; Eluent: 5:95 v/v acetonitrile/water+0.02% trifluoroacetic acid in 20 min; Flow Rate: 1.2 mL/min; Detection: VWD, 190-600 nm.
LC2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μM OBD™ 19×250 mm column with a mobile phase of water/MeCN or water (0.1% TFA)/MeCN (0.1% TFA).

Preparative HPLC were carried out with one of the following two conditions:

Condition 1: GILSON Preparative HPLC System; Column: Ultimate XB-C18, 21.2 mm×250 mm, 5 μm; Mobile phase: Water with 0.1% trifluoroacetic acid; MeCN with 0.1% trifluoroacetic acid; Method: 15 minutes gradient elution; Initial organic: 10% to 30%; Final organic: 60% to 80%; UV1: 240; UV2: 230; Flow: 15 mL/min.

Condition 2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 uM OBD™ 19×250 mm column with a mobile phase of water/MeCN or water (0.1% TFA)/MeCN (0.1% TFA).

Compound names were generated with ChemDraw Professional.

The compounds provided herein, or alternate forms thereof (e.g., salt, ester, tautomer, prodrug, zwitterionic form, stereoisomer, etc.), may be prepared according to various methods including those set forth in the following examples.

### Synthetic Example 1: Synthesis of 7-fluoro-4-(8-fluoro-4-(((3S,4R)-4-fluoropyrrolidin-3-yl)oxy)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine (Compound 22)

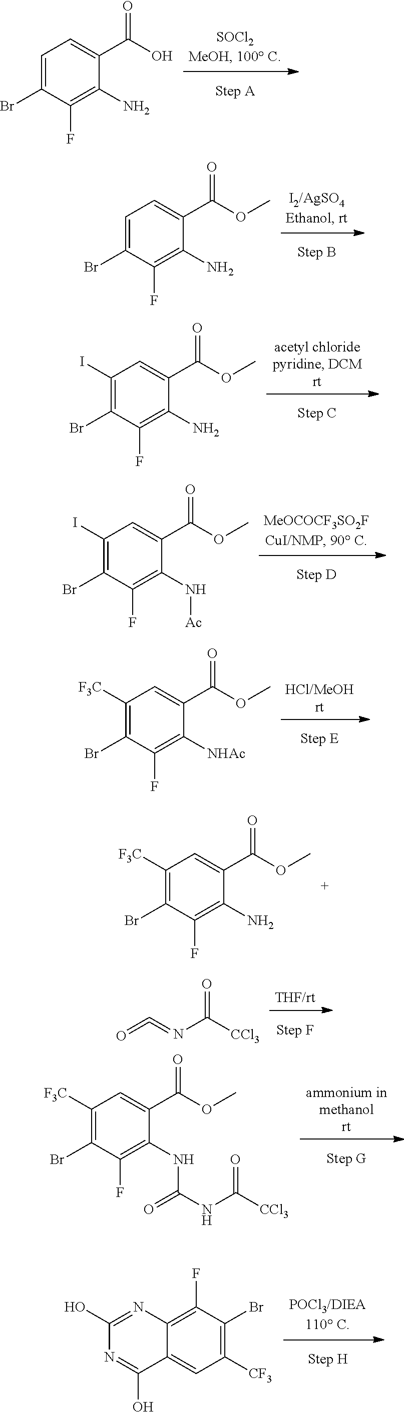

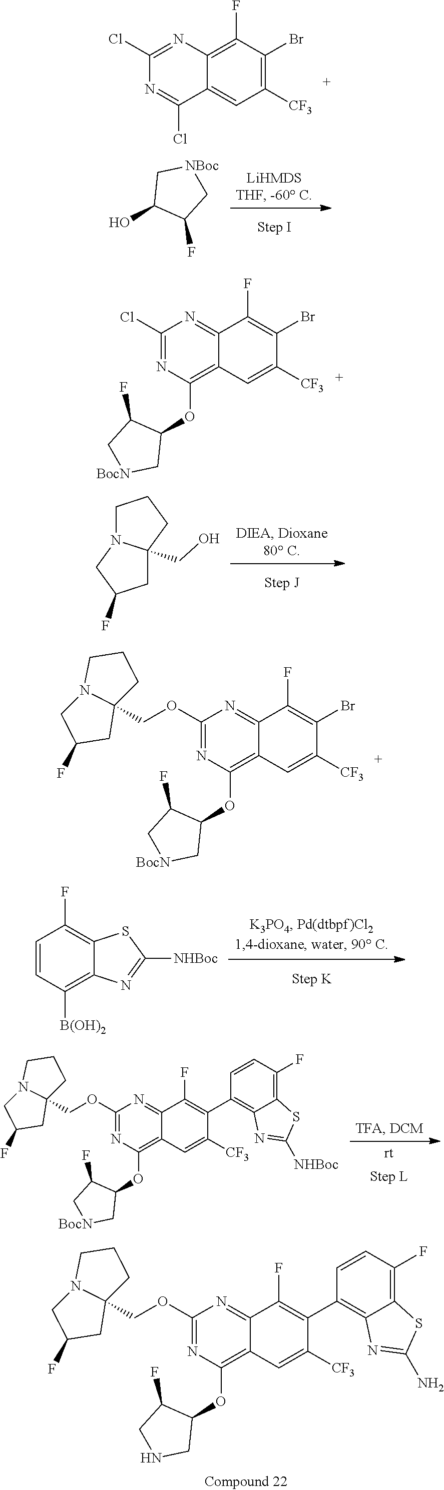

Step A: Preparation of methyl 2-amino-4-bromo-3-fluorobenzoate: To stirring solution of 2-amino-4-bromo-3-fluorobenzoic acid (5.0 g, 21.4 mmol) in MeOH (30 mL) was added dropwise thionyl chloride (15.6 ml, 21 mmol) at 0° C. under argon. The resulting mixture was heated to 100° C. for 16 hours. The solvent was evaporated, and the residue was dissolved in ethyl acetate (100 mL). The organic layer was washed with a saturated aqueous NaHCO₃ solution then dried over Na₂SO₄, filtered, and concentrated under vacuum. The resulting crude material was purified by silica gel column chromatography using EtOAc in hexanes (0% to 20%) as eluent to give methyl 2-amino-4-bromo-3-fluorobenzoate (5.0 g, 94%) as a solid. LCMS ESI (+) m/z 249 (M+H). ¹HNMR (300 MHz, CDCl₃) δ 7.53 (dd, J=8.8, 1.8 Hz, 1H), 6.78 (dd, J=8.8, 6.3 Hz, 1H), 5.93 (s, 1H), 3.90 (s, 1H).

Step B: Preparation of methyl 2-amino-4-bromo-3-fluoro-5-iodobenzoate: To a mixture of iodine (7.16 g, 28 mmol) and silver sulfate (5.3 g, 17 mmol) in EtOH (200 mL), methyl 2-amino-4-bromo-3-fluorobenzoate (5.0 g, 20 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution, brine and the resulting organic solution was dried over Na₂SO₄, filtered and concentrated under vacuum to give methyl 2-amino-4-bromo-3-fluoro-5-iodobenzoate: the title compound (6.66 g, 88% yield) as a yellow solid. LCMS ESI (+) m/z 373 (M+H). ¹HNMR (300 MHz, CDCl₃) δ 8.14 (d, J=1.9 Hz, 1H), 5.94 (s, 2H), 3.91 (s, 3H).

Step C: Preparation of methyl 2-acetamido-4-bromo-3-fluoro-5-iodobenzoate: The methyl 2-amino-4-bromo-3-fluoro-5-iodobenzoate (3.50 g, 9.4 mmol) and pyridine (2.3 ml, 28 mmol) were dissolved in DCM at 0° C. Acetyl chloride (0.79 ml, 11 mmol) was added and the reaction was warmed to ambient temperature and stirred at this temperature for 16 hours. The reaction mixture was concentrated under vacuum and the residue obtained was purified by silica gel column chromatography using ethyl acetate in hexanes (0% to 30%) as eluent to give methyl 2-acetamido-4-bromo-3-fluoro-5-iodobenzoate (2.7 g, 69%) as solid. LCMS ESI (+) m/z 417 (M+H). ¹HNMR (300 MHz, CDCl₃) δ 8.87 (s, 1H), 8.25 (s, 1H), 3.95 (s, 3H), 2.26 (s, 3H).

Step D: Preparation of methyl 2-acetamido-4-bromo-3-fluoro-5-(trifluoromethyl)benzoate: To a stirred solution of methyl 4-bromo-2-acetamido-3-fluoro-5-iodobenzoate (1.0 g, 2.4 mmol) and methyl fluorosulfonyldifluoroacetate (0.92 g, 0.72 mmol) in NMP (22.0 mL) at ambient temperature, CuI (0.14 g, 0.73 mmol) was added and the resulting mixture was stirred at 80° C. for 16 hours. Once cooled to ambient temperature, the mixture was quenched with water and extracted with ethyl acetate. The organic layer was dried over Na₂SO₄, filtered, concentrated and the crude material was purified by silica gel column chromatography using ethyl acetate in hexanes (0% to 20%) as eluent to give methyl 2-acetamido-4-bromo-3-fluoro-5-(trifluoromethyl)benzoate (0.64 g, 74%) as solid. LCMS ESI (+) m/z 358 (M+H). ¹HNMR (300 MHz, CDCl₃) δ 9.23 (s, 1H), 8.10 (s, 1H), 3.93 (s, 3H), 2.28 (s, 3H).

Step E: Preparation of 2-amino-4-bromo-3-fluoro-5-(trifluoromethyl)benzoate: A mixture of methyl 2-acetamido-4-bromo-3-fluoro-5-(trifluoromethyl)benzoate (1.20 g, 3.35 mmol) in 3 M HCl in MeOH was heated at 60° C.; for 2 hours. Once cooled to ambient temperature, the solvent was evaporated, and the crude product was partitioned between EtOAc and saturated NaHCO₃. The organic layer was separated, dried over sodium sulfate, filtered, and evaporated to provide 2-amino-4-bromo-3-fluoro-5-(trifluoromethyl)benzoate (1.00 g, 94%) as an oil. LCMS ESI (+) m/z 316.9 (M+H). ¹H NMR (300 MHz, CDCl₃) δ 8.01 (s, 1H), 6.28 (s, 2H), 3.94 (s, 3H).

Step F: Preparation of methyl 4-bromo-3-fluoro-2-(3-(2,2,2-trichloroacetyl)ureido)-5-(trifluoromethyl)benzoate: To a mixture of methyl 2-amino-4-bromo-3-fluoro-5-(trifluoromethyl)benzoate (0.80 g, 2.53 mmol) in THE (4.2 mL) was added trichloroethanecarbonyl isocyanate (0.45 mL, 3.79 mmol) at ambient temperature. After 15 minutes, the reaction mixture was evaporated, followed by the addition of MTBE and the solid formed was collected and washed with MTBE to provide methyl 4-bromo-3-fluoro-2-(3-(2,2,2-trichloroacetyl)ureido)-5-(trifluoromethyl)benzoate (0.71 g, 56%). LCMS ESI (+) m/z 529.99 (M+Na). ¹H NMR (300 MHz, CDCl₃) δ 10.91 (s, 1H), 8.65 (s, 1H), 8.23 (s, 1H), 4.03 (s, 3H).

Step G: Preparation of 7-bromo-8-fluoro-6-(trifluoromethyl)quinazoline-2,4-diol: To a solution of methyl 4-bromo-3-fluoro-2-(3-(2,2,2-trichloroacetyl)ureido)-5-(trifluoromethyl)benzoate (0.71 g, 1.40 mmol) in methanol (7.0 mL) was added 7 M solution of ammonia in methanol (0.46 mL, 3.23 mmol) at ambient temperature and stirred at ambient temperature for 1 hour. The mixture was concentrated under reduced pressure to provide a solid which was co-evaporated with ether to provide 7-bromo-8-fluoro-6-(trifluoromethyl)quinazoline-2,4-diol (0.60 g, 100%) as solid LCMS ESI (+) m/z 260.0 (M+H). ¹H NMR (300 MHz, DMSO-d₆) δ 8.41 (s, 1H), 8.28 (s, 1H), 7.99 (s, 1H).

Step H: Preparation of 7-bromo-2,4-dichloro-8-fluoro-6-(trifluoromethyl)quinazoline: To a stirring solution of phosphorus oxychloride (0.97 mL, 10.5 mmol) and Hunig's base (0.40 mL, 2.29 mmol) was added 7-bromo-8-fluoro-6-(trifluoromethyl)quinazoline-2,4-diol (0.15 g, 0.46 mmol) at 0° C. After addition, the resulting mixture was stirred at 110° C. for 1 hour. Once cooled down to ambient temperature, the mixture was evaporated and co-evaporated with chloroform to give 7-bromo-2,4-dichloro-8-fluoro-6-(trifluoromethyl)quinazoline which used as such for the next step. ¹H NMR (300 MHz, CDCl₃) δ 8.45 (s, 1H).

Step I: Preparation of tert-butyl (3S,4R)-3-[7-bromo-2-chloro-8-fluoro-6-(trifluoromethyl)quinazolin-4-yl]oxy-4-fluoro-pyrrolidine-1-carboxylate: To a solution of tert-butyl (3S,4R)-3-fluoro-4-hydroxy-pyrrolidine-1-carboxylate (200 mg, 0.97 mmol) in THE (1.0 mL) was added LiHMDS (1M in THF) (1.02 mL, 1.02 mmol) dropwise at −70° C. under Ar and then stirred at −70° C. for 1 hour. 7-bromo-2,4-dichloro-8-fluoro-6-(trifluoromethyl)quinazoline (295 mg, 0.81 mmol) in THE (1 mL) was added at 70° C. After stirring for 1 hour at −70° C., the mixture was quenched with NH₄Cl aqueous solution and extracted with EtOAc. The combined organics were dried (sodium sulfate), filtered and concentrated to dry under vacuum and the residue was purified by column chromatography on silica gel, eluting with 25% EtOAc in petroleum ether, to give tert-butyl (3S,4R)-3-[7-bromo-2-chloro-8-fluoro-6-(trifluoromethyl)quinazolin-4-yl]oxy-4-fluoro-pyrrolidine-1-carboxylate (64 mg, 14%). LCMS ESI (+) m/z 532.0 (M+H).

Step J: Preparation of tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]oxy-4-fluoro-pyrrolidine-1-carboxylate: To a solution of tert-butyl (3S,4R)-3-[7-bromo-2-chloro-8-fluoro-6-(trifluoromethyl)quinazolin-4-yl]oxy-4-fluoro-pyrrolidine-1-carboxylate (64 mg, 0.12 mmol) in 1,4-dioxzne (1 mL) was added [(2R,8S)-2-fluoro-1,2,3,5,6,7-hexahydropyrrolizin-8-yl]methanol (38 mg, 0.24 mmol) and DIEA (0.04 mL, 0.83 mmol) at ambient temperature. The mixture was sealed and the stirred at 80° C. for 8 hours. After cooling to ambient temperature, the mixture was diluted with brine, extracted with EtOAc and washed with brine. The organics were separated, dried (sodium sulfate), filtered and concentrated under vacuum. The residue was purified by column chromatography on silica gel, eluting with 50% EtOAc in petroleum ether, to give tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]oxy-4-fluoro-pyrrolidine-1-carboxylate (65 mg, 82%). LCMS ESI (+) m/z 655.1 (M+H).

Step K: Preparation of tert-butyl (3S,4R)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)oxy)-4-fluoropyrrolidine-1-carboxylate: To a solution of [2-(tert-butoxycarbonylamino)-7-fluoro-1,3-benzothiazol-4-yl]boronic acid (34 mg, 0.11 mmol) in 1,4-Dioxane (2 mL)/Water (0.40 mL) was added tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]oxy-4-fluoro-pyrrolidine-1-carboxylate (60 mg, 0.092 mmol), Pd(dtbpf)Cl₂ (6.0 mg, 0.01 mmol) and K₃PO₄ (39 mg, 0.18 mmol) at room temperature. The mixture was bubbled with Ar for 1-2 minutes and then sealed. After that, the mixture was stirred at 90° C. for 4 hours. After cooling to ambient temperature, water and ethyl acetate were added. The organic layer was separated, dried (sodium sulfate), filtered and concentrated under reduced pressure. The residue was purified by preparative-TLC (50% EtOAc in petroleum ether) to give tert-butyl (3S,4R)-3-((7-(2-((tert-butoxycarbonyl)amino)-7-fluorobenzo [d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)oxy)-4-fluoropyrrolidine-1-carboxylate (51 mg, 66%). LCMS ESI (+) m/z 843.3 (M+H).

Step L: Preparation of 7-fluoro-4-(8-fluoro-4-(((3 S,4R)-4-fluoropyrrolidin-3-yl)oxy)-2-(((2R,7aS)-2-fluorotetrahydro-1H-pyrrolizin-7a(5H)-yl)methoxy)-6-(trifluoromethyl)quinazolin-7-yl)benzo [d]thiazol-2-amine: To a solution of tert-butyl (3S,4R)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)oxy)-4-fluoropyrrolidine-1-carboxylate (25 mg, 0.030 mmol) in DCM (3 mL) was added trifluoroacetic acid (1.0 mL, 13.0 mmol) at room temperature and stirred for 2 hours. After concentrated under vacuum, the residue was suspended in DCM and water and adjusted pH with saturated sodium bicarbonate to pH-9. It was extracted with DCM. Organic layer was separated, dried (sodium sulfate), filtered, concentrated under reduced pressure, and dried to give 7-fluoro-4-(8-fluoro-4-(((3S,4R)-4-fluoropyrrolidin-3-yl)oxy)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine (20 mg, 100%). LCMS ESI (+) m/z 643.2 (M+H). 1H NMR (400 MHz, MeOD) δ 8.37 (s, 1H), 7.20 (s, 1H), 6.98 (t, J=8.8 Hz, 1H), 5.80-5.81 (m, 1H), 5.52 (d, J=56.4 Hz, 1H), 5.38 (d, J=56.4 Hz, 1H), 4.41-4.58 (m, 2H), 3.60-3.61 (m, 1H), 3.36-3.43 (m, 6H), 3.14-3.15 (m, 1H), 1.97-2.46 (m, 6H).

### Synthetic Example 2: 7-fluoro-4-(8-fluoro-2-(((2R,7aS)-2-fluorotetrahydro-1H-pyrrolizin-7a(5H)-yl)methoxy)-4-(((R)-pyrrolidin-3-yl)oxy)pyrido[4,3-d]pyrimidin-7-yl)benzo[d]thiazol-2-amine (Compound 24)

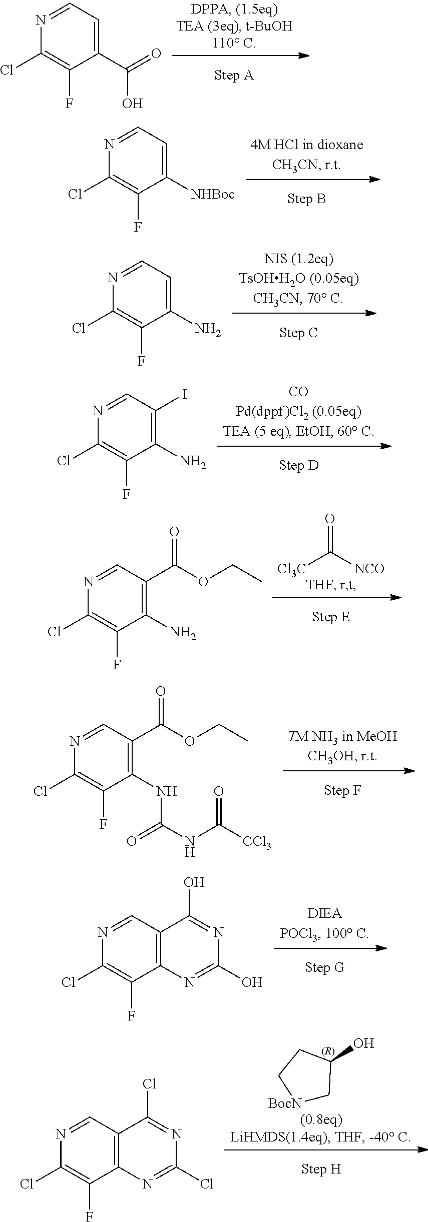

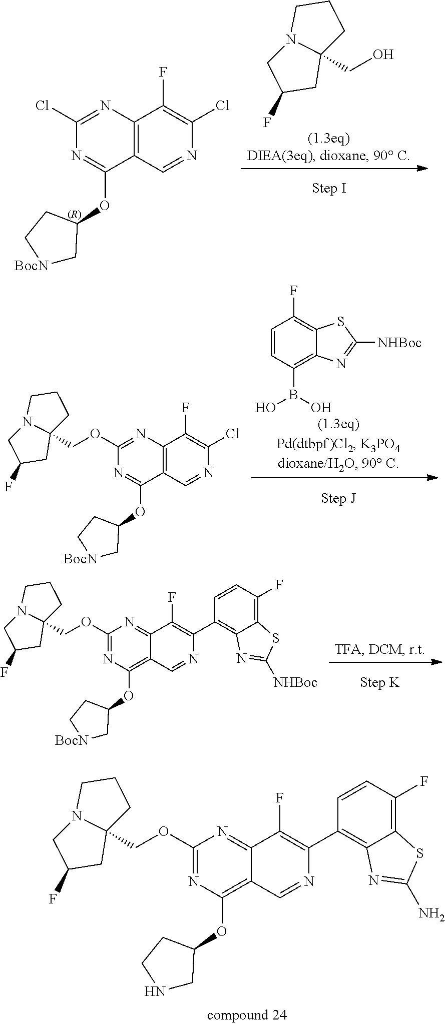

Step A: Preparation of tert-butyl N-(2-chloro-3-fluoro-4-pyridyl)carbamate: To a mixed solution of 2-chloro-3-fluoro-pyridine-4-carboxylic acid (1.00 eq, 38.50 g, 219 mmol) in tert-butanol (150 mL) was added TEA (3.00 eq, 92 mL, 658 mmol) and DPPA (1.50 eq, 90.47 g, 329 mmol) in portions. After stirring for 3 hours at room temperature, the mixture was stirred for 10 hours at 110° C. The reaction was quenched with water (1 L) and extracted with ethyl acetate (1000 mL×3). The combined organic layers were washed with an aqueous solution of NaCl (800 mL×3), dried over Na₂SO₄ and evaporated to dry under vacuum. The residue was purified by column chromatography on silica gel (eluting with petroleum ether/ethyl acetate 100:1→50:1→10:1→5:1) to give the tert-butyl N-(2-chloro-3-fluoro-4-pyridyl)carbamate (41.50 g, 118 mmol, 53.70% yield) as a yellow solid. LCMS ESI (+) m/z 247.1 (M+H).

Step B: Preparation of 2-chloro-3-fluoro-pyridin-4-amine: To a solution of tert-butyl N-(2-chloro-3-fluoro-4-pyridyl)carbamate (1.00 eq, 41.00 g, 167 mmol) in MeCN (150 mL) was added 4M HCl in 1,4-dioxane (150 mL). The mixture was stirred for 5 hours at room temperature. Water (1000 ml) was added, the mixture was adjusted pH=8 with aqueous solution of Na₂CO₃ and was extracted with ethyl acetate (1000 mL×3). The combined organic layers were washed with an aqueous solution of NaCl (800 mL×3), dried over Na₂SO₄ and evaporated to dry under vacuum. The crude product was purified by recrystallized from petroleum ether/ethyl acetate (5/1) to afford 2-chloro-3-fluoro-pyridin-4-amine (22.00 g, 150.06 mmol, 90.42% yield) as light-yellow solid. LCMS ESI (+) m/z 147.1 (M+H).

Step C: Preparation of 2-chloro-3-fluoro-5-iodo-pyridin-4-amine: To a solution of 2-chloro-3-fluoro-pyridin-4-amine (1.00 eq, 22.00 g, 150 mmol) and TsOH·H₂a (0.1000 eq, 2852 mg, 15.0 mmol) in MeCN (300 mL) was added NIS (1.00 eq, 33.77 g, 150 mmol). The mixture was stirred for 5 hours at 70° C. The reaction was quenched with Na₂SO₃ solution (1000 ml) and extracted with ethyl acetate (1.5 L×3). The combined organic layers were washed with an aqueous solution of NaCl (1000 mL×3), dried over Na₂SO₄ and evaporated to dryness. The crude product was purified by column chromatography on silica gel eluted with (petroleum ether ethyl acetate 20:1→10:1→8:1) to give 2-chloro-3-fluoro-5-iodo-pyridin-4-amine (23.00 g, 76.0 mmol, 50.61% yield) as a white solid. LCMS ESI (+) m/z 272.9 (M+H).

Step D: Preparation of ethyl 4-amino-6-chloro-5-fluoro-pyridine-3-carboxylate: Under CO, to a mixed solution of 2-chloro-3-fluoro-5-iodo-pyridin-4-amine (1.00 eq, 10.00 g, 36.7 mmol) and TEA (5.00 eq, 26 mL, 184 mmol) in Ethanol (80 mL) was added Pd(dppf)Cl₂ (0.100 eq, 2.69 g, 3.67 mmol). The mixture was stirred for 24 hours at 60° C. The reaction was quenched with water (1000 ml) and extracted with ethyl acetate (1.2 L×3). The combined organic layers were washed with an aqueous solution of NaCl (1000 mL), dried over Na₂SO₄ and evaporated to dryness. The crude product was purified by column chromatography on silica gel (eluting with petroleum ether/ethyl acetate 20:1→10:1→5:1) to give ethyl 4-amino-6-chloro-5-fluoro-pyridine-3-carboxylate (6.30 g, 24.5 mmol, 66.74% yield) as a grey solid. LCMS: (ES+): m/z 219.0 [M+1].

Step E: Preparation of ethyl 6-chloro-5-fluoro-4-[(2,2,2-trichloroacetyl)carbamoylamino]pyridine-3-carboxylate: To a solution of ethyl 4-amino-6-chloro-5-fluoro-pyridine-3-carboxylate (1.00 eq, 5.80 g, 26.5 mmol) in THE (60 mL) was added 2,2,2-trichloroacetyl isocyanate (1.50 eq, 7.50 g, 39.8 mmol) at 0° C. The mixture was stirred for 2 hours at 25° C. The reaction was filtered through a Celite pad, and the solid was concentrated to give the product ethyl 6-chloro-5-fluoro-4-[(2,2,2-trichloroacetyl)carbamoylamino]pyridine-3-carboxylate (9.10 g, 20.1 mmol, 75.84% yield) directly for the next step. LCMS ESI (+) m/z 406.0 (M+H).

Step F: Preparation of 7-chloro-8-fluoro-pyrido[4,3-d]pyrimidine-2,4-diol: To a solution of ethyl 6-chloro-5-fluoro-4-[(2,2,2-trichloroacetyl)carbamoylamino]pyridine-3-carboxylate (1.00 eq, 9.10 g, 22.4 mmol) in Methanol (50 mL) was add NH₃ in MeOH (20 mL). The mixture was stirred for 4 hours at 25° C. The reaction was filtered through a Celite pad, and the solid was concentrated to give the product 7-chloro-8-fluoro-pyrido[4,3-d]pyrimidine-2,4-diol (4.80 g, 20.0 mmol, 89.63% yield) directly for the next step. LCMS ESI (+) m/z 216.1 (M+H).

Step G: Preparation of 2,4,7-trichloro-8-fluoro-pyrido[4,3-d]pyrimidine: 7-chloro-8-fluoro-pyrido[4,3-d]pyrimidine-2,4-diol (1.00 eq, 1 g, 4.64 mmol) was added in portions to the mixed solution of POCl₃ (23.0 eq, 10 mL, 106.6 mmol) and DIEA (5.00 eq, 4.2 mL, 23.2 mmol) at 0-5° C. The mixture was stirred for 20 minutes at room temperature and then stirred for another 2 hours at 100° C. After completion, the reaction was evaporated to dry under vacuum. The residue was purified by silica gel column (eluting with 25% EtOAc in Petroleum ether) to give 2,4,7-trichloro-8-fluoro-pyrido[4,3-d]pyrimidine (800 mg, 3.57 mmol) as a crude.

Step H: Preparation of tert-butyl (3S,4R)-3-[7-bromo-2-chloro-8-fluoro-6-(trifluoromethyl)quinazolin-4-yl]oxy-4-fluoro-pyrrolidine-1-carboxylate: To the solution of tert-butyl (3R,4S)-3-fluoro-4-hydroxy-pyrrolidine-1-carboxylate (1.20 eq, 200 mg, 0.974 mmol) in THE (1 mL). Cooled to −70° C. and to this was added LiHMDS (1.00 eq, 170 mg, 1.02 mmol) at −70° C. The mixture was stirred at −70° C. for 1 h. The solution of 7-bromo-2,4-dichloro-8-fluoro-6-(trifluoromethyl)quinazoline (1.00 eq, 295 mg, 0.812 mmol) in THE (1 mL) was added and then reacted for 1 h. After quenched with H₂O, the crude product was purified by column chromatography on silica gel, eluting with 25% EtOAc in petroleum ether, to give the product tert-butyl (3 S,4R)-3-[7-bromo-2-chloro-8-fluoro-6-(trifluoromethyl)quinazolin-4-yl]oxy-4-fluoro-pyrrolidine-1-carboxylate (64 mg, 14.8% yield). LCMS ESI (+) m/z 532.0 (M+H).

Step I: Preparation of tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]oxy-4-fluoro-pyrrolidine-1-carboxylate: To the solution of tert-butyl (3S,4R)-3-[7-bromo-2-chloro-8-fluoro-6-(trifluoromethyl)quinazolin-4-yl]oxy-4-fluoro-pyrrolidine-1-carboxylate (1.00 eq, 64 mg, 0.120 mmol) in 1,4-dioxane (1 mL) was added [(2R,8S)-2-fluoro-1,2,3,5,6,7-hexahydropyrrolizin-8-yl]methanol (2.00 eq, 38 mg, 0.240 mmol) at room temperature, The mixture was stirred for 8 hours at 80° C. and then extracted with EtOAc (30 ml×5). The organics was washed with 2×40 ml saturated brine solution and the organics were dried over Na₂SO₄ before concentration to dryness. The crude product was purified by column chromatography on silica gel, eluting with 50% EtOAc in petroleum ether, to give the product tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]oxy-4-fluoro-pyrrolidine-1-carboxylate (65 mg, 82.54% yield). LCMS ESI (+) m/z 655.1 (M+H).

Step J: Preparation of tert-butyl (3R)-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]pyrido [4,3-d]pyrimidin-4-yl]oxypyrrolidine-1-carboxylate: tert-butyl (3R)-3-[7-chloro-8-fluoro-2-[[(2R,8S)-2-fluoro-1,2,3,5,6,7-hexahydropyrrolizin-8-yl]methoxy]pyrido [4,3-d]pyrimidin-4-yl]oxypyrrolidine-1-carboxylate (1.00 eq, 96 mg, 0.183 mmol), K₃PO₄ (2.00 eq, 77 mg, 0.365 mmol) was dissolved in 1,4-dioxane (2 mL) and water (0.4000 mL), and to this was added [2-(tert-butoxycarbonylamino)-7-fluoro-1,3-benzothiazol-4-yl]boronic acid (1.30 eq, 74 mg, 0.237 mmol) under Ar. The mixture was stirred for 4 hrs at 90° C. and extracted with EtOAc (3×30 ml). The organics were washed with 1×30 mL saturated brine solution and dried over Na₂SO₄ before concentration to dryness. The crude product was purified by Prep-TLC, eluting with PE:EA=1:1, to give product tert-butyl (3R)-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]pyrido[4,3-d]pyrimidin-4-yl]oxypyrrolidine-1-carboxylate (90 mg, 0.119 mmol, 65.07% yield). LCMS ESI (+) m/z 758.4 (M+H).

Step K: Preparation of 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-pyrido[4,3-d]pyrimidin-7-yl]-1,3-benzothiazol-2-amine: To the solution of tert-butyl (3R)-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]pyrido [4,3-d]pyrimidin-4-yl]oxypyrrolidine-1-carboxylate (1.00 eq, 45 mg, 0.0594 mmol) in DCM (3 mL) was added trifluoroacetic acid (438 eq, 1.0 mL, 13.0 mmol) and the mixture was stirred for 2 h at room temperature. After concentrating to dry under vacuum, to this was added 15 mL of EtOAc and 20 mL water. The mixture was adjusted pH=8-9 with saturated sodium bicarbonate solution. The mixture was extracted (20 mL×3) and concentrated to dry under vacuum to give the crude. The residue was purified by Prep-HPLC to give 7-fluoro-4-(8-fluoro-2-(((2R,7aS)-2-fluorotetrahydro-1H-pyrrolizin-7a(5H)-yl)methoxy)-4-(((R)-pyrrolidin-3-yl)oxy)pyrido [4,3-d]pyrimidin-7-yl)benzo[d]thiazol-2-amine (13 mg, 0.0217 mmol, 36.63% yield). LCMS ESI (+) m/z 558.2 (M+H). ¹H NMR (400 MHz, MeOD) δ 9.20 (s, 1H), 7.45-7.48 (m, 1H), 7.03 (t, J=8.8 Hz, 1H), 6.04-6.05 (m, 1H), 5.32 (d, J=53.6 Hz, 1H), 4.42-4.43 (m, 1H), 4.37-4.40 (m, 2H), 3.04-3.70 (m, 7H), 2.48-2.51 (m, 2H), 1.93-2.34 (m, 6H).

### Synthetic Example 3: 2-amino-7-fluoro-4-(8-fluoro-2-(((2R,7aS)-2-fluorotetrahydro-1H-pyrrolizin-7a(5H)-yl)methoxy)-4-(((2R,3R)-2-methylpyrrolidin-3-yl)oxy)-6-(trifluoromethyl)quinazolin-7-yl)benzo[b]thiophene-3-carbonitrile (Compound 47)

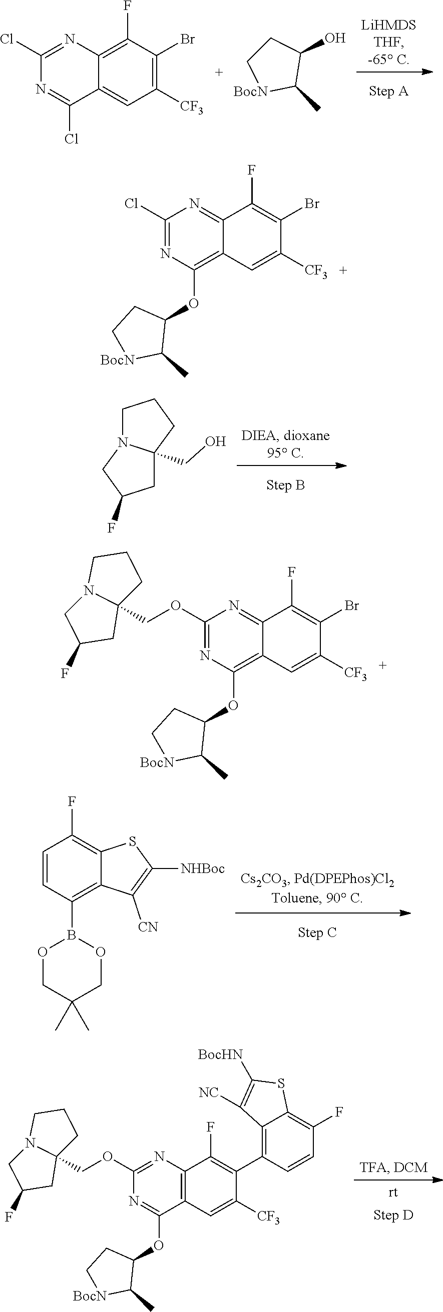

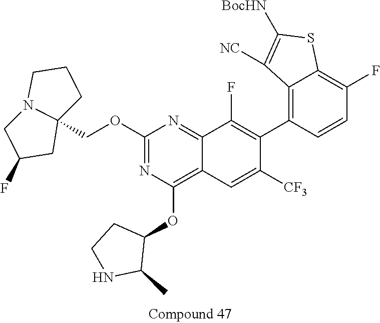 [SMILES: C[C@H]1NCC[C@H]1Oc1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12]

Step A: Preparation of tert-butyl (2R,3R)-3-((7-bromo-2-chloro-8-fluoro-6-(trifluoromethyl)quinazolin-4-yl)oxy)-2-methylpyrrolidine-1-carboxylate: To a solution of tert-butyl (2R,3R)-3-hydroxy-2-methyl-pyrrolidine-1-carboxylate (143 mg, 0.71 mmol) in THE (3 mL) was added LiHMDS (0.5 mL, 0.61 mmol) at −65° C. dropwise under argon. The mixture was kept at this temperature for 1 hour and then 7-bromo-2,4-dichloro-8-fluoro-6-(trifluoromethyl)quinazoline (200 mg, 0.55 mmol) in THE (3 mL) was added dropwise. Then the mixture was warmed to ambient temperature and kept at this temperature for 2 hours. The mixture was quenched with water and extracted with EA. The organics were washed with saturated brine solution. The organics were then separated and dried (MgSO₄) before concentration to dryness. The crude was then purified by preparative-TLC (EA:PE=1:3) to give tert-butyl (2R,3R)-3-((7-bromo-2-chloro-8-fluoro-6-(trifluoromethyl)quinazolin-4-yl)oxy)-2-methylpyrrolidine-1-carboxylate (115 mg, 39% yield) as solid.

Step B: Preparation of tert-butyl (2R,3R)-3-((7-bromo-8-fluoro-2-(((2R,7aS)-2-fluorotetrahydro-1H-pyrrolizin-7a(5H)-yl)methoxy)-6-(trifluoromethyl)quinazolin-4-yl)oxy)-2-methylpyrrolidine-1-carboxylate: To a stirred solution of tert-butyl (2R,3R)-3-((7-bromo-2-chloro-8-fluoro-6-(trifluoromethyl)quinazolin-4-yl)oxy)-2-methylpyrrolidine-1-carboxylate (115 mg, 0.21 mmol) in 1,4-dioxane (4 mL) were added [(2R,8S)-2-fluoro-1,2,3,5,6,7-hexahydropyrrolizin-8-yl]methanol (100 mg, 0.63 mmol) and DIEA (0.47 mL, 2.66 mmol). The mixture was heated to 95° C. and kept at this temperature for 8 hours under argon. The mixture was cooled to room temperature and concentrated to dryness. The residue was purified by preparative-TLC (EA:PE=1:1) to give tert-butyl (2R,3R)-3-((7-bromo-8-fluoro-2-(((2R,7aS)-2-fluorotetrahydro-1H-pyrrolizin-7a(5H)-yl)methoxy)-6-(trifluoromethyl)quinazolin-4-yl)oxy)-2-methylpyrrolidine-1-carboxylate (100 mg, 72.8% yield) as solid.

Step C: Preparation of tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)oxy)-2-methylpyrrolidine-1-carboxylate: A mixture of tert-butyl (2R,3R)-3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]oxy-2-methyl-pyrrolidine-1-carboxylate (100 mg, 0.15 mmol), tert-butyl N-[3-cyano-4-(5,5-dimethyl-1,3,2-dioxaborinan-2-yl)-7-fluoro-benzothiophen-2-yl]carbamate (186 mg, 0.46 mmol), Cs₂CO₃ (100 mg, 0.31 mmol) and DPEPhosPdCl₂ (44 mg, 0.062 mmol) in dioxane (1.5 mL) under Ar was stirred at 90° C. for 2 hours. After cooled to ambient temperature, the reaction was purified by preparative-TLC (EtOAc:PE=1:1) to give tert-butyl (2R,3R)-3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]oxy-2-methyl-pyrrolidine-1-carboxylate (63 mg, 47%). LCMS ESI (+) m/z 863.4 (M+H).

Step D: Preparation of 2-amino-7-fluoro-4-(8-fluoro-2-(((2R,7aS)-2-fluorotetrahydro-1H-pyrrolizin-7a(5H)-yl)methoxy)-4-(((2R,3R)-2-methylpyrrolidin-3-yl)oxy)-6-(trifluoromethyl)quinazolin-7-yl)benzo[b]thiophene-3-carbonitrile: To a solution of tert-butyl (2R,3R)-3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]oxy-2-methyl-pyrrolidine-1-carboxylate (31 mg, 0.036 mmol) in DCM (1 mL) was added TFA (0.50 mL) at 25° C., the mixture was stirred at 25° C. for 1.5 hours. The reaction mixture was dissolved in EtOAc and adjust pH to ˜8 with saturated NaHCO₃ solution, the organics were concentrated to dryness and the crude product was purified by RP preparative-HPLC to give 2-amino-7-fluoro-4-[8-fluoro-2-[[(2R,8S)-2-fluoro-1,2,3,5,6,7-hexahydropyrrolizin-8-yl]methoxy]-4-[(2R,3R)-2-methylpyrrolidin-3-yl]oxy-6-(trifluoromethyl)quinazolin-7-yl]benzothiophene-3-carbonitrile (10 mg, 38%) as solid. LCMS ESI (+) m/z 663.3 (M+H). ¹H NMR (400 MHz, CD₃OD) δ 8.48 (s, 1H), 7.19-7.24 (m, 1H), 7.03 (t, J=8.0 Hz, 1H), 6.07-6.09 (m, 1H), 5.36 (d, J=52 Hz, 1H), 4.43-4.49 (m, 2H), 3.95-3.96 (m, 1H), 3.31-3.53 (m, 5H), 3.17-3.19 (m, 1H), 1.85-2.62 (m, 8H), 1.53-1.55 (m, 3H).

### Synthetic Example 4: 2-amino-7-fluoro-4-(2-(((2R,7aS)-2-fluorotetrahydro-1H-pyrrolizin-7a(5H)-yl)methoxy)-4-(((R)-pyrrolidin-3-yl)oxy)-6-(trifluoromethyl)pyrido[3,2-d]pyrimidin-7-yl)benzo[b]thiophene-3-carbonitrile (Compound 93)

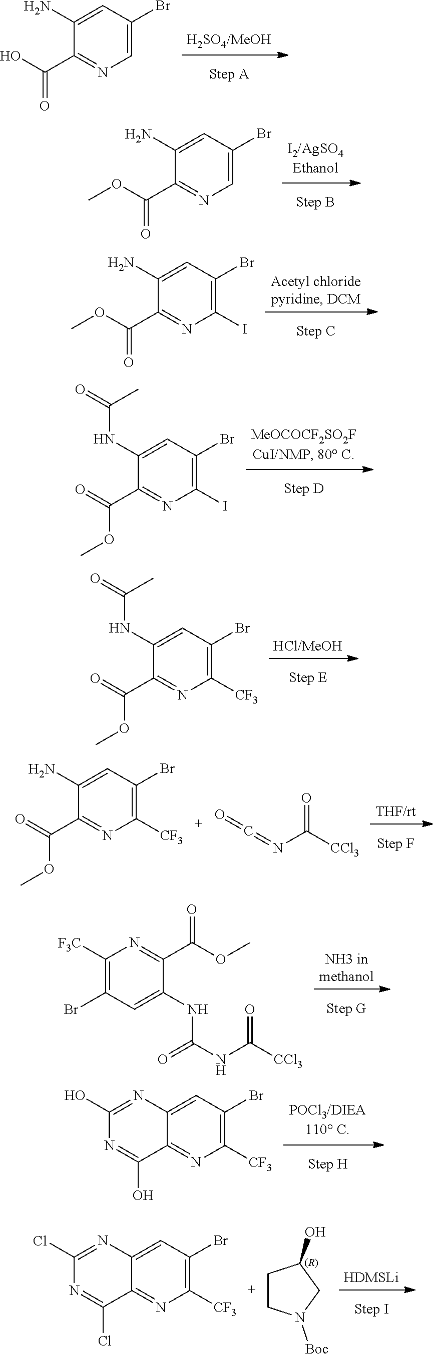

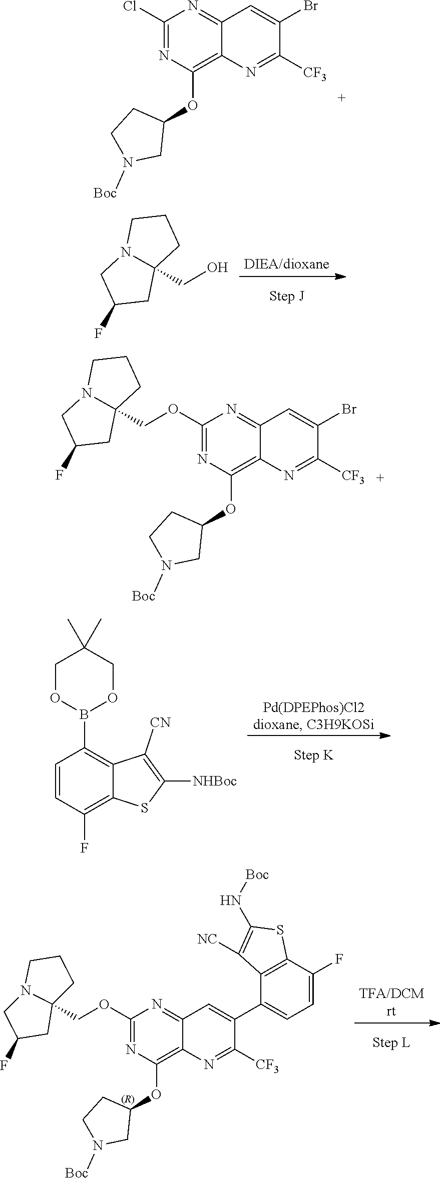

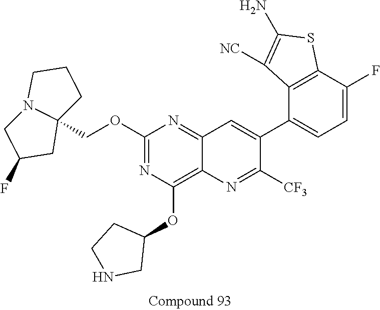 [SMILES: N#Cc1c(N)sc2c(F)ccc(-c3cc4nc(OC[C@@]56CCCN5C[C@H](F)C6)nc(O[C@@H]5CCNC5)c4nc3C(F)(F)F)c12]

Step A: Preparation of methyl 3-amino-5-bromo-pyridine-2-carboxylate: To stirring solution of 3-amino-5-bromo-pyridine-2-carboxylic acid (1.00 eq, 23.00 g, 106 mmol) in methanol (200 mL) was added dropwise H₂SO₄ (7 eq, 40 mL, 745 mmol) at 30° C. The resulting mixture was heated to 100° C. for 5 hours. The solvent was evaporated, and the residue was added water. The solid was filtered, the filter cake dissolved in DCM, washed with saturated sodium bicarbonate aqueous solution, and the organic phase was dried with Na₂SO₄, filtered and concentrated under vacuum to give methyl 3-amino-5-bromo-pyridine-2-carboxylate (20.00 g, 84.0 mmol, 79.23% yield) as a solid. LCMS ESI (+) m/z 230.9 (M+H). ¹HNMR (400 MHz, CDCl₃) δ 8.08 (s, 1H), 7.26 (s, 1H), 5.84 (s, 2H), 3.99 (s, 3H).

Step B: Preparation of methyl 3-amino-5-bromo-6-iodo-pyridine-2-carboxylate: To a mixture of iodine (1.40 eq, 30.78 g, 121 mmol) and silver sulfate (0.850 eq, 22.94 g, 73.6 mmol) in ethanol (800 mL), methyl 3-amino-5-bromo-pyridine-2-carboxylate (1.00 eq, 20.00 g, 86.6 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution, brine and the resulting organic solution was dried over Na₂SO₄, filtered, and concentrated under vacuum to give methyl 3-amino-5-bromo-6-iodo-pyridine-2-carboxylate (25 g, 67.9 mmol, 78.49% yield) as a yellow solid. LCMS ESI (+) m/z 357 (M+H). ¹HNMR (400 MHz, CDCl₃) δ 7.30 (s, 1H), 5.83 (s, 2H), 3.96 (s, 3H).

Step C: Preparation of methyl 3-acetamido-5-bromo-6-iodo-pyridine-2-carboxylate: The methyl 3-amino-5-bromo-6-iodo-pyridine-2-carboxylate (1.00 eq, 27.00 g, 75.6 mmol) and pyridine (3.00 eq, 17.93 g, 227 mmol) were dissolved in DCM (270 mL) at 0° C. Acetyl chloride (1.30 eq, 7.77 g, 98.3 mmol) was added and the reaction was warmed to ambient temperature and stirred at this temperature for 3 hours. the reaction mixture was washed with 1N hydrochloric acid to pH<7, then washed with saturated sodium bicarbonate to pH>8, the organic phase was washed with water and brine, dried and concentrated under vacuum to give methyl 3-acetamido-5-bromo-6-iodo-pyridine-2-carboxylate (30 g, 73.7 mmol, 97.42% yield) as a solid. LCMS ESI (+) m/z 398.8 (M+H). ¹HNMR (400 MHz, CDCl₃) δ 10.88 (s, 1H), 9.37 (s, 1H), 4.02 (s, 3H), 2.28 (s, 3H).

Step D: Preparation of methyl 3-acetamido-5-bromo-6-(trifluoromethyl)pyridine-2-carboxylate: To a stirred solution of methyl 3-acetamido-5-bromo-6-iodo-pyridine-2-carboxylate (1.00 eq, 20.00 g, 50.1 mmol) and methyl 2,2-difluoro-2-(fluorosulfonyl)acetate (5.00 eq, 48.12 g, 251 mmol) in NMP (260 mL) at ambient temperature, CuI (2.50 eq, 23.87 g, 125 mmol) was added and the resulting mixture was stirred at 80° C. for 1 hour. Once cooled to ambient temperature, the mixture was quenched with water, filtered, the filtrate was extracted with ethyl acetate. The organic layer was dried over Na₂SO₄, filtered, concentrated and the crude material was purified by silica gel column chromatography using ethyl acetate in hexanes (0% to 80%) as eluent to give methyl 3-acetamido-5-bromo-6-(trifluoromethyl)pyridine-2-carboxylate (14.00 g, 36.9 mmol, 73.69% yield) as a solid. LCMS ESI (+) m/z 340.9 (M+H). ¹HNMR (400 MHz, CDCl₃) δ 11.12 (s, 1H), 9.60 (s, 1H), 4.05 (s, 3H), 2.32 (s, 3H).

Step E: Preparation of methyl 3-amino-5-bromo-6-(trifluoromethyl)pyridine-2-carboxylate: A mixture of 3-acetamido-5-bromo-6-(trifluoromethyl)pyridine-2-carboxylate (1.00 eq, 14.00 g, 41.0 mmol) in 3M HCl in methanol (10.0 eq, 140 mL, 410 mmol) was heated at 60° C. for 2 hours. Once cooled to ambient temperature, the solvent was added saturated NaHCO₃ to pH>7, and the crude product was extracted with EtOAc. The organic layer was separated, dried over sodium sulfate, filtered, and evaporated to provide methyl 3-amino-5-bromo-6-(trifluoromethyl)pyridine-2-carboxylate (14.00 g, 42.1 mmol, 100% yield). LCMS ESI (+) m/z 298.9 (M+H). ¹HNMR (400 MHz, CDCl₃) δ 7.41 (s, 1H), 6.16 (s, 2H), 3.99 (s, 3H).

Step F: Preparation of methyl 5-bromo-3-[(2,2,2-trichloroacetyl)carbamoylamino]-6-(trifluoromethyl)pyridine-2-carboxylate: To a mixture of methyl 3-amino-5-bromo-6-(trifluoromethyl)pyridine-2-carboxylate (1.00 eq, 14.00 g, 46.8 mmol) in THE (150 mL) was added 2,2,2-trichloroacetyl isocyanate (1.50 eq, 13.23 g, 70.2 mmol) at ambient temperature. After 5 hours, the reaction mixture was evaporated to provide methyl 5-bromo-3-[(2,2,2-trichloroacetyl)carbamoylamino]-6-(trifluoromethyl)pyridine-2-carboxylate (26.30 g, 46.4 mmol, 99.11% yield). LCMS ESI (+) m/z 485.7 (M+H). ¹H NMR (400 MHz, CDCl₃) δ 12.56 (s, 1H), 9.51 (s, 1H), 8.87 (s, 1H), 4.12 (s, 3H).

Step G: Preparation of 7-bromo-6-(trifluoromethyl)pyrido[3,2-d]pyrimidine-2,4-diol: To a solution of methyl 5-bromo-3-[(2,2,2-trichloroacetyl)carbamoylamino]-6-(trifluoromethyl)pyridine-2-carboxylate (1.00 eq, 27.00 g, 55.4 mmol) in methanol (300 mL) was added NH₃ in CH₃OH (7M) (4.00 eq, 27 mL, 222 mmol) at ambient temperature and stirred at ambient temperature for 1 hour. The mixture was concentrated under reduced pressure to provide a solid. The crude product was purified by silica gel column chromatography using ethyl acetate in hexanes (50% to 100%) as eluent to give 7-bromo-6-(trifluoromethyl)pyrido[3,2-d]pyrimidine-2,4-diol (9.90 g, 29.4 mmol, 53.04% yield). LCMS ESI (+) m/z 309.9 (M+H). ¹H NMR (400 MHz, DMSO-d₆) δ 11.80 (s, 2H), 7.99 (s, 1H).

Step H: Preparation of 7-bromo-2,4-dichloro-6-(trifluoromethyl)pyrido[3,2-d]pyrimidine: To a stirring solution of phosphorus oxychloride (33.2 eq, 5.0 mL, 53.6 mmol) and DIPEA (5.00 eq, 1.4 mL, 8.06 mmol) was added 7-bromo-6-(trifluoromethyl)pyrido[3,2-d]pyrimidine-2,4-diol (1.00 eq, 500 mg, 1.61 mmol) at 0° C. After addition, the resulting mixture was stirred at 110° C. for 1 hour. Once cooled down to ambient temperature, the mixture was evaporated and co-evaporated with chloroform to give 7-bromo-2,4-dichloro-6-(trifluoromethyl)pyrido[3,2-d]pyrimidine (2.50 g crude, 1.44 mmol, 89.37% yield) which used as such for the next step. LCMS ESI (+) m/z 346 (M+H).

Step I: Preparation of tert-butyl (R)-3-((7-bromo-2-chloro-6-(trifluoromethyl)pyrido[3,2-d]pyrimidin-4-yl)oxy)pyrrolidine-1-carboxylate: To a solution of tert-butyl (3R)-3-hydroxypyrrolidine-1-carboxylate (378 mg, 2.02 mmol) in anhydrous THE (6 mL) was added was added LiHMDS (2.8 mL, 3.03 mmol) dropwise at −78° C. under Ar and then stirred at −78° C. for 1 hour. 7-bromo-2,4-dichloro-6-(trifluoromethyl)pyrido[3,2-d]pyrimidine (1.00 eq, 700 mg, 2.02 mmol) in THE (2 mL) was added at −78° C. After stirred for 1 hour at −78° C. water, extracted with EA (3*10 mL), the combined organic phase was washed brine, dried over anhydrous sodium sulfate, concentrated under vacuum. The residue was purified with preparative-TLC (petroleum ether/ethyl acetate=5:1) to afford tert-butyl (3R)-3-[7-bromo-2-chloro-6-(trifluoromethyl)pyrido[3,2-d]pyrimidin-4-yl]oxypyrrolidine-1-carboxylate (350 mg, 31%) as solid. LCMS ESI (+) m/z 497.1 (M+H).

Step J: Preparation of tert-butyl (R)-3-((7-bromo-2-(((2R,7aS)-2-fluorotetrahydro-1H-pyrrolizin-7a(5H)-yl)methoxy)-6-(trifluoromethyl)pyrido[3,2-d]pyrimidin-4-yl)oxy)pyrrolidine-1-carboxylate: To a solution of tert-butyl (3R)-3-[7-bromo-2-chloro-6-(trifluoromethyl)pyrido[3,2-d]pyrimidin-4-yl]oxypyrrolidine-1-carboxylate (350 mg, 0.70 mmol) in dioxane (5 mL) was added [(2R,8S)-2-fluoro-1,2,3,5,6,7-hexahydropyrrolizin-8-yl]methanol (179 mg, 1.13 mmol) and DIEA (0.63 mL, 3.52 mmol) at room temperature. The resulting reaction mixture was stirred at 80° C. for 16 hours under nitrogen atmosphere. After cooled to ambient temperature, the resulting mixture was diluted with water and extracted with EA (3*20 mL). The combined organic phase was washed brine, dried over anhydrous sodium sulfate, concentrated under vacuum. The residue was purified with preparative-TLC (petroleum ether/ethyl acetate=1:1) to afford tert-butyl (R)-3-((7-bromo-2-(((2R,7aS)-2-fluorotetrahydro-1H-pyrrolizin-7a(5H)-yl)methoxy)-6-(trifluoromethyl)pyrido[3,2-d]pyrimidin-4-yl)oxy)pyrrolidine-1-carboxylate (298 mg, 68% yield) as solid. LCMS ESI (+) m/z 620.1 (M+H).

Step K: Preparation tert-butyl (R)-3-((7-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-6-(trifluoromethyl)pyrido[3,2-d]pyrimidin-4-yl)oxy)pyrrolidine-1-carboxylate: To a mixture of tert-butyl (3R)-3-[7-bromo-2-[[(2R,8S)-2-fluoro-1,2,3,5,6,7-hexahydropyrrolizin-8-yl]methoxy]-6-(trifluoromethyl)pyrido[3,2-d]pyrimidin-4-yl]oxypyrrolidine-1-carboxylate (110 mg, 0.18 mmol) and tert-butyl N-[3-cyano-4-(5,5-dimethyl-1,3,2-dioxaborinan-2-yl)-7-fluoro-benzothiophen-2-yl]carbamate (143 mg, 0.36 mmol) in dioxane (2 mL) was added potassium trimethylsilanolate (0.11 mL, 0.53 mmol) and Pd(DPEPhos)Cl₂ (63 mg, 0.089 mmol) at room temperature. The resulting mixture was stirred at 95° C. for 6 hours under nitrogen atmosphere. After cooled to ambient temperature, the resulting mixture was diluted with water and extracted with EA (3*20 mL). The combined organic phase was washed brine, dried over anhydrous sodium sulfate, concentrated under vacuum. The residue was purified with preparative-TLC (petroleum ether/ethyl acetate=1:1) to afford tert-butyl (3R)-3-[7-[2-(tert-butoxycarbonylamino)-3-cyano-7-fluoro-benzothiophen-4-yl]-2-[[(2R,8S)-2-fluoro-1,2,3,5,6,7-hexahydropyrrolizin-8-yl]methoxy]-6-(trifluoromethyl)pyrido[3,2-d]pyrimidin-4-yl]oxypyrrolidine-1-carboxylate (110 mg, 74% yield) as solid. LCMS ESI (+) m/z 832.1 (M+H).

Step L: Preparation of 2-amino-7-fluoro-4-(2-(((2R,7aS)-2-fluorotetrahydro-1H-pyrrolizin-7a(5H)-yl)methoxy)-4-(((R)-pyrrolidin-3-yl)oxy)-6-(trifluoromethyl)pyrido[3,2-d]pyrimidin-7-yl)benzo[b]thiophene-3-carbonitrile: To a solution of tert-butyl (3R)-3-[7-[2-(tert-butoxycarbonylamino)-3-cyano-7-fluoro-benzothiophen-4-yl]-2-[[(2R,8S)-2-fluoro-1,2,3,5,6,7-hexahydropyrrolizin-8-yl]methoxy]-6-(trifluoromethyl)pyrido[3,2-d]pyrimidin-4-yl]oxypyrrolidine-1-carboxylate (200 mg, 0.24 mmol) in DCM (5 mL) was added TFA (2.5 mL, 32.5 mmol) at room temperature under nitrogen atmosphere. The resulting solution was stirred for 16 hours at room temperature under nitrogen atmosphere. The solvent was removed and the residue was purified by RP preparative-HPLC to give 2-amino-4-[4-(3,8-diazabicyclo [3.2.1] octan-3-yl)-2-[[(2R,8S)-2-fluoro-1,2,3,5,6,7-hexahydropyrrolizin-8-yl]methoxy]pyrido[2,3-d]pyrimidin-7-yl]-7-fluoro-benzothiophene-3-carbonitrile (14 mg, 9% yield) as solid. LCMS ESI (+) m/z 632.0 (M+H). ¹H NMR (400 MHz, CD₃OD) δ 8.32 (d, 1H), 8.16-8.20 (m, 1H), 7.22-7.25 (m, 1H), 7.02-7.06 (m, 1H), 6.15 (s, 1H), 5.58-5.64 (m, 0.5H), 5.45-5.51 (m, 0.5H), 4.62-4.69 (m, 2H), 4.29 (s, 1H), 3.59-3.85 (m, 6H), 2.05-2.62 (m, 9H).

### Synthetic Example 5: Synthesis of 2-amino-7-fluoro-4-(8-fluoro-2-(((2R,7aS)-2-fluorotetrahydro-1H-pyrrolizin-7a(5H)-yl)methoxy)-4-(((2R,3R)-2-methylpyrrolidin-3-yl)oxy)pyrido[4,3-d]pyrimidin-7-yl)benzo[b]thiophene-3-carbonitrile (Compound 98)

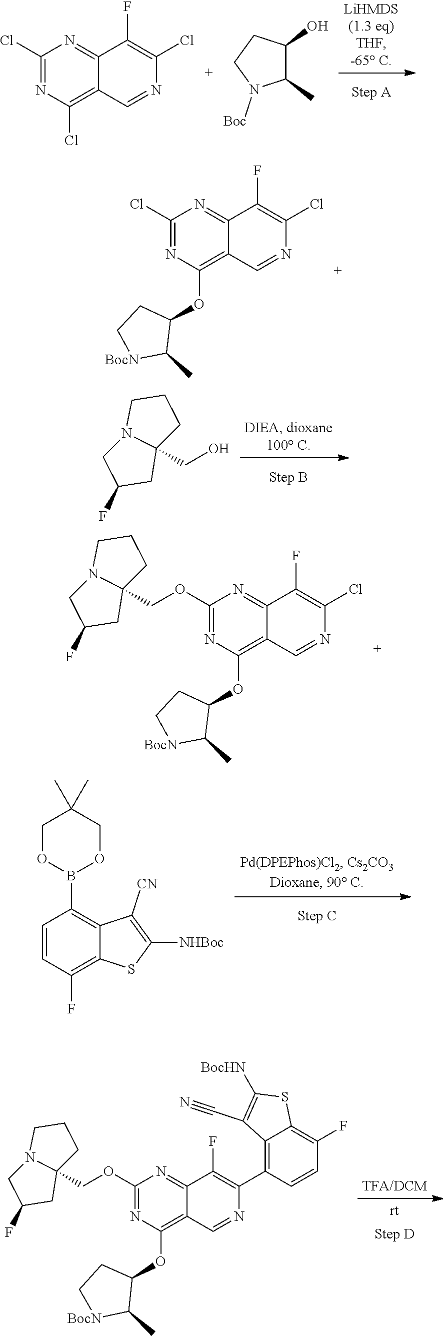

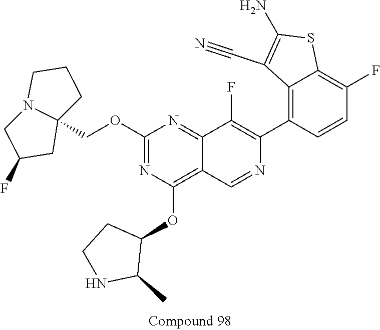 [SMILES: C[C@H]1NCC[C@H]1Oc1nc(OC[C@@]23CCCN2C[C@H](F)C3)nc2c(F)c(-c3ccc(F)c4sc(N)c(C#N)c34)ncc12]

Step A: Preparation of tert-butyl (2R,3R)-3-((2,7-dichloro-8-fluoropyrido[4,3-d]pyrimidin-4-yl)oxy)-2-methylpyrrolidine-1-carboxylate: To a solution of tert-butyl (2R,3R)-3-hydroxy-2-methyl-pyrrolidine-1-carboxylate (106 mg, 0.53 mmol) in THE (3 mL) at −65° C. was added LiHMDS (0.45 mL, 0.45 mmol) dropwise under argon. The mixture was kept at this temperature for 1 hour and then 2,4,7-trichloro-8-fluoropyrido[4,3-d]pyrimidine (102 mg, 0.41 mmol) in THE (3 mL) was added dropwise. Then the mixture was warmed to ambient temperature and kept at this temperature for 2 hours. The mixture was quenched with water and extracted with EA. The organics were washed with brine solution. The organics were then separated and dried (MgSO₄) before concentration to dryness. The crude was then purified by preparative-TLC (EA:PE=1:3) to give tert-butyl (2R,3R)-3-((2,7-dichloro-8-fluoropyrido[4,3-d]pyrimidin-4-yl)oxy)-2-methylpyrrolidine-1-carboxylate (93 mg, 54% yield) as solid.

Step B: Preparation of tert-butyl (2R,3R)-3-((7-chloro-8-fluoro-2-(((2R,7aS)-2-fluorotetrahydro-1H-pyrrolizin-7a(5H)-yl)methoxy)pyrido[4,3-d]pyrimidin-4-yl)oxy)-2-methylpyrrolidine-1-carboxylate: To a stirred solution of tert-butyl (2R,3R)-3-((2,7-dichloro-8-fluoropyrido[4,3-d]pyrimidin-4-yl)oxy)-2-methylpyrrolidine-1-carboxylate (93 mg, 0.223 mmol) in 1,4-dioxane was added [(2R,8S)-2-fluoro-1,2,3,5,6,7-hexahydropyrrolizin-8-yl]methanol (106 mg, 0.67 mmol) and DIEA (0.47 mL, 2.66 mmol). The mixture was heated to 95° C. and kept at this temperature for 8 hours under argon. The mixture was cooled to room temperature and concentrated to dryness. The residue was purified by preparative-TLC(EA:PE=1:1) to give tert-butyl (2R,3R)-3-((7-chloro-8-fluoro-2-(((2R,7aS)-2-fluorotetrahydro-1H-pyrrolizin-7a(5H)-yl)methoxy)pyrido[4,3-d]pyrimidin-4-yl)oxy)-2-methylpyrrolidine-1-carboxylate (98 mg, 81% yield) as solid.

Step C: Preparation tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)pyrido[4,3-d]pyrimidin-4-yl)oxy)-2-methylpyrrolidine-1-carboxylate: To a stirred solution of tert-butyl (2R,3R)-3-((7-chloro-8-fluoro-2-(((2R,7aS)-2-fluorotetrahydro-1H-pyrrolizin-7a(5H)-yl)methoxy)pyrido[4,3-d]pyrimidin-4-yl)oxy)-2-methylpyrrolidine-1-carboxylate (98 mg, 0.18 mmol) in 1,4-dioxane (4 mL) were added tert-butyl N-[3-cyano-4-(5,5-dimethyl-1,3,2-dioxaborinan-2-yl)-7-fluoro-benzothiophen-2-yl]carbamate (219 mg, 0.54 mmol), Pd(DPEPhos)Cl₂ (13 mg, 0.018 mmol) and Cs₂CO₃ (118 mg, 0.36 mmol). The mixture was heated to 95° C. and stirred at this temperature for 8 hours under argon. The mixture was cooled to room temperature and diluted with EA, then filtered. The filtrate was concentrated and the residue was purified by preparative-TLC (MeOH:DCM=1:20) to give tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)pyrido[4,3-d]pyrimidin-4-yl)oxy)-2-methylpyrrolidine-1-carboxylate (75 mg, 52% yield) as solid. LCMS ESI (+) m/z 796.3 (M+H).

Step D: Preparation of 2-amino-7-fluoro-4-(8-fluoro-2-(((2R,7aS)-2-fluorotetrahydro-1H-pyrrolizin-7a(5H)-yl)methoxy)-4-(((2R,3R)-2-methylpyrrolidin-3-yl)oxy)pyrido[4,3-d]pyrimidin-7-yl)benzo[b]thiophene-3-carbonitrile: To a stirred solution of tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)pyrido[4,3-d]pyrimidin-4-yl)oxy)-2-methylpyrrolidine-1-carboxylate (75 mg, 0.094 mmol) in DCM (3 mL) was added TFA (1.0 mL, 13.0 mmol). The mixture was stirred at temperature for 4 hours. The mixture was concentrated, and the residue was dissolved in EtOAc (20 mL) and the organics washed with 10 ml NaHCO₃ solution then 10 mL saturated brine solution. The organics were then separated and dried (MgSO₄) before concentration to dryness. The crude was then used in the next step without further purification. LCMS ESI (+) m/z 596.2 (M+H). ¹H NMR (400 MHz, CD₃OD) δ 9.31 (s, 1H), 7.40-7.43 (m, 1H), 7.08 (t, J=8.8 Hz, 1H), 6.10 (s, 1H), 5.45 (d, J=52.8 Hz, 1H), 4.56-4.63 (m, 3H), 4.01-4.02 (m, 1H), 3.53-3.60 (m, 5H), 2.05-2.64 (m, 8H), 1.55 (d, J=6.4 Hz, 3H).

A compound of present disclosure, such as a compound of a formula included in Table 2 or Table 3, may be synthesized according to one of the general routes outlined in Synthetic Examples 1-5 or by various other methods generally known in the art.

Table 2 includes selected compounds of the present disclosure.

TABLE 2
Selected compounds of the present disclosure.

| Compound | | |
|---|---|---|
| No. | Structure | Analytical data |
| 1 | 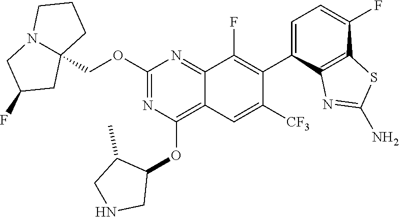 | LCMS m/z [M + 1]: 639.2 ¹HNMR(400 MHz, MeOD): δ 8.36 (s, 1H), 7.15-7.22 (m, 1H), 6.97 (t, J = 8.8 Hz, 1H), 5.58-2.69 (m, 1H), 5.32 (d, J = 52.4 Hz, 1H), 4.55-4.63 (m, 2H), 4.31-4.43 (m, 2H), 3.56-3.77 (m, 3H), 3.19-3.27 (m, 1H), 2.96- 3.08 (m, 2H), 2.74-2.86 (m, 1H), 2.11-2.36 (m, 3H), 1.85-2.06 (m, 3H), 1.30 (d, J = 7.6 Hz, 3H) |
| 2 | 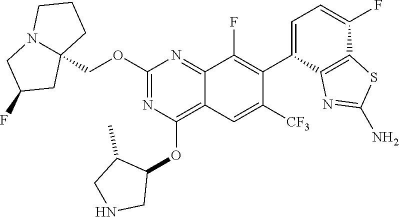 | LCMS m/z [M + 1]: 639.2 ¹HNMR(400 MHz, MeOD): δ 8.37 (s, 1H), 7.16-7.22 (m, 1H), 6.98 (d, J = 8.8 Hz, 1H), 5.64-5.72 (m, 1H), 5.35 (d, J = 54 Hz, 1H), 4.49-4.65 (m, 2H), 4.33-4.47 (m, 2H), 3.81-3.88 (m, 1H), 3.64-3.75 (m, 2H), 3.34- 3.41 (m, 1H), 3.05-3.16 (m, 2H), 2.81-2.92 (m, 1H), 2.14-2.43 (m, 3H), 1.91-2.12 (m, 3H), 1.31 (d, J = 8.0 Hz, 3H) |
| 2a | 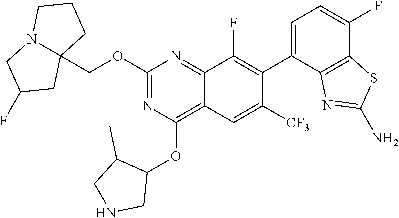 [SMILES: CC1CNCC1Oc1nc(OCC23CCCN2CC(F)C3)nc2c(F)c(-c3ccc(F)c4sc(N)nc34)c(C(F)(F)F)cc12] | |
| 3 | 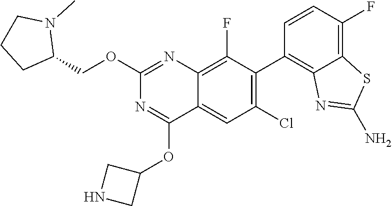 [SMILES: CN1CCC[C@H]1COc1nc(OC2CNC2)c2cc(Cl)c(-c3ccc(F)c4sc(N)nc34)c(F)c2n1] | LCMS m/z [M + 1]: 533.2 ¹H NMR (400 MHz, CD₃OD) δ 8.20 (s, 1H), 7.19-7.25 (m, 1H), 7.02 (t, J = 8.8 Hz, 1H), 5.78-5.86 (m, 1H), 4.63-4.74 (m, 3H), 4.41-4.48 (m, 2H), 3.87-3.95 (m, 1H), 3.70-3.79 (m, 1H), 3.07-3.15 (m, 3H), 2.38-2.47 (m, 1H), 2.05-2.26 (m, 4H). |
| 3a | 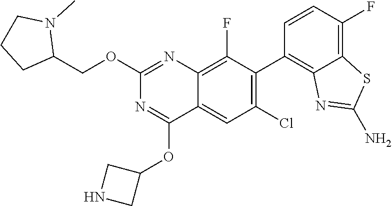 [SMILES: CN1CCCC1COc1nc(OC2CNC2)c2cc(Cl)c(-c3ccc(F)c4sc(N)nc34)c(F)c2n1] | |
| 4 | 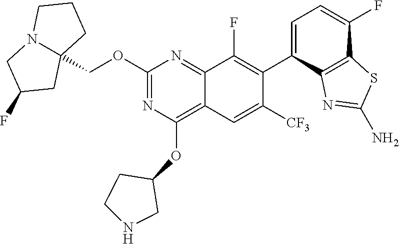 | LCMS m/z [M+1]: 625.2 ¹H NMR (400 MHz, MeOD) δ 8.52 (s, 1H), 8.37 (s, 1H), 7.12-7.25 (m, 1H), 6.97 (t, J = 8.8 Hz, 1H), 5.98- 6.09 (m, 1H), 5.32 (d, J = 54.4 Hz, 1H), 4.25-4.41 (dd, J = 22.8, 10.8 Hz, 2H), 3.58-3.61 (m, 2H), 3.47-3.58 (m, 2H), 3.20-3.30 (m, 3H), 3.03-3.11 (m, 1H), 2.39- 2.45 (m, 2H), 2.08-2.38 (m, 3H), 1.9-2.03 (m, 3H). |
| 5 | 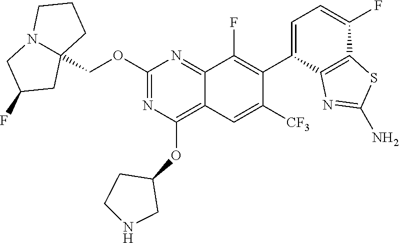 | LCMS m/z [M + 1]: 625.2 ¹H NMR (400 MHz, MeOD) δ 8.53 (s, 1H), 8.36 (s, 1H), 7.12-7.25 (m, 1H), 6.97 (t, J = 8.8 Hz, 1H), 5.98- 6.09 (m, 1H), 5.32 (d, J = 54.4 Hz, 1H), 4.25-4.41 (dd, J = 22.8, 10.8 Hz, 2H), 3.58-3.61 (m, 2H), 3.47-3.58 (m, 2H), 3.20-3.30 (m, 3H), 3.03-3.11 (m, 1H), 2.39- 2.45 (m, 2H), 2.08-2.38 (m, 3H), 1.9-2.03 (m, 3H). |
| 5a | 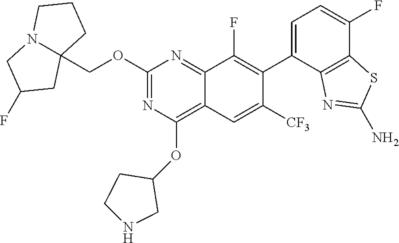 [SMILES: Nc1nc2c(-c3c(C(F)(F)F)cc4c(OC5CCNC5)nc(OCC56CCCN5CC(F)C6)nc4c3F)ccc(F)c2s1] | |
| 6 | 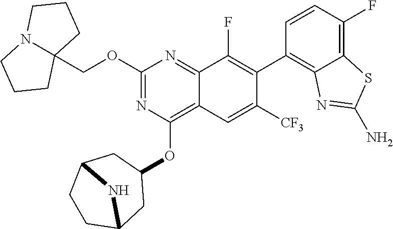 | LCMS m/z [M + 1]: 647.3 ¹H NMR (400 MHz, CD₃OD) δ 8.28 (s, 1H), 7.17-7.23 (m, 1H), 6.96-7.03 (m, 1H), 5.80-5.90 (m, 1H), 4.75 (s, 2H), 4.26 (s, 2H), 3.66-3.77 (m, 2H), 3.32-3.37 (m, 2H), 2.57-2.67 (m, 2H), 2.08-2.40 (m, 14H). |
| 6a | 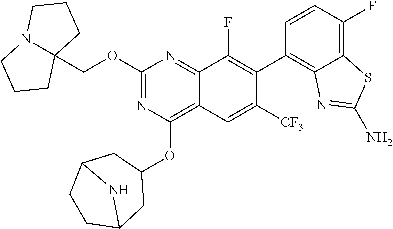 [SMILES: Nc1nc2c(-c3c(C(F)(F)F)cc4c(OC5CC6CCC(C5)N6)nc(OCC56CCCN5CCC6)nc4c3F)ccc(F)c2s1] | |
| 7 | 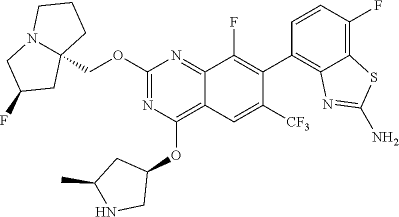 [SMILES: C[C@H]1C[C@@H](Oc2nc(OC[C@@]34CCCN3C[C@H](F)C4)nc3c(F)c(-c4ccc(F)c5sc(N)nc45)c(C(F)(F)F)cc23)CN1] | LCMS m/z [M+1]: 639.2 1H NMR (400 MHz, CD₃OD) δ 8.38 (s, 1H), 7.13- 7.24 (m, 1H), 6.97 (t, J = 8.8 Hz, 1H), 5.99 (s, 1H), 5.32 (d, J = 54.0 Hz, 1H), 4.44-4.29 (m, 2H), 3.58- 3.81 (m, 3H), 3.31-3.37 (m, 2H), 3.21-3.27 (m, 1H), 3.00-3.12 (m, 1H), 2.80-2.93 (m, 1H), 1.89-2.36 (m, 7H), 1.51 (d, J = 6.6 Hz, 3H). |
| 7a | 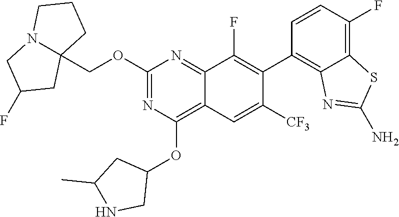 [SMILES: CC1CC(Oc2nc(OCC34CCCN3CC(F)C4)nc3c(F)c(-c4ccc(F)c5sc(N)nc45)c(C(F)(F)F)cc23)CN1] | |
| 8 | 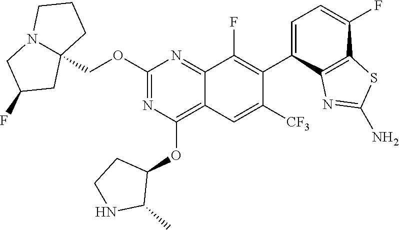 | LCMS m/z [M + 1]: 639.3 1H NMR (400 MHz, CD₃OD) δ 8.33 (s, 1H), 7.13- 7.23 (m, 1H), 6.96 (t, J = 8.8 Hz, 1H), 5.50-5.63 (m, 1H), 5.31 (d, J = 53.1 Hz, 1H), 4.21-4.41 (m, 2H), 3.70-3.80 (m, 1H), 3.32-3.38 (m, 2H), 3.13-3.29 (m, 3H), 2.93-3.07 (m, 1H), 1.82-2.58 (m, 8H), 1.41 (d, J = 7.0 Hz, 3H). |
| 9 | 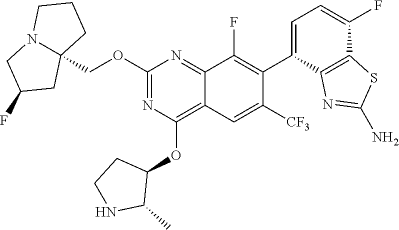 | LCMS m/z [M + 1]: 639.3 1H NMR (400 MHz, CD₃OD) δ 8.37 (s, 1H), 7.13- 7.22 (m, 1H), 6.95 (t, J = 8.8 Hz, 1H), 5.66-5.74 (m, 1H), 5.34 (d, J = 53.2 Hz, 1H), 4.32-4.47 (m, 2H), 4.03-4.16 (m, 1H), 3.52-3.63 (m, 2H), 3.32-3.39 (m, 2H), 3.21-3.28 (m, 1H), 3.05-3.13 (m, 1H), 2.59-2.71 (m, 1H), 2.42-2.53 (m, 1H), 2.14-2.38 (m, 3H), 1.91- 2.10 (m, 3H), 1.53 (d, J = 7.1 Hz, 3H). |
| 9a | 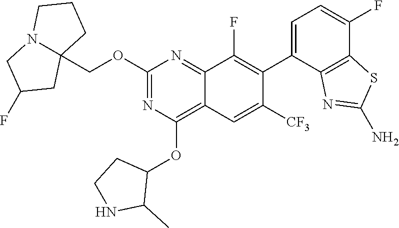 [SMILES: CC1NCCC1Oc1nc(OCC23CCCN2CC(F)C3)nc2c(F)c(-c3ccc(F)c4sc(N)nc34)c(C(F)(F)F)cc12] | |
| 10 | 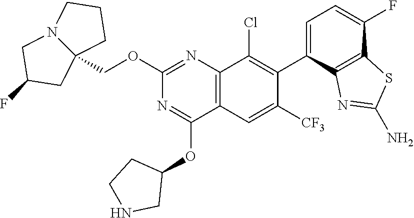 | LCMS m/z [M + 1]: 641.3 1H NMR (400 MHz, CD₃OD) δ 8.50 (s, 1H), 7.03- 7.18 (m, 1H), 6.95 (t, J = 8.8 Hz, 1H), 6.00 (s, 1H), 5.30 (d, J = 53.2 Hz, 1H), 4.22-4.53 (m, 2H), 3.39- 3.75 (m, 3H), 3.32-3.39 (m, 2H), 3.10-3.27 (m, 2H), 2.94-3.08 (m, 1H), 1.64-2.67 (m, 8H). |
| 11 | 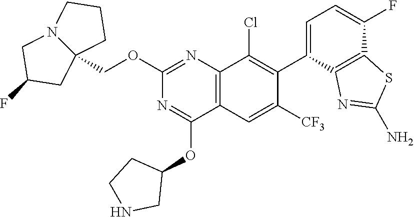 | LCMS m/z [M + 1]: 641.3 1H NMR (400 MHz, CD₃OD) δ 8.50 (s, 1H), 7.04- 7.16 (m, 1H), 6.96 (t, J = 8.8 Hz, 1H), 5.99 (s, 1H), 5.30 (d, J = 54.2 Hz, 1H), 4.25-4.51 (m, 2H), 3.38- 3.67 (m, 3H), 3.32-3.37 (m, 2H), 3.10-3.26 (m, 2H), 2.97-3.08 (m, 1H), 1.77-2.55 (m, 8H). |
| 11a | 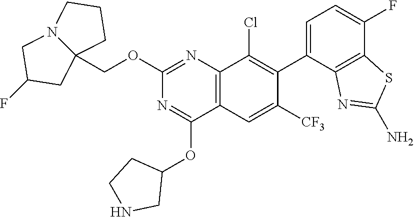 [SMILES: Nc1nc2c(-c3c(C(F)(F)F)cc4c(OC5CCNC5)nc(OCC56CCCN5CC(F)C6)nc4c3Cl)ccc(F)c2s1] | |
| 12 | 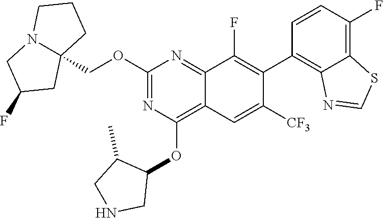 | LCMS m/z [M + 1]: 624.3 1H NMR (400 MHz, CD₃OD): δ 9.25 (s, 1H), 8.47(s, 2H), 8.44 (s, 1H), 7.56 (dd, J = 8.0, 5.2 Hz, 1H), 7.43 (t, J = 8.8 Hz, 1H), 5.68-5.75 (m, 1H), 5.40 (d, J = 52.0 Hz, 1H), 4.50-4.60 (m, 2H), 3.85-3.94 (m, 1H), 3.74 (dd, J = 11.2, 7.2 Hz, 2H) , 3.45-3.65 (m, 3H), 3.10- 3.29 (m, 2H), 2.85-2.94 (m, 1H), 2.44-2.65 (m, 3H), 2.08-2.39 (m, 3H), 1.32 (d, J = 7.2 Hz, 3H). |
| 12a | 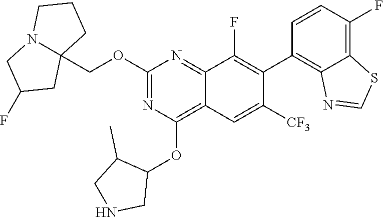 [SMILES: CC1CNCC1Oc1nc(OCC23CCCN2CC(F)C3)nc2c(F)c(-c3ccc(F)c4scnc34)c(C(F)(F)F)cc12] | |
| 13 | 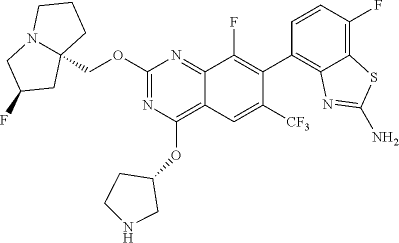 [SMILES: Nc1nc2c(-c3c(C(F)(F)F)cc4c(O[C@H]5CCNC5)nc(OC[C@@]56CCCN5C[C@H](F)C6)nc4c3F)ccc(F)c2s1] | LCMS m/z [M + 1]: 625.3 1H NMR (400 MHz, CD₃OD): δ 8.47 (s, 1 H), 8.39 (s, 1H), 7.19 (dd, J = 8.4, 5.6 Hz, 1H), 6.97 (t, J = 8.8 Hz, 1H), 6.05-6.12 (m, 1H), 5.40 (d, J = 52.8 Hz, 1H), 4.41-4.55 (m, 2H), 3.59-3.79 (m, 4H), 3.35-3.45 (m, 3H), 3.12-3.20 (m, 1H), 2.50-2.55 (m, 2H), 1.97-2.40 (m, 6H). |
| 14 | 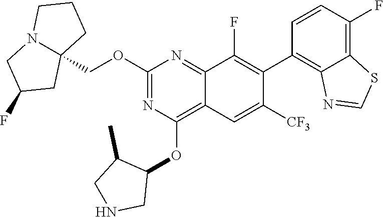 | LCMS m/z [M + 1]: 624.3 1H NMR (400 MHz, MeOD-d4): δ 9.26 (s, 1H), 8.42 (d, J = 3.42 Hz, 1H), 7.55-7.59 (m, 1H), 7.44 (t, J = 8.73 Hz, 1H), 6.09 (t, J = 3.80 Hz, 1H), 5.36 (d, J = 53.15 Hz, 1H), 4.38-4.60 (m, 2H), 3.65-3.84 (m, 3H), 3.35-3.42 (m, 2H), 3.25-3.30 (m, 2H), 3.08-3.14 (m, 1H), 2.77-2.85 (m, 1H), 1.95-2.42 (m, 6H), 1.28 (d, J = 6.73 Hz, 3H). |
| 15 | 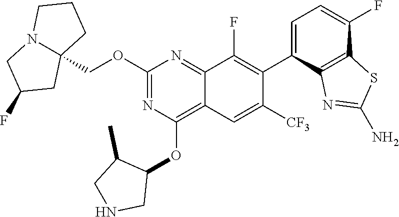 | LCMS m/z [M + 1]: 639.2 1H NMR (400 MHz, MeOD) δ 8.37 (s, 1H), 7.17-7.21 (m, 1H), 6.98 (t, J = 9.2 Hz, 1H), 6.08 (m, 1H), 5.38 (d, J = 54 Hz, 1H), 4.41-4.48 (m, 2H), 3.68-3.79 (m, 3H), 3.24-3.44 (m, 5H), 2.80-2.81 (m, 1H), 2.01-2.42 (m, 6H), 1.21-1.45 (m, 3H ). |
| 16 | 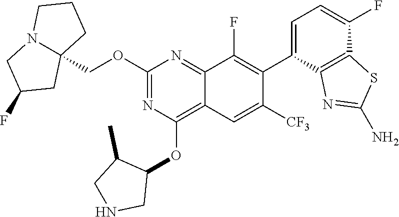 | LCMS m/z [M + 1]: 639.2 1H NMR (400 MHz, MeOD) δ 8.36 (s, 1H), 7.18-7.22 (m, 1H), 6.98 (t, J = 8.8 Hz, 1H), 6.08 (m, 1H), 5.34 (d, J = 52.4 Hz, 1H), 4.39-4.45 (m, 2H), 3.69-3.79 (m, 3H), 3.10-3.49 (m, 5H), 2.79 (m, 1H), 2.01-2.37 (m, 6H), 1.21-1.45 (m, 3H). |
| 17 | 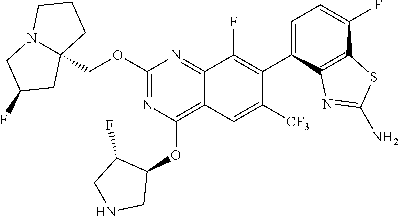 | LCMS m/z [M + 1]: 643.2 1H NMR (400 MHz, MeOD) δ 8.35 (s, 1H), 7.16-7.20 (m, 1H), 6.97 (t, J = 8.8 Hz, 1H), 5.86-5.90 (m, 1H), 5.46 (d, J = 50.8 Hz, 1H), 5.45 (d, J = 50.4 Hz, 1H), 4.41-4.48 (m, 2H), 3.60-3.64 (m, 1H), 3.36-3.41 (m, 5H), 3.10-3.13 (m, 1H), 2.01-2.41 (m, 7H). |
| 18 | 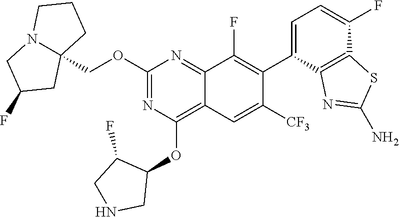 | LCMS m/z [M + 1]: 643.2 1H NMR (400 MHz, MeOD) δ 8.36 (s, 1H), 7.16-7.20 (m, 1H), 6.97 (t, J = 8.8 Hz, 1H), 5.86-5.90 (m, 1H), 5.46 (d, J = 50.8 Hz, 1H), 5.45 (d, J = 50.4 Hz, 1H), 4.41-4.48 (m, 2H), 3.62-3.65 (m, 1H), 3.53-3.57 (m, 5H), 3.11-3.13 (m, 1H), 2.01-2.41 (m, 7H). |
| 18a | 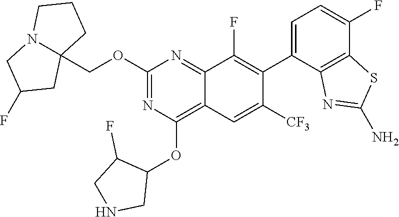 [SMILES: Nc1nc2c(-c3c(C(F)(F)F)cc4c(OC5CNCC5F)nc(OCC56CCCN5CC(F)C6)nc4c3F)ccc(F)c2s1] | |
| 19 | 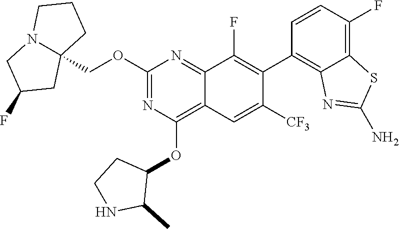 | LCMS m/z [M + 1]: 639.2 1H NMR (400 MHz, MeOD) δ 8.46 (s, 1H), 7.18-7.21 (m, 1H), 6.97 (t, J = 9.2 Hz, 1H), 6.07 (m, 1H), 5.32 (d, J = 52.8 Hz, 1H), 4.65 (m, 2H), 4.34-4.40 (m, 2H), 3.87-3.90 (m, 1H), 3.05-3.57 (m, 4H), 2.30-2.34 (m, 1H), 1.99-2.26 (m, 7H), 1.50-1.51 (m, 3H ). |
| 20 | 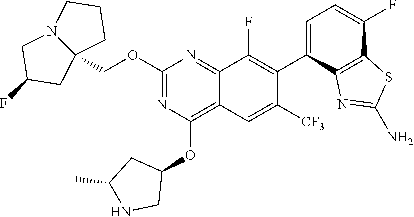 | LCMS m/z [M + 1]: 639.2 1H NMR (400 MHz, MeOD) δ 8.34 (s, 1H), 7.17-7.20 (m, 1H), 6.97 (t, J = 8.8 Hz, 1H), 6.01 (m, 1H), 5.32 (d, J = 53.2 Hz, 1H), 4.58 (m, 2H), 4.30-4.36 (m, 2H), 3.77-3.82 (m, 2H), 3.49-3.52 (m, 1H), 3.20-3.27 (m, 2H), 2.59 (m, 1H), 1.99-2.32 (m, 7H), 1.42-1.44 (m, 3H). |
| 21 | 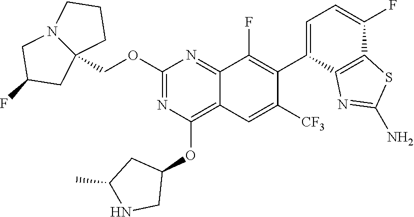 | LCMS m/z [M + 1]: 639.2 1H NMR (400 MHz, MeOD) δ 8.34 (s, 1H), 7.17-7.20 (m, 1H), 6.97 (t, J = 8.8 Hz, 1H), 6.01(m, 1H), 5.32 (d, J = 53.2 Hz, 1H), 4.57 (m, 2H), 4.33-4.40 (m, 2H), 3.81-3.86 (m, 2H), 3.56-3.59 (m, 1H), 3.04-3.22 (m, 2H), 2.61-2.63 (m, 1H), 1.99-2.32 (m, 7H), 1.45-1.47 (m, 3H). |
| 22 | 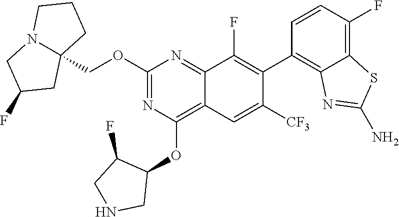 [SMILES: Nc1nc2c(-c3c(C(F)(F)F)cc4c(O[C@H]5CNC[C@H]5F)nc(OC[C@@]56CCCN5C[C@H](F)C6)nc4c3F)ccc(F)c2s1] | LCMS m/z [M + 1]: 643.2 1H NMR (400 MHz, MeOD) δ 8.37 (s, 1H), 7.20 (s, 1H), 6.98 (t, J = 8.8 Hz, 1H), 5.80-5.81 (m, 1H), 5.52 (d, J = 56.4 Hz, 1H), 5.38 (d, J = 56.4 Hz, 1H) , 4.41- 4.58 (m, 2H), 3.60-3.61 (m, 1H), 3.36-3.43(m, 6H), 3.14-3.15 (m, 1H), 1.97-2.46 (m, 6H). |
| 23 | 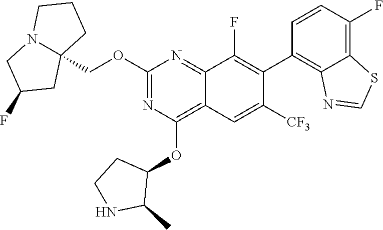 [SMILES: C[C@H]1NCC[C@H]1Oc1nc(OC[C@@]23CCCN2C[C@H](F)C3)nc2c(F)c(-c3ccc(F)c4scnc34)c(C(F)(F)F)cc12] | LCMS m/z [M + 1]: 624.4 1H NMR (400 MHz, MeOD) δ 9.26 (s , 1H), 8.54 (s, 1H), 7.57 (s, , 1H), 7.43 (t, J = 8.8 Hz, 1H), 6.08-6.11 (m, 1H), 5.33 (d, J = 53.2 Hz, 1H), 4.39-4.43 (m, 2H), 3.93-3.94 (m, 1H), 3.26-3.59 (m, 6H), 3.07-3.08 (m, 1H), 2.62-2.64 (m, 1H) 2.04-2.36 (m, 6H), 1.50-1.51 (m, 3H). |
| 23a | 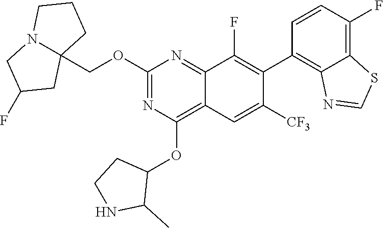 [SMILES: CC1NCCC1Oc1nc(OCC23CCCN2CC(F)C3)nc2c(F)c(-c3ccc(F)c4scnc34)c(C(F)(F)F)cc12] | |
| 24 | 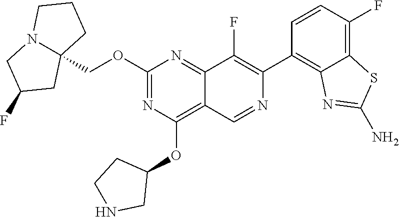 [SMILES: Nc1nc2c(-c3ncc4c(O[C@@H]5CCNC5)nc(OC[C@@]56CCCN5C[C@H](F)C6)nc4c3F)ccc(F)c2s1] | LCMS m/z [M + 1]: 558.2 1H NMR (400 MHz, MeOD) δ 9.20 (s, 1H), 7.45-7.48 (m, 1H), 7.03 (t, J = 8.8 Hz, 1H), 6.04-6.05 (m, 1H), 5.32 (d, J = 53.6 Hz, 1H), 4.42-4.43 (m, 1H), 4.37-4.40 (m, 2H), 3.04-3.70 (m, 7H), 2.48-2.51 (m, 2H), 1.93- 2.34 (m, 6H). |
| 24a | 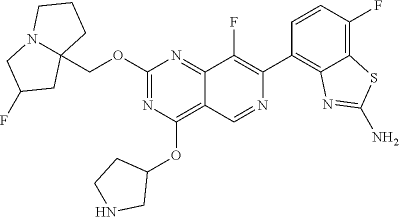 [SMILES: Nc1nc2c(-c3ncc4c(OC5CCNC5)nc(OCC56CCCN5CC(F)C6)nc4c3F)ccc(F)c2s1] | |
| 25 | 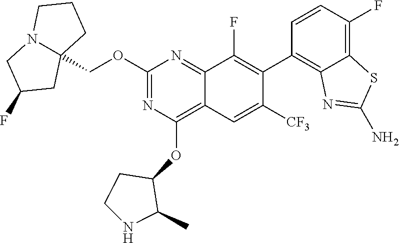 | LCMS m/z [M + 1]: 639.2 1H NMR (400 MHz, MeOD) δ 8.47 (s, 1H), 7.00-7.19 (m, 1H), 6.97 (t, J = 8.8 Hz, 1H), 6.10-6.12 (m, 1H), 5.38 (d, J = 52.8 Hz, 1H), 4.46 (m, 1H) 4.44-4.45 (m, 2H), 3.97-3.98 (m, 1H), 3.13-3.62 (m, 6H), 2.31-2.48 (m, 1H), 2.01-2.22 (m, 6H), 1.53-1.54 (m, 3H ). |
| 26 | 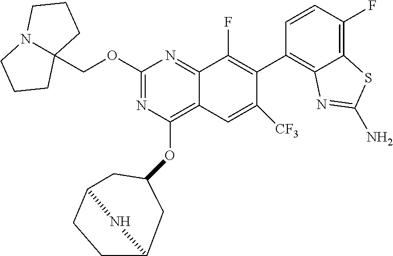 | LCMS m/z [M + 1]: 647.2 1H NMR (400 MHz, CD₃OD) δ 8.28 (s, 1H), 7.21- 7.24 (m, 1H), 7.01 (t, J = 8.0 Hz, 1H), 5.79-5.81 (m, 1H), 4.70-4.74 (m, 2H), 4.15-4.20 (m, 2H), 3.66-3.72 (m, 2H), 3.32-3.37 (m, 2H), 2.43-2.56 (m, 6H), 2.08- 2.34 (m, 10H). |
| 27 | 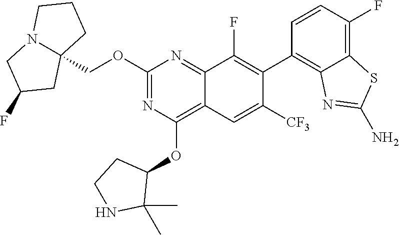 [SMILES: CC1(C)NCC[C@H]1Oc1nc(OC[C@@]23CCCN2C[C@H](F)C3)nc2c(F)c(-c3ccc(F)c4sc(N)nc34)c(C(F)(F)F)cc12] | LCMS m/z [M + 1]: 653.3 1H NMR (400 MHz, Methanol-d4) δ 8.44 (d, J = 4.19 Hz, 1H), 7.17-7.21 (m, 1H), 6.97 (t, J = 8.8 Hz, 1H), 5.73-5.77 (m, 1H), 5.34 (d, J = 54 Hz, 1H), 4.29-4.59 (m, 2H), 3.35-3.50 (m, 2H), 3.20-3.27 (m, 2H), 3.00- 3.07 (m, 1H), 2.72-2.79 (m, 1H), 1.89-2.32 (m, 8H), 1.51 (s, 3H), 1.44 (s, 3H). |
| 27a | 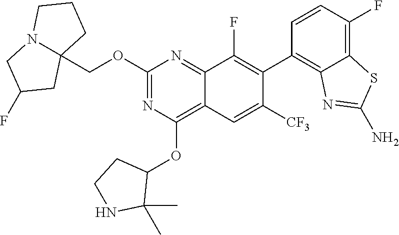 [SMILES: CC1(C)NCCC1Oc1nc(OCC23CCCN2CC(F)C3)nc2c(F)c(-c3ccc(F)c4sc(N)nc34)c(C(F)(F)F)cc12] | |
| 28 | 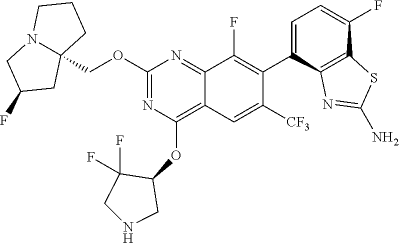 | LCMS m/z [M + 1]: 661.30 1H NMR (400 MHz, MeOD) δ 8.40 (s, 1H), 7.19-7.23 (m, 1H), 6.98 (t, J = 8.8 Hz, 1H), 5.85-5.90 (m, 1H), 5.36 (d, J = 54 Hz, 1H), 4.59 (s, 1H), 4.40-4.47 (m, 2H), 3.64-3.72 (m, 1H), 3.30- 3.44 (m, 5H), 3.08-3.15 (m, 1H), 1.96-2.44 (m, 6H). |
| 29 | 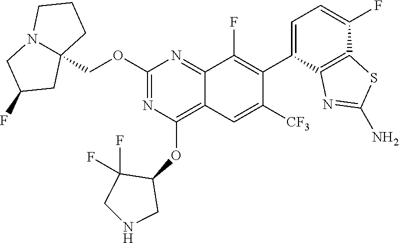 | LCMS m/z [M + 1]: 661.30 1H NMR (400 MHz, MeOD) δ 8.40 (s, 1H), 7.19-7.23 (m, 1H), 6.98 (t, J = 8.8 Hz, 1H), 5.85-5.90 (m, 1H), 5.36 (d, J = 54 Hz, 1H), 4.59 (s, 1H), 4.40-4.47 (m, 2H), 3.64-3.72 (m, 1H), 3.30-3.44 (m, 5H), 3.08-3.15 (m, 1H), 1.96-2.44 (m, 6H). |
| 29a | 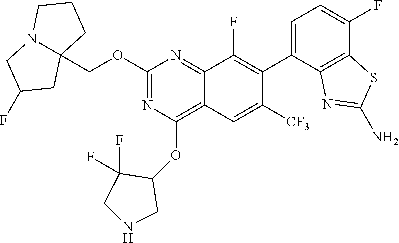 [SMILES: Nc1nc2c(-c3c(C(F)(F)F)cc4c(OC5CNCC5(F)F)nc(OCC56CCCN5CC(F)C6)nc4c3F)ccc(F)c2s1] | |
| 30 | 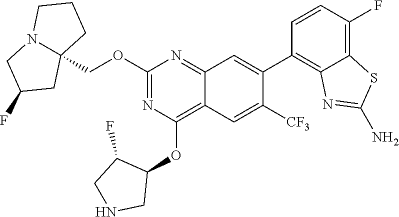 [SMILES: Nc1nc2c(-c3cc4nc(OC[C@@]56CCCN5C[C@H](F)C6)nc(O[C@H]5CNC[C@@H]5F)c4cc3C(F)(F)F)ccc(F)c2s1] | LCMS m/z [M + 1]: 625.3 1H NMR (400 MHz, MeOD) δ 8.53 (s, 1H), 7.64 (s, 1H), 7.15-7.19 (m, 1H), 6.93 (t, J = 8.8 Hz, 1H), 5.86- 5.89 (m, 1H), 5.45 (d, J = 50.8 Hz, 1H), 5.36 (d, J = 53.6 Hz, 1H), 4.37-4.43 (m, 2H), 3.62-3.66 (m, 1H), 3.36 -3.57 (m, 5H), 3.07-3.23 (m, 2H), 1.96-2.44 (m, 6H). |
| 31 | 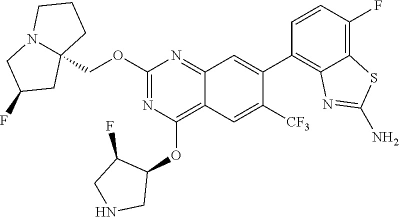 [SMILES: Nc1nc2c(-c3cc4nc(OC[C@@]56CCCN5C[C@H](F)C6)nc(O[C@H]5CNC[C@H]5F)c4cc3C(F)(F)F)ccc(F)c2s1] | LCMS m/z [M + 1]: 625.20 1H NMR (400 MHz, MeOD) δ 8.53 (s, 1H), 7.63 (s, 1H), 7.16-7.20 (m, 1H), 6.93 (t, J = 8.8 Hz, 1H), 5.67- 5.78 (m, 1H), 5.48 (d, J = 54 Hz, 1H), 5.32 (d, J = 53.2 Hz, 1H), 4.32 (dd, J = 26.8, 10.8 Hz, 2H), 3.51-3.56 (m, 1H), 3.32-3.41 (m, 2H), 3.27-3.30 (m, 3H), 3.20- 3.24 (m, 1H), 3.02-3.08 (m, 1H), 2.14-2.37 (m, 3H), 1.90-2.05 (m, 3H). |
| 31a | 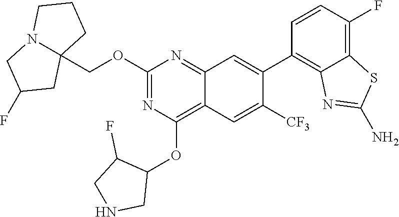 [SMILES: Nc1nc2c(-c3cc4nc(OCC56CCCN5CC(F)C6)nc(OC5CNCC5F)c4cc3C(F)(F)F)ccc(F)c2s1] | |
| 32 | 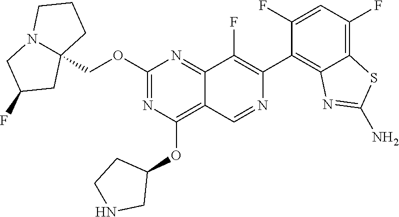 [SMILES: Nc1nc2c(-c3ncc4c(O[C@@H]5CCNC5)nc(OC[C@@]56CCCN5C[C@H](F)C6)nc4c3F)c(F)cc(F)c2s1] | LCMS m/z [M + 1]: 576.2 1H NMR (400 MHz, MeOD) δ 9.23 (s, 1H), 8.47 (s, 1H, HCO2H), 6.93 (t, J = 8.8 Hz, 1H), 6.01-6.18 (m, 1H), 5.35 (d, J = 52 Hz, 1H), 4.32-4.53 (m, 2H), 3.36- 3.73 (m, 8H), 3.02-3.21 (m, 1H), 1.82-2.60 (m, 8H). |
| 32a | 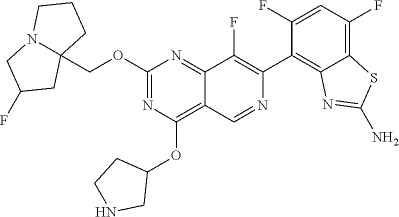 [SMILES: Nc1nc2c(-c3ncc4c(OC5CCNC5)nc(OCC56CCCN5CC(F)C6)nc4c3F)c(F)cc(F)c2s1] | |
| 33 | 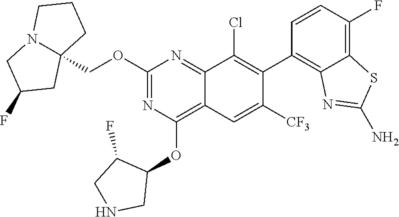 [SMILES: Nc1nc2c(-c3c(C(F)(F)F)cc4c(O[C@H]5CNC[C@@H]5F)nc(OC[C@@]56CCCN5C[C@H](F)C6)nc4c3Cl)ccc(F)c2s1] | LCMS m/z [M + 1]: 659.2 1H NMR (400 MHz, MeOD) δ 8.49 (s, 1H), 7.08-7.11 (m, 1H), 6.97 (t, J = 8.8 Hz, 1H), 5.84-5.87 (m, 1H), 5.44 (d, J = 50.8 Hz, 1H), 5.34 (d, J = 53.2 Hz, 1H), 4.38-4.50 (m, 2H), 3.60-3.63 (m, 1H), 3.25-3.47 (m, 6H), 3.05-3.11 (m, 1H), 1.93-2.43 (m, 6H). |
| 33a | 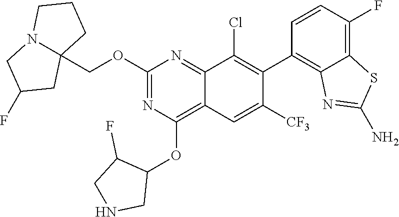 [SMILES: Nc1nc2c(-c3c(C(F)(F)F)cc4c(OC5CNCC5F)nc(OCC56CCCN5CC(F)C6)nc4c3Cl)ccc(F)c2s1] | |
| 34 | 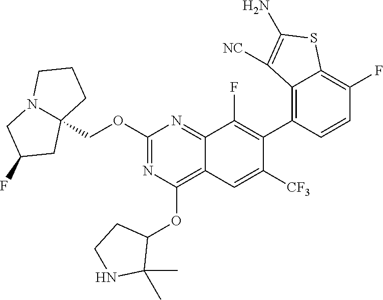 [SMILES: CC1(C)NCCC1Oc1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.2 1H NMR (400 MHz, Methanol-d4) δ 8.46 (s, 1H), 7.18-7.24 (m, 1H), 7.02 (t, J = 8.8 Hz, 1H), 5.79-5.83 (m, 1H), 5.34 (d, J = 53.2 Hz, 1H), 4.34-4.45 (m, 2H), 3.48-3.58 (m, 2H), 3.22-3.27 (m, 2H), 3.05-3.13 (m, 1H), 2.78-2.88 (m, 1H), 1.94-2.43 (m, 8H), 1.61 (s, 3H), 1.52 (s, 3H). |
| 34a | 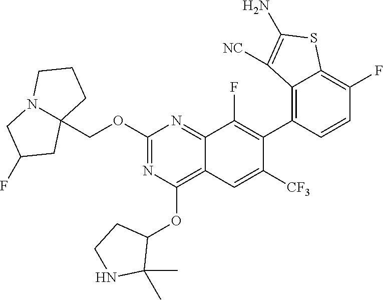 [SMILES: CC1(C)NCCC1Oc1nc(OCC23CCCN2CC(F)C3)nc2c(F)c(-c3ccc(F)c4sc(N)c(C#N)c34)c(C(F)(F)F)cc12] | |
| 35 | 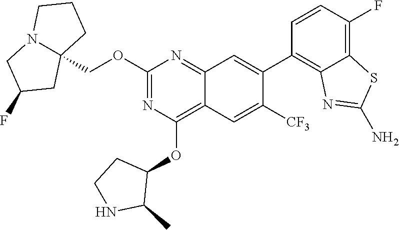 [SMILES: C[C@H]1NCC[C@H]1Oc1nc(OC[C@@]23CCCN2C[C@H](F)C3)nc2cc(-c3ccc(F)c4sc(N)nc34)c(C(F)(F)F)cc12] | LCMS m/z [M + 1]: 621.3 1H NMR (400 MHz, MeOD) δ 8.64 (s, 1H), 8.52 (s, 1 H, HCO2H) 7.65 (s, 1H), 7.18 (dd, J = 8.5, 5.6 Hz, 1H), 6.94 (d, J = 8.8 Hz, 1H), 6.03-6.15 (m, 1H), 5.32 (d, J = 52 Hz, 1H), 4.50-4.72 (m, 1H), 4.25-4.41 (m, 2H), 3.81-3.98(m, 1H), 3.38-3,62 (m, 2H), 3.07-3.29 (m, 3H), 1.79-2.72 (m, 8H), 1.51 (d, J = 8.0 Hz, 3H). |
| 35a | 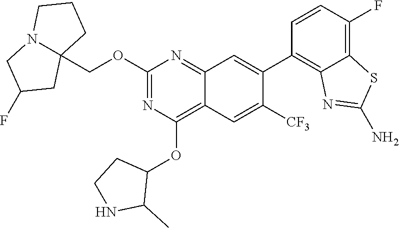 [SMILES: CC1NCCC1Oc1nc(OCC23CCCN2CC(F)C3)nc2cc(-c3ccc(F)c4sc(N)nc34)c(C(F)(F)F)cc12] | |
| 36 | 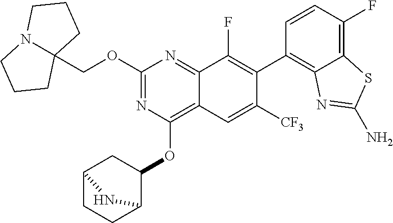 | LCMS m/z [M + 1]: 633.3 1H NMR (400 MHz, CD₃OD): δ 8.53(s, 1H, HCO₂H), 8.31 (s, 1H), 7.17-7.29 (m, 1H), 6.98 (t, J = 8.8 Hz, 1H), 5.49-5.60 (m, 1H), 4.60 (s, 2H), 4.11-4.20 (m, 1H), 3.72-3.81 (m, 1H), 3.50-3.55 (m, 2H), 3.10-3.28 (m, 2H), 2.41-2.52 (m, 1H), 2.08-2.35 (m, 9H), 1.60- 1.95 (m, 4H) |
| 36a | 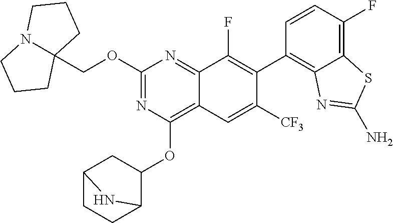 [SMILES: Nc1nc2c(-c3c(C(F)(F)F)cc4c(OC5CC6CCC5N6)nc(OCC56CCCN5CCC6)nc4c3F)ccc(F)c2s1] | |
| 37 | 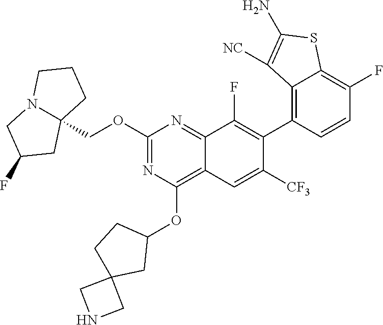 [SMILES: N#Cc1c(N)sc2c(F)ccc(-c3c(C(F)(F)F)cc4c(OC5CCC6(CNC6)C5)nc(OC[C@@]56CCCN5C[C@H](F)C6)nc4c3F)c12] | |
| 37a | 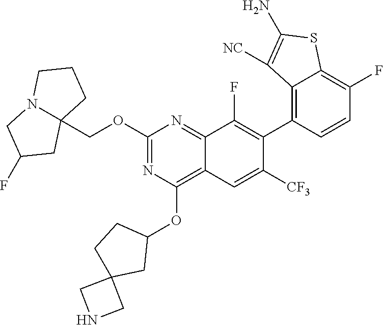 [SMILES: N#Cc1c(N)sc2c(F)ccc(-c3c(C(F)(F)F)cc4c(OC5CCC6(CNC6)C5)nc(OCC56CCCN5CC(F)C6)nc4c3F)c12] | |
| 38 | 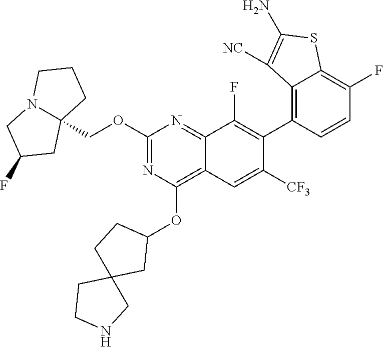 [SMILES: N#Cc1c(N)sc2c(F)ccc(-c3c(C(F)(F)F)cc4c(OC5CCC6(CCNC6)C5)nc(OC[C@@]56CCCN5C[C@H](F)C6)nc4c3F)c12] | |
| 38a | 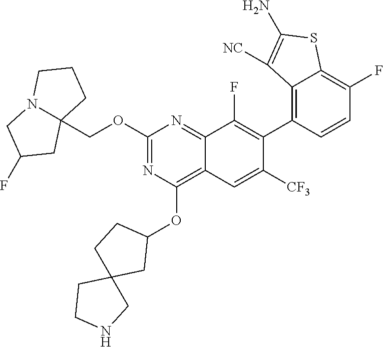 [SMILES: N#Cc1c(N)sc2c(F)ccc(-c3c(C(F)(F)F)cc4c(OC5CCC6(CCNC6)C5)nc(OCC56CCCN5CC(F)C6)nc4c3F)c12] | |
| 39 | 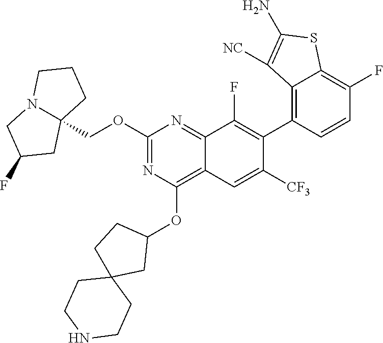 [SMILES: N#Cc1c(N)sc2c(F)ccc(-c3c(C(F)(F)F)cc4c(OC5CCC6(CCNCC6)C5)nc(OC[C@@]56CCCN5C[C@H](F)C6)nc4c3F)c12] | |
| 39a | 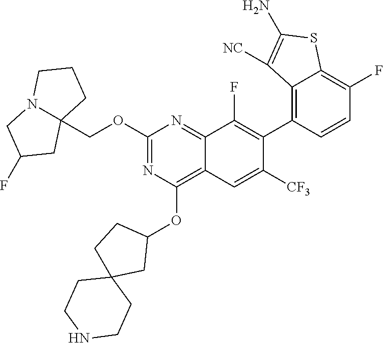 [SMILES: N#Cc1c(N)sc2c(F)ccc(-c3c(C(F)(F)F)cc4c(OC5CCC6(CCNCC6)C5)nc(OCC56CCCN5CC(F)C6)nc4c3F)c12] | |
| 40 | 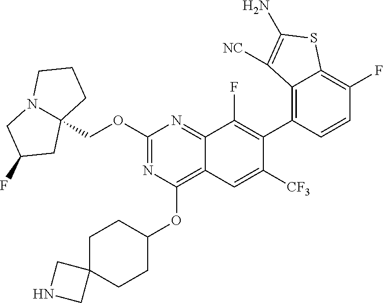 [SMILES: N#Cc1c(N)sc2c(F)ccc(-c3c(C(F)(F)F)cc4c(OC5CCC6(CC5)CNC6)nc(OC[C@@]56CCCN5C[C@H](F)C6)nc4c3F)c12] | LCMS m/z [M + 1]: 702.7. 1H NMR (400 MHz, CD₃OD) δ 8.32 (s, 1H), 7.22- 7.25 (m, 1H), 7.03-7.07 (m, 1H), 5.66 (s, 0.5H), 5.54 (s, 0.5H), 4.70-4.84 (m, 3H), 3.91-3.95 (d, 4H), 3.50 (s, 1H), 3.26-3.32 (m, 2H), 2.38-2.76 (m, 8H), 2.12- 2.20 (m, 4H), 1.92-1.94 (m, 3H). |
| 40a | 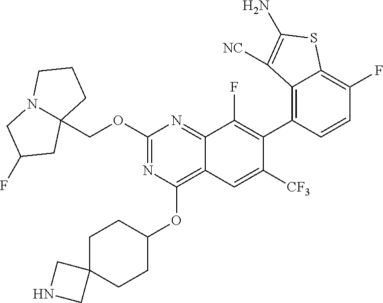 [SMILES: N#Cc1c(N)sc2c(F)ccc(-c3c(C(F)(F)F)cc4c(OC5CCC6(CC5)CNC6)nc(OCC56CCCN5CC(F)C6)nc4c3F)c12] | |
| 41 | 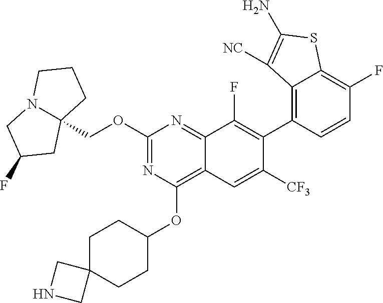 [SMILES: N#Cc1c(N)sc2c(F)ccc(-c3c(C(F)(F)F)cc4c(OC5CCC6(CC5)CNC6)nc(OC[C@@]56CCCN5C[C@H](F)C6)nc4c3F)c12] | |
| 41a | 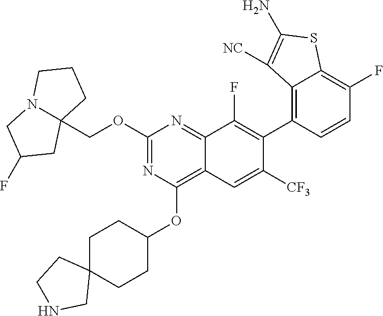 [SMILES: N#Cc1c(N)sc2c(F)ccc(-c3c(C(F)(F)F)cc4c(OC5CCC6(CCNC6)CC5)nc(OCC56CCCN5CC(F)C6)nc4c3F)c12] | |
| 42 | 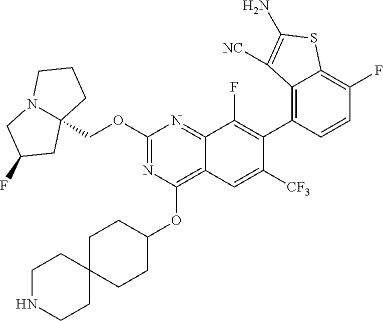 [SMILES: N#Cc1c(N)sc2c(F)ccc(-c3c(C(F)(F)F)cc4c(OC5CCC6(CCNCC6)CC5)nc(OC[C@@]56CCCN5C[C@H](F)C6)nc4c3F)c12] | |
| 42a | 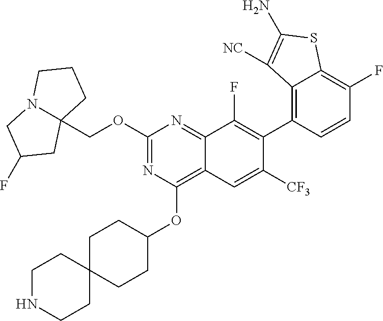 [SMILES: N#Cc1c(N)sc2c(F)ccc(-c3c(C(F)(F)F)cc4c(OC5CCC6(CCNCC6)CC5)nc(OCC56CCCN5CC(F)C6)nc4c3F)c12] | |
| 43 | 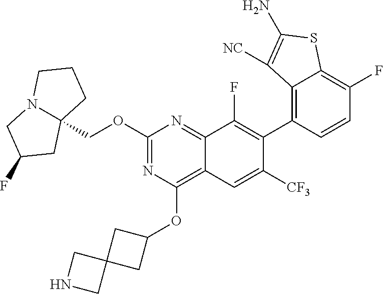 [SMILES: N#Cc1c(N)sc2c(F)ccc(-c3c(C(F)(F)F)cc4c(OC5CC6(CNC6)C5)nc(OC[C@@]56CCCN5C[C@H](F)C6)nc4c3F)c12] | LCMS m/z [M + 1]: 675.1. 1HNMR (400 MHz, CD₃OD) δ 8.33 (s, 1H), 7.22 (t, 1H), 7.05 (t, 1H), 5.65 (s, 0.5H), 5.45-5.55 (m, 1.5H), 4.65-4.8 (m, 2H), 4.21 (d, 4H), 3.8-4.05 (m, 3H), 3.48 (s, 1H), 2.95-3.1 (m, 2H), 2.55-2.8 (m, 4H), 2.15-2.5 (m, 4H). |
| 43a | 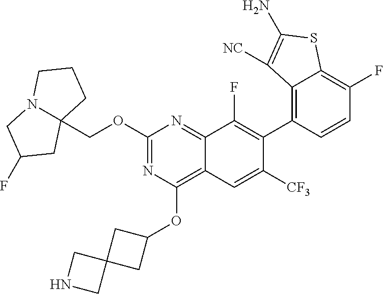 [SMILES: N#Cc1c(N)sc2c(F)ccc(-c3c(C(F)(F)F)cc4c(OC5CC6(CNC6)C5)nc(OCC56CCCN5CC(F)C6)nc4c3F)c12] | |
| 44 | 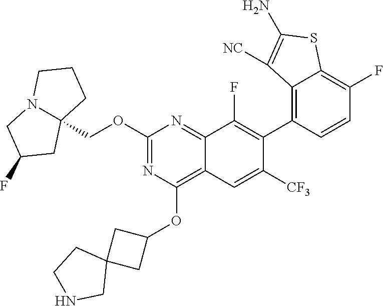 [SMILES: N#Cc1c(N)sc2c(F)ccc(-c3c(C(F)(F)F)cc4c(OC5CC6(CCNC6)C5)nc(OC[C@@]56CCCN5C[C@H](F)C6)nc4c3F)c12] | |
| 44a | 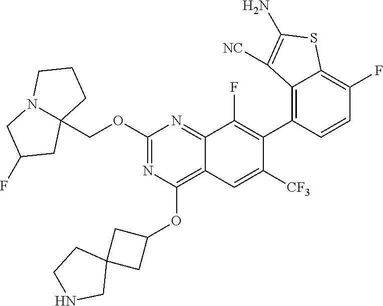 [SMILES: N#Cc1c(N)sc2c(F)ccc(-c3c(C(F)(F)F)cc4c(OC5CC6(CCNC6)C5)nc(OCC56CCCN5CC(F)C6)nc4c3F)c12] | |
| 45 | 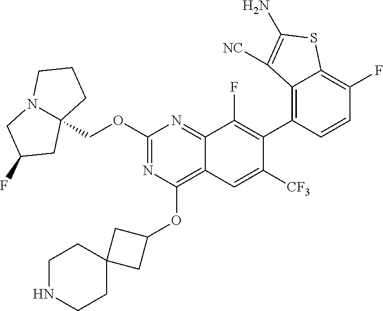 [SMILES: N#Cc1c(N)sc2c(F)ccc(-c3c(C(F)(F)F)cc4c(OC5CC6(CCNCC6)C5)nc(OC[C@@]56CCCN5C[C@H](F)C6)nc4c3F)c12] | |
| 45a | 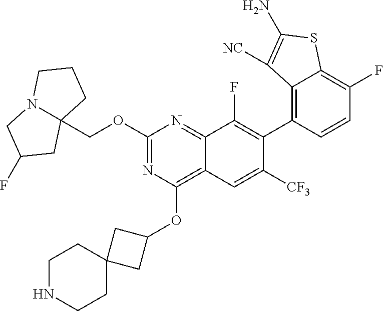 [SMILES: N#Cc1c(N)sc2c(F)ccc(-c3c(C(F)(F)F)cc4c(OC5CC6(CCNCC6)C5)nc(OCC56CCCN5CC(F)C6)nc4c3F)c12] | |
| 46 | 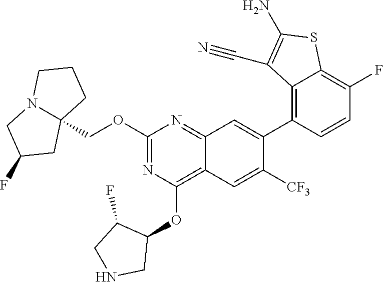 [SMILES: N#Cc1c(N)sc2c(F)ccc(-c3cc4nc(OC[C@@]56CCCN5C[C@H](F)C6)nc(O[C@H]5CNC[C@@H]5F)c4cc3C(F)(F)F)c12] | LCMS m/z [M + 1]: 649.2 1H NMR (400 MHz, MeOD) δ 8.54 (s, 1H), 7.64 (s, 1H), 7.16-7.20 (m, 1H), 6.97 (t, J = 8.8 Hz, 1H), 5.91- 5.93 (m, 1H), 5.50 (d, J = 54 Hz, 1H), 5.40 (d, J = 52.4 Hz, 1H), 4.43-4.50 (m, 2H), 3.66-3.71 (m, 1H), 3.36-3.52 (m, 6H), 3.13-3.21 (m, 1H), 1.96-2.51 (m, 6H). |
| 46a | 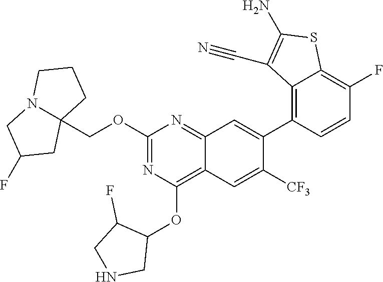 [SMILES: N#Cc1c(N)sc2c(F)ccc(-c3cc4nc(OCC56CCCN5CC(F)C6)nc(OC5CNCC5F)c4cc3C(F)(F)F)c12] | |
| 47 | 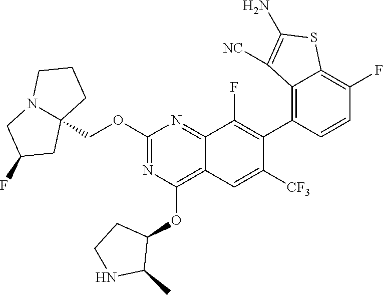 [SMILES: C[C@H]1NCC[C@H]1Oc1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 663.3 1H NMR (400 MHz, CD₃OD) δ 8.48 (s, 1H), 7.19- 7.24 (m, 1H), 7.03 (t, J = 8.0 Hz, 1H), 6.07-6.09 (m, 1H), 5.36 (d, J = 52 Hz, 1H), 4.43-4.49 (m, 2H), 3.95- 3.96 (m, 1H), 3.31-3.53 (m, 5H), 3.17-3.19 (m, 1H), 1.85-2.62 (m, 8H), 1.53-1.55 (m, 3H). |
| 47a | 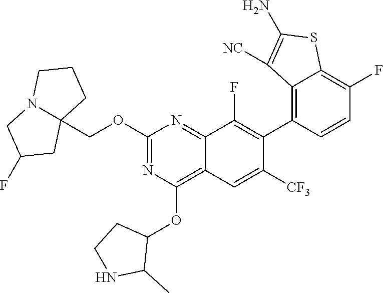 [SMILES: CC1NCCC1Oc1nc(OCC23CCCN2CC(F)C3)nc2c(F)c(-c3ccc(F)c4sc(N)c(C#N)c34)c(C(F)(F)F)cc12] | |
| 48 | 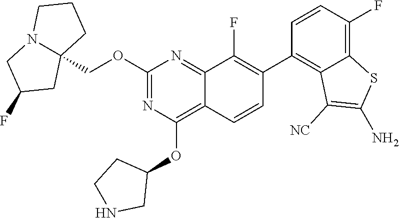 [SMILES: N#Cc1c(N)sc2c(F)ccc(-c3ccc4c(O[C@@H]5CCNC5)nc(OC[C@@]56CCCN5C[C@H](F)C6)nc4c3F)c12] | LCMS m/z [M + 1]: 581.2 1H NMR (400 MHz, CD₃OD) δ 8.04-8.06 (m, 1H), 7.45-7.47 (m, 1H), 7.25-7.29 (m, 1H), 7.05 (t, J = 8.8 Hz, 1H), 6.07 (s, 1H), 5.60 (d, J = 52.8 Hz, 1H), 4.67- 4.80 (m, 2H), 3.47-3.98 (m, 8H), 2.05-2.80 (m, 8H). |
| 48a | 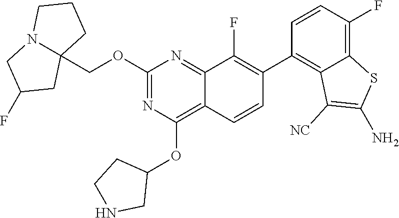 [SMILES: N#Cc1c(N)sc2c(F)ccc(-c3ccc4c(OC5CCNC5)nc(OCC56CCCN5CC(F)C6)nc4c3F)c12] | |
| 49 | 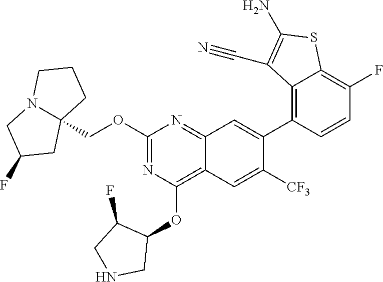 [SMILES: N#Cc1c(N)sc2c(F)ccc(-c3cc4nc(OC[C@@]56CCCN5C[C@H](F)C6)nc(O[C@H]5CNC[C@H]5F)c4cc3C(F)(F)F)c12] | LCMS m/z [M + 1]: 649.3 1H NMR (400 MHz, CD₃OD) δ 8.54(s, 1H), 7.65(s, 1H), 7.15-7.19 (m, 1H), 6.97 (t, J = 8.8 Hz, 1H), 5.77- 5.85 (m, 1H), 5.42-5.66 (m, 2H), 4.42-4.52 (m, 2H), 3.21-3.75 (m, 8H), 2.11-2.42 (m, 6H). |
| 50 | 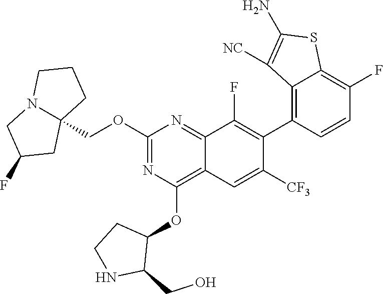 [SMILES: N#Cc1c(N)sc2c(F)ccc(-c3c(C(F)(F)F)cc4c(O[C@@H]5CCN[C@@H]5CO)nc(OC[C@@]56CCCN5C[C@H](F)C6)nc4c3F)c12] | LCMS m/z [M + 1]: 679.3 1H NMR (400 MHz, CD₃OD): δ 8.39 (s, 1H), 7.18- 7.23 (m, 1H), 7.00 (t, J = 8.8 Hz, 1H), 5.35 (d, J = 54.4 Hz, 1H), 4.10-4.65 (m, 9H), 3.38-3.48 (m, 1H), 3.07- 3.13 (m, 1H), 1.94-2.43 (m, 9H). |
| 50a | 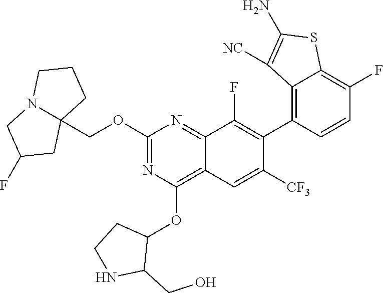 [SMILES: N#Cc1c(N)sc2c(F)ccc(-c3c(C(F)(F)F)cc4c(OC5CCNC5CO)nc(OCC56CCCN5CC(F)C6)nc4c3F)c12] | |
| 51 | 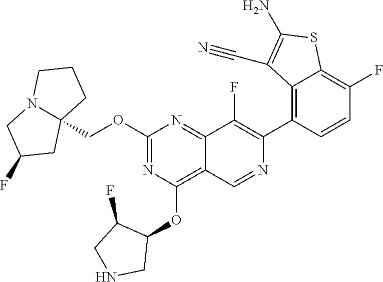 [SMILES: N#Cc1c(N)sc2c(F)ccc(-c3ncc4c(O[C@H]5CNC[C@H]5F)nc(OC[C@@]56CCCN5C[C@H](F)C6)nc4c3F)c12] | LCMS m/z [M + 1]: 600.3 |
| 51a | 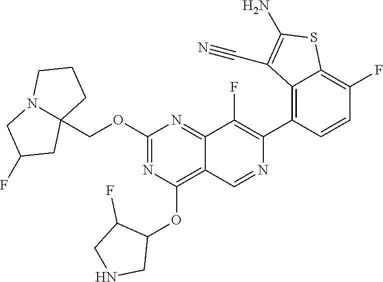 [SMILES: N#Cc1c(N)sc2c(F)ccc(-c3ncc4c(OC5CNCC5F)nc(OCC56CCCN5CC(F)C6)nc4c3F)c12] | |
| 52 | 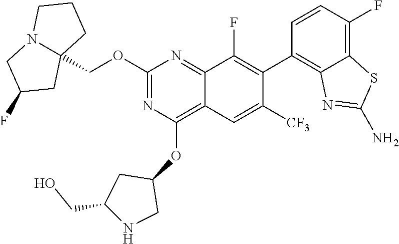 | LCMS m/z [M + 1]: 655.3 1H NMR (400 MHz, CD₃OD) δ 8.42 (s, 1H), 7.18- 7.22 (m, 1H), 6.98 (t, J = 8 Hz, 1H), 6.02 (s, 1H), 5.36 (d, J = 52 Hz, 1H), 4.36-4.60 (m, 4H), 3.11-3.89 (m, 7H), 1.96-2.80 (m, 8H). |
| 52a | 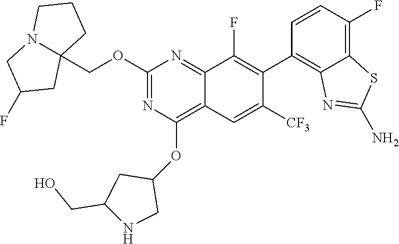 [SMILES: Nc1nc2c(-c3c(C(F)(F)F)cc4c(OC5CNC(CO)C5)nc(OCC56CCCN5CC(F)C6)nc4c3F)ccc(F)c2s1] | |
| 53 | 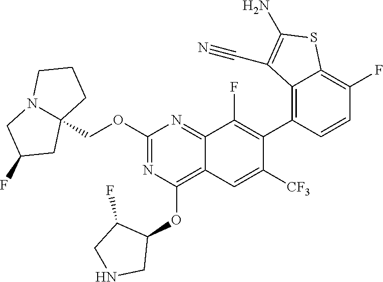 [SMILES: N#Cc1c(N)sc2c(F)ccc(-c3c(C(F)(F)F)cc4c(O[C@H]5CNC[C@@H]5F)nc(OC[C@@]56CCCN5C[C@H](F)C6)nc4c3F)c12] | LCMS m/z [M + 1]: 667.2 1H NMR (400 MHz, CD₃OD): δ 8.38 (s, 1H), 7.19- 7.24 (m, 1H), 7.00-7.06 (m, 1H), 5.93-6.00 (m, 1H), 5.53 (d, J = 50 Hz, 1H), 5.45 (d, J = 51.6 Hz, 1H), 4.52- 4.66 (m, 2H), 3.40-3.80 (m, 6H), 3.22-3.29 (m, 2H), 1.99-2.61 (m, 6H). |
| 53a | 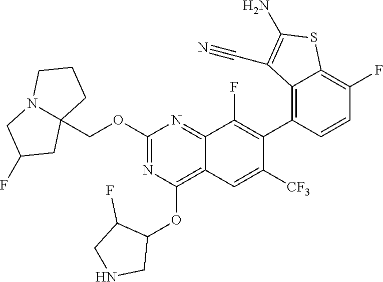 [SMILES: N#Cc1c(N)sc2c(F)ccc(-c3c(C(F)(F)F)cc4c(OC5CNCC5F)nc(OCC56CCCN5CC(F)C6)nc4c3F)c12] | |
| 54 | 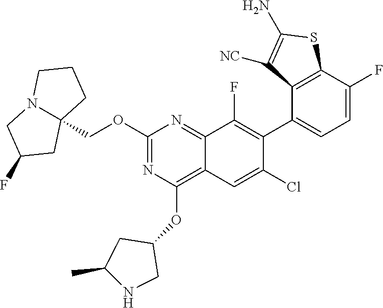 | LCMS m/z [M + 1]: 629.0 ¹H NMR (400 MHz, CD₃OD) δ 8.18 (s, 1H), 7.24- 7.21 (m, 1H), 7.10-7.06 (m, 1H), 6.06 (s, 1H), 5.66 (s, 0.5H), 5.53 (s, 0.5H), 4.76 (s, 2H), 4.17-4.15 (m, 1H), 3.99-3.77 (m, 5H), 3.51-3.50 (m, 1H), 2.76-2.62 (m, 3H), 2.47-2.34 (m, 3H), 2.22-2.15 (m, 2H), 1.56- 1.52 (m, 3H). |
| 55 | 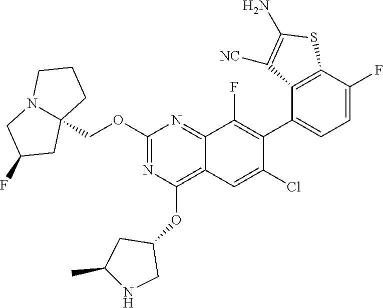 | LCMS m/z [M + 1]: 629.0 ¹H NMR (400 MHz, CD₃OD) δ 8.19 (s, 1H), 7.25- 7.21 (m, 1H), 7.10-7.06 (m, 1H), 6.06 (s, 1H), 5.66 (s, 0.5H), 5.54 (s, 0.5H), 4.81-4.71 (s, 2H), 4.18-4.15 (m, 1H), 4.00-3.92 (m, 4H), 3.77-3.73(m, 1H), 3.51-3.49 (m, 1H), 2.77-2.62 (m, 3H), 2.48-2.33 (m, 3H), 2.22- 2.15 (m, 2H), 1.56-1.53 (m, 3H). |
| 55a | 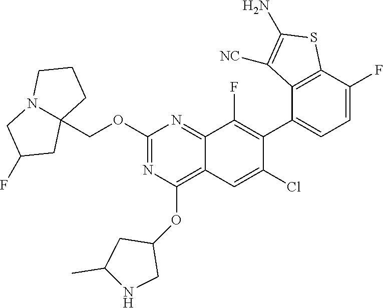 [SMILES: CC1CC(Oc2nc(OCC34CCCN3CC(F)C4)nc3c(F)c(-c4ccc(F)c5sc(N)c(C#N)c45)c(Cl)cc23)CN1] | |
| 56 | 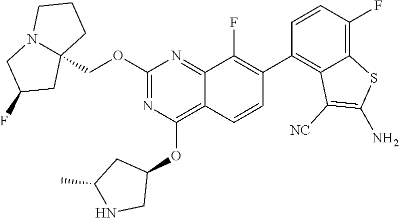 [SMILES: C[C@@H]1C[C@@H](Oc2nc(OC[C@@]34CCCN3C[C@H](F)C4)nc3c(F)c(-c4ccc(F)c5sc(N)c(C#N)c45)ccc23)CN1] | LCMS m/z [M + 1]: 595.2 1H NMR (400 MHz, CD₃OD) δ 8.00-8.01 (m, 1H), 7.38-7.42 (m, 1H), 7.23-7.26 (m, 1H), 7.03 (t, J = 8.8 Hz, 1H), 5.99 (s, 1H), 5.60 (d, J = 52.8 Hz, 1H), 4.44- 4.51 (m, 2H), 3.17-3.88 (m, 7H), 2.05-2.66 (m, 8H), 1.49-1.52 (m, 3H). |
| 56a | 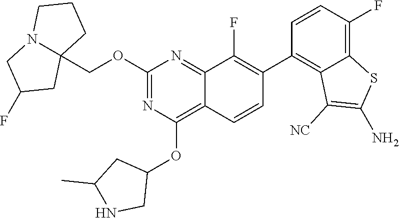 [SMILES: CC1CC(Oc2nc(OCC34CCCN3CC(F)C4)nc3c(F)c(-c4ccc(F)c5sc(N)c(C#N)c45)ccc23)CN1] | |
| 57 | 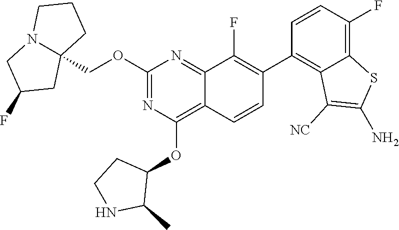 [SMILES: C[C@H]1NCC[C@H]1Oc1nc(OC[C@@]23CCCN2C[C@H](F)C3)nc2c(F)c(-c3ccc(F)c4sc(N)c(C#N)c34)ccc12] | |
| 57a | 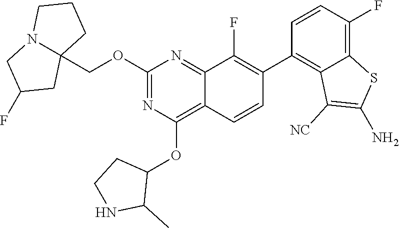 [SMILES: CC1NCCC1Oc1nc(OCC23CCCN2CC(F)C3)nc2c(F)c(-c3ccc(F)c4sc(N)c(C#N)c34)ccc12] | |
| 58 | 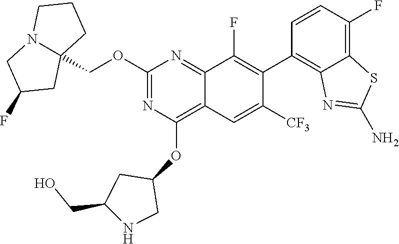 | LCMS m/z [M + 1]: 655.3 1HNMR (400 MHz, CD₃OD): δ 8.45 (s, 1H), 7.15- 7.22 (m, 1H), 6.94-7.01 (m, 1H), 6.09 (s, 1H), 5.42 (d, J = 52.8 Hz, 1H), 4.45-4.57 (m, 2H), 4.05-4.16 (m, 1H), 3.91-3.97 (m, 1H), 3.70-3.84 (m, 3H), 3.46-3.64 (m, 3H), 3.16-3.26 (m, 1H), 1.95-2.65 (m, 8H). |
| 59 | 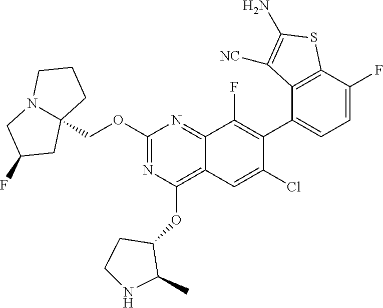 [SMILES: C[C@H]1NCC[C@@H]1Oc1nc(OC[C@@]23CCCN2C[C@H](F)C3)nc2c(F)c(-c3ccc(F)c4sc(N)c(C#N)c34)c(Cl)cc12] | LCMS m/z [M + 1]: 629.0 ¹H NMR (400 MHz, CD₃OD) δ 8.20(s, 1H), 7.22 (t, 1H), 7.08 (t, 1H), 5.75 (d, 1H), 5.66 (s, 0.5H), 5.53 (s, 0.5H), 4.77-4.71 (m, 2H), 4.23-4.20 (m, 1H), 3.92 (d, 3H), 3.64 (t, 2H), 3.55-3.50 (m, 1H), 2.68-2.21 (m, 8H), 1.56 (d, 2H), 1.30 (d, 1H). |
| 59a | 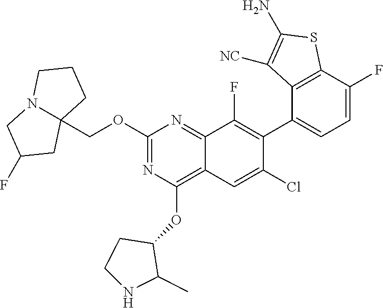 [SMILES: CC1NCC[C@@H]1Oc1nc(OCC23CCCN2CC(F)C3)nc2c(F)c(-c3ccc(F)c4sc(N)c(C#N)c34)c(Cl)cc12] | |
| 60 | 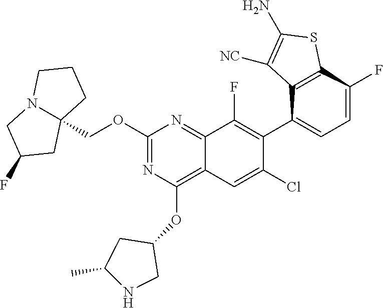 | LCMS m/z [M + 1]: 629.0 ¹H NMR (400 MHz, CD₃OD) δ 8.17 (s, 1H), 7.22 (t, 1H), 7.07 (t, 1H), 6.02 (s, 1H), 5.66 (s, 0.5H), 5.53 (s, 0.5H), 4.76 (s, 2H), 3.95-3.80 (m, 6H), 3.51-3.50 (m, 1H), 2.95-2.90 (m, 1H), 2.80-2.50 (m, 2H), 2.47-2.35 (m, 3H), 2.22-2.15 (m, 2H), 1.58-1.52 (d, 3H). |
| 61 | 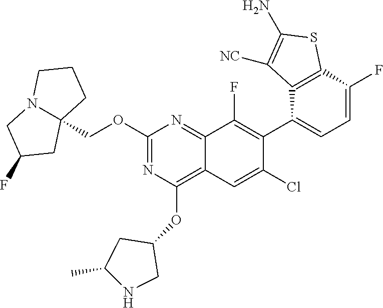 | LCMS m/z [M + 1]: 629.0 ¹H NMR (400 MHz, CD3OD) δ 8.14(s, 1H), 7.19 (t, 1H), 7.05 (t, 1H), 5.99 (s, 1H), 5.50 (d, 1H), 4.73-4.68 (s, 2H), 4.0-3.65 (m, 6H), 3.5 (s, 1H), 3.91 (s, 1H), 2.61-2.50 (m, 2H), 2.44-2.17 (m, 5H), 1.54 (d, 3H). |

Table 3 includes selected compounds of the present disclosure.

TABLE 3
Selected compounds of the present disclosure.

| Compound | | |
|---|---|---|
| No. | Structure | Analytical data |
| 62 | 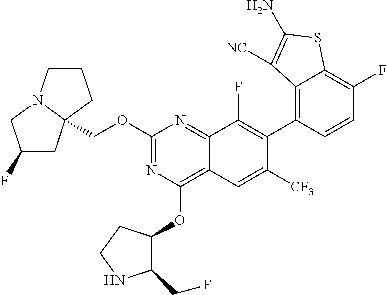 [SMILES: N#Cc1c(N)sc2c(F)ccc(-c3c(C(F)(F)F)cc4c(O[C@@H]5CCN[C@@H]5CF)nc(OC[C@@]56CCCN5C[C@H](F)C6)nc4c3F)c12] | LCMS m/z [M + 1]: 681.3 1H NMR (400 MHz, CD₃OD): δ 8.59 (s, 1H), 7.20- 7.23 (m, 1H), 7.02 (t, J = 8.8 Hz, 1H), 5.01-5.48 (m, 3H), 3.48-4.70 (m, 9H), 3.13-3.20 (m, 1H), 1.96-2.62 (m, 8H). |
| 62a | 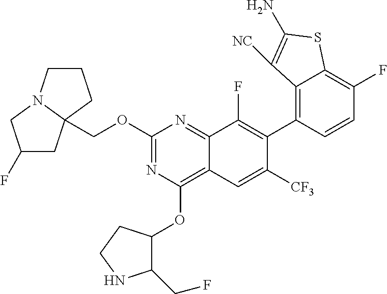 [SMILES: N#Cc1c(N)sc2c(F)ccc(-c3c(C(F)(F)F)cc4c(OC5CCNC5CF)nc(OCC56CCCN5CC(F)C6)nc4c3F)c12] | |
| 63 | 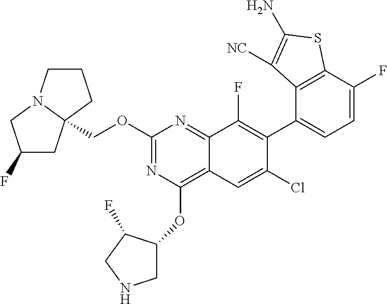 [SMILES: N#Cc1c(N)sc2c(F)ccc(-c3c(Cl)cc4c(O[C@@H]5CNC[C@@H]5F)nc(OC[C@@]56CCCN5C[C@H](F)C6)nc4c3F)c12] | LCMS m/z [M + 1]: 633.0. 1HNMR (400 MHz, CD₃OD) δ 8.08 (s, 1H), 7.20-7.25 (m, 1H), 7.06 (t, 1H), 5.63-5.70 (m, 1H), 5.54 (s, 0.5H), 5.40 (s, 1H), 5.26 (s, 0.5H), 4.30-4.42 (m, 2H), 3.48-3.55 (m, 1H), 3.37-3.41 (m, 1H), 3.23-3.32 (m, 4H), 3.02-3.06 (m, 1H), 2.15-2.45 (m, 4H), 1.90-2.06 (m, 3H). |
| 64 | 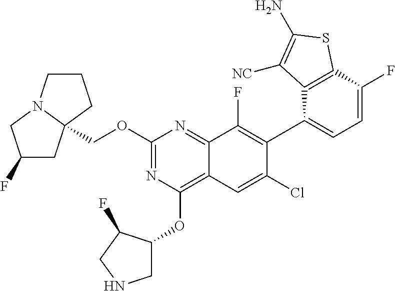 | LCMS m/z [M + 1]: 633.0 1HNMR (400 MHz, CD₃OD) δ 8.18 (s, 1H), 7.20-7.24 (m, 1H), 7.08 (t, 1H), 6.14 (d, 1H), 5.73 (s, 0.5H), 5.61-5.67 (m, 1H), 5.54 (s, 0.5H), 4.70-4.82 (m, 2H), 3.81-4.01 (m, 6H), 3.48-3.54 (m, 1H), 2.59-2.79 (m, 3H), 2.47-2.51 (m, 1H), 2.34-2.41 (m, 2H), 2.18-2.23 (m, 1H). |
| 64a | 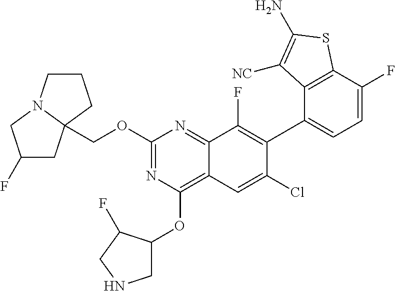 [SMILES: N#Cc1c(N)sc2c(F)ccc(-c3c(Cl)cc4c(OC5CNCC5F)nc(OCC56CCCN5CC(F)C6)nc4c3F)c12] | |
| 65 | 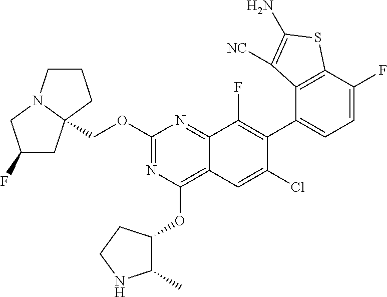 [SMILES: C[C@@H]1NCC[C@@H]1Oc1nc(OC[C@@]23CCCN2C[C@H](F)C3)nc2c(F)c(-c3ccc(F)c4sc(N)c(C#N)c34)c(Cl)cc12] | LCMS m/z [M + 1]: 629.0 1H NMR (400 MHz, CD₃OD) δ 8.26 (s, 1H), 7.21 (t, 1H), 7.07 (t, 1H), 6.09 (d, 1H), 5.66 (s, 0.5H), 5.53 (s, 0.5H), 4.76-4.70 (m, 2H), 4.05-3.91 (m, 3H), 3.59- 3.48 (m, 3H), 2.62-2.20 (m, 9H), 1.69-1.55 (m, 3H). |
| 66 | 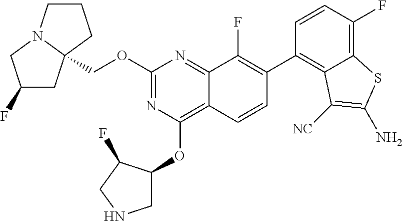 [SMILES: N#Cc1c(N)sc2c(F)ccc(-c3ccc4c(O[C@H]5CNC[C@H]5F)nc(OC[C@@]56CCCN5C[C@H](F)C6)nc4c3F)c12] | LCMS m/z [M + 1]: 599.2 1H NMR (400 MHz, CD₃OD): δ 8.04-8.05 (m, 1H), 7.42-7.46 (m, 1H), 7.24-7.27 (m, 1H), 7.03 (t, J = 8.8 Hz, 1H), 5.77-5.85 (m, 1H), 5.30-5.62 (m, 2H), 4.49- 4.59 (m, 2H), 3.30-3.70 (m, 8H), 2.19-2.49 (m, 6H). |
| 66a | 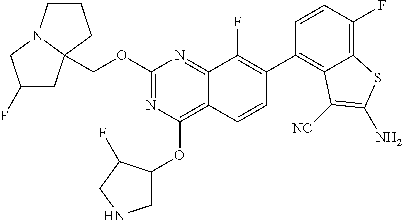 [SMILES: N#Cc1c(N)sc2c(F)ccc(-c3ccc4c(OC5CNCC5F)nc(OCC56CCCN5CC(F)C6)nc4c3F)c12] | |
| 67 | 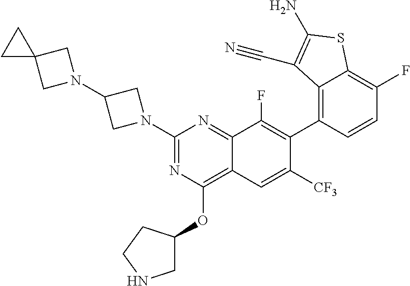 [SMILES: N#Cc1c(N)sc2c(F)ccc(-c3c(C(F)(F)F)cc4c(O[C@@H]5CCNC5)nc(N5CC(N6CC7(CC7)C6)C5)nc4c3F)c12] | LCMS m/z [M + 1]: 628.3 1H NMR (400 MHz, CD₃OD): δ 8.47 (s, 1H), 8.20 (s, 1H), 7.18 (s, 1H), 7.00 (t, J = 8.8 Hz, 1H), 5.98 (s, 1H), 4.06-4.38 (m, 4H), 3.54-3.79 (m, 9H), 2.52 (s, 1H), 0.63 (s, 4H). |
| 67a | 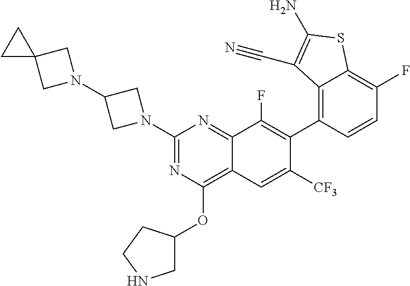 [SMILES: N#Cc1c(N)sc2c(F)ccc(-c3c(C(F)(F)F)cc4c(OC5CCNC5)nc(N5CC(N6CC7(CC7)C6)C5)nc4c3F)c12] | |
| 68 | 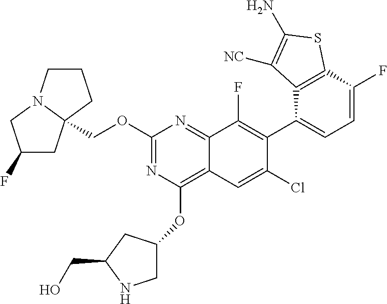 | LCMS m/z [M + 1]: 645.0 1H NMR (400 MHz, CD₃OD) δ 8.14 (s, 1H), 7.02- 7.16 (m, 2H), 6.04 (s, 1H), 5.60 (s, 0.5H), 5.46 (s, 0.5H), 4.81-4.83 (m, 4H), 4.12 (s, 1H), 3.73-3.76 (m, 5H), 3.43 (s, 1H), 2.16-2.56 (m, 8H). |
| 69 | 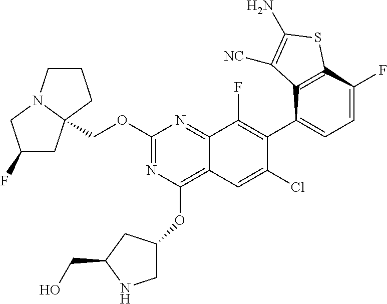 | LCMS m/z [M + 1]: 645.0 1H NMR (400 MHz, CD₃OD) δ 8.16 (s, 1H), 7.19 (s, 1H), 7.04 (s, 1H), 6.07 (s, 1H), 5.60 (s, 0.5H), 5.50 (s, 0.5H), 4.73-4.87 (m, 4H), 4.16 (s, 1H), 3.73-3.76 (m, 5H), 3.46 (s, 1H), 2.17-2.71 (m, 8H). |
| 70 | 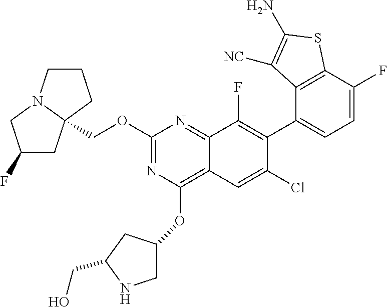 [SMILES: N#Cc1c(N)sc2c(F)ccc(-c3c(Cl)cc4c(O[C@@H]5CN[C@H](CO)C5)nc(OC[C@@]56CCCN5C[C@H](F)C6)nc4c3F)c12] | LCMS m/z [M + 1]: 645.1 1H NMR (400 MHz, CD₃OD) δ 8.35 (s, 1H), 8.18 (s, 1H), 7.24-7.21 (m, 1H), 7.10-7.05 (m, 1H), 6.03 (s, 1H), 5.53 (s, 0.5H), 5.40 (s, 0.5H), 4.61-4.50 (m, 2H), 3.93-3.90 (m, 1H), 3.82-3.79 (m, 2H), 3.73-3.58 (m, 4H), 3.50 (s, 1H), 2.80-2.79 (m, 1H), 2.54-2.44 (m, 2H), 2.35-2.21 (m, 4H), 2.07 (s, 1H). |
| 70a | 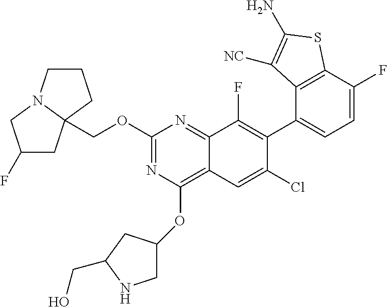 [SMILES: N#Cc1c(N)sc2c(F)ccc(-c3c(Cl)cc4c(OC5CNC(CO)C5)nc(OCC56CCCN5CC(F)C6)nc4c3F)c12] | |
| 71 | 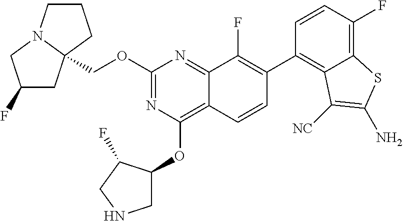 [SMILES: N#Cc1c(N)sc2c(F)ccc(-c3ccc4c(O[C@H]5CNC[C@@H]5F)nc(OC[C@@]56CCCN5C[C@H](F)C6)nc4c3F)c12] | LCMS m/z [M + 1]: 599.3 1H NMR (400 MHz, CD₃OD): δ 8.40 (s, 1H), 8.01 (d, J = 8.4 Hz, 1H), 7.43 (t, J = 7.4 Hz, 1H), 7.24-7.28 (m, 1H), 7.04 (t, J = 8.8 Hz, 1H), 5.90-5.93 (m, 1H), 5.49 (d, J = 52.8 Hz, 1H), 4.56-4.70 (m, 2H), 3.42-3.73 (m, 8H), 2.03-2.53 (m, 6H). |
| 72 | 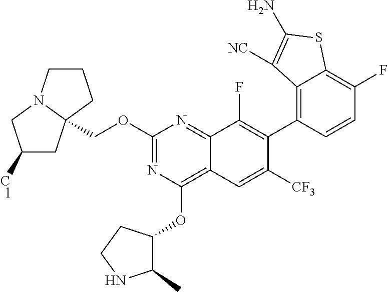 [SMILES: C[C@H]1NCC[C@@H]1Oc1nc(OC[C@@]23CCCN2C[C@H](Cl)C3)nc2c(F)c(-c3ccc(F)c4sc(N)c(C#N)c34)c(C(F)(F)F)cc12] | |
| 73 | 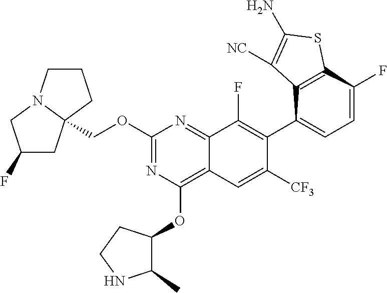 | LCMS m/z [M + 1]: 663.3 1H NMR (400 MHz, CD₃OD) δ 8.46 (s, 1H), 7.18- 7.22 (m, 1H), 7.02 (t, J = 9.28 Hz, 1H), 5.98 (s, 1H), 5.30 (d, J = 52.88 Hz, 1H), 4.60 (s, 1H), 4.31-4.37 (m, 2H), 3.01-3.66 (m, 6H), 1.86-2.57 (m, 8H), 1.45 (d, J = 6.68 Hz, 3H). |
| 74 | 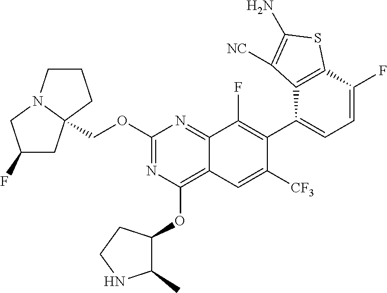 | LCMS m/z [M + 1]: 663.3 1H NMR (400 MHz, CD₃OD) δ 8.46 (s, 1H), 7.20- 7.23 (m, 1H), 7.02 (t, J = 9.28 Hz, 1H), 5.93 (s, 1H), 5.30 (d, J = 53.6 Hz, 1H), 4.61 (s, 1H), 4.31-4.37 (m, 2H), 3.01-3.66 (m, 6H), 1.90-2.54 (m, 8H), 1.38 (d, J = 6.52 Hz, 3H). |
| 75 | 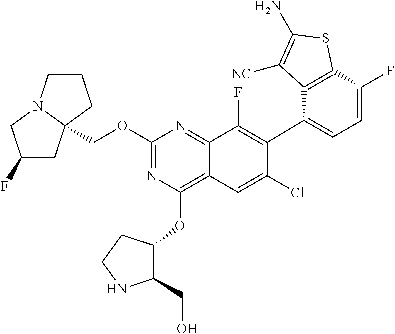 | LCMS m/z [M + 1]: 645 1H NMR (400 MHz, CD₃OD) δ 8.40 (s, 1H), 8.15 (s, 1H), 7.22 (t, 1H), 7.07 (s, 1H), 5.85 (d, 1H), 5.55 (s, 0.5H), 5.42 (s, 0.5H), 4.60-4.56 (m, 2H), 4.30-3.97 (m, 3H), 3.75-5.55 (m, 5H), 2.65-2.05 (m, 9H). |
| 76 | 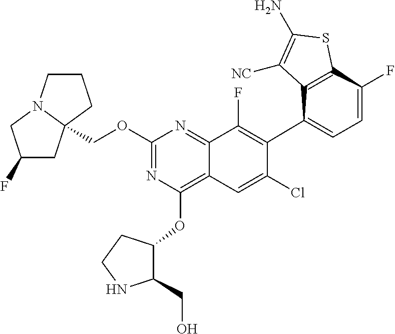 | LCMS m/z [M + 1]: 645 1H NMR (400 MHz, CD₃OD) δ 8.43 (s, 1H), 8.15 (s, 1H), 7.22 (t, 1H), 7.07 (s, 1H), 5.85 (d, 1H), 5.55 (s, 0.5H), 5.41 (s, 0.5H), 4.60-4.52 (m, 2H), 4.14-3.95 (m, 3H), 3.75-5.55 (m, 5H), 2.65-2.05 (m, 9H). |
| 76a | 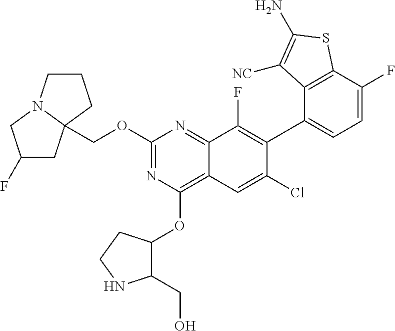 [SMILES: N#Cc1c(N)sc2c(F)ccc(-c3c(Cl)cc4c(OC5CCNC5CO)nc(OCC56CCCN5CC(F)C6)nc4c3F)c12] | |
| 77 | 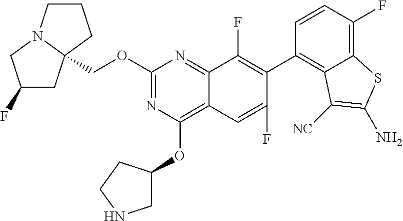 [SMILES: N#Cc1c(N)sc2c(F)ccc(-c3c(F)cc4c(O[C@@H]5CCNC5)nc(OC[C@@]56CCCN5C[C@H](F)C6)nc4c3F)c12] | LCMS m/z [M + 1]: 599.2 1H NMR (400 MHz, CD₃OD) δ 7.75-7.78 (m, 1H), 7.26-7.29 (m, 1H), 7.05 (t, J = 8.8 Hz, 1H), 6.03 (s, 1H), 5.40 (d, J = 53.2 Hz, 1H), 4.45-4.46 (m, 2H), 3.11-3.73 (m, 8H), 1.99-2.51 (m, 8H). |
| 78 | 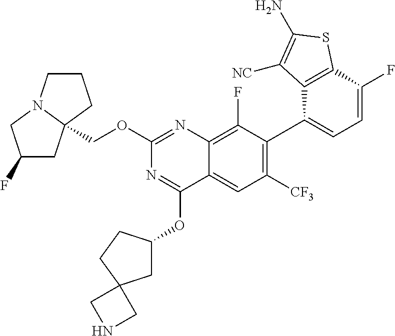 | LCMS m/z [M + 1]: 689.1 1H NMR (400 MHz, CD₃OD) δ 8.47 (s, 1H), 8.24 (s, 1H), 7.23 (t, 1H), 7.04 (t, 1H), 5.82-5.87 (m, 1H), 5.56 (s, 0.5H), 5.43 (s, 0.5H), 4.61 (s, 2H), 4.03-4.25 (m, 4H), 3.6-3.76 (m, 3H), 3.33-3.37 (m, 1H), 2.45-2.65 (m, 4H), 2.20-2.40 (m, 5H), 2.07-2.18 (m, 3H). |
| 79 | 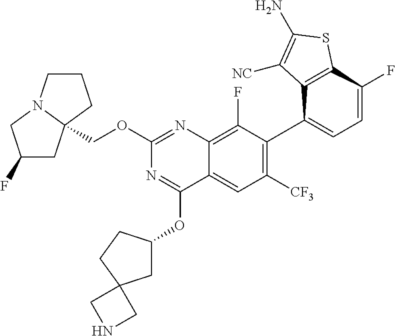 | LCMS m/z [M + 1]: 689.0 1HNMR (400 MHz, CD₃OD) δ 8.41 (s, 1H), 8.24 (s, 1H), 7.23 (t, 1H), 7.04 (t, 1H), 5.85 (s, 1H), 5.60 (s, 0.5H), 5.47 (s, 0.5H), 4.61-4.72 (m, 2H), 4.00-4.25 (m, 4H), 3.70-3.90 (m, 3H), 3.35-3.42 (m, 1H), 2.47- 2.70 (m, 4H), 2.22-2.45 (m, 5H), 2.02-2.22 (m, 3H). |
| 79a | 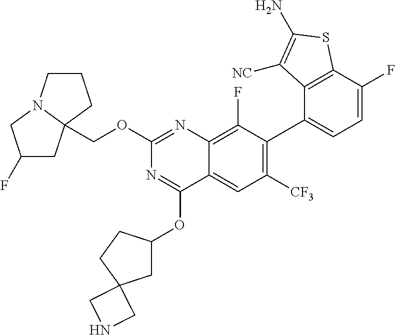 [SMILES: N#Cc1c(N)sc2c(F)ccc(-c3c(C(F)(F)F)cc4c(OC5CCC6(CNC6)C5)nc(OCC56CCCN5CC(F)C6)nc4c3F)c12] | |
| 80 | 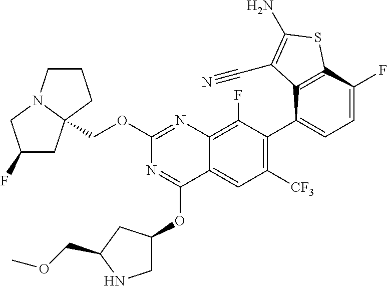 | LCMS m/z [M + 1]: 693.1 1H NMR (400 MHz, CD₃OD) δ 8.45 (s, 2H), 7.21- 7.25 (m, 1H), 7.04 (t, 1H), 6.06 (s, 1H), 5.52 (s, 0.5H), 5.39 (s, 0.5H), 4.51-4.61 (m, 2H), 4.01 (s, 1H), 3.60- 3.82 (m, 7H), 3.45 (s, 3H), 3.21-3.30 (m, 1H), 2.75- 2.90 (m, 1H), 2.05-2.58 (m, 7H), 1.34-1.58 (m, 1H). |
| 81 | 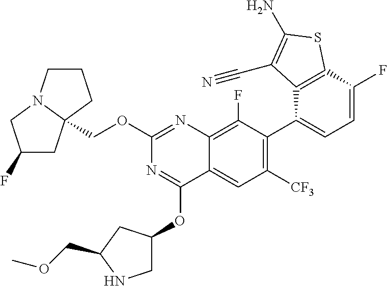 | LCMS m/z [M + 1]: 693.1 1H NMR (400 MHz, CD₃OD) δ 8.44 (s, 2H), 7.22- 7.25 (m, 1H), 7.04 (t, 1H), 6.07 (s, 1H), 5.53 (s, 0.5H), 5.40 (s, 0.5H), 4.45-4.70 (m, 2H), 4.04 (s, 1H), 3.52- 3.94 (m, 7H), 3.46 (s, 3H), 3.21-3.30 (m, 1H), 2.75- 2.90 (m, 1H), 1.98-2.61 (m, 7H), 1.38-1.51 (m, 1H). |
| 81a | 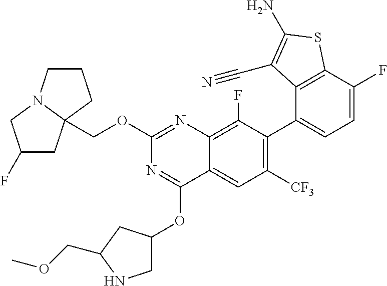 [SMILES: COCC1CC(Oc2nc(OCC34CCCN3CC(F)C4)nc3c(F)c(-c4ccc(F)c5sc(N)c(C#N)c45)c(C(F)(F)F)cc23)CN1] | |
| 82 | 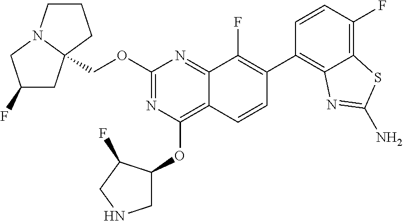 [SMILES: Nc1nc2c(-c3ccc4c(O[C@H]5CNC[C@H]5F)nc(OC[C@@]56CCCN5C[C@H](F)C6)nc4c3F)ccc(F)c2s1] | LCMS m/z [M + 1]: 575.2 1H NMR (400 MHz, CD₃OD): δ 7.98-8.00 (m, 1H), 7.60-7.63 (m, 1H), 7.34-7.38 (m, 1H), 6.98 (t, J = 8.8 Hz, 1H), 5.76-5.85 (m, 1H), 5.44-5.70 (m, 2H), 4.69- 4.89 (m, 2H), 3.34-3.89 (m, 8H), 2.12-2.56 (m, 6H). |
| 82a | 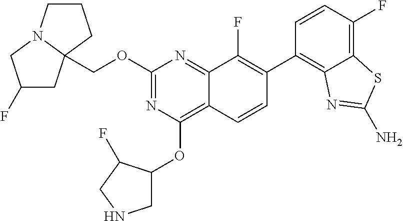 [SMILES: Nc1nc2c(-c3ccc4c(OC5CNCC5F)nc(OCC56CCCN5CC(F)C6)nc4c3F)ccc(F)c2s1] | |
| 83 | 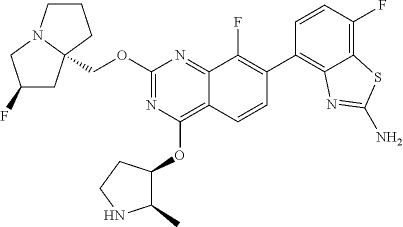 [SMILES: C[C@H]1NCC[C@H]1Oc1nc(OC[C@@]23CCCN2C[C@H](F)C3)nc2c(F)c(-c3ccc(F)c4sc(N)nc34)ccc12] | LCMS m/z [M + 1]: 571.2 1H NMR (400 MHz, CD₃OD): δ 8.02 (d, J = 8.48 Hz, 1H), 7.55-7.59 (m, 1H), 7.33-7.37 (m, 1H), 6.98 (t, J = 8.78 Hz, 1H), 6.04 (s, 1H), 5.37 (d, J = 54.10 Hz, 1H), 4.38-4.45 (m, 2H), 3.92-3.95 (m, 1H), 3.35-3.60 (m, 5H), 3.10-3.16 (m, 1H), 1.96-2.66 (m, 8H), 1.51 (d, J = 6.76 Hz, 3H). |
| 83a | 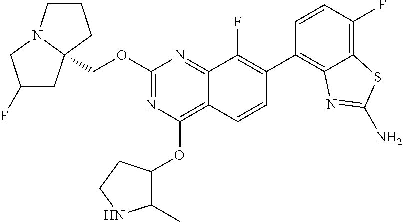 [SMILES: CC1NCCC1Oc1nc(OC[C@@]23CCCN2CC(F)C3)nc2c(F)c(-c3ccc(F)c4sc(N)nc34)ccc12] | |
| 84 | 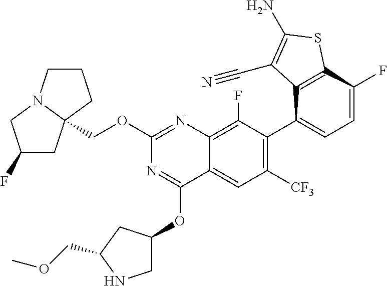 | LCMS m/z [M + 1]: 693.0. 1H NMR (400 MHz, CD₃OD) δ 8.44 (s, 1H), 7.21- 7.25 (t, 1H), 7.30-7.07 (t, 1H), 6.11 (s, 1H), 5.54 (s, 0.5H), 5.41 (s, 0.5H), 4.54-4.65 (m, 2H), 4.24-4.25 (m, 1H), 3.77-3.80 (m, 3H), 3.60-3.63 (m, 4H), 3.49 (s, 3H), 2.05-2.44 (m, 9H). |
| 85 | 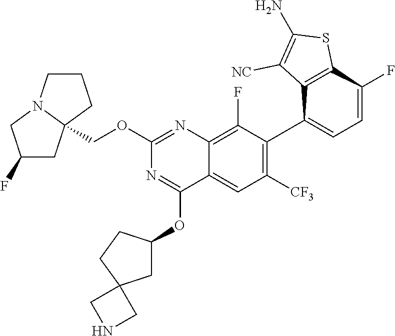 | LCMS m/z [M + 1]: 689.1. 1HNMR (400 MHz, CD₃OD) δ 8.50 (s, 1H), 8.23 (s, 1H), 7.18-7.25 (m, 1H), 7.01-7.07 (t, 1H), 5.84 (s, 1H), 5.53 (s, 0.5H), 5.39 (s, 0.5H), 4.51-4.62 (m, 2H), 4.01-4.26 (m, 4H), 3.53-3.71 (m, 3H), 3.22-3.28 (m, 1H), 2.28-2.60 (m, 7H), 2.03-2.23 (m, 5H). |
| 86 | 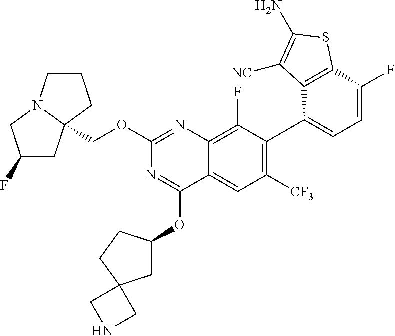 | LCMS m/z [M + 1]: 689.1 1HNMR (400 MHz, CD₃OD) δ 8.47 (s, 1H, 8.23 (s, 1H), 7.18-7.27 (m, 1H), 6.99-7.07 (t, 1H), 5.14 (s, 1H), 5.53 (s, 0.5H), 5.40 (s, 0.5H), 4.47-4.60 (m, 2H), 3.95-4.28 (m, 4H), 3.50-3.73 (m, 3H), 3.25-3.30 (m, 1H), 2.25-2.65 (m, 7H), 2.01-2.24 (m, 5H). |
| 87 | 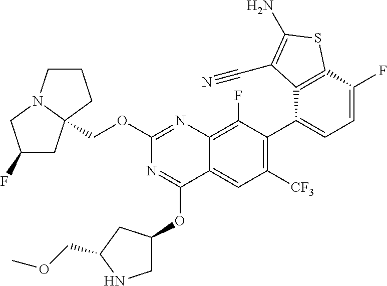 | LCMS m/z [M + 1]: 693.0 1H NMR (400 MHz, CD₃OD) δ 8.42 (s, 1H), 7.22- 7.23 (t, 1H), 7.03-7.07 (t, 1H), 6.09 (s, 1H), 5.47 (s, 0.5H), 5.33 (s, 0.5H), 4.47-4.49 (m, 2H), 4.21 (m, 1H), 3.76 (s, 2H), 3.59-3.64 (m, 1H), 3.49 (s, 3H), 3.40 (m, 3H), 3.15 (m, 1H), 2.61-2.67 (m, 1H), 2.34-2.44 (m, 3H), 1.99-2.26 (m, 5H). |
| 88 | 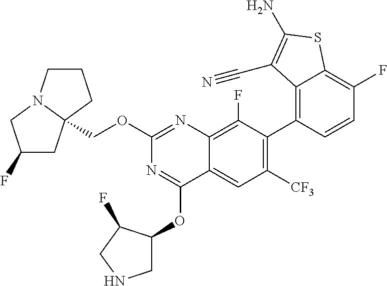 | LCMS m/z [M + 1]: 667.1 1H NMR (400 MHz, CD₃OD) δ 8.61-8.26 (m, 2H), 7.32-7.14 (m, 1H), 7.11-6.95 (m, 1H), 6.02-5.84 (m, 1H), 5.79-5.39 (m, 2H), 4.76-4.53 (m, 2H), 3.96-3.49 (m, 6H), 2.69-2.03 (m, 6H), 1.56-1.38 (m, 1H), 7.11- 6.94 (m, 1H). |
| 89 | 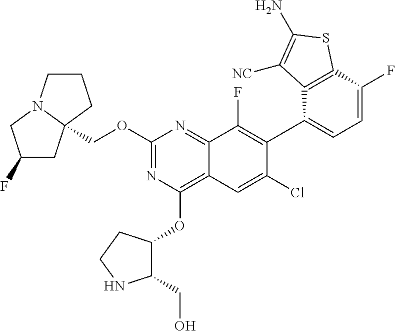 | LCMS m/z [M + 1]: 645.1 1H NMR (400 MHz, CD₃OD) δ 8.22 (s, 1H), 7.22- 7.25 (t, 1H), 7.05-7.09 (t, 1H), 5.59 (s, 0.5H), 5.37 (s, 0.5H), 5.22 (d, 1H), 4.74-4.49 (m, 2H), 4.11 (m, 1H), 3.76 (m, 2H), 3.59-3.64 (m, 5H), 2.04-2.22 (m, 9H)). |
| 90 | 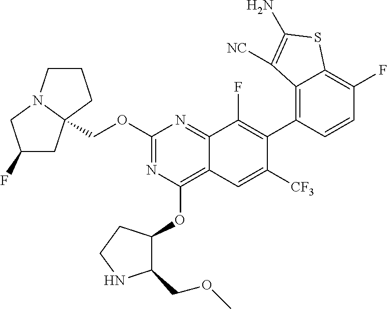 | LCMS m/z [M + 1]: 693.2 1H NMR (400 MHz, CD₃OD): δ 8.42 (s, 1H), 7.19- 7.24 (m, 1H), 7.03 (t, J = 8.78 Hz, 1H), 6.16-6.18 (m, 1H), 5.37 (d, J = 53.72 Hz, 1H), 4.40-4.49 (m, 2H), 3.83-4.04 (m, 3H), 3.34-3.61 (m, 8H), 3.10-3.15 (m, 1H), 1.97-2.63 (m, 8H). |
| 90a | 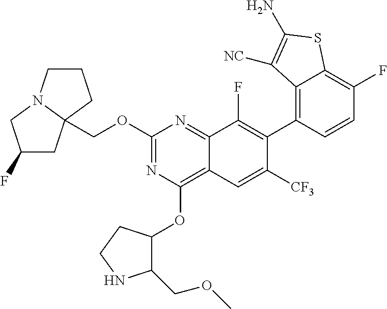 | |
| 91 | 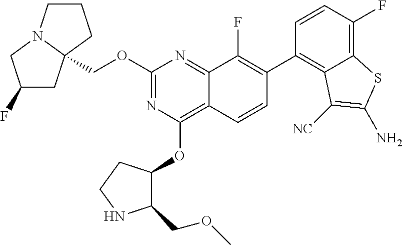 | LCMS m/z [M + 1]: 625.3 1H NMR (400 MHz, CD₃OD) δ 8.04-8.06 (m, 1H), 7.47-7.50 (m, 1H), 7.25-7.28 (m, 1H), 7.05 (t, J = 8.8 Hz, 1H), 6.17 (s, 1H), 5.60 (d, J = 52.8 Hz, 1H), 4.67- 4.84 (m, 2H), 3.87-4.16 (m, 5H), 3.48-3.63 (m, 7H), 2.36-2.67 (m, 8H). |
| 91a | 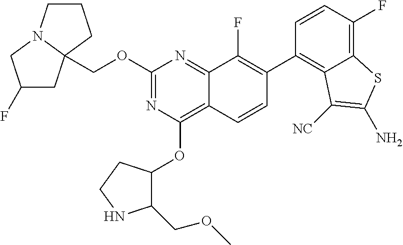 | |
| 92 | 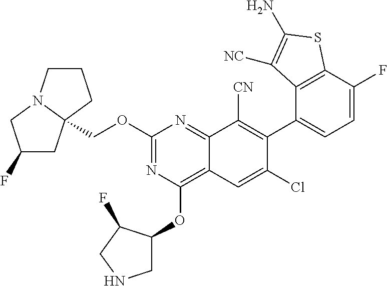 | LCMS m/z [M + 1]: 640.3 1H NMR (400 MHz, CD₃OD) δ 8.58-8.49 (m, 1H), 8.49-8.27 (m, 1H), 7.31-7.22 (m, 1H), 7.09 (t, J = 9.2 Hz, 1H), 5.86-5.74 (m, 1H), 5.63 (s, 0.5H), 5.49 (s, 1H), 5.36 (s, 0.5H), 4.62-4.49 (m, 2H), 3.78-3.35 (m, 7H), 3.29-3.17 (m, 2H), 2.57-1.97 (m, 6H). |
| 92a | 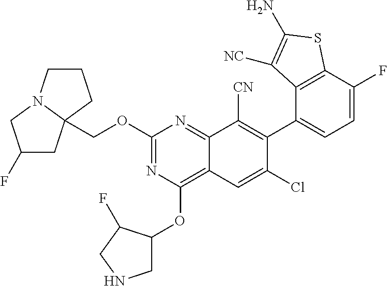 | |
| 93 | 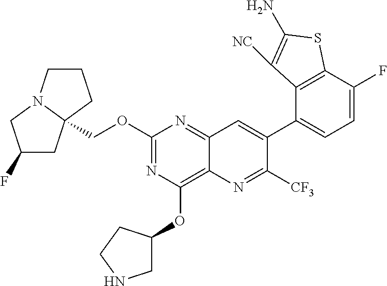 | LCMS m/z [M + 1]: 632.0 1H NMR (400 MHz, CD₃OD) δ 8.32 (d, 1H), 8.16- 8.20 (m, 1H), 7.22-7.25 (m, 1H), 7.02-7.06 (m, 1H), 6.15 (s, 1H), 5.58-5.64 (m, 0.5H), 5.45-5.51 (m, 0.5H), 4.62-4.69 (m, 2H), 4.29 (s, 1H), 3.59-3.85 (m, 6H), 2.05-2.62 (m, 9H). |
| 93a | 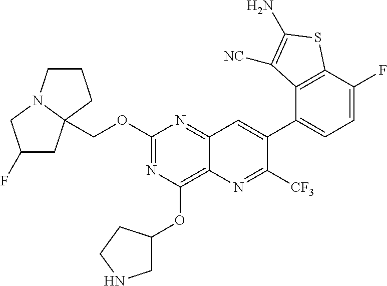 | |
| 94 | 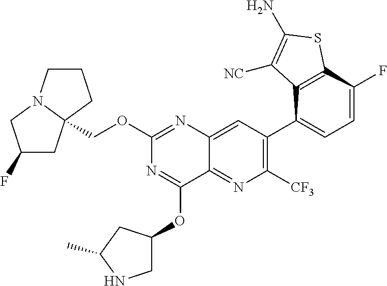 | LCMS m/z [M + 1]: 646.1 1HNMR (400 MHz, CD₃OD) δ 8.36 (s, 2H), 8.15 (s, 1H), 7.20-7.26 (m, 1H), 7.04 (t, 1H), 6.10 (t, 1H), 5.55 (s, 0.5H), 5.42 (s, 0.5H), 4.52-4.62 (m, 2H), 4.14-4.20 (m, 1H), 3.92-3.96 (m, 1H), 3.82 (d, 1H), 3.65 (d, 3H), 2.77-2.82 (m, 1H), 2.42-2.61 (m, 2H), 2.09-2.33 (m, 6H), 1.54 (d, 3H). |
| 95 | 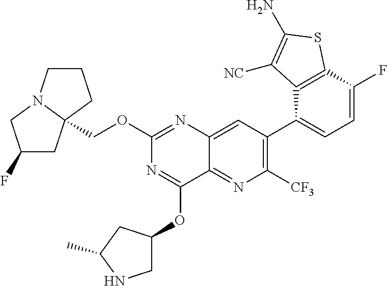 | LCMS m/z [M + 1]: 646.1 1HNMR (400 MHz, CD₃OD) δ 8.31 (s, 2H), 8.15 (s, 1H), 7.23-7.26 (m, 1H), 7.04 (t, 1H), 6.11 (t, 1H), 5.56 (s, 0.5H), 5.43 (s, 0.5H), 4.58-4.64 (m, 2H), 4.15-4.21 (m, 1H), 3.92-3.96 (m, 1H), 3.81 (d, 1H), 3.63 (d, 3H), 2.77-2.82 (m, 1H), 2.48-2.62 (m, 2H), 1.97-2.37 (m, 6H), 1.54 (d, 3H). |
| 95a | 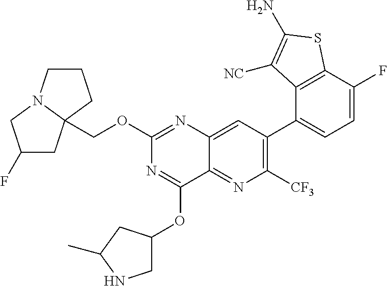 | |
| 96 | 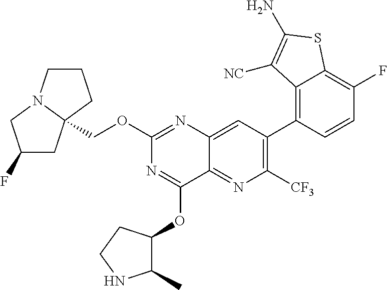 | LCMS m/z [M + 1]: 646.0 1HNMR (400 MHz, CD₃OD) δ 8.32 (s, 1H), 8.18 (s, 1H), 7.23-7.28 (m, 1H), 7.01-7.07 (t, 1H), 6.07-6.17 (m, 1H), 5.50 (s, 0.5H), 5.38 (s, 0.5H), 4.47-4.60 (m, 2H), 3.97-4.05 (m, 1H), 3.60-3.70 (m, 1H), 3.46-3.60 (m, 3H), 3.22-3.28 (m, 1H), 2.35-2.70 (m, 5H), 2.02- 2.33 (m, 4H), 1.58-1.61 (t, 3H). |
| 96a | 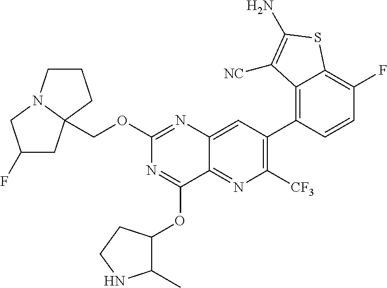 | |
| 97 | 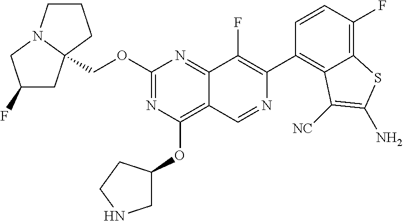 | LCMS m/z [M + 1]: 582.2 1H NMR (400 MHz, CD₃OD) δ 9.23 (s, 1H), 7.38- 7.42 (m, 1H), 7.07 (t, J = 8.8 Hz, 1H), 6.09 (s, 1H), 5.38 (d, J = 52.8 Hz, 1H), 4.41-4.61 (m, 2H), 3.10- 3.80 (m, 8H), 1.95-2.56 (m, 8H). |
| 97a | 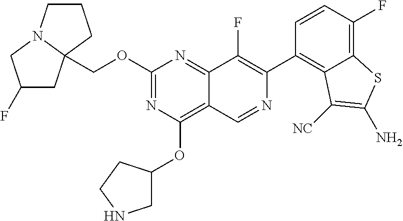 | |
| 98 | 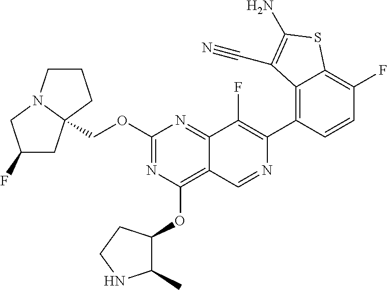 | LCMS m/z [M + 1]: 596.2 1H NMR (400 MHz, CD₃OD) δ 9.31 (s, 1H), 7.40- 7.43 (m, 1H), 7.08 (t, J = 8.8 Hz, 1H), 6.10 (s, 1H), 5.45 (d, J = 52.8 Hz, 1H), 4.56-4.63 (m, 3H), 4.01- 4.02 (m, 1H), 3.53-3.60 (m, 5H), 2.05-2.64 (m, 8H), 1.55 (d, J = 6.4 Hz, 3H). |
| 98a | 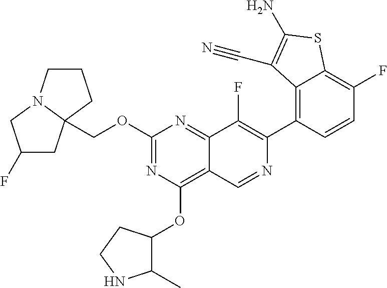 | |
| 99 | 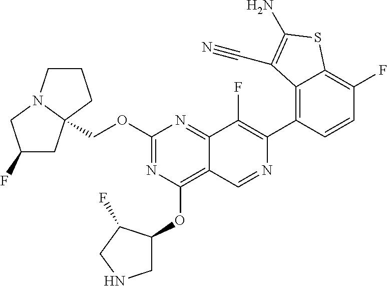 | LCMS m/z [M + 1]: 600.2 1H NMR (400 MHz, CD₃OD) δ 9.27 (s, 1H), 7.42- 7.45 (m, 1H), 7.10 (t, J = 8.8 Hz, 1H), 5.97-5.99 (m, 1H), 5.40-5.61 (m, 2H), 4.57-4.64 (m, 2H), 3.15-3.76 (m, 8H), 1.95-2.56 (m, 6H). |

### Biological Example 1: Determination of Compounds' Binding Affinity to RAS Ligands by SPR

The affinity of compounds of the present disclosure to RAS ligands was assessed using surface plasmon resonance (SPR) binding analysis using a Biacore S200 (GE Healthcare Life Sciences) instrument. The SPR binding analysis employed the following reagents and protocols:

Buffer preparation: To make 0.5 L add required amounts of stocks for: 20 mM Hepes, 150 mM NaCl, 5 mM MgCl₂, 1 mM TCEP, 0.05% P20, and 5 μM nucleotide. Adjust pH to 7.4, then bring volume up to 475 mL 1.05×Filter through 0.2 μm cellulose acetate membrane. Remove a volume of buffer as No DMSO Buffer 1.05× and set aside. Add appropriate volume of DMSO to buffer to a final concentration 5% DMSO.

Chip preparation: Prime 2× with 5% DMSO buffer. Allow system to flow on standby throughout the day.

Sample preparation: Make a 20× compound solution in 100% DMSO of highest compound concentration to be screened (2 mM for final 100 μM compound). Add appropriate volume of 1.05× buffer without DMSO to give 5% DMSO and mix by pipetting. Centrifuge sample 18,000×g 5 min 25° C. Make appropriate serial dilutions from the highest concentration into 5% DMSO running buffer for the concentrations needed. Prepare 5.8% DMSO and 4.5% DMSO solvent correction solutions using buffer without DMSO. Make two more solvent corrections by mixing 200 μL 4.5% 500 μL 5.8%, and 400 μL 4.5% 300 μL 5.8%

Capture Avi-taggedprotein: Thaw protein stock on ice, aliquot into single use size and freeze at −80° C. for future use. Dilute protein (avi-KRAS WT, avi-KRAS-G12D, avi-KRAS-G12V, or avi-RhoA) appropriately, to 20 μg/mL to begin in 5% DMSO buffer. Manually inject at 10 μL/min 12 second contact time. Adjust flow and or contact time to achieve desired level of captured protein. When desired level of protein is captured, flow for 10 minutes to stabilize signal.

General Binding Level Screen Method: Choose LMW screen template in Fragment/LMW subfolder in Binding screen folder. 30 μL/min high performance injection, 60 sec contact and dissociation time, carry over control, extra wash with 50% DMSO for each injection. Fifteen Startup cycles to condition. Control samples before and after compound samples. For GppNHp loaded KRAS, control is 20 μM Raf1 RBD domain. Adjust this sample to final 5% DMSO to match running buffer. This process is performed at a temperature of 25° C. using a single chip per experiment.

Binding affinity determination: Compounds selected from the binding level screen are evaluated in a dose response to determine the binding affinity (K_{D}). Serial dilutions of compounds are prepared and injected over the sensor chip using a flow rate of 30 μL/min with a 60 second contact and dissociation time. After double referencing of the sensorgrams the total level of compound binding at each concentration is calculated. The steady state binding affinity is calculated with a Langmuir binding model assuming a 1:1 interaction with a constrained Rmax using the BIAevaluation software.

### Biological Example 2: Disrupting RAS-Effector Binding HTRF Assay

A protein:protein interaction (PPI) Homogeneous Time Resolved Fluorescence (HTRF) assay was used to determine the effectiveness of compounds of the present disclosure in disrupting KRAS protein and effector (RAF1 or PIK3 CA) binding.

The HTRF assay used the following reagents and proteins: (i) 375 nM Avi-KRAS G12D/Q25A (1-169) GppNHp/3×FLAG-PI3KCA (157-299); (ii) 100 nM Avi-KRAS G12D (1-169) GppNHp/RAF1 RBD-3×FLAG (52-151); (iii) 50 nM Avi-KRAS G12V (1-169) GppNHp/RAF1 RBD-3×FLAG (52-151); (iv) 50 nM Avi-KRAS WT (1-169) GppNHp/RAF1 RBD-3×FLAG (52-151); and (v) 35 nM Avi-PI3K RBD CA-3×FLAG; Assay Buffer: 50 mM Tris pH 7.5, 100 mM NaCl, 5 mM MgCl₂, 0.1% BSA, 0.01% Tween, 1 mM TCEP, 10% DMSO; Bead Buffer: 50 mM Tris pH 7.5, 0.01% Tween 20; Assay volume: 20 μL (384 well plate-low volume format); and Compound titration: 100-0.02 μM, 3× dilution series.

The HTRF assay employed the following protocol:

Compounds were dispensed in assay plate (384-well, Grenier Bione #784075) using Echo (model 555) with dose response settings: 200 nL final volume, titration from 100 μM as a 10-point dilution series. Protein mix was prepared in assay buffer, and dispensed on plates, 10 μL per well, then incubated for 1 h at room temperature, with 700 rpm shaking. Reagents mix was prepared and dispensed on plates, 10 μL per well, then incubated for 1 h at room temperature, with 700 rpm shaking.

Plates were analyzed on an Envision plate reader using the following setting: Excitation 320 nM, Bandwidth 75 nM; Emission 615 nM, Bandwidth 85 nM, Gain 100%, Flashes 100, Lag 60 μs. Data was reported as percentage of activity, with DMSO as 100%. Data was plotted and analyzed using Prism 8. The SPR Kd, biochemical Raf1-KRAS G12D/G12V/WT-GppNHP and PI3KCA-KRAS G12D/Q25A-GppNHp disruption assay IC₅₀ of selected compounds described herein are shown in Table 4.

### Biological Example 3: Cell-Based pERK HTRF Assay in GP2d Cell

A pERK assay (Perkin Elmer) was used to determine the effectiveness of compounds in disrupting KRAS G12D protein/effector signaling in cells.

On Day 1, cells (GP2d) were seeded into 96-well plates at 3×10⁴ cells/well in 80 μL complete growth media (DMEM, 10% FBS).

On Day 2, cells were treated with compounds at 0.25% DMSO. The source plate was created with compounds diluted in media at 5-fold the final assay concentration. The compounds were run in a 9-point concentration curve starting at 1 μM, with a half-log dilution between concentrations. 20 μL was transferred onto the cell plates (final volume in wells was 100 μL). Plates were harvested after 60 min incubation by aspirating media and adding kit-supplied 1× supplemented lysis buffer to all wells (50 μL per well). Plates were then placed on a plate shaker and incubated at 850 rpm for an additional 30 min.

Antibody mixture solution is prepared by diluting aliquoted d2 and Eu Cryptate antibodies 1:20 in kit supplied detection buffer, mix the diluted antibodies solutions (1:1 v:v). 4 μL of this solution is then added to a 384-well detection plate (Perkin Elmer; 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. Maximum signal is reached after 4 h incubation time and remains stable over a period of 24 hours. Therefore, readings can be made between 4 and 24 h of incubation. Plates were centrifuged again (524 g for Imin), then analyzed on the EnVision plate reader using the following settings: Excitation 320 nm, Bandwidth 75 nm; Emission 615 nm, Bandwidth 85 nm; Gain 100%; Flashes 100; Lag 60 μs.

In Table 4: For KRAS G12D binding affinity: A: K_{D}≤0.1 μM; B: 0.1 μM≤K_{D}<1 μM; C: 1 μM<K_{D}≤10 μM; D: K_{D}>10 μM. For Raf1-KRAS G12D, G12V, WT-GppNHP and PI3KCA-KRAS G12D/Q25A-GppNHp disruption assay: A: IC₅₀≤0.1 μM; B: 0.1 μM<IC₅₀≤1 μM; C: 1 μM<IC₅₀≤10 μM; D: IC₅₀>10 μM. For GP2d pERK HTRF assay: A: IC₅₀≤0.1 μM; B: 0.1 μM<IC₅₀≤1 μM; C: IC₅₀>1 μM. Blanks in the table represent that a compound was not tested in the indicated assay.

TABLE 4
Biological characterization of selected compounds of the present disclosure.

| | GDP | GppNHp | Raf1- | PI3KCA- | Raf1- | Raf1- | |
|---|---|---|---|---|---|---|---|
| | KRAS | KRAS | GppNHp- | GppNHp- | GppNHp- | GppNHp- | |
| | G12D | G12D | KRAS | KRAS | KRAS | KRAS | pERK- |
| Compound | binding | binding | G12D | G12D/Q25A | G12V | WT | Gp2D- |
| No. | affinity | affinity | disruption | disruption | disruption | disruption | 1 hr |
| 1 | | | C | C | D | C | |
| 2 | | | C | C | D | C | |
| 3 | | | D | | | | |
| 4 | | | B | B | C | C | A |
| 5 | | | D | D | D | D | |
| 6 | | | C | C | D | C | |
| 7 | | C | B | C | D | C | A |
| 8 | | | B | B | C | C | B |
| 9 | | | D | D | D | D | |
| 10 | | | B | B | C | C | B |
| 11 | | | D | D | D | D | C |
| 12 | | | D | D | D | D | C |
| 13 | | | B | C | C | B | A |
| 14 | | | D | D | D | D | C |
| 15 | | | B | C | C | C | B |
| 16 | | | C | C | C | C | B |
| 17 | A | B | B | B | C | C | B |
| 18 | B | D | D | D | D | D | |
| 19 | A | B | B | B | C | B | A |
| 20 | A | B | B | C | C | C | A |
| 21 | C | D | D | D | D | D | |
| 22 | | | D | C | D | C | |
| 23 | | | D | D | D | D | C |
| 24 | | | D | D | D | D | C |
| 25 | | | B | B | C | B | A |
| 26 | A | A | A | B | C | A | C |
| 27 | | | B | D | C | C | B |
| 28 | | | C | D | D | C | B |
| 29 | | | D | D | D | D | C |
| 30 | | | C | D | D | D | C |
| 31 | | | C | D | D | D | C |
| 32 | | | D | D | D | D | C |
| 33 | | | C | C | D | C | B |
| 34 | | | B | B | B | B | A |
| 35 | | | B | D | C | C | B |
| 36 | | | B | D | D | C | C |
| 40 | | | A | | B | A | B |
| 43 | | | B | | B | B | B |
| 46 | | | B | D | D | D | C |
| 47 | | | A | D | B | A | A |
| 48 | | | B | B | D | C | B |
| 49 | | | B | B | C | C | A |
| 50 | | | B | B | B | B | A |
| 51 | | | C | C | D | D | C |
| 52 | | | B | C | D | C | B |
| 53 | | | B | B | C | B | A |
| 54 | | | B | B | C | B | B |
| 55 | | | C | C | D | C | C |
| 56 | | | C | B | D | C | B |
| 58 | | | C | B | D | C | B |
| 59 | | | B | B | B | B | A |
| 60 | | | B | B | B | B | A |
| 61 | | | D | D | D | D | C |
| 62 | | | B | C | C | B | A |
| 63 | | | B | C | C | C | B |
| 64 | | | D | D | D | D | C |
| 65 | | | B | B | B | B | A |
| 66 | | | C | C | D | C | B |
| 67 | | | B | D | C | B | B |
| 68 | | | D | D | D | D | C |
| 69 | | | A | B | B | B | B |
| 70 | | | A | B | B | B | B |
| 71 | | | C | C | D | C | C |
| 72 | | | A | B | B | A | A |
| 73 | | | A | A | A | A | A |
| 74 | | | D | D | D | D | C |
| 75 | | | D | D | D | D | C |
| 76 | | | A | A | A | A | A |
| 77 | | | D | D | D | D | C |
| 78 | | | C | | D | C | C |
| 79 | | | A | | A | A | A |
| 80 | | | A | | B | B | A |
| 81 | | | D | | D | D | C |
| 82 | | | D | | D | D | C |
| 83 | | | D | | D | D | C |
| 84 | | | A | | B | B | A |
| 85 | | | A | | B | A | B |
| 86 | | | D | | D | D | C |
| 87 | | | D | | D | D | B |
| 88 | | | B | | C | B | A |
| 89 | | | D | | D | D | C |
| 90 | | | B | | C | B | A |
| 91 | | | D | | D | D | B |
| 92 | | | C | | D | C | B |
| 93 | | | C | | C | C | A |
| 94 | | | C | | C | C | A |
| 95 | | | D | | D | D | B |
| 96 | | | B | | C | B | A |
| 97 | | | C | D | D | C | B |
| 98 | | | C | D | C | C | B |
| 99 | | | D | D | D | D | C |

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents. It is intended that the following claims define the scope of the invention and that methods and structures within the scope of these claims and their equivalents be covered thereby.

1. A compound according to Formula I: 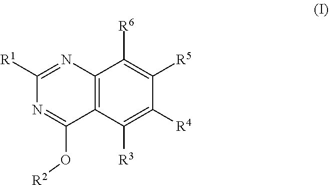
or a salt (e.g., pharmaceutically acceptable salt) thereof, wherein:
R¹ is selected from —OR⁷, 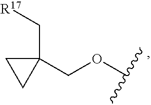
and a 4-6 membered heterocycle comprising a nitrogen atom, wherein the heterocycle is unsubstituted or substituted with one or more R¹⁵;
R² is a 4-11 membered heterocycle containing one or more nitrogen atoms, wherein the heterocycle is unsubstituted or is substituted with one or more R⁸;
R³ is H;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is independently selected from halogen and C₁₋₆alkyl, wherein any C₁₋₆alkyl is unsubstituted or substituted with one or more R¹¹;
each R⁹ is independently selected from halogen, N(R¹²)₂, —CN, and C₁₋₆alkyl, wherein any C₁₋₆alkyl is unsubstituted or substituted with one or more R¹³;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen; and
each R^{a} and R^{b} is independently selected from halogen, C₁₋₆ alkyl, —OR¹², and H, wherein an W and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

2. The compound of claim 1, wherein R² is a 4-6 membered heterocycle containing one nitrogen atom, wherein the heterocycle is unsubstituted or is substituted with one or more R⁸.

3. The compound of claim 2, wherein R² is a heterocycle selected from azetidine and pyrrolidine, wherein the heterocycle is unsubstituted or is substituted with one or more R⁸.

4. The compound of claim 1, wherein R² is a bridged heterocycle containing one nitrogen atom, wherein the heterocycle is unsubstituted or is substituted with one or more R⁸.

5. The compound of claim 1, wherein R² is a spirocyclic heterocycle containing one or more nitrogen atoms, wherein the heterocycle is unsubstituted or substituted with one or more R⁸.

6. The compound of claim 1, wherein R² is a spirocycle including a first ring that is a carbocycle and a second ring that is a heterocycle containing a nitrogen atom, wherein the spirocycle is unsubstituted or is substituted with one or more R⁸.

7. The compound of any one of claims 1-6, wherein R² is unsubstituted.

8. The compound of any one of claims 1-6, wherein R² is substituted with one or more R⁸, wherein the one or more R⁸ are each independently selected from halogen and C₁₋₆alkyl, wherein any C₁₋₆alkyl is unsubstituted.

9. The compound of claim 1, wherein the compound is a compound according to Formula IA: 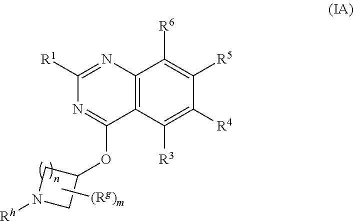
or a salt (e.g., pharmaceutically acceptable salt) thereof, wherein:
n is 1, 2, or 3;
m is 0, 1, 2, 3, or 4;
each R⁹ is independently selected from halogen and C₁₋₆alkyl, wherein any C₁₋₆alkyl is unsubstituted or substituted with one or more R¹¹, or two R⁸, together with the atoms to which they are attached, join to form a 5-7 membered ring;
R^{h} is selected from H and C₁₋₆alkyl, wherein any C₁₋₆alkyl is unsubstituted or substituted with one or more R¹¹.

10. The compound of claim 9, wherein the compound has the structure: 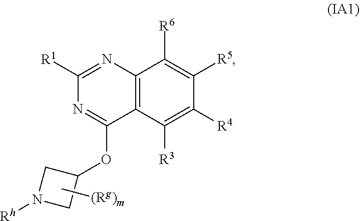
or a salt (e.g., pharmaceutically acceptable salt) thereof.

11. The compound of claim 9, wherein the compound has the structure: 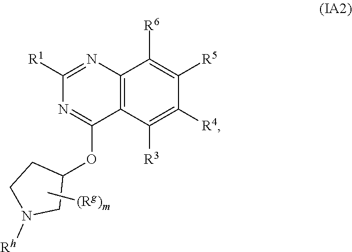
or a salt (e.g., pharmaceutically acceptable salt) thereof.

12. The compound of claim 9, wherein the compound has the structure: 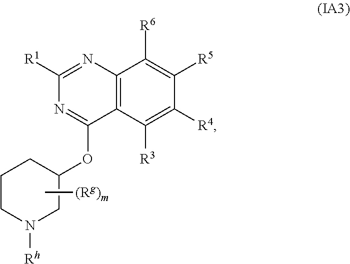
or a salt (e.g., pharmaceutically acceptable salt) thereof.

13. The compound of claim 9, wherein the compound has the structure: 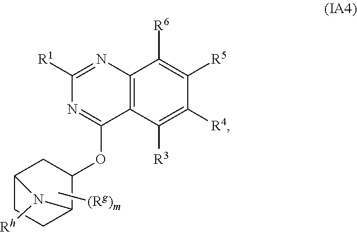
or a salt (e.g., pharmaceutically acceptable salt) thereof.

14. The compound of claim 9, wherein the compound has the structure: 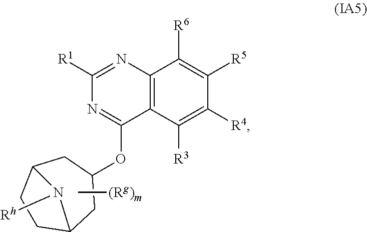
or a salt (e.g., pharmaceutically acceptable salt) thereof.

15. The compound of any one of claims 9-14, wherein R¹ is H.

16. The compound of any one of claims 9-15, wherein m is 1 or 2, and each R^{g} is independently selected from halogen and C₁₋₆alkyl, wherein any C₁₋₆alkyl is unsubstituted.

17. The compound of claim 1, wherein the compound is a compound according to Formula IB: 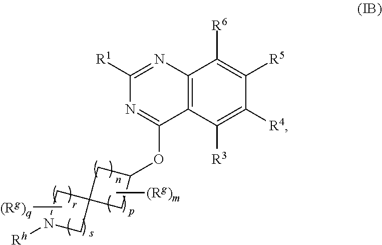
or a salt (e.g., a pharmaceutically acceptable salt) thereof, wherein:
m and q are each independently selected from 0, 1, 2, 3, and 4;
n and p are each independently selected from 1 and 2;
r and s are each independently selected from 1 and 2; each R⁹ is independently selected from halogen and C₁₋₆alkyl, wherein any C₁₋₆alkyl is unsubstituted or substituted with one or more R¹¹; and
R^{h} is selected from H and C₁₋₆alkyl, wherein any C₁₋₆alkyl is unsubstituted or substituted with one or more R¹¹.

18. The compound of claim 17, wherein the compound has the structure: 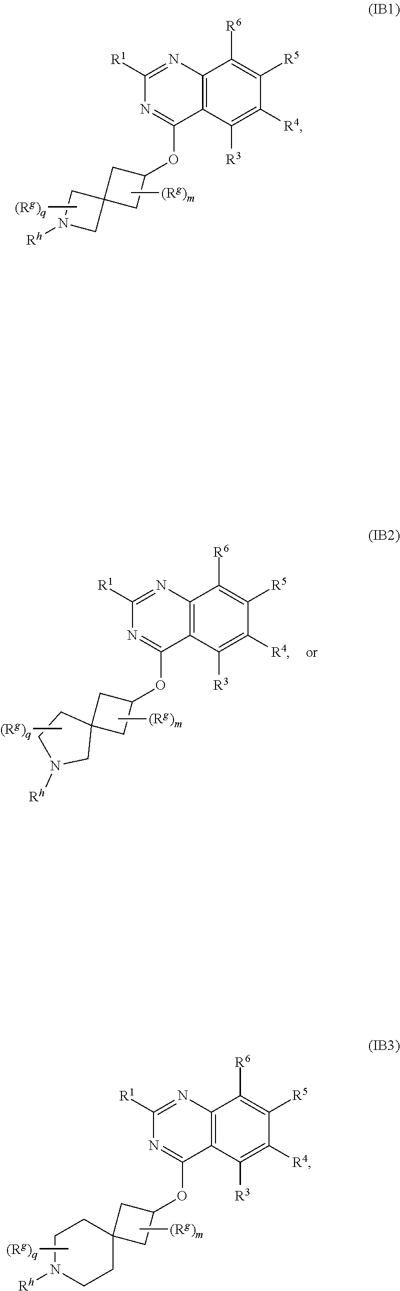
or a salt (e.g., a pharmaceutically acceptable salt) thereof.

19. The compound of claim 17, wherein the compound has the structure: 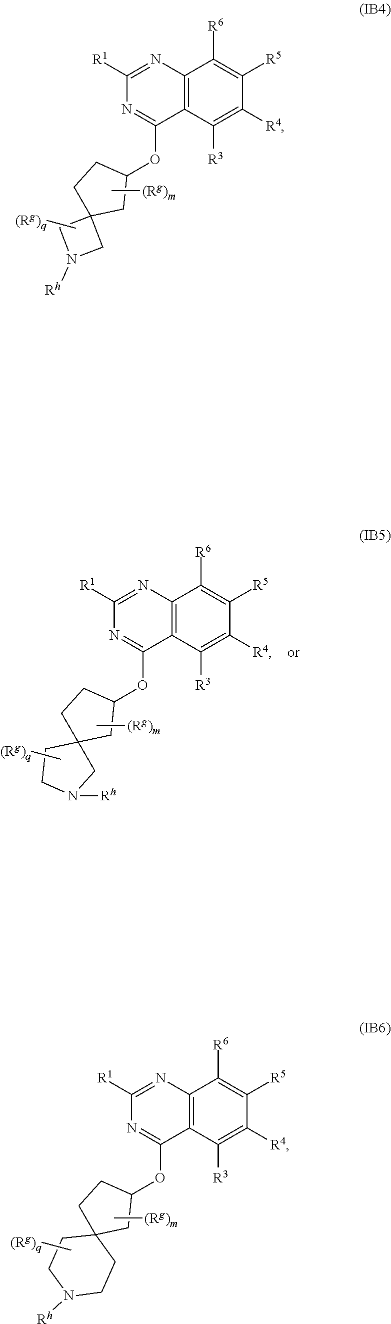
or a salt (e.g., a pharmaceutically acceptable salt) thereof.

20. The compound of claim 17, wherein the compound has the structure: 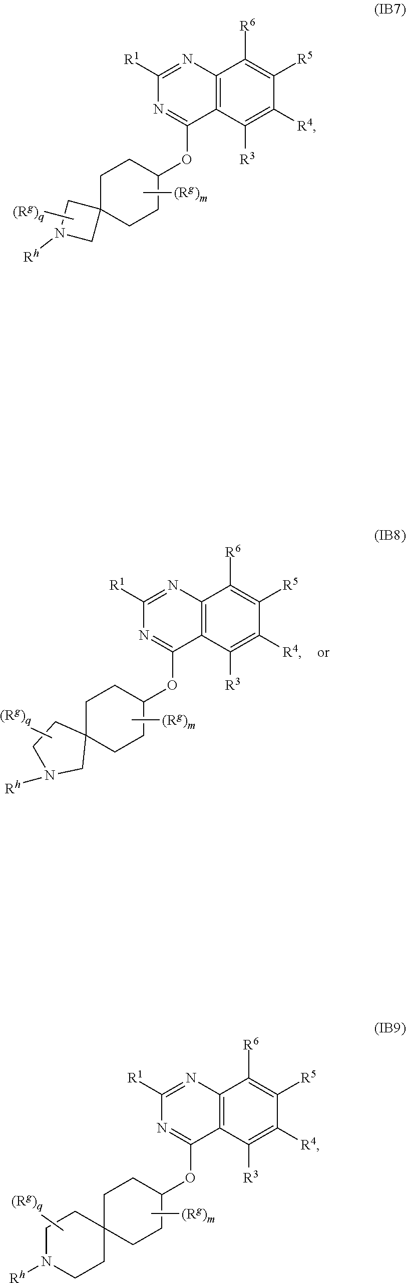
or a salt (e.g., a pharmaceutically acceptable salt) thereof.

21. The compound of any one of claims 17-20, wherein R^{h} is H.

22. The compound of any one of claims 17-21, wherein m and q are 0.

23. The compound of any one of claims 1-22, wherein R¹ is —OR⁷.

24. The compound of claim 23, wherein R¹ is: 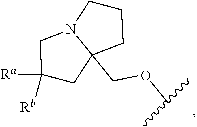
wherein R^{a} and R^{b} are each independently selected from halogen, C₁₋₆alkyl, —OR¹², and H, wherein any C₁₋₆alkyl is unsubstituted or is substituted with one or more R¹³.

25. The compound of claim 24, wherein R^{b} is H.

26. The compound of claim 24, wherein R¹ is selected from:

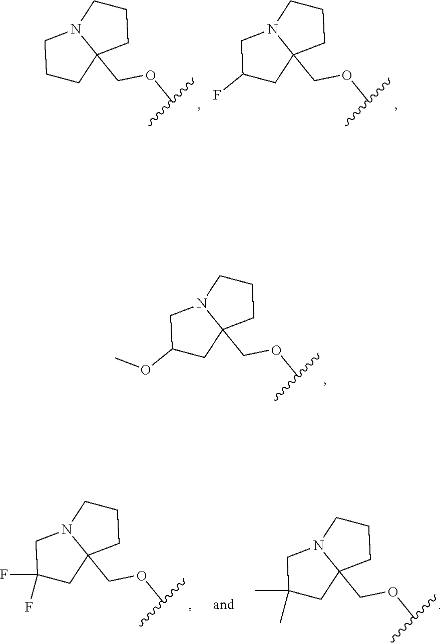

27. The compound of claim 23, wherein R¹ is selected from: 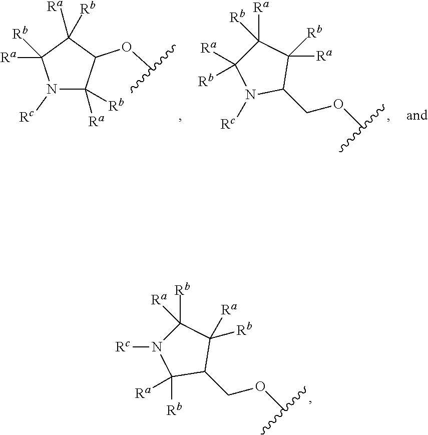
wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, wherein any C₁₋₆ alkyl or 3-6 membered carbocycle or heterocycle is unsubstituted or is substituted with one or more R¹³.

28. The compound of claim 27, wherein R¹ is selected from:

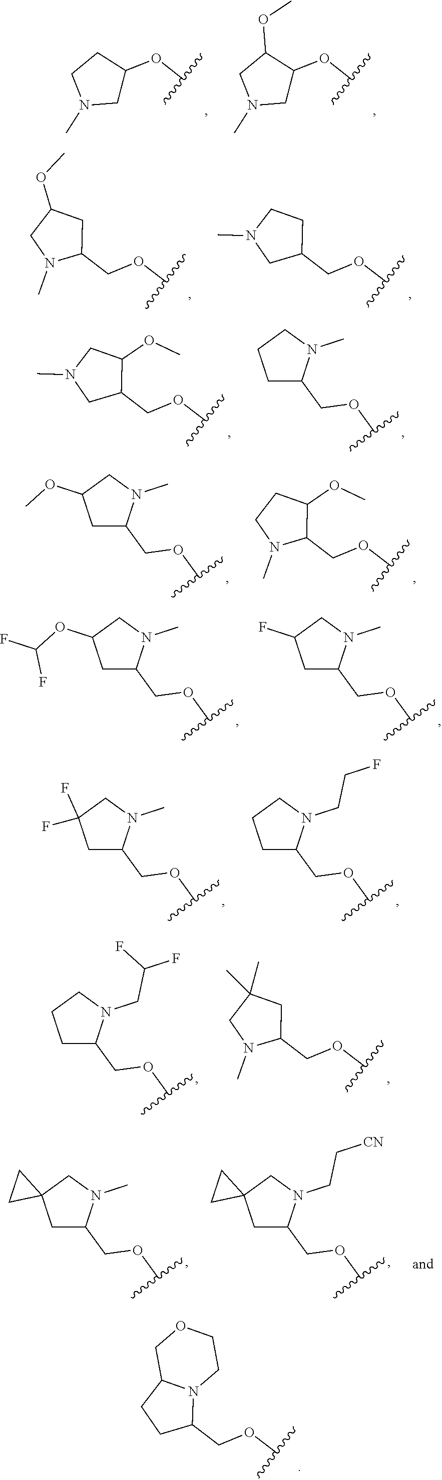

29. The compound of any one of claims 1-22, wherein R¹ is

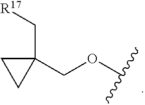

30. The compound of claim 29, wherein R¹ is selected from:

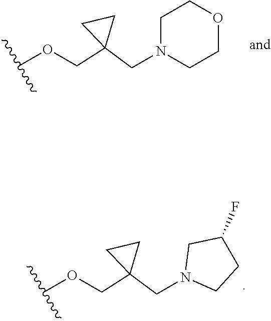 [SMILES: *OCC1(CN2CCOCC2)CC1 ; *OCC1(CN2CC[C@@H](F)C2)CC1]

31. The compound of any one of claims 1-22, wherein R¹ is a 4-6 membered heterocycle comprising a nitrogen atom, wherein the heterocycle is unsubstituted or substituted with one or more R¹⁵.

32. The compound of claim 31, wherein R¹ is selected from:

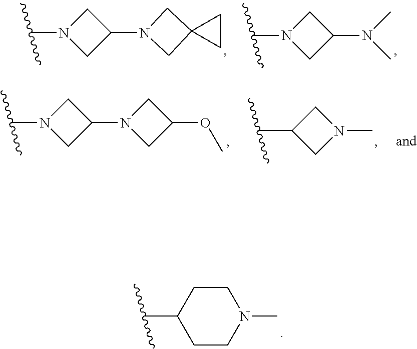

33. The compound of claim 1, wherein the compound is a compound according to Formula IC: 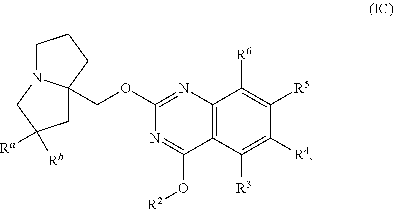
or a salt (e.g., pharmaceutically acceptable salt) thereof.

34. The compound of claim 33, wherein R^{a} is a halogen or —OR¹².

35. The compound of claim 33 or 34, wherein R^{b} is H.

36. The compound of any one of claims 33-35, wherein R² is a 4-6 membered heterocycle containing one nitrogen atom, wherein the heterocycle is unsubstituted or is substituted with one or more R⁸.

37. The compound of claim 36, wherein R² is a heterocycle selected from azetidine, pyrrolidine, and piperidine, wherein the heterocycle is unsubstituted or is substituted with one or more R⁸.

38. The compound of any one of claims 33-35, wherein R² is a bridged heterocycle containing one nitrogen atom, wherein the heterocycle is unsubstituted or is substituted with one or more R⁸.

39. The compound of any one of claims 33-35, wherein R² is a spirocyclic heterocycle containing one or more nitrogen atoms, wherein the heterocycle is unsubstituted or substituted with one or more R⁸.

40. The compound of claim 39, wherein R² is a spirocycle including a first ring that is a carbocycle and a second ring that is a heterocycle containing a nitrogen atom, wherein the spirocycle is unsubstituted or is substituted with one or more R⁸.

41. The compound of any one of claims 33-40, wherein R² is unsubstituted.

42. The compound of any one of claims 33-40, wherein R² is substituted with one or more R⁸.

43. The compound of claim 42, wherein the one or more R⁸ are each independently selected from C₁₋₆alkyl, wherein any C₁₋₆alkyl is unsubstituted.

44. The compound of any one of claims 1-43, wherein R⁴ is H.

45. The compound of any one of claims 1-43, wherein R⁴ is selected from halogen, —CN, —OR¹², and C₁₋₆alkyl, wherein any C₁₋₆alkyl is unsubstituted or substituted with one or more R¹.

46. The compound of claim 45, wherein R⁴ is —CN.

47. The compound of claim 45, wherein R⁴ is a halogen.

48. The compound of claim 45, wherein R⁴ is C₁₋₆alkyl that is substituted with one or more R¹¹.

49. The compound of claim 48, wherein R⁴ is C₁₋₆alkyl that is substituted with one or more halogens or —CN.

50. The compound of claim 49, wherein R⁴ is selected from —CF₂H, —CF₃, —CF₂CH₃, and —CH₂CN.

51. The compound of claim 50, wherein R⁴ is —CF₃.

52. The compound of any one of claims 1-51, wherein R⁶ is selected from halogen, —CN, and H.

53. The compound of claim 52, wherein R⁶ is a halogen.

54. The compound of claim 52, wherein R⁶ is —CN.

55. The compound of claim 52, wherein R⁶ is H.

56. The compound of any one of claims 1-55, wherein R⁵ is: 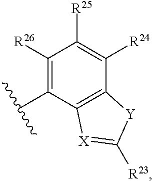
wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

57. The compound of any one of claims 1-55, wherein R⁵ is selected from: 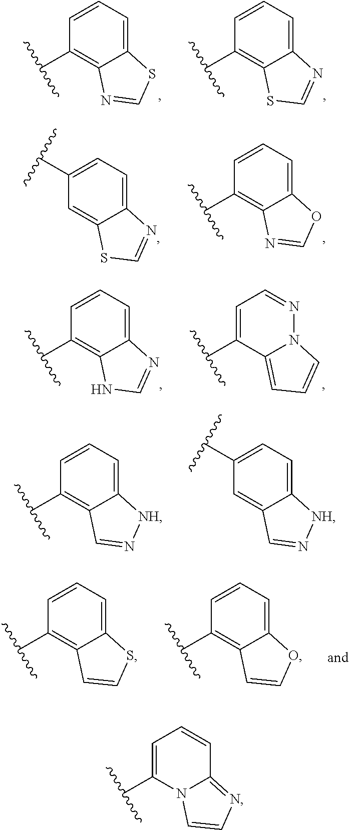
any of which is substituted with one or more R⁹.

58. The compound of any one of claims 1-55, wherein R⁵ is selected from:

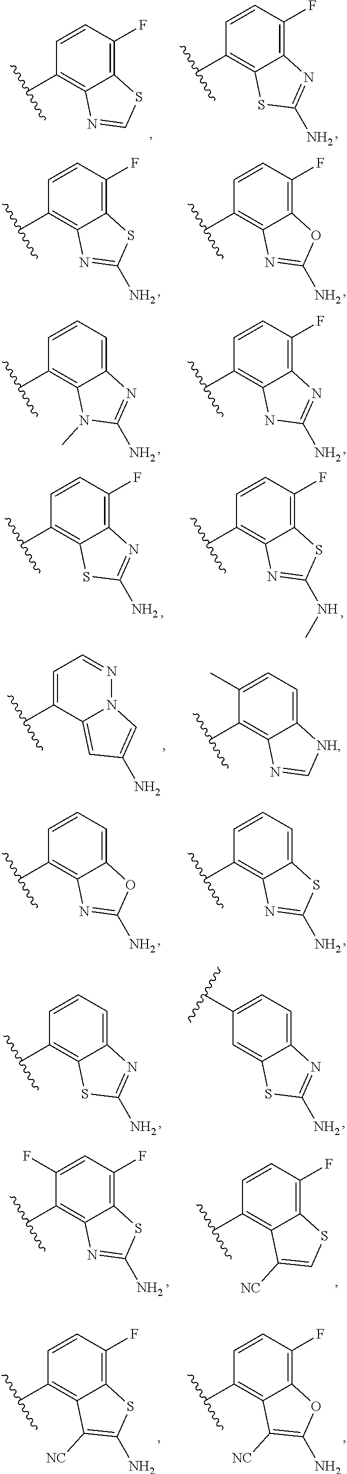

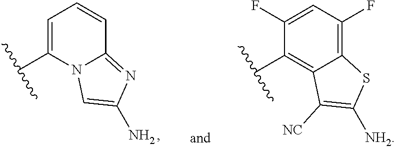 [SMILES: *c1cccc2nc(N)cn12 ; *c1c(F)cc(F)c2sc(N)c(C#N)c12]

59. The compound of claim 58, wherein R⁵ is selected from:

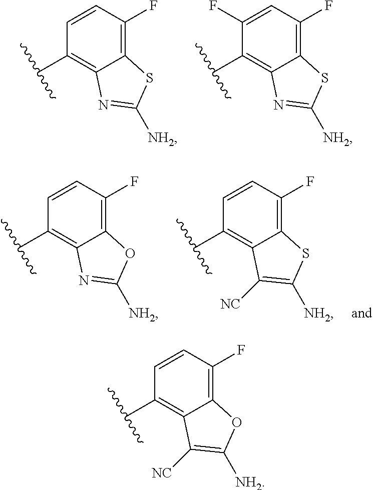

60. A compound according to Formula II: 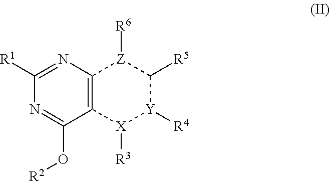
or a salt (e.g., pharmaceutically acceptable salt) thereof, wherein:
each dashed line represents a single or double bond;
X, Y, and Z are selected from N and C, wherein one and only one of X, Y, and Z is N;
R¹ is selected from —OR⁷, 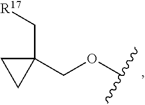
and a 4-6 membered heterocycle comprising a nitrogen atom, wherein the heterocycle is unsubstituted or substituted with one or more R¹⁵;
R² is a 4-6 membered heterocycle containing one or more nitrogen atoms, wherein the heterocycle is unsubstituted or is substituted with one or more R⁸;
when X is C, R³ is H, and when X is N, R³ is absent;
when Y is C, R⁴ is selected from H, halogen, —CN, —OR¹², and C₁₋₆alkyl, wherein any C₁₋₆alkyl is unsubstituted or substituted with one or more R¹¹, and when Y is N, R⁴ is absent;
R⁵ is a bicyclic heteroaryl substituted with one or more R⁹;
when Z is C, R⁶ is selected from halogen, —OR¹², —CN, and H, and when Z is N, R⁶ is absent;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S,
wherein the heterocycle is unsubstituted or substituted with one or more R¹⁸;
each R¹⁸ is independently selected from C₁₋₆alkyl and halogen; and
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

61. The compound of claim 60, wherein X is N, and Y and Z are C, and the compound is a compound according to Formula IIA: 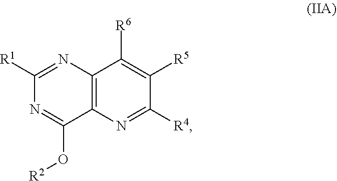
or a salt (e.g., pharmaceutically acceptable salt) thereof.

62. The compound of claim 61, wherein R⁴ is H.

63. The compound of claim 61, wherein R⁴ is selected from halogen, —CN, —OR¹², and C₁₋₆alkyl, wherein any C₁₋₆alkyl is unsubstituted or substituted with one or more R¹¹.

64. The compound of claim 63, wherein R⁴ is halogen (e.g., Cl or F).

65. The compound of claim 63, wherein R⁴ is C₁₋₆alkyl that is substituted with one or more halogens or —CN.

66. The compound of claim 65, wherein R⁴ is selected from —CF₂H, —CF₃, —CF₂CH₃, and —CH₂CN.

67. The compound of claim 66, wherein R⁴ is —CF₃.

68. The compound of any one of claims 61-67, wherein R⁶ is selected from halogen, —CN, and H.

69. The compound of claim 68, wherein R⁶ is halogen (e.g., Cl or F).

70. The compound of claim 68, wherein R⁶ is H.

71. The compound of claim 60, wherein Y is N, and X and Z are C, and the compound is a compound according to Formula IIB: 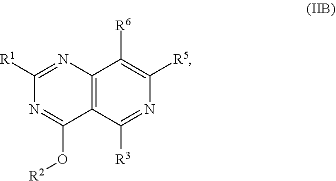
or a salt (e.g., pharmaceutically acceptable salt) thereof.

72. The compound of claim 71, wherein R⁶ is selected from halogen, —CN, and H.

73. The compound of claim 72, wherein R⁶ is a halogen (e.g., Cl or F).

74. The compound of claim 72, wherein R⁶ is H.

75. The compound of claim 60, wherein Z is N, and X and Y are C, and the compound is a compound according to Formula IIC: 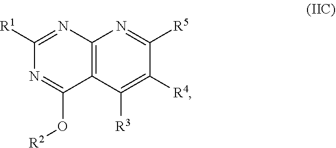
or a salt (e.g., pharmaceutically acceptable salt) thereof.

76. The compound of claim 75, wherein R⁴ is H.

77. The compound of claim 75, wherein R⁴ is selected from halogen, —CN, —OR¹², and C₁₋₆alkyl, wherein any C₁₋₆alkyl is unsubstituted or substituted with one or more R¹¹.

78. The compound of claim 77, wherein R⁴ is halogen (e.g., Cl or F).

79. The compound of claim 77, wherein R⁴ is C₁₋₆alkyl that is substituted with one or more halogens or —CN.

80. The compound of claim 79, wherein R⁴ is selected from —CF₂H, —CF₃, —CF₂CH₃, and —CH₂CN.

81. The compound of claim 80, wherein R⁴ is —CF₃.

82. The compound of any one of claims 60-81, wherein R² is a 4-6 membered heterocycle containing one nitrogen atom, wherein the heterocycle is unsubstituted or is substituted with one or more R⁸.

83. The compound of claim 82, wherein R² is a heterocycle selected from azetidine, pyrrolidine, and piperidine, wherein the heterocycle is unsubstituted or is substituted with one or more R⁸.

84. The compound of any one of claims 60-83, wherein R² is unsubstituted.

85. The compound of any one of claims 60-83, wherein R² is substituted with one or more R⁸.

86. The compound of claim 85, wherein the one or more R⁸ are each independently selected from C₁₋₆alkyl, wherein any C₁₋₆alkyl is unsubstituted.

87. The compound of any one of claims 60-86, wherein R¹ is —OR⁷.

88. The compound claim 87, wherein R¹ is: 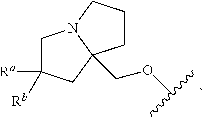
wherein R^{a} and R^{b} are each independently selected from halogen, C₁₋₆alkyl, —OR¹², and H, wherein any C₁₋₆alkyl is unsubstituted or is substituted with one or more R¹³.

89. The compound of claim 88, wherein R^{b} is H.

90. The compound of claim 88, wherein R1 is selected from:

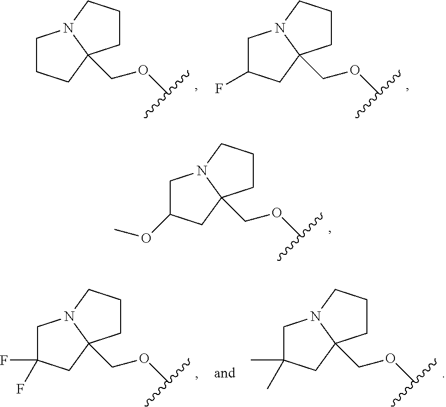 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

91. The compound of claim 87, wherein R¹ is selected from: 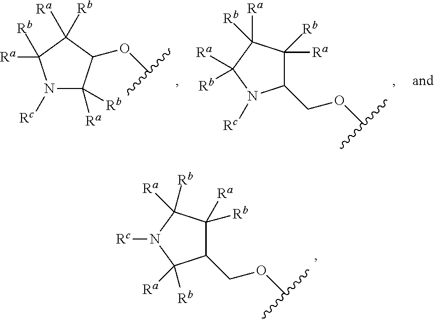
wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, wherein any C₁₋₆ alkyl or 3-6 membered carbocycle or heterocycle is unsubstituted or is substituted with one or more R¹³.

92. The compound of claim 91, wherein R¹ is selected from:

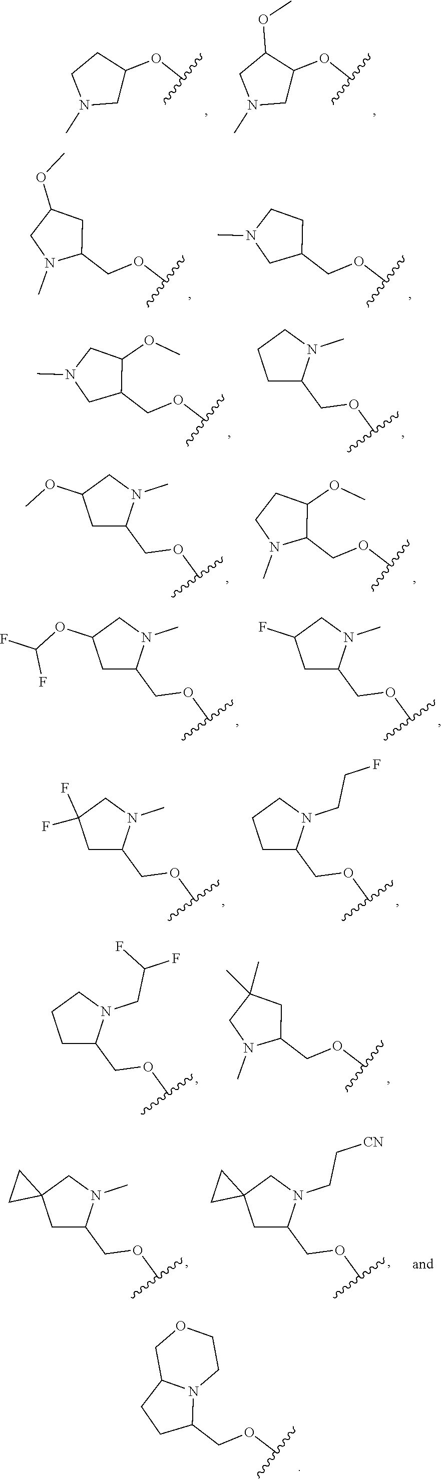

93. The compound of any one of claims 60-86, wherein R¹ is

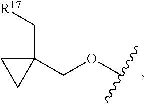

94. The compound of claim 93, wherein R¹ is selected from:

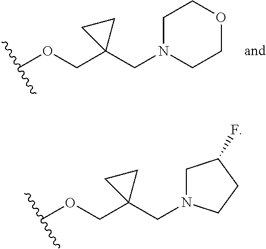 [SMILES: *OCC1(CN2CCOCC2)CC1 ; *OCC1(CN2CC[C@@H](F)C2)CC1]

95. The compound of any one of claims 60-86, wherein R¹ is a 4-6 membered heterocycle comprising a nitrogen atom, wherein the heterocycle is unsubstituted or substituted with one or more R¹⁵.

96. The compound of claim 95, wherein R¹ is selected from:

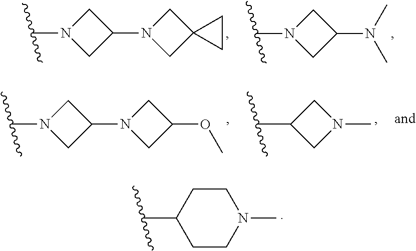

97. The compound of any one of claims 60-96, wherein R⁵ is 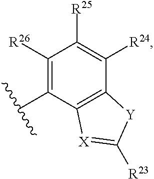
wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

98. The compound of any one of claims 60-97, wherein R⁵ is selected from: 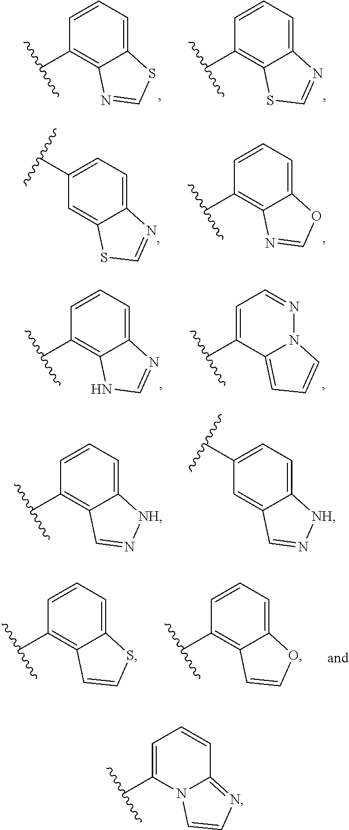
any of which is substituted with one or more R⁹.

99. The compound of any one of claims 60-98, wherein R⁵ is selected from:

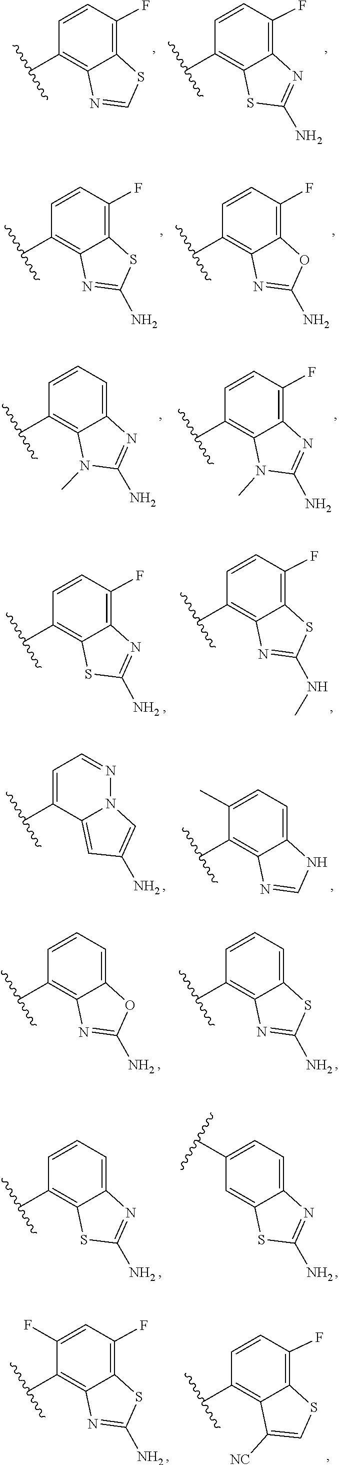

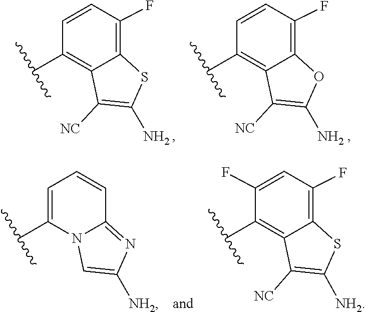

100. The compound of claim 99, wherein R⁵ is selected from:

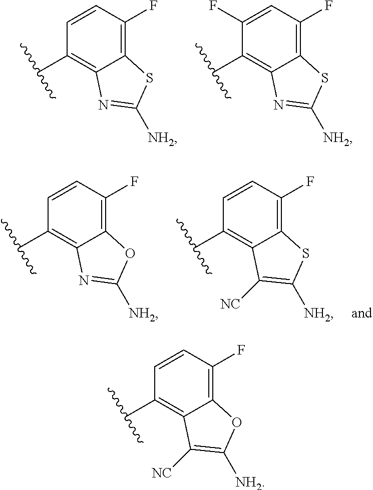

101. A compound shown in Table 2, or a salt (e.g., pharmaceutically acceptable salt) thereof.

102. A compound shown in Table 3, or a salt (e.g., pharmaceutically acceptable salt) thereof.

103. A pharmaceutical composition comprising a compound of any one of claims 1-102, or a salt (e.g., pharmaceutically acceptable salt), ester, tautomer, prodrug, zwitterionic form, or stereoisomer thereof, and a pharmaceutically acceptable excipient.

104. A compound of any one of claims 1-102, or a salt (e.g., pharmaceutically acceptable salt) thereof, for use as a medicament.

105. The compound of claim 104, wherein the medicament is useful in the prevention or treatment of a disease, disorder, or condition ameliorated by the inhibition of KRAS having a G12D mutation.

106. The compound of claim 104, wherein the medicament is useful in the prevention or treatment of a disease, disorder, or condition ameliorated by the inhibition of KRAS having a G12V mutation.

107. The compound of claim 104, wherein the medicament is useful in the prevention or treatment of a disease, disorder, or condition ameliorated by the inhibition of wild-type KRAS.

108. The compound of any one of claims 104-107, wherein the medicament is useful in the prevention or treatment of a cancer.

109. The compound of claim 108, wherein the cancer is selected from the group consisting of pancreatic cancer, colorectal cancer, and lung cancer.

110. A compound of any one of claims 1-102, or a salt (e.g., pharmaceutically acceptable salt) thereof, for use in the treatment of a disease, disorder, or condition.

111. The compound of claim 110, wherein the disease, disorder, or condition is a cancer.

112. The compound of claim 111, wherein the cancer is selected from the group consisting of pancreatic cancer, colorectal cancer, and lung cancer.

113. The compound of any one of claims 110-112, wherein the compound is used in the treatment of a disease, disorder, or condition in a subject in need thereof.

114. A compound of any one of claims 1-102, or a salt (e.g., pharmaceutically acceptable salt) thereof, for use in the manufacture of a medicament.

115. The compound of claim 114, wherein the medicament is useful in the prevention or treatment of a disease, disorder, or condition ameliorated by the inhibition of KRAS having a G12D mutation.

116. The compound of claim 114, wherein the medicament is useful in the prevention or treatment of a disease, disorder, or condition ameliorated by the inhibition of KRAS having a G12V mutation.

117. The compound of claim 114, wherein the medicament is useful in the prevention or treatment of a disease, disorder, or condition ameliorated by the inhibition of wild-type KRAS.

118. The compound of any one of claims 114-117, wherein the medicament is useful in the treatment of a cancer.

119. The compound of claim 118, wherein the cancer is selected from the group consisting of pancreatic cancer, colorectal cancer, and lung cancer.

120. A method, comprising administering a therapeutically effective amount of a compound of any one of claims 1-102, or a salt (e.g., pharmaceutically acceptable salt) thereof, to a subject in need thereof.

121. The method of claim 120, wherein the subject has a disease, disorder, or condition ameliorated by the inhibition of KRAS having a G12D mutation.

122. The method of claim 120, wherein the subject has a disease, disorder, or condition ameliorated by the inhibition of KRAS having a G12V mutation.

123. The method of claim 120, wherein the subject has a disease, disorder, or condition ameliorated by the inhibition of wild-type KRAS.

124. The method of any one of claims 120-123, wherein the subject has a cancer.

125. The method of claim 124, wherein the subject was previously diagnosed with the cancer.

126. The method of claim 124, wherein the subject has previously undergone a treatment regimen for the cancer.

127. The method of claim 124, wherein the subject has previously entered remission from the cancer.

128. The method of any one of claims 124-127, wherein the cancer is selected from the group consisting of pancreatic cancer, colorectal cancer, and lung cancer.

129. The method of any one of claims 120-128, wherein the compound, or the salt thereof, is administered in combination with an additional therapeutic agent.

130. The use of a compound of any one of claims 1-102, or a salt (e.g., pharmaceutically acceptable salt) thereof, for the manufacture of a medicament for the treatment of a cancer.

131. The use of claim 130, wherein the cancer is selected from the group consisting of pancreatic cancer, colorectal cancer, and lung cancer.

132. A method, comprising contacting a KRAS protein with a compound of any one of claims 1-102, or a salt (e.g., pharmaceutically acceptable salt) thereof.

133. The method of claim 132, wherein contacting the KRAS protein with the compound modulates KRAS.

134. The method of claim 132 or 133, wherein the KRAS protein has a G12D mutation.

135. The method of claim 132 or 133, wherein the KRAS protein has a G12V mutation.

136. The method of claim 132 or 133, wherein the KRAS protein is wild-type KRAS.

137. The method of any one of claims 132-136, wherein the KRAS protein is in an active (GTP-bound) state.

138. The method of any one of claims 132-136, wherein the KRAS protein is in an inactive (GDP-bound) state.

139. The method of any one of claims 132-138, wherein the KRAS protein is located within a cell.

140. The method of claim 139, wherein the cell is located within a subject.

141. The method of claim 140, wherein the subject is a human.

142. The method of claim 140 or 141, wherein the subject has a cancer.

143. The method of claim 142, wherein the cancer is selected from the group consisting of pancreatic cancer, colorectal cancer, and lung cancer.

144. A method of inhibiting the function of a KRAS protein having a G12D mutation, comprising contacting the KRAS protein with a compound of any one of claims 1-102, or a salt (e.g., pharmaceutically acceptable salt) thereof.

145. A method of inhibiting the function of a KRAS protein having a G12V mutation, comprising contacting the KRAS protein with a compound of any one of claims 1-102, or a salt (e.g., pharmaceutically acceptable salt) thereof.

146. A method of inhibiting the function of a wild-type KRAS protein, comprising contacting the KRAS protein with a compound of any one of claims 1-102, or a salt (e.g., pharmaceutically acceptable salt) thereof.

147. The method of any one of claims 144-146, wherein the KRAS protein is in an active (GTP-bound) state.

148. The method of any one of claims 144-146, wherein the KRAS protein is in an inactive (GDP-bound) state.

149. The method of any one of claims 144-148, wherein the KRAS protein is located within a cell.

150. The method of claim 149, wherein the cell is located within a subject.

151. The method of claim 150, wherein the subject is a human.

152. The method of claim 150 or 151, wherein the subject has a cancer.

153. The method of claim 152, wherein the cancer is selected from the group consisting of pancreatic cancer, colorectal cancer, and lung cancer.

154. A compound of any one of claims 1-102, or a salt (e.g., pharmaceutically acceptable salt) thereof, wherein the compound:
(i) has K_{D} of ≤0.1 μM, or 0.1 μM<K_{D}≤1 μM, or 1 μM<K_{D}≤10 μM in the assay of Biological Example 1 (e.g., a surface plasmon resonance binding assay);
(ii) has IC₅₀≤0.1 μM, 0.1 μM<IC₅₀≤1 μM, or 1 μM<IC₅₀≤120 μM in the assay of Biological Example 2 (e.g., a protein:protein interaction (PPI) Homogenous Time Resoled Fluorescence (HTRF) analysis of (i) 375 nM Avi-KRAS G12D/Q25A (1-169) GppNHp/3×FLAG-PI3KCA (157-299); (ii) 100 nM Avi-KRAS G12D (1-169) GppNHp/RAF1 RBD-3×FLAG (52-151); (iii) 50 nM Avi-KRAS G12V (1-169) GppNHp/RAF1 RBD-3×FLAG (52-151); (iv) 50 nM Avi-KRAS WT (1-169) GppNHp/RAF1 RBD-3×FLAG (52-151); and (v) 35 nM Avi-PI3K RBD CA-3×FLAG); and/or
(iii) has IC₅₀≤0.1 μM or 0.1 μM<IC₅₀≤1 μM in the assay of Biological Example 3 (e.g., cell-based pERK assay).
